SEQUENCE LISTING (662 sequences)

SEQ ID NO: 1 - DNA, 568 bp, Meloidogyne incognita
ctccgtcacgttattatgatccattcggacagcaagccctaccagtgcttcgactgtgattttatcgggatcaagagcaatgtggtcagtcatgcgagacagagcggacaccggaatgacgatgccctggacattaccactgatgacatgagggccgaatggaataccatgctccatgcttgtttccctgactatgttaaggcaaagagccgtggatggcagcccgaaaccgaggtcaaagacgagcctctggaatccaacaacgaacaggacactaccgtcaaaattgaagtctaatcatcttactcaaaagaacatatctcaagagttgctttcctttagtagattttctatataactgggtctctgtctctctcccgccttgccccaactgtgatccccctaccgtcttgccttttttcttccaacgagccccgggtgtatcttctcaaccagccacaaaagtatttccactttgatatcgcatattagtgctatacaccgagcatacaaacaacctatacataataaaagttattatttcaaaaaaaaaaaaaaaaaaaaaaaa

SEQ ID NO: 2 - protein, 98 aa, Meloidogyne incognita
LRHVIMIHSDSKPYQCFDCDFIGIKSNVVSHARQSGHRNDDALDITTDDMRAEWNTMLHACFPDYVKAKSRGWQPETEVKDEPLESNNEQDTTVKIEV

SEQ ID NO: 3 - DNA, 35 bp, Artificial Sequence
  primer
ccaacaacactgaattctacaggawsnmgntggaa

SEQ ID NO: 4 - DNA, 30 bp, Artificial Sequence
  primer
tcgcagattccattcagctckcatytcrtc

SEQ ID NO: 5 - DNA, 334 bp, Meloidogyne incognita
aggcaaagagccgtggatggcagcccgaaaccgaggtcaaagacgagcctctggaatccaacaacgaacaggacactaccgtcaaaattgaagtctaatcatcttactcaaaagaacatatctcaagagttgctttcctttagtagattttctatataactgggtctctgtctctctcccgccttgccccaactgtgatccccctaccgtcttgccttttttcttccaacgagccccgggtgtatcttctcaaccagccacaaaagtatttccactttgatatcgcatattagtgctatacaccgagcatacaaacaacctatacataataaaa

SEQ ID NO: 6 - DNA, 100 bp, Meloidogyne incognita
taccggctcaaggcaaagagccgtggatggcagcccgaaaccgaggtcaaagacgagcctctggaatccaacaacgaacaggacactaccgtcaaaatta

SEQ ID NO: 7 - DNA, 1894 bp, Meloidogyne incognita
aggcaaagagccgtggatggcagcccgaaaccgaggtcaaagacgagcctctggaatccaacaacgaacaggacactaccgtcaaaattgaagtctaatcatcttactcaaaagaacatatctcaagagttgctttcctttagtagattttctatataactgggtctctgtctctctcccgccttgccccaactgtgatccccctaccgtcttgccttttttcttccaacgagccccgggtgtatcttctcaaccagccacaaaagtatttccactttgatatcgcatattagtgctatacaccgagcatacaaacaacctatacataataaaaatcgtctctagacccagctttcttgtacaaagtggttgatcctgcagggtccgtcgcttctcttccatttcttctcattttcgattttgattcttatttctttccagtagctcctgctctgtgaatttctccgctcacgatagatctgcttatactccttacattcaaccttagatctggtctcgattctctgtttctctgtttttttcttttggtcgagaatctgatgtttgtttatgttctgtcaccattaataataatgaactctctcattcatacaatgattagtttctctcgtctacaaaacgatatgttgcattttcacttttcttctttttttctaagatgatttgctttgaccaatttgtttagatctttattctattttattttctggtgggttggtggaaattgaaaaaaaaaaaacagcataaattgttatttgttaatgtattcattttttggctatttgttctgggtaaaaatctgcttctactattgaatctttcctggattttttactcctattgggtttttatagtaaaaatacataataaaaggaaaacaaaagttttatagattctcttaaaccccttacgataaaagttggaatcaaaataattcaggatcagatgctctttgattgattcagatgcgattacagttgcatggcaaattttctagatccgtcgtcacattttattttctgtttaaatatctaaatctgatatatgatgtcgacaaattctggtggcttatacatcacttcaactgttttcttttggctttgtttgtcaacttggttttcaatacgatttgtgatttcgatcgctgaatttttaatacaagcaaactgatgttaaccacaagcaagagatgtgacctgccttattaacatcgtattacttactactagtcgtattctcaacgcaatcgtttttgtatttctcacattatgccgcttctctactctttattccttttggtccacgcattttctatttgtggcaatccctttcacaacctgatttcccactttggatcatttgtctgaagactctcttgaatcgttaccacttgtttcttgtgcatgctctgttttttagaattaatgataaaactattccatagtcttgagttttcagcttgttgattcttttgcttttggttttctgcaggtttaaacatcaaccactttgtacaagaaagctgggtctagagacgatttttattatgtataggttgtttgtatgctcggtgtatagcactaatatgcgatatcaaagtggaaatacttttgtggctggttgagaagatacacccggggctcgttggaagaaaaaaggcaagacggtagggggatcacagttggggcaaggcgggagagagacagagacccagttatatagaaaatctactaaaggaaagcaactcttgagatatgttcttttgagtaagatgattagacttcaattttgacggtagtgtcctgttcgttgttggattccagaggctcgtctttgacctcggtttcgggctgccatccacggctctttgcct

SEQ ID NO: 8 - DNA, 505 bp, Meloidogyne incognita
ccattgctcgtcgtaaaaaggaaatcaataaaatgaaggatcgtccaacttatgagcgttacatagaacaagttccaaaggatgacaggaagaaaggtgttcatcctaaaacacctaacaaatttctcaattggagtagacgatcatgggacaaacagatcaaattatggaaacttgcaatatatgaatgggcaggggaatcgccatcttcaagtgtttacggctcttattgttcatcggaatgtagtgacgaggatcgagcgcagtctttggaacggccgcattcagcggagccttcaacatcaaaaaagattaaaattgagagggacgttgaaccgaagaataggattgttaatattccaccatcagccgacactatggcttcattgttaggtcactttgatttggacacgcttcgtgaagcaccagcacctattatgaacacaactggagatgaatcaacattgaaagatattcctaaacctaatggccctgttactcgttc

SEQ ID NO: 9 - protein, 167 aa, Meloidogyne incognita
IARRKKEINKMKDRPTYERYIEQVPKDDRKKGVHPKTPNKFLNWSRRSWDKQIKLWKLAIYEWAGESPSSSVYGSYCSSECSDEDRAQSLERPHSAEPSTSKKIKIERDVEPKNRIVNIPPSADTMASLLGHFDLDTLREAPAPIMNTTGDESTLKDIPKPNGPVTR

SEQ ID NO: 10 - DNA, 21 bp, Artificial Sequence
  primer
ccattgctcgtcgtaaaaagg

SEQ ID NO: 11 - DNA, 23 bp, Artificial Sequence
  primer
gaacgagtaacagggccattagg

SEQ ID NO: 12 - DNA, 164 bp, Meloidogyne incognita
tttgatctgtttgtcccatgatcgtctactccaattgagaaatttgttaggtgttttaggatgaacacctttcttcctgtcatcctttggaacttgttctatgtaacgctcataagttggacgatccttcattttattgatttcctttttacgacgagcaatgg

SEQ ID NO: 13 - DNA, 1554 bp, Meloidogyne incognita
tttgatctgtttgtcccatgatcgtctactccaattgagaaatttgttaggtgttttaggatgaacacctttcttcctgtcatcctttggaacttgttctatgtaacgctcataagttggacgatccttcattttattgatttcctttttacgacgagcaatggatcgtctctagacccagctttcttgtacaaagtggttgatcctgcagggtccgtcgcttctcttccatttcttctcattttcgattttgattcttatttctttccagtagctcctgctctgtgaatttctccgctcacgatagatctgcttatactccttacattcaaccttagatctggtctcgattctctgtttctctgtttttttcttttggtcgagaatctgatgtttgtttatgttctgtcaccattaataataatgaactctctcattcatacaatgattagtttctctcgtctacaaaacgatatgttgcattttcacttttcttctttttttctaagatgatttgctttgaccaatttgtttagatctttattctattttattttctggtgggttggtggaaattgaaaaaaaaaaaacagcataaattgttatttgttaatgtattcattttttggctatttgttctgggtaaaaatctgcttctactattgaatctttcctggattttttactcctattgggtttttatagtaaaaatacataataaaaggaaaacaaaagttttatagattctcttaaaccccttacgataaaagttggaatcaaaataattcaggatcagatgctctttgattgattcagatgcgattacagttgcatggcaaattttctagatccgtcgtcacattttattttctgtttaaatatctaaatctgatatatgatgtcgacaaattctggtggcttatacatcacttcaactgttttcttttggctttgtttgtcaacttggttttcaatacgatttgtgatttcgatcgctgaatttttaatacaagcaaactgatgttaaccacaagcaagagatgtgacctgccttattaacatcgtattacttactactagtcgtattctcaacgcaatcgtttttgtatttctcacattatgccgcttctctactctttattccttttggtccacgcattttctatttgtggcaatccctttcacaacctgatttcccactttggatcatttgtctgaagactctcttgaatcgttaccacttgtttcttgtgcatgctctgttttttagaattaatgataaaactattccatagtcttgagttttcagcttgttgattcttttgcttttggttttctgcaggtttaaacatcaaccactttgtacaagaaagctgggtctagagacgatccattgctcgtcgtaaaaaggaaatcaataaaatgaaggatcgtccaacttatgagcgttacatagaacaagttccaaaggatgacaggaagaaaggtgttcatcctaaaacacctaacaaatttctcaattggagtagacgatcatgggacaaacagatcaaa

SEQ ID NO: 14 - DNA, 533 bp, Meloidogyne incognita
cgagatggtttcgtaaattattcagatggcaaaaacaaagaaggcttgtattattttctttgatgaagttgatgctattggcggagctcgttttgatgatggaatggggggagataatgaagttcaacggacaatgctcgaattgatcaatcagctggatggatttgactcgcgtggtgcaatcaaagttttaatggccacaaatcgtcctgatactttggatcctgcacttgtacggccaggacgtattgatcgtcgtatcgaattcgccgtgcctgatttggaagctcgtgcaaacattctaaagattcatacaaagcggatgagcgtcgatcggaatattcgttatgaattgatatcgcgtctttgccctaatacaacaggagctgatattcgctctgtctgtactgaagccggaatgtttgcattacgtgctcgtcgtaaagcaataaccgaaaaggattttattgacgctgttcagaaagtggtgaaaggatatgcaaagtttagttcaactccaagttatatgacac

SEQ ID NO: 15 - protein, 172 aa, Meloidogyne incognita
IIQMAKTKKACIIFFDEVDAIGGARFDDGMGGDNEVQRTMLELINQLDGFDSRGAIKVLMATNRPDTLDPALVRPGRIDRRIEFAVPDLEARANILKIHTKRMSVDRNIRYELISRLCPNTTGADIRSVCTEAGMFALRARRKAITEKDFIDAVQKVVKGYAKFSSTPSYMT

SEQ ID NO: 16 - DNA, 34 bp, Artificial Sequence
  primer
gtgtcatataacttggagttgaactaaactttgc

SEQ ID NO: 17 - DNA, 29 bp, Artificial Sequence
  primer
cgagatggtttcgtaaattattcagatgg

SEQ ID NO: 18 - DNA, 143 bp, Meloidogyne incognita
cgatcgacgctcatccgctttgtatgaatctttagaatgtttgcacgagcttccaaatcaggcacggcgaattcgatacgacgatcaatacgtcctggccgtacaagtgcaggatccaaagtatcaggacgatttgtggccat

SEQ ID NO: 19 - DNA, 1512 bp, Meloidogyne incognita
cgatcgacgctcatccgctttgtatgaatctttagaatgtttgcacgagcttccaaatcaggcacggcgaattcgatacgacgatcaatacgtcctggccgtacaagtgcaggatccaaagtatcaggacgatttgtggccatatcgtctctagacccagctttcttgtacaaagtggttgatcctgcagggtccgtcgcttctcttccatttcttctcattttcgattttgattcttatttctttccagtagctcctgctctgtgaatttctccgctcacgatagatctgcttatactccttacattcaaccttagatctggtctcgattctctgtttctctgtttttttcttttggtcgagaatctgatgtttgtttatgttctgtcaccattaataataatgaactctctcattcatacaatgattagtttctctcgtctacaaaacgatatgttgcattttcacttttcttctttttttctaagatgatttgctttgaccaatttgtttagatctttattctattttattttctggtgggttggtggaaattgaaaaaaaaaaaacagcataaattgttatttgttaatgtattcattttttggctatttgttctgggtaaaaatctgcttctactattgaatctttcctggattttttactcctattgggtttttatagtaaaaatacataataaaaggaaaacaaaagttttatagattctcttaaaccccttacgataaaagttggaatcaaaataattcaggatcagatgctctttgattgattcagatgcgattacagttgcatggcaaattttctagatccgtcgtcacattttattttctgtttaaatatctaaatctgatatatgatgtcgacaaattctggtggcttatacatcacttcaactgttttcttttggctttgtttgtcaacttggttttcaatacgatttgtgatttcgatcgctgaatttttaatacaagcaaactgatgttaaccacaagcaagagatgtgacctgccttattaacatcgtattacttactactagtcgtattctcaacgcaatcgtttttgtatttctcacattatgccgcttctctactctttattccttttggtccacgcattttctatttgtggcaatccctttcacaacctgatttcccactttggatcatttgtctgaagactctcttgaatcgttaccacttgtttcttgtgcatgctctgttttttagaattaatgataaaactattccatagtcttgagttttcagcttgttgattcttttgcttttggttttctgcaggtttaaacatcaaccactttgtacaagaaagctgggtctagagacgatatggccacaaatcgtcctgatactttggatcctgcacttgtacggccaggacgtattgatcgtcgtatcgaattcgccgtgcctgatttggaagctcgtgcaaacattctaaagattcatacaaagcggatgagcgtcgatcg

SEQ ID NO: 20 - DNA, 657 bp, Meloidogyne incognita
ctggaagaatacagattgcgaaattcttaaagctgctattatgaaatatggaaaaaatcaatggagccgaattgcctcattacttcatcgaaaatctgcgaaacaatgtaaagcccgttggtatgaatggcttgatcctcgaattaaaaagacggaatggtccagagaagaggacgaaaaacttctccatttggccaaaattatgcctactcaatggcgtacaattgctcctattgttggaagaactgctgctcagtgtttggaaagatatgaatatcttcttgacgaagctcaacgtcgtgctgaaggtattgaagaggatgattcattaaaagaggctaagaagttaaagccaggcgatgttgatccaacgccagaaactaaaccggctaggcctgatcctattgatatggatgaagatgaattggaaatgttatcagaagcccgagctcgtttggccaatacacaaggaaagaaagcaaaacgcaaagctcgtgaaaagcaattaggcgaggctcgaagacttgcttctctacaaaaacgaagagaacttcgggctgctggaataattattggatctggttgggcttataaaatgaagaagcatcgtctaatggattataataatggaataccttttgaaaagcaagttccagc

SEQ ID NO: 21 - protein, 218 aa, Meloidogyne incognita
WKNTDCEILKAAIMKYGKNQWSRIASLLHRKSAKQCKARWYEWLDPRIKKTEWSREEDEKLLHLAKIMPTQWRTIAPIVGRTAAQCLERYEYLLDEAQRRAEGIEEDDSLKEAKKLKPGDVDPTPETKPARPDPIDMDEDELEMLSEARARLANTQGKKAKRKAREKQLGEARRLASLQKRRELRAAGIIIGSGWAYKMKKHRLMDYNNGIPFEKQVP

SEQ ID NO: 22 - DNA, 32 bp, Artificial Sequence
  primer
ctggaagaatacagattgcgaaattcttaaag

SEQ ID NO: 23 - DNA, 28 bp, Artificial Sequence
  primer
gctggaacttgcttttcaaaaggtattc

SEQ ID NO: 24 - DNA, 177 bp, Meloidogyne incognita
aagaagatattcatatctttccaaacactgagcagcagttcttccaacaataggagcaattgtacgccattgagtaggcataattttggccaaatggagaagtttttcgtcctcttctctggaccattccgtctttttaattcgaggatcaagccattcataccaacgggctttaca

SEQ ID NO: 25 - DNA, 1580 bp, Meloidogyne incognita
aagaagatattcatatctttccaaacactgagcagcagttcttccaacaataggagcaattgtacgccattgagtaggcataattttggccaaatggagaagtttttcgtcctcttctctggaccattccgtctttttaattcgaggatcaagccattcataccaacgggctttacaatcgtctctagacccagctttcttgtacaaagtggttgatcctgcagggtccgtcgcttctcttccatttcttctcattttcgattttgattcttatttctttccagtagctcctgctctgtgaatttctccgctcacgatagatctgcttatactccttacattcaaccttagatctggtctcgattctctgtttctctgtttttttcttttggtcgagaatctgatgtttgtttatgttctgtcaccattaataataatgaactctctcattcatacaatgattagtttctctcgtctacaaaacgatatgttgcattttcacttttcttctttttttctaagatgatttgctttgaccaatttgtttagatctttattctattttattttctggtgggttggtggaaattgaaaaaaaaaaaacagcataaattgttatttgttaatgtattcattttttggctatttgttctgggtaaaaatctgcttctactattgaatctttcctggattttttactcctattgggtttttatagtaaaaatacataataaaaggaaaacaaaagttttatagattctcttaaaccccttacgataaaagttggaatcaaaataattcaggatcagatgctctttgattgattcagatgcgattacagttgcatggcaaattttctagatccgtcgtcacattttattttctgtttaaatatctaaatctgatatatgatgtcgacaaattctggtggcttatacatcacttcaactgttttcttttggctttgtttgtcaacttggttttcaatacgatttgtgatttcgatcgctgaatttttaatacaagcaaactgatgttaaccacaagcaagagatgtgacctgccttattaacatcgtattacttactactagtcgtattctcaacgcaatcgtttttgtatttctcacattatgccgcttctctactctttattccttttggtccacgcattttctatttgtggcaatccctttcacaacctgatttcccactttggatcatttgtctgaagactctcttgaatcgttaccacttgtttcttgtgcatgctctgttttttagaattaatgataaaactattccatagtcttgagttttcagcttgttgattcttttgcttttggttttctgcaggtttaaacatcaaccactttgtacaagaaagctgggtctagagacgattgtaaagcccgttggtatgaatggcttgatcctcgaattaaaaagacggaatggtccagagaagaggacgaaaaacttctccatttggccaaaattatgcctactcaatggcgtacaattgctcctattgttggaagaactgctgctcagtgtttggaaagatatgaatatcttctt

SEQ ID NO: 26 - DNA, 638 bp, Meloidogyne incognita
ggttattacccatgcttgagacaaaagcacaatattctgagaaattttaaacaacaacaatgcatttgtctttagctaaagttgttgctattaaagagccgcctctatatgatcgtcgacaaggatttgtgccccgaacgcaagatgattttggtgatggtggggcttttccagaaatacatattgctcagtttccaatcggtatgggcgccgataagcccggcactggggcaaagaacacagttgcgcttcagtttgactctgagggcaaacttcgatttgatgagcttacacgaattgggcatggaaaagacaagattgttcattcccgcctttctgatatgaaatccaaccatattgatgatgaagacgaaagtttcaaaaaaccaacagatgaagaaatacatgaaacagccgaagcaactcgtgtttccttagagaaaattacagctgttaagatcgccgcatcattaccagtccagcatgcaaagaaaacagcgcctgcacaatatattcgttacactcctagccagcaggctggtggttttcatacaagtggagcacaacaaagaattattcgtttggttgaagagcagaaggatccaatggagccgccacgttttcaaataaaccagaaa

SEQ ID NO: 27 - protein, 196 aa, Meloidogyne incognita
TTTMHLSLAKVVAIKEPPLYDRRQGFVPRTQDDFGDGGAFPEIHIAQFPIGMGADKPGTGAKNTVALQFDSEGKLRFDELTRIGHGKDKIVHSRLSDMKSNHIDDEDESFKKPTDEEIHETAEATRVSLEKITAVKIAASLPVQHAKKTAPAQYIRYTPSQQAGGFHTSGAQQRIIRLVEEQKDPMEPPRFQINQK

SEQ ID NO: 28 - DNA, 422 bp, Meloidogyne incognita
gagccgcctctatatgatcgtcgacaaggatttgtgccccgaacgcaagatgattttggtgatggtggggcttttccagaaatacatattgctcagtttccaatcggtatgggcgccgataagcccggcactggggcaaagaacacagttgcgcttcagtttgactctgagggcaaacttcgatttgatgagcttacacgaattgggcatggaaaagacaagattgttcattcccgcctttctgatatgaaatccaaccatattgatgatgaagacgaaagtttcaaaaaaccaacagatgaagaaatacatgaaacagccgaagcaactcgtgtttccttagagaaaattacagctgttaagatcgccgcatcattaccagtccagcatgcaaagaaaacagcgcctgcacaatatattcg

SEQ ID NO: 29 - DNA, 150 bp, Meloidogyne incognita
actcgtgtttccttagagaaaattacagctgttaagatcgccgcatcattaccagtccagcatgcaaagaaaacagcgcctgcacaatatattcgttacactcctagccagcaggctggtggttttcatacaagtggagcacaacaaaga

SEQ ID NO: 30 - DNA, 2070 bp, Meloidogyne incognita
gagccgcctctatatgatcgtcgacaaggatttgtgccccgaacgcaagatgattttggtgatggtggggcttttccagaaatacatattgctcagtttccaatcggtatgggcgccgataagcccggcactggggcaaagaacacagttgcgcttcagtttgactctgagggcaaacttcgatttgatgagcttacacgaattgggcatggaaaagacaagattgttcattcccgcctttctgatatgaaatccaaccatattgatgatgaagacgaaagtttcaaaaaaccaacagatgaagaaatacatgaaacagccgaagcaactcgtgtttccttagagaaaattacagctgttaagatcgccgcatcattaccagtccagcatgcaaagaaaacagcgcctgcacaatatattcgatcgtctctagacccagctttcttgtacaaagtggttgatcctgcagggtccgtcgcttctcttccatttcttctcattttcgattttgattcttatttctttccagtagctcctgctctgtgaatttctccgctcacgatagatctgcttatactccttacattcaaccttagatctggtctcgattctctgtttctctgtttttttcttttggtcgagaatctgatgtttgtttatgttctgtcaccattaataataatgaactctctcattcatacaatgattagtttctctcgtctacaaaacgatatgttgcattttcacttttcttctttttttctaagatgatttgctttgaccaatttgtttagatctttattctattttattttctggtgggttggtggaaattgaaaaaaaaaaaacagcataaattgttatttgttaatgtattcattttttggctatttgttctgggtaaaaatctgcttctactattgaatctttcctggattttttactcctattgggtttttatagtaaaaatacataataaaaggaaaacaaaagttttatagattctcttaaaccccttacgataaaagttggaatcaaaataattcaggatcagatgctctttgattgattcagatgcgattacagttgcatggcaaattttctagatccgtcgtcacattttattttctgtttaaatatctaaatctgatatatgatgtcgacaaattctggtggcttatacatcacttcaactgttttcttttggctttgtttgtcaacttggttttcaatacgatttgtgatttcgatcgctgaatttttaatacaagcaaactgatgttaaccacaagcaagagatgtgacctgccttattaacatcgtattacttactactagtcgtattctcaacgcaatcgtttttgtatttctcacattatgccgcttctctactctttattccttttggtccacgcattttctatttgtggcaatccctttcacaacctgatttcccactttggatcatttgtctgaagactctcttgaatcgttaccacttgtttcttgtgcatgctctgttttttagaattaatgataaaactattccatagtcttgagttttcagcttgttgattcttttgcttttggttttctgcaggtttaaacatcaaccactttgtacaagaaagctgggtctagagacgatcgaatatattgtgcaggcgctgttttctttgcatgctggactggtaatgatgcggcgatcttaacagctgtaattttctctaaggaaacacgagttgcttcggctgtttcatgtatttcttcatctgttggttttttgaaactttcgtcttcatcatcaatatggttggatttcatatcagaaaggcgggaatgaacaatcttgtcttttccatgcccaattcgtgtaagctcatcaaatcgaagtttgccctcagagtcaaactgaagcgcaactgtgttctttgccccagtgccgggcttatcggcgcccataccgattggaaactgagcaatatgtatttctggaaaagccccaccatcaccaaaatcatcttgcgttcggggcacaaatccttgtcgacgatcatatagaggcggctc

SEQ ID NO: 31 - DNA, 615 bp, Meloidogyne incognita
gcgtttggcggcctcgcttcaaaagtgcggcaaaggcaaaatttggttggatcctaatgaggtcaacgtgatttccaacgcaaattctcgtcagaacgttcgtcgtttggtcaaagatggtcttataattcgtaagcctggcaaagttcattcgcgtttccgagctcgtgaacgtttggaagcacgcagaaagggtcgtcatatgggttttggaaaacgacgcggaactcgtaacgctcgaatgccggagaaggtcttatggatgcgtttttttgttgtactccgtcatcttttaaagcgctatcgagctgcgaagaagattgacaagcatctttaccacgagctttacttgaaggctaaaggaaacgcatttaagaataagagaaatttgatggaatatatcttcaaaaagaagactgagaaccagcgttctaaacaacttgctgaacaggctgaggctcgccgaaacaagaacaaggaaacgagaaagaggcgtgaggagcgtttgattttgaaacgtaaggaattgctccacaaactttctgagagcgaaaagcagaagactccacagcttcaagaagaggaagcgaaggaagcttcgccttctccaccacc

SEQ ID NO: 32 - protein, 204 aa, Meloidogyne incognita
RLAASLQKCGKGKIWLDPNEVNVISNANSRQNVRRLVKDGLIIRKPGKVHSRFRARERLEARRKGRHMGFGKRRGTRNARMPEKVLWMRFFVVLRHLLKRYRAAKKIDKHLYHELYLKAKGNAFKNKRNLMEYIFKKKTENQRSKQLAEQAEARRNKNKETRKRREERLILKRKELLHKLSESEKQKTPQLQEEEAKEASPSPP

SEQ ID NO: 33 - DNA, 417 bp, Meloidogyne incognita
gttggatcctaatgaggtcaacgtgatttccaacgcaaattctcgtcagaacgttcgtcgtttggtcaaagatggtcttataattcgtaagcctggcaaagttcattcgcgtttccgagctcgtgaacgtttggaagcacgcagaaagggtcgtcatatgggttttggaaaacgacgcggaactcgtaacgctcgaatgccggagaaggtcttatggatgcgtttttttgttgtactccgtcatcttttaaagcgctatcgagctgcgaagaagattgacaagcatctttaccacgagctttacttgaaggctaaaggaaacgcatttaagaataagagaaatttgatggaatatatcttcaaaaagaagactgagaaccagcgttctaaacaacttgctgaacaggctgaggctcg

SEQ ID NO: 34 - DNA, 2060 bp, Meloidogyne incognita
gttggatcctaatgaggtcaacgtgatttccaacgcaaattctcgtcagaacgttcgtcgtttggtcaaagatggtcttataattcgtaagcctggcaaagttcattcgcgtttccgagctcgtgaacgtttggaagcacgcagaaagggtcgtcatatgggttttggaaaacgacgcggaactcgtaacgctcgaatgccggagaaggtcttatggatgcgtttttttgttgtactccgtcatcttttaaagcgctatcgagctgcgaagaagattgacaagcatctttaccacgagctttacttgaaggctaaaggaaacgcatttaagaataagagaaatttgatggaatatatcttcaaaaagaagactgagaaccagcgttctaaacaacttgctgaacaggctgaggctcgatcgtctctagacccagctttcttgtacaaagtggttgatcctgcagggtccgtcgcttctcttccatttcttctcattttcgattttgattcttatttctttccagtagctcctgctctgtgaatttctccgctcacgatagatctgcttatactccttacattcaaccttagatctggtctcgattctctgtttctctgtttttttcttttggtcgagaatctgatgtttgtttatgttctgtcaccattaataataatgaactctctcattcatacaatgattagtttctctcgtctacaaaacgatatgttgcattttcacttttcttctttttttctaagatgatttgctttgaccaatttgtttagatctttattctattttattttctggtgggttggtggaaattgaaaaaaaaaaaacagcataaattgttatttgttaatgtattcattttttggctatttgttctgggtaaaaatctgcttctactattgaatctttcctggattttttactcctattgggtttttatagtaaaaatacataataaaaggaaaacaaaagttttatagattctcttaaaccccttacgataaaagttggaatcaaaataattcaggatcagatgctctttgattgattcagatgcgattacagttgcatggcaaattttctagatccgtcgtcacattttattttctgtttaaatatctaaatctgatatatgatgtcgacaaattctggtggcttatacatcacttcaactgttttcttttggctttgtttgtcaacttggttttcaatacgatttgtgatttcgatcgctgaatttttaatacaagcaaactgatgttaaccacaagcaagagatgtgacctgccttattaacatcgtattacttactactagtcgtattctcaacgcaatcgtttttgtatttctcacattatgccgcttctctactctttattccttttggtccacgcattttctatttgtggcaatccctttcacaacctgatttcccactttggatcatttgtctgaagactctcttgaatcgttaccacttgtttcttgtgcatgctctgttttttagaattaatgataaaactattccatagtcttgagttttcagcttgttgattcttttgcttttggttttctgcaggtttaaacatcaaccactttgtacaagaaagctgggtctagagacgatcgagcctcagcctgttcagcaagttgtttagaacgctggttctcagtcttctttttgaagatatattccatcaaatttctcttattcttaaatgcgtttcctttagccttcaagtaaagctcgtggtaaagatgcttgtcaatcttcttcgcagctcgatagcgctttaaaagatgacggagtacaacaaaaaaacgcatccataagaccttctccggcattcgagcgttacgagttccgcgtcgttttccaaaacccatatgacgaccctttctgcgtgcttccaaacgttcacgagctcggaaacgcgaatgaactttgccaggcttacgaattataagaccatctttgaccaaacgacgaacgttctgacgagaatttgcgttggaaatcacgttgacctcattaggatccaac

SEQ ID NO: 35 - DNA, 656 bp, Meloidogyne incognita
gggaaatcagcaattccaacaatttaccccaggttcaggagataagcaaggagaaggagacaagaagcgtcgttttgggcctccaatccctacacgctttgggaagcgaaagaagggaagtaaaggacctgaagctagcaataaaatgcctaatgtgacaccagtaactcgttgcaaattgcgtcttcttaaatatgaacggattaaagattatttgttaatggaagaagaatttattcgaaatcaggaaagacataaacctcaagaggaaaggcaggaagaagaacggacaaaagtagatgaaatgagagggtcaccaatggctgtgggtacattggaggagattattgacgatcaacacgcagtcgtttctacgaatgttggcagcgaacattatgttaacattctttcatttgtggacaaggaacaattggagccaggctgtgcagttcttctaaatcacaaaacacacgcagttgttggtgttttggctgatgacacggatccaatggtttctgtaatgaaattggaaaaggctccaactgaaacgtacgctgatgttggcggtttggaacaacaaattcaagaaattaaggaggcagtggaattgccacttacacatccagagtactacgaagaaattggaatcaagcctc

SEQ ID NO: 36 - protein, 218 aa, Meloidogyne incognita
GNQQFQQFTPGSGDKQGEGDKKRRFGPPIPTRFGKRKKGSKGPEASNKMPNVTPVTRCKLRLLKYERIKDYLLMEEEFIRNQERHKPQEERQEEERTKVDEMRGSPMAVGTLEEIIDDQHAVVSTNVGSEHYVNILSFVDKEQLEPGCAVLLNHKTHAVVGVLADDTDPMVSVMKLEKAPTETYADVGGLEQQIQEIKEAVELPLTHPEYYEEIGIKP

SEQ ID NO: 37 - DNA, 406 bp, Meloidogyne incognita
ggcctccaatccctacacgctttgggaagcgaaagaagggaagtaaaggacctgaagctagcaataaaatgcctaatgtgacaccagtaactcgttgcaaattgcgtcttcttaaatatgaacggattaaagattatttgttaatggaagaagaatttattcgaaatcaggaaagacataaacctcaagaggaaaggcaggaagaagaacggacaaaagtagatgaaatgagagggtcaccaatggctgtgggtacattggaggagattattgacgatcaacacgcagtcgtttctacgaatgttggcagcgaacattatgttaacattctttcatttgtggacaaggaacaattggagccaggctgtgcagttcttctaaatcacaaaacacacgcagttgttgg

SEQ ID NO: 38 - DNA, 2038 bp, Meloidogyne incognita
ggcctccaatccctacacgctttgggaagcgaaagaagggaagtaaaggacctgaagctagcaataaaatgcctaatgtgacaccagtaactcgttgcaaattgcgtcttcttaaatatgaacggattaaagattatttgttaatggaagaagaatttattcgaaatcaggaaagacataaacctcaagaggaaaggcaggaagaagaacggacaaaagtagatgaaatgagagggtcaccaatggctgtgggtacattggaggagattattgacgatcaacacgcagtcgtttctacgaatgttggcagcgaacattatgttaacattctttcatttgtggacaaggaacaattggagccaggctgtgcagttcttctaaatcacaaaacacacgcagttgttggatcgtctctagacccagctttcttgtacaaagtggttgatcctgcagggtccgtcgcttctcttccatttcttctcattttcgattttgattcttatttctttccagtagctcctgctctgtgaatttctccgctcacgatagatctgcttatactccttacattcaaccttagatctggtctcgattctctgtttctctgtttttttcttttggtcgagaatctgatgtttgtttatgttctgtcaccattaataataatgaactctctcattcatacaatgattagtttctctcgtctacaaaacgatatgttgcattttcacttttcttctttttttctaagatgatttgctttgaccaatttgtttagatctttattctattttattttctggtgggttggtggaaattgaaaaaaaaaaaacagcataaattgttatttgttaatgtattcattttttggctatttgttctgggtaaaaatctgcttctactattgaatctttcctggattttttactcctattgggtttttatagtaaaaatacataataaaaggaaaacaaaagttttatagattctcttaaaccccttacgataaaagttggaatcaaaataattcaggatcagatgctctttgattgattcagatgcgattacagttgcatggcaaattttctagatccgtcgtcacattttattttctgtttaaatatctaaatctgatatatgatgtcgacaaattctggtggcttatacatcacttcaactgttttcttttggctttgtttgtcaacttggttttcaatacgatttgtgatttcgatcgctgaatttttaatacaagcaaactgatgttaaccacaagcaagagatgtgacctgccttattaacatcgtattacttactactagtcgtattctcaacgcaatcgtttttgtatttctcacattatgccgcttctctactctttattccttttggtccacgcattttctatttgtggcaatccctttcacaacctgatttcccactttggatcatttgtctgaagactctcttgaatcgttaccacttgtttcttgtgcatgctctgttttttagaattaatgataaaactattccatagtcttgagttttcagcttgttgattcttttgcttttggttttctgcaggtttaaacatcaaccactttgtacaagaaagctgggtctagagacgatccaacaactgcgtgtgttttgtgatttagaagaactgcacagcctggctccaattgttccttgtccacaaatgaaagaatgttaacataatgttcgctgccaacattcgtagaaacgactgcgtgttgatcgtcaataatctcctccaatgtacccacagccattggtgaccctctcatttcatctacttttgtccgttcttcttcctgcctttcctcttgaggtttatgtctttcctgatttcgaataaattcttcttccattaacaaataatctttaatccgttcatatttaagaagacgcaatttgcaacgagttactggtgtcacattaggcattttattgctagcttcaggtcctttacttcccttctttcgcttcccaaagcgtgtagggattggaggcc

SEQ ID NO: 39 - DNA, 916 bp, Meloidogyne incognita
cagccagtggtctttccccttcaatcacaccaaattccttgatattttcttttaacatttttttcaaactgtgggtctccaagcaataagttttcagggtataggtcaggtggtgactcatttttcaatatttgatagtggtttgtatttttgactttacttaacttgtttactgaaatctgttcctgttcttcaagatgaccgcacaattcgaaagatacacaaattggatgatcacgtaatggctgcttttgctggtttgagtgcagatgctcgtgttttgattgaccgtgccagagttgcttgtgaaaattacaaattgactttggaggatcccgtcacacttgagcacatctcccgcactattgcggacttgaaacatgaatttacgcaaactacaggtcgtcgtccttttggtgtctctttacttgttggcggttttgatcccgatggaactcctcatctttttattactgaaccatctggtgtgtattacgaattacttgctggctcaatcggccggaatgagaaggttgtcaaagaatatttggaggagaattataatgatggcaatgtttcaaacgaaaacttggtcttgaagttggtggtcaaagctcttgtcccagttgtgcaaactggagcgcgtaatatggagatatcggtgttgaagcgtgataatgaggggaatattgttcaacgtgttctactcactgaggagttggaggaaattttacgtcaaattgaagctgaaactatggagactgtttagactttctttgatttttttaaaaattcagtcgaacctcatttttagatactcttggggctgaaaaatcgtatcttatttgaggtcttataatgaggtccaactatacatacatttgctatttttgttaataaatgtttcgctattctg

SEQ ID NO: 40 - protein, 192 aa, Meloidogyne incognita
KSVPVLQDDRTIRKIHKLDDHVMAAFAGLSADARVLIDRARVACENYKLTLEDPVTLEHISRTIADLKHEFTQTTGRRPFGVSLLVGGFDPDGTPHLFITEPSGVYYELLAGSIGRNEKVVKEYLEENYNDGNVSNENLVLKLVVKALVPVVQTGARNMEISVLKRDNEGNIVQRVLLTEELEEILRQIEAE

SEQ ID NO: 41 - DNA, 450 bp, Meloidogyne incognita
tcacaccaaattccttgatattttcttttaacatttttttcaaactgtgggtctccaagcaataagttttcagggtataggtcaggtggtgactcatttttcaatatttgatagtggtttgtatttttgactttacttaacttgtttactgaaatctgttcctgttcttcaagatgaccgcacaattcgaaagatacacaaattggatgatcacgtaatggctgcttttgctggtttgagtgcagatgctcgtgttttgattgaccgtgccagagttgcttgtgaaaattacaaattgactttggaggatcccgtcacacttgagcacatctcccgcactattgcggacttgaaacatgaatttacgcaaactacaggtcgtcgtccctttggtgtctctttacttgttggcggttttgatcccgatggaactcctcatctttttattac

SEQ ID NO: 42 - DNA, 2386 bp, Meloidogyne incognita
tcacaccaaattccttgatattttcttttaacatttttttcaaactgtgggtctccaagcaataagttttcagggtataggtcaggtggtgactcatttttcaatatttgatagtggtttgtatttttgactttacttaacttgtttactgaaatctgttcctgttcttcaagatgaccgcacaattcgaaagatacacaaattggatgatcacgtaatggctgcttttgctggtttgagtgcagatgctcgtgttttgattgaccgtgccagagttgcttgtgaaaattacaaattgactttggaggatcccgtcacacttgagcacatctcccgcactattgcggacttgaaacatgaatttacgcaaactacaggtcgtcgtccctttggtgtctctttacttgttggcggttttgatcccgatggaactcctcatctttttattacacccagctttcttgtacaaagtggtgatatcccgcgggatcagaagcaacctcatggaaatgatgaggtaaggtttcatactcttgcctcttcttacggctttctgtgtcttcactgtaagtttctatgatttgagccaccaatatatatgctctggtgtgctgagttatgtttatctggtcacgcttagtgggtaaaattatgcttattttagcataaactttaatgagattaggttttgtatcacaccgatctttagttgtttagtaagatgacagaaattcttggtaaaacactctaaatcgtcttctttagtgaagttttccttagagtagcataaattttggcttttttcttgatggttgaataaggtggcacttgttggtatgagactttattgagagtcatattaagctgatccacgcgtttacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgccggatcagcttagcgttcattgaatttgatggccataggggtttagatgcaactgtttctttgaacattgtagaaatatataaagattttacattagcctactcttgaaagtcaaattgtcgaatttgattatattatactctagaggtgatattagttaatgagtttatactcggttatttacagcttattcatataccagttaacgtgtctcatatattctaacttcttagcatttaacgtgtttgcaggtcagcttgacactgaacataacagcatcactagtgcggccgcctgcaggtcgaccatatggtcgacctgcaggcggccgcactagtgatatcaccactttgtacaagaaagctgggtgtaataaaaagatgaggagttccatcgggatcaaaaccgccaacaagtaaagagacaccaaagggacgacgacctgtagtttgcgtaaattcatgtttcaagtccgcaatagtgcgggagatgtgctcaagtgtgacgggatcctccaaagtcaatttgtaattttcacaagcaactctggcacggtcaatcaaaacacgagcatctgcactcaaaccagcaaaagcagccattacgtgatcatccaatttgtgtatctttcgaattgtgcggtcatcttgaagaacaggaacagatttcagtaaacaagttaagtaaagtcaaaaatacaaaccactatcaaatattgaaaaatgagtcaccacctgacctataccctgaaaacttattgcttggagacccacagtttgaaaaaaatgttaaaagaaaatatcaaggaatttggtgtga

SEQ ID NO: 43 - DNA, 711 bp, Meloidogyne incognita
ggtttaattactccagtttgagcttaaaaagtaaaacaagatgcctcgcataaagcatgtattttctaaagtttatgacgtgcctcgtcgtccattcgagaaggagcgtttagatcaggaattaaaattaattggcgagtttggtttacgaaataaacgtgaagtttggcgtgtcaaattaactttggcaaagattcgtaaagctgctcgtgaacttttaacattggaagacaaggatcctcgacgcttatttgaaggaaatgctcttcttcgtcgtttggttcgtattggagttcttttggaggataagatgaaattggactatgttcttggtttgcgtgttgaagactttttggagcgtcgtttgcagacgcaagttttcaaattgggattggctaaatcaattcaccatgctcgtgttcttattcgtcaaaagcatattcgaattcgtcgacaagtagtcaatattccatcattcattgttcgtctcgactctcaaaagcacattgacttctccacaaaatcttatcttgctggtggacctcctggacgtgtcaagagaaagaatttgaagaaggctgcttcgggaggtggaggaggaggcggtgacgatgaagaggattgaataatatctaaaggtttataaaacgttgaaagttaattctgttaagttgttaaaagattttggatttgaaaaaatgtttttgttga

SEQ ID NO: 44 - protein, 194 aa, Meloidogyne incognita
MPRIKHVFSKVYDVPRRPFEKERLDQELKLIGEFGLRNKREVWRVKLTLAKIRKAARELLTLEDKDPRRLFEGNALLRRLVRIGVLLEDKMKLDYVLGLRVEDFLERRLQTQVFKLGLAKSIHHARVLIRQKHIRIRRQVVNIPSFIVRLDSQKHIDFSTKSYLAGGPPGRVKRKNLKKAASGGGGGGGDDEED

SEQ ID NO: 45 - DNA, 543 bp, Meloidogyne incognita
atcctcttcatcgtcaccgcctcctcctccacctcccgaagcagccttcttcaaattctttctcttgacacgtccaggaggtccaccagcaagataagattttgtggagaagtcaatgtgcttttgagagtcgagacgaacaatgaatgatggaatattgactacttgtcgacgaattcgaatatgcttttgacgaataagaacacgagcatggtgaattgatttagccaatcccaatttgaaaacttgcgtctgcaaacgacgctccaaaaagtcttcaacacgcaaaccaagaacatagtccaatttcatcttatcctccaaaagaactccaatacgaaccaaacgacgaagaagagcatttccttcaaataagcgtcgaggatccttgtcttccaatgttaaaagttcacgagcagctttacgaatctttgccaaagttaatttgacacgccaaacttcacgtttatttcgtaaaccaaactcgccaattaattttaattcctgatctaaacgctccttctcgaatggacgacgaggcac

SEQ ID NO: 46 - DNA, 200 bp, Meloidogyne incognita
ttcatcttatcctccaaaagaactccaatacgaaccaaacgacgaagaagagcatttccttcaaataagcgtcgaggatccttgtcttccaatgttaaaagttcacgagcagctttacgaatctttgccaaagttaatttgacacgccaaacttcacgtttatttcgtaaaccaaactcgccaattaattttaattcctg

SEQ ID NO: 47 - DNA, 2312 bp, Meloidogyne incognita
atcctcttcatcgtcaccgcctcctcctccacctcccgaagcagccttcttcaaattctttctcttgacacgtccaggaggtccaccagcaagataagattttgtggagaagtcaatgtgcttttgagagtcgagacgaacaatgaatgatggaatattgactacttgtcgacgaattcgaatatgcttttgacgaataagaacacgagcatggtgaattgatttagccaatcccaatttgaaaacttgcgtctgcaaacgacgctccaaaaagtcttcaacacgcaaaccaagaacatagtccaatttcatcttatcctccaaaagaactccaatacgaaccaaacgacgaagaagagcatttccttcaaataagcgtcgaggatccttgtcttccaatgttaaaagttcacgagcagctttacgaatctttgccaaagttaatttgacacgccaaacttcacgtttatttcgtaaaccaaactcgccaattaattttaattcctgatctaaacgctccttctcgaatggacgacgaggcacatcgtctctagacccagctttcttgtacaaagtggttgatcctgcagggtccgtcgcttctcttccatttcttctcattttcgattttgattcttatttctttccagtagctcctgctctgtgaatttctccgctcacgatagatctgcttatactccttacattcaaccttagatctggtctcgattctctgtttctctgtttttttcttttggtcgagaatctgatgtttgtttatgttctgtcaccattaataataatgaactctctcattcatacaatgattagtttctctcgtctacaaaacgatatgttgcattttcacttttcttctttttttctaagatgatttgctttgaccaatttgtttagatctttattctattttattttctggtgggttggtggaaattgaaaaaaaaaaaacagcataaattgttatttgttaatgtattcattttttggctatttgttctgggtaaaaatctgcttctactattgaatctttcctggattttttactcctattgggtttttatagtaaaaatacataataaaaggaaaacaaaagttttatagattctcttaaaccccttacgataaaagttggaatcaaaataattcaggatcagatgctctttgattgattcagatgcgattacagttgcatggcaaattttctagatccgtcgtcacattttattttctgtttaaatatctaaatctgatatatgatgtcgacaaattctggtggcttatacatcacttcaactgttttcttttggctttgtttgtcaacttggttttcaatacgatttgtgatttcgatcgctgaatttttaatacaagcaaactgatgttaaccacaagcaagagatgtgacctgccttattaacatcgtattacttactactagtcgtattctcaacgcaatcgtttttgtatttctcacattatgccgcttctctactctttattccttttggtccacgcattttctatttgtggcaatccctttcacaacctgatttcccactttggatcatttgtctgaagactctcttgaatcgttaccacttgtttcttgtgcatgctctgttttttagaattaatgataaaactattccatagtcttgagttttcagcttgttgattcttttgcttttggttttctgcaggtttaaacatcaaccactttgtacaagaaagctgggtctagagacgatgtgcctcgtcgtccattcgagaaggagcgtttagatcaggaattaaaattaattggcgagtttggtttacgaaataaacgtgaagtttggcgtgtcaaattaactttggcaaagattcgtaaagctgctcgtgaacttttaacattggaagacaaggatcctcgacgcttatttgaaggaaatgctcttcttcgtcgtttggttcgtattggagttcttttggaggataagatgaaattggactatgttcttggtttgcgtgttgaagactttttggagcgtcgtttgcagacgcaagttttcaaattgggattggctaaatcaattcaccatgctcgtgttcttattcgtcaaaagcatattcgaattcgtcgacaagtagtcaatattccatcattcattgttcgtctcgactctcaaaagcacattgacttctccacaaaatcttatcttgctggtggacctcctggacgtgtcaagagaaagaatttgaagaaggctgcttcgggaggtggaggaggaggcggtgacgatgaagaggat

SEQ ID NO: 48 - DNA, 1626 bp, Meloidogyne incognita
ttcatcttatcctccaaaagaactccaatacgaaccaaacgacgaagaagagcatttccttcaaataagcgtcgaggatccttgtcttccaatgttaaaagttcacgagcagctttacgaatctttgccaaagttaatttgacacgccaaacttcacgtttatttcgtaaaccaaactcgccaattaattttaattcctgatcgtctctagacccagctttcttgtacaaagtggttgatcctgcagggtccgtcgcttctcttccatttcttctcattttcgattttgattcttatttctttccagtagctcctgctctgtgaatttctccgctcacgatagatctgcttatactccttacattcaaccttagatctggtctcgattctctgtttctctgtttttttcttttggtcgagaatctgatgtttgtttatgttctgtcaccattaataataatgaactctctcattcatacaatgattagtttctctcgtctacaaaacgatatgttgcattttcacttttcttctttttttctaagatgatttgctttgaccaatttgtttagatctttattctattttattttctggtgggttggtggaaattgaaaaaaaaaaaacagcataaattgttatttgttaatgtattcattttttggctatttgttctgggtaaaaatctgcttctactattgaatctttcctggattttttactcctattgggtttttatagtaaaaatacataataaaaggaaaacaaaagttttatagattctcttaaaccccttacgataaaagttggaatcaaaataattcaggatcagatgctctttgattgattcagatgcgattacagttgcatggcaaattttctagatccgtcgtcacattttattttctgtttaaatatctaaatctgatatatgatgtcgacaaattctggtggcttatacatcacttcaactgttttcttttggctttgtttgtcaacttggttttcaatacgatttgtgatttcgatcgctgaatttttaatacaagcaaactgatgttaaccacaagcaagagatgtgacctgccttattaacatcgtattacttactactagtcgtattctcaacgcaatcgtttttgtatttctcacattatgccgcttctctactctttattccttttggtccacgcattttctatttgtggcaatccctttcacaacctgatttcccactttggatcatttgtctgaagactctcttgaatcgttaccacttgtttcttgtgcatgctctgttttttagaattaatgataaaactattccatagtcttgagttttcagcttgttgattcttttgcttttggttttctgcaggtttaaacatcaaccactttgtacaagaaagctgggtctagagacgatcaggaattaaaattaattggcgagtttggtttacgaaataaacgtgaagtttggcgtgtcaaattaactttggcaaagattcgtaaagctgctcgtgaacttttaacattggaagacaaggatcctcgacgcttatttgaaggaaatgctcttcttcgtcgtttggttcgtattggagttcttttggaggataagatgaa

SEQ ID NO: 49 - DNA, 708 bp, Meloidogyne incognita
ggtttaattacccaagtttgagctatcattttaaaaaaatcttttaaaaatgtctaaagctaacgctgtcggaattgatctgggaacgacttatttatgtgtcggtgtattccagcatggaaaagtcgagatcattgccaatgatcaaggaaatcgcacgactccatcttatgtggcttttacagacacggaacgtctgattggtgatgctgcgaaaaatcaagtggcaatgaacccgtcgaatactgtttttgacgcaaagcgcttgattggacgcaaatttgacgatccagctgttcaatctgacatgaagcattggccgttcaaggttattcaaggagaaggtgctcgtccaaaaatccaagtggaggttaagggtgaaatgaaggcgttcttccctgaggaagtctccgccatggttttgactaaaatgaaggaaacggctgaggcatttttgggccaaaccgttaaggatgctgtcatcactgtgcctgcgtactttaatgacagtcaacgccaagccacaaaggatgctggaacgatttctggacttaatgttttgcgtatcatcaacgagccaactgctgcggctattgcttatggtttggataagaaaggccaaggcgagcgcaacgtcttgatttttgacttgggcggtggaacttttgatgtgtctattttgactattgaagatggtatttttgaggt

SEQ ID NO: 50 - protein, 219 aa, Meloidogyne incognita
MSKANAVGIDLGTTYLCVGVFQHGKVEIIANDQGNRTTPSYVAFTDTERLIGDAAKNQVAMNPSNTVFDAKRLIGRKFDDPAVQSDMKHWPFKVIQGEGARPKIQVEVKGEMKAFFPEEVSAMVLTKMKETAEAFLGQTVKDAVITVPAYFNDSQRQATKDAGTISGLNVLRIINEPTAAAIAYGLDKKGQGERNVLIFDLGGGTFDVSILTIEDGIFE

SEQ ID NO: 51 - DNA, 150 bp, Meloidogyne incognita
atttttcgcagcatcaccaatcagacgttccgtgtctgtaaaagccacataagatggagtcgtgcgatttccttgatcattggcaatgatctcgacttttccatgctggaatacaccgacacataaataagtcgttcccagatcaattcc

SEQ ID NO: 52 - DNA, 452 bp, Meloidogyne incognita
ggtttaattacctacgtttgagaaatttaaagaatgacaaagttacaacatccagctgttgagggtcttgctgttggtttggatcgtggccatcctgtgactaaaaatgttcgcaagccacgtcaatctcgtaaaaaaggccgcattacgaagaagactcgaattgttcgtgaagttgttcgcgaggttgttggtttttcaccttatgagcgtcgaactatggaattgcttcgtatcagtaaggacaagaaggctttgaagtttttgaagaagcgaattggacaacatgtccgtgcaaagagaaagcgtgacgaaatccaacgaatactcgttgagatgagaaagcatcataagtgagaaaatataagggaaggaaaacattcaatcacagttttaattgttaaaaataatgttttatgcgattgaataaatttgttagaattcaaacaaaa

SEQ ID NO: 53 - protein, 107 aa, Meloidogyne incognita
MTKLQHPAVEGLAVGLDRGHPVTKNVRKPRQSRKKGRITKKTRIVREVVREVVGFSPYERRTMELLRISKDKKALKFLKKRIGQHVRAKRKRDEIQRILVEMRKHHK

SEQ ID NO: 54 - DNA, 150 bp, Meloidogyne incognita
tgagaaatttaaagaatgacaaagttacaacatccagctgttgagggtcttgctgttggtttggatcgtggccatcctgtgactaaaaatgttcgcaagccacgtcaatctcgtaaaaaaggccgcattacgaagaagactcgaattgtt

SEQ ID NO: 55 - DNA, 1522 bp, Meloidogyne incognita
aggtttaattacccaagtttgagttttactatttaatttatctaacaaaagatctcaaaaaatgcgtgaaatcgtccatatccaggctggacaatgtggtaaccaaattggttccaagttctgggaagtcatttcggatgaacacggaattcaacctgatggatcatacaagggagaatctgacttgcaattggaacgaatcaatgtttactacaatgaggctcatggtggaaagtatgtcccaagagctgttcttgttgacttggagccgggaaccatggactcgattagagctgggccatacggagaactcttccgtcctgacaactttgtctttggccagagcggtgcaggaaacaattgggccaaaggacattacactgagggagcggagttggtcgacaatgttttggatgttgttcgaaaagaggctgagggttgtgattgtcttcaaggctttcaattgactcactcgcttggtggtggaactggctctggaatgggaactttgctcatttcaaagattcgtgaggagtatccggaccgcattatgtcttccttctctgttgttccgtcgcctaaggtctctgacacggtcgttgagccctacaacgcaactctttctgttcatcaacttgttgagaacaccgacgagacttactgcatcgacaacgaggctttgtacgacatctgcttccgaactctgaagcttcaaaatccaacatatggagatttgaatcatctcgtatccgtaaccatgtctggagtgactacttgtcttcgcttcccaggccaattgaacgctgatcttcgcaaattggctgtcaacatggttccattcccacgtcttcactttttcatgcctggtttcgctccactttcggctaagggcgcagctgcctaccaggcccaaaatgttgctgaattgaccaagcaaatgtttgatgccaagaatatgatggctgcttgtgatccacgccatggacgctatttaactgttgccgctatcttccgtggacgtatgtcaatgcgtgaagtagacgatcaaatgatgtcggttcaaaacaagaattcgtcatactttgttgaatggattccaaacaacgttaagactgcggtttgtgacattcctccgcgtggtctcaagatgtctgcgaccttcattggcaactccacggccattcaagaactgttcaaacgcatctctgagcaattcacggccatgttccgtcgcaaggctttccttcactggtacactggcgaaggtatggacgagatggaattcactgaagccgagtccaacatgaacgacttgatttctgaataccagcaatatcaagatgcgactgttgaagacgaaggggaatttgaaggtgaagataccaatcaagcaactgttgaacaagaataaaaaagagagaagtttttgttttgaattaccacgtatttttttaattttattttgcttttaaccgcatatttccgaaataattcctcgcttaactttatttatgccaaaaaa

SEQ ID NO: 56 - protein, 449 aa, Meloidogyne incognita
MREIVHIQAGQCGNQIGSKFWEVISDEHGIQPDGSYKGESDLQLERINVYYNEAHGGKYVPRAVLVDLEPGTMDSIRAGPYGELFRPDNFVFGQSGAGNNWAKGHYTEGAELVDNVLDVVRKEAEGCDCLQGFQLTHSLGGGTGSGMGTLLISKIREEYPDRIMSSFSVVPSPKVSDTVVEPYNATLSVHQLVENTDETYCIDNEALYDICFRTLKLQNPTYGDLNHLVSVTMSGVTTCLRFPGQLNADLRKLAVNMVPFPRLHFFMPGFAPLSAKGAAAYQAQNVAELTKQMFDAKNMMAACDPRHGRYLTVAAIFRGRMSMREVDDQMMSVQNKNSSYFVEWIPNNVKTAVCDIPPRGLKMSATFIGNSTAIQELFKRISEQFTAMFRRKAFLHWYTGEGMDEMEFTEAESNMNDLISEYQQYQDATVEDEGEFEGEDTNQATVEQE

SEQ ID NO: 57 - DNA, 395 bp, Meloidogyne incognita
ccgacgagacttactgcatcgacaacgaggctttgtacgacatctgcttccgaactctgaagcttcaaaatccaacatatggagatttgaatcatctcgtatccgtaaccatgtctggagtgactacttgtcttcgcttcccaggccaattgaacgctgatcttcgcaaattggctgtcaacatggttccattcccacgtcttcactttttcatgcctggtttcgctccactttcggctaagggcgcagctgcctaccaggcccaaaatgttgctgaattgaccaagcaaatgtttgatgccaagaatatgatggctgcttgtgatccacgccatggacgctatttaactgttgccgctatcttccgtggacgtatgtcaatgcgtgaagtagac

SEQ ID NO: 58 - DNA, 2016 bp, Meloidogyne incognita
ccgacgagacttactgcatcgacaacgaggctttgtacgacatctgcttccgaactctgaagcttcaaaatccaacatatggagatttgaatcatctcgtatccgtaaccatgtctggagtgactacttgtcttcgcttcccaggccaattgaacgctgatcttcgcaaattggctgtcaacatggttccattcccacgtcttcactttttcatgcctggtttcgctccactttcggctaagggcgcagctgcctaccaggcccaaaatgttgctgaattgaccaagcaaatgtttgatgccaagaatatgatggctgcttgtgatccacgccatggacgctatttaactgttgccgctatcttccgtggacgtatgtcaatgcgtgaagtagacatcgtctctagacccagctttcttgtacaaagtggttgatcctgcagggtccgtcgcttctcttccatttcttctcattttcgattttgattcttatttctttccagtagctcctgctctgtgaatttctccgctcacgatagatctgcttatactccttacattcaaccttagatctggtctcgattctctgtttctctgtttttttcttttggtcgagaatctgatgtttgtttatgttctgtcaccattaataataatgaactctctcattcatacaatgattagtttctctcgtctacaaaacgatatgttgcattttcacttttcttctttttttctaagatgatttgctttgaccaatttgtttagatctttattctattttattttctggtgggttggtggaaattgaaaaaaaaaaaacagcataaattgttatttgttaatgtattcattttttggctatttgttctgggtaaaaatctgcttctactattgaatctttcctggattttttactcctattgggtttttatagtaaaaatacataataaaaggaaaacaaaagttttatagattctcttaaaccccttacgataaaagttggaatcaaaataattcaggatcagatgctctttgattgattcagatgcgattacagttgcatggcaaattttctagatccgtcgtcacattttattttctgtttaaatatctaaatctgatatatgatgtcgacaaattctggtggcttatacatcacttcaactgttttcttttggctttgtttgtcaacttggttttcaatacgatttgtgatttcgatcgctgaatttttaatacaagcaaactgatgttaaccacaagcaagagatgtgacctgccttattaacatcgtattacttactactagtcgtattctcaacgcaatcgtttttgtatttctcacattatgccgcttctctactctttattccttttggtccacgcattttctatttgtggcaatccctttcacaacctgatttcccactttggatcatttgtctgaagactctcttgaatcgttaccacttgtttcttgtgcatgctctgttttttagaattaatgataaaactattccatagtcttgagttttcagcttgttgattcttttgcttttggttttctgcaggtttaaacatcaaccactttgtacaagaaagctgggtctagagacgatgtctacttcacgcattgacatacgtccacggaagatagcggcaacagttaaatagcgtccatggcgtggatcacaagcagccatcatattcttggcatcaaacatttgcttggtcaattcagcaacattttgggcctggtaggcagctgcgcccttagccgaaagtggagcgaaaccaggcatgaaaaagtgaagacgtgggaatggaaccatgttgacagccaatttgcgaagatcagcgttcaattggcctgggaagcgaagacaagtagtcactccagacatggttacggatacgagatgattcaaatctccatatgttggattttgaagcttcagagttcggaagcagatgtcgtacaaagcctcgttgtcgatgcagtaagtctcgtcgg

SEQ ID NO: 59 - DNA, 450 bp, Meloidogyne incognita
atttttcgcagcatcaccaatcagacgttccgtgtctgtaaaagccacataagatggagtcgtgcgatttccttgatcattggcaatgatctcgacttttccatgctggaatacaccgacacataaataagtcgttcccagatcaattcctgagaaatttaaagaatgacaaagttacaacatccagctgttgagggtcttgctgttggtttggatcgtggccatcctgtgactaaaaatgttcgcaagccacgtcaatctcgtaaaaaaggccgcattacgaagaagactcgaattgttactcgtgtttccttagagaaaattacagctgttaagatcgccgcatcattaccagtccagcatgcaaagaaaacagcgcctgcacaatatattcgttacactcctagccagcaggctggtggttttcatacaagtggagcacaacaaaga

SEQ ID NO: 60 - DNA, 2386 bp, Meloidogyne incognita
atttttcgcagcatcaccaatcagacgttccgtgtctgtaaaagccacataagatggagtcgtgcgatttccttgatcattggcaatgatctcgacttttccatgctggaatacaccgacacataaataagtcgttcccagatcaattcctgagaaatttaaagaatgacaaagttacaacatccagctgttgagggtcttgctgttggtttggatcgtggccatcctgtgactaaaaatgttcgcaagccacgtcaatctcgtaaaaaaggccgcattacgaagaagactcgaattgttactcgtgtttccttagagaaaattacagctgttaagatcgccgcatcattaccagtccagcatgcaaagaaaacagcgcctgcacaatatattcgttacactcctagccagcaggctggtggttttcatacaagtggagcacaacaaagaacccagctttcttgtacaaagtggtgatatcccgcgggatcagaagcaacctcatggaaatgatgaggtaaggtttcatactcttgcctcttcttacggctttctgtgtcttcactgtaagtttctatgatttgagccaccaatatatatgctctggtgtgctgagttatgtttatctggtcacgcttagtgggtaaaattatgcttattttagcataaactttaatgagattaggttttgtatcacaccgatctttagttgtttagtaagatgacagaaattcttggtaaaacactctaaatcgtcttctttagtgaagttttccttagagtagcataaattttggcttttttcttgatggttgaataaggtggcacttgttggtatgagactttattgagagtcatattaagctgatccacgcgtttacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgccggatcagcttagcgttcattgaatttgatggccataggggtttagatgcaactgtttctttgaacattgtagaaatatataaagattttacattagcctactcttgaaagtcaaattgtcgaatttgattatattatactctagaggtgatattagttaatgagtttatactcggttatttacagcttattcatataccagttaacgtgtctcatatattctaacttcttagcatttaacgtgtttgcaggtcagcttgacactgaacataacagcatcactagtgcggccgcctgcaggtcgaccatatggtcgacctgcaggcggccgcactagtgatatcaccactttgtacaagaaagctgggttctttgttgtgctccacttgtatgaaaaccaccagcctgctggctaggagtgtaacgaatatattgtgcaggcgctgttttctttgcatgctggactggtaatgatgcggcgatcttaacagctgtaattttctctaaggaaacacgagtaacaattcgagtcttcttcgtaatgcggccttttttacgagattgacgtggcttgcgaacatttttagtcacaggatggccacgatccaaaccaacagcaagaccctcaacagctggatgttgtaactttgtcattctttaaatttctcaggaattgatctgggaacgacttatttatgtgtcggtgtattccagcatggaaaagtcgagatcattgccaatgatcaaggaaatcgcacgactccatcttatgtggcttttacagacacggaacgtctgattggtgatgctgcgaaaaat

SEQ ID NO: 61 - DNA, 22 bp, Meloidogyne incognita
aaaaggaaatcaataaaatgaa

SEQ ID NO: 62 - DNA, 41 bp, Meloidogyne incognita
atgaacacaactggagatgaatcaacattgaaagatattcc

SEQ ID NO: 63 - DNA, 26 bp, Meloidogyne incognita
gcgcagtctttggaacggccgcattc

SEQ ID NO: 64 - DNA, 44 bp, Meloidogyne incognita
ggggaatcgccatcttcaagtgtttacggctcttattgttcatc

SEQ ID NO: 65 - DNA, 34 bp, Meloidogyne incognita
tagaacaagttccaaaggatgacaggaagaaagg

SEQ ID NO: 66 - DNA, 47 bp, Meloidogyne incognita
tgggacaaacagatcaaattatggaaacttgcaatatatgaatgggc

SEQ ID NO: 67 - DNA, 64 bp, Meloidogyne incognita
aacttggagttgaactaaactttgcatatcctttcaccactttctgaacagcgtcaataaaatc

SEQ ID NO: 68 - DNA, 32 bp, Meloidogyne incognita
acttcatcaaagaaaataatacaagccttctt

SEQ ID NO: 69 - DNA, 35 bp, Meloidogyne incognita
atacgacgatcaatacgtcctggccgtacaagtgc

SEQ ID NO: 70 - DNA, 26 bp, Meloidogyne incognita
cccattccatcatcaaaacgagctcc

SEQ ID NO: 71 - DNA, 26 bp, Meloidogyne incognita
gcacgtaatgcaaacattccggcttc

SEQ ID NO: 72 - DNA, 26 bp, Meloidogyne incognita
ggacgatttgtggccattaaaacttt

SEQ ID NO: 73 - DNA, 23 bp, Meloidogyne incognita
gggcaaagacgcgatatcaattc

SEQ ID NO: 74 - DNA, 23 bp, Meloidogyne incognita
gtacagacagagcgaatatcagc

SEQ ID NO: 75 - DNA, 47 bp, Meloidogyne incognita
gtgtcatataacttggagttgaactaaactttgcatatcctttcacc

SEQ ID NO: 76 - DNA, 73 bp, Meloidogyne incognita
gtgtcatataacttggagttgaactaaactttgcatatcctttcaccactttctgaacagcgtcaataaaatc

SEQ ID NO: 77 - DNA, 28 bp, Meloidogyne incognita
atacaagccttctttgtttttgccatct

SEQ ID NO: 78 - DNA, 168 bp, Meloidogyne incognita
ccgtacaagtgcaggatccaaagtatcaggacgatttgtggccattaaaactttgattgcaccacgcgagtcaaatccatccagctgattgatcaattcgagcattgtccgttgaacttcattatctccccccattccatcatcaaaacgagctccgccaatagcatc

SEQ ID NO: 79 - DNA, 29 bp, Meloidogyne incognita
gatcctattgatatggatgaagatgaatt

SEQ ID NO: 80 - DNA, 35 bp, Meloidogyne incognita
cagtttgactctgagggcaaacttcgatttgatga

SEQ ID NO: 81 - DNA, 26 bp, Meloidogyne incognita
cattcccgcctttctgatatgaaatc

SEQ ID NO: 82 - DNA, 34 bp, Meloidogyne incognita
gaacgcaagatgattttggtgatggtggggcttt

SEQ ID NO: 83 - DNA, 24 bp, Meloidogyne incognita
cgcagaaagggtcgtcatatgggt

SEQ ID NO: 84 - DNA, 27 bp, Meloidogyne incognita
gacaagcatctttaccacgagctttac

SEQ ID NO: 85 - DNA, 23 bp, Meloidogyne incognita
gacaagcatctttaccacgagct

SEQ ID NO: 86 - DNA, 22 bp, Meloidogyne incognita
aagattgacaagcatctttacc

SEQ ID NO: 87 - DNA, 22 bp, Meloidogyne incognita
acaagcatctttaccacgagct

SEQ ID NO: 88 - DNA, 29 bp, Meloidogyne incognita
aagattgacaagcatctttaccacgagct

SEQ ID NO: 89 - DNA, 33 bp, Meloidogyne incognita
aagattgacaagcatctttaccacgagctttac

SEQ ID NO: 90 - DNA, 22 bp, Meloidogyne incognita
agattgacaagcatctttacca

SEQ ID NO: 91 - DNA, 22 bp, Meloidogyne incognita
attgacaagcatctttaccacg

SEQ ID NO: 92 - DNA, 21 bp, Meloidogyne incognita
caagcatctttaccacgagct

SEQ ID NO: 93 - DNA, 22 bp, Meloidogyne incognita
gacaagcatctttaccacgagc

SEQ ID NO: 94 - DNA, 22 bp, Meloidogyne incognita
gattgacaagcatctttaccac

SEQ ID NO: 95 - DNA, 22 bp, Meloidogyne incognita
tgacaagcatctttaccacgag

SEQ ID NO: 96 - DNA, 29 bp, Meloidogyne incognita
aagattgacaagcatctttaccacgagct

SEQ ID NO: 97 - DNA, 28 bp, Meloidogyne incognita
agattgacaagcatctttaccacgagct

SEQ ID NO: 98 - DNA, 21 bp, Meloidogyne incognita
gctcgtgaacgtttggaagca

SEQ ID NO: 99 - DNA, 32 bp, Meloidogyne incognita
gctcgtgaacgtttggaagcacgcagaaaggg

SEQ ID NO: 100 - DNA, 27 bp, Meloidogyne incognita
gctcgtgaacgtttggaagcacgcaga

SEQ ID NO: 101 - DNA, 170 bp, Meloidogyne incognita
tgaacaggctgaggctcgccgaaacaagaacaaggaaacgagaaagaggcgtgaggagcgtttgattttgaaacgtaaggaattgctccacaaactttctgagagcgaaaagcagaagactccacagcttcaagaagaggaagcgaaggaagcttcgccttctccaccac

SEQ ID NO: 102 - DNA, 26 bp, Meloidogyne incognita
agaaagggtcgtcatatgggttttgg

SEQ ID NO: 103 - DNA, 77 bp, Meloidogyne incognita
aaagttcattcgcgtttccgagctcgtgaacgtttggaagcacgcagaaagggtcgtcatatgggttttggaaaacg

SEQ ID NO: 104 - DNA, 71 bp, Meloidogyne incognita
aagagaaatttgatggaatatatcttcaaaaagaagactgagaaccagcgttctaaacaacttgctgaaca

SEQ ID NO: 105 - DNA, 59 bp, Meloidogyne incognita
aagcagaagactccacagcttcaagaagaggaagcgaaggaagcttcgccttctccacc

SEQ ID NO: 106 - DNA, 32 bp, Meloidogyne incognita
aaggaattgctccacaaactttctgagagcga

SEQ ID NO: 107 - DNA, 27 bp, Meloidogyne incognita
atgccggagaaggtcttatggatgcgt

SEQ ID NO: 108 - DNA, 141 bp, Meloidogyne incognita
attcgtaagcctggcaaagttcattcgcgtttccgagctcgtgaacgtttggaagcacgcagaaagggtcgtcatatgggttttggaaaacgacgcggaactcgtaacgctcgaatgccggagaaggtcttatggatgcgt

SEQ ID NO: 109 - DNA, 63 bp, Meloidogyne incognita
catcttttaaagcgctatcgagctgcgaagaagattgacaagcatctttaccacgagctttac

SEQ ID NO: 110 - DNA, 39 bp, Meloidogyne incognita
gatttccaacgcaaattctcgtcagaacgttcgtcgttt

SEQ ID NO: 111 - DNA, 40 bp, Meloidogyne incognita
gatttccaacgcaaattctcgtcagaacgttcgtcgtttg

SEQ ID NO: 112 - DNA, 199 bp, Meloidogyne incognita
gatttccaacgcaaattctcgtcagaacgttcgtcgtttggtcaaagatggtcttataattcgtaagcctggcaaagttcattcgcgtttccgagctcgtgaacgtttggaagcacgcagaaagggtcgtcatatgggttttggaaaacgacgcggaactcgtaacgctcgaatgccggagaaggtcttatggatgcgt

SEQ ID NO: 113 - DNA, 32 bp, Meloidogyne incognita
gtcaaagatggtcttataattcgtaagcctgg

SEQ ID NO: 114 - DNA, 21 bp, Meloidogyne incognita
tggatcctaatgaggtcaacg

SEQ ID NO: 115 - DNA, 172 bp, Meloidogyne incognita
tgtactccgtcatcttttaaagcgctatcgagctgcgaagaagattgacaagcatctttaccacgagctttacttgaaggctaaaggaaacgcatttaagaataagagaaatttgatggaatatatcttcaaaaagaagactgagaaccagcgttctaaacaacttgctgaa

SEQ ID NO: 116 - DNA, 338 bp, Meloidogyne incognita
tgtactccgtcatcttttaaagcgctatcgagctgcgaagaagattgacaagcatctttaccacgagctttacttgaaggctaaaggaaacgcatttaagaataagagaaatttgatggaatatatcttcaaaaagaagactgagaaccagcgttctaaacaacttgctgaacaggctgaggctcgccgaaacaagaacaaggaaacgagaaagaggcgtgaggagcgtttgattttgaaacgtaaggaattgctccacaaactttctgagagcgaaaagcagaagactccacagcttcaagaagaggaagcgaaggaagcttcgccttctccaccac

SEQ ID NO: 117 - DNA, 24 bp, Meloidogyne incognita
cgcagaaagggtcgtcatatgggt

SEQ ID NO: 118 - DNA, 24 bp, Meloidogyne incognita
cgcagaaagggtcgtcatatgggt

SEQ ID NO: 119 - DNA, 23 bp, Meloidogyne incognita
aattctcgtcagaacgttcgtcg

SEQ ID NO: 120 - DNA, 42 bp, Meloidogyne incognita
cttacacatccagagtactacgaagaaattggaatcaagcct

SEQ ID NO: 121 - DNA, 84 bp, Meloidogyne incognita
ggaaaaggctccaactgaaacgtacgctgatgttggcggtttggaacaacaaattcaagaaattaaggaggcagtggaattgcc

SEQ ID NO: 122 - DNA, 29 bp, Meloidogyne incognita
gctgaggctcgccgaaacaagaacaagga

SEQ ID NO: 123 - DNA, 47 bp, Meloidogyne incognita
aagcgaaagaagggaagtaaaggacctgaagctagcaataaaatgcc

SEQ ID NO: 124 - DNA, 34 bp, Meloidogyne incognita
atggaagaagaatttattcgaaatcaggaaagac

SEQ ID NO: 125 - DNA, 24 bp, Meloidogyne incognita
gaacattatgttaacattctttca

SEQ ID NO: 126 - DNA, 29 bp, Meloidogyne incognita
gaacattatgttaacattctttcatttgt

SEQ ID NO: 127 - DNA, 23 bp, Meloidogyne incognita
gaattgccacttacacatccaga

SEQ ID NO: 128 - DNA, 38 bp, Meloidogyne incognita
gatgaaatgagagggtcaccaatggctgtgggtacatt

SEQ ID NO: 129 - DNA, 29 bp, Meloidogyne incognita
ggtttggaacaacaaattcaagaaattaa

SEQ ID NO: 130 - DNA, 23 bp, Meloidogyne incognita
tgtgcagttcttctaaatcacaa

SEQ ID NO: 131 - DNA, 113 bp, Meloidogyne incognita
aacattctttcatttgtggacaaggaacaattggagccaggctgtgcagttcttctaaatcacaaaacacacgcagttgttggtgttttggctgatgacacggatccaatggt

SEQ ID NO: 132 - DNA, 74 bp, Meloidogyne incognita
aagaagggaagtaaaggacctgaagctagcaataaaatgcctaatgtgacaccagtaactcgttgcaaattgcg

SEQ ID NO: 133 - DNA, 61 bp, Meloidogyne incognita
aaggacctgaagctagcaataaaatgcctaatgtgacaccagtaactcgttgcaaattgcg

SEQ ID NO: 134 - DNA, 89 bp, Meloidogyne incognita
caggaagaagaacggacaaaagtagatgaaatgagagggtcaccaatggctgtgggtacattggaggagattattgacgatcaacacgc

SEQ ID NO: 135 - DNA, 98 bp, Meloidogyne incognita
caggaagaagaacggacaaaagtagatgaaatgagagggtcaccaatggctgtgggtacattggaggagattattgacgatcaacacgcagtcgtttc

SEQ ID NO: 136 - DNA, 62 bp, Meloidogyne incognita
ccaggttcaggagataagcaaggagaaggagacaagaagcgtcgttttgggcctccaatccc

SEQ ID NO: 137 - DNA, 83 bp, Meloidogyne incognita
cttcttaaatatgaacggattaaagattatttgttaatggaagaagaatttattcgaaatcaggaaagacataaacctcaaga

SEQ ID NO: 138 - DNA, 210 bp, Meloidogyne incognita
gttggcagcgaacattatgttaacattctttcatttgtggacaaggaacaattggagccaggctgtgcagttcttctaaatcacaaaacacacgcagttgttggtgttttggctgatgacacggatccaatggtttctgtaatgaaattggaaaaggctccaactgaaacgtacgctgatgttggcggtttggaacaacaaattcaagaa

SEQ ID NO: 139 - DNA, 119 bp, Meloidogyne incognita
tctgtaatgaaattggaaaaggctccaactgaaacgtacgctgatgttggcggtttggaacaacaaattcaagaaattaaggaggcagtggaattgccacttacacatccagagtacta

SEQ ID NO: 140 - DNA, 21 bp, Meloidogyne incognita
attgtgcggtcatcttgaaga

SEQ ID NO: 141 - DNA, 35 bp, Meloidogyne incognita
acgggatcctccaaagtcaatttgtaattttcaca

SEQ ID NO: 142 - DNA, 129 bp, Meloidogyne incognita
gcaactctggcacggtcaatcaaaacacgagcatctgcactcaaaccagcaaaagcagccattacgtgatcatccaatttgtgtatctttcgaattgtgcggtcatcttgaagaacaggaacagatttc

SEQ ID NO: 143 - DNA, 118 bp, Meloidogyne incognita
ggttcagtaataaaaagatgaggagttccatcgggatcaaaaccgccaacaagtaaagagacaccaaaaggacgacgacctgtagtttgcgtaaattcatgtttcaagtccgcaatag

SEQ ID NO: 144 - DNA, 357 bp, Meloidogyne incognita
ttctcattccggccgattgagccagcaagtaattcgtaatacacaccagatggttcagtaataaaaagatgaggagttccatcgggatcaaaaccgccaacaagtaaagagacaccaaaaggacgacgacctgtagtttgcgtaaattcatgtttcaagtccgcaatagtgcgggagatgtgctcaagtgtgacgggatcctccaaagtcaatttgtaattttcacaagcaactctggcacggtcaatcaaaacacgagcatctgcactcaaaccagcaaaagcagccattacgtgatcatccaatttgtgtatctttcgaattgtgcggtcatcttgaagaacaggaacagatttc

SEQ ID NO: 145 - DNA, 23 bp, Meloidogyne incognita
aaaaaaatgttaaaagaaaatat

SEQ ID NO: 146 - DNA, 22 bp, Meloidogyne incognita
aaaaaatgttaaaagaaaatat

SEQ ID NO: 147 - DNA, 21 bp, Meloidogyne incognita
aaaaatgttaaaagaaaatat

SEQ ID NO: 148 - DNA, 21 bp, Meloidogyne incognita
gaaaaaaatgttaaaagaaaa

SEQ ID NO: 149 - DNA, 22 bp, Meloidogyne incognita
gaaaaaaatgttaaaagaaaat

SEQ ID NO: 150 - DNA, 23 bp, Meloidogyne incognita
gaaaaaaatgttaaaagaaaata

SEQ ID NO: 151 - DNA, 24 bp, Meloidogyne incognita
gaaaaaaatgttaaaagaaaatat

SEQ ID NO: 152 - DNA, 23 bp, Meloidogyne incognita
gacaagcatctttaccacgagct

SEQ ID NO: 153 - DNA, 39 bp, Meloidogyne incognita
ataagaacacgagcatggtgaattgatttagccaatccc

SEQ ID NO: 154 - DNA, 41 bp, Meloidogyne incognita
ggaggtccaccagcaagataagattttgtggagaagtcaat

SEQ ID NO: 155 - DNA, 45 bp, Meloidogyne incognita
tccaaaagaactccaatacgaaccaaacgacgaagaagagcattt

SEQ ID NO: 156 - DNA, 51 bp, Meloidogyne incognita
tcctccacctcccgaagcagccttcttcaaattctttctcttgacacgtcc

SEQ ID NO: 157 - DNA, 65 bp, Meloidogyne incognita
tgcttttgagagtcgagacgaacaatgaatgatggaatattgactacttgtcgacgaattcgaat

SEQ ID NO: 158 - DNA, 37 bp, Meloidogyne incognita
ttttataaacctttagatattattcaatcctcttcat

SEQ ID NO: 159 - DNA, 23 bp, Meloidogyne incognita
aaaagttcacgagcagctttacg

SEQ ID NO: 160 - DNA, 45 bp, Meloidogyne incognita
aagaactccaatacgaaccaaacgacgaagaagagcatttccttc

SEQ ID NO: 161 - DNA, 25 bp, Meloidogyne incognita
aatgatggaatattgactacttgtc

SEQ ID NO: 162 - DNA, 26 bp, Meloidogyne incognita
aattttaattcctgatctaaacgctc

SEQ ID NO: 163 - DNA, 38 bp, Meloidogyne incognita
acacgtccaggaggtccaccagcaagataagattttgt

SEQ ID NO: 164 - DNA, 23 bp, Meloidogyne incognita
ccaagaacatagtccaatttcat

SEQ ID NO: 165 - DNA, 21 bp, Meloidogyne incognita
ctcaaactggagtaattaaac

SEQ ID NO: 166 - DNA, 22 bp, Meloidogyne incognita
gctcaaactggagtaattaaac

SEQ ID NO: 167 - DNA, 28 bp, Meloidogyne incognita
gtctgcaaacgacgctccaaaaagtctt

SEQ ID NO: 168 - DNA, 38 bp, Meloidogyne incognita
gtctgcaaacgacgctccaaaaagtcttcaacacgcaa

SEQ ID NO: 169 - DNA, 23 bp, Meloidogyne incognita
agctcaaactggagtaattaaac

SEQ ID NO: 170 - DNA, 21 bp, Meloidogyne incognita
ctcaaactggagtaattaaac

SEQ ID NO: 171 - DNA, 22 bp, Meloidogyne incognita
gctcaaactggagtaattaaac

SEQ ID NO: 172 - DNA, 25 bp, Meloidogyne incognita
taagctcaaactggagtaattaaac

SEQ ID NO: 173 - DNA, 47 bp, Meloidogyne incognita
aataagcgtcgaggatccttgtcttccaatgttaaaagttcacgagc

SEQ ID NO: 174 - DNA, 41 bp, Meloidogyne incognita
aattttaattcctgatctaaacgctccttctcgaatggacg

SEQ ID NO: 175 - DNA, 39 bp, Meloidogyne incognita; identical to SEQ ID NO: 153

SEQ ID NO: 176 - DNA, 53 bp, Meloidogyne incognita
ataagaacacgagcatggtgaattgatttagccaatcccaatttgaaaacttg

SEQ ID NO: 177 - DNA, 38 bp, Meloidogyne incognita
cgaggcacgtcataaactttagaaaatacatgctttat

SEQ ID NO: 178 - DNA, 52 bp, Meloidogyne incognita
ctcctccacctcccgaagcagccttcttcaaattctttctcttgacacgtcc

SEQ ID NO: 179 - DNA, 25 bp, Meloidogyne incognita
gaatctttgccaaagttaatttgac

SEQ ID NO: 180 - DNA, 37 bp, Meloidogyne incognita
gacgctccaaaaagtcttcaacacgcaaaccaagaac

SEQ ID NO: 181 - DNA, 47 bp, Meloidogyne incognita
gctttacgaatctttgccaaagttaatttgacacgccaaacttcacg

SEQ ID NO: 182 - DNA, 41 bp, Meloidogyne incognita; identical to SEQ ID NO: 154

SEQ ID NO: 183 - DNA, 48 bp, Meloidogyne incognita
tcaacaaaaacattttttcaaatccaaaatcttttaacaacttaacag

SEQ ID NO: 184 - DNA, 50 bp, Meloidogyne incognita
tccaaaagaactccaatacgaaccaaacgacgaagaagagcatttccttc

SEQ ID NO: 185 - DNA, 44 bp, Meloidogyne incognita
tgcaaacgacgctccaaaaagtcttcaacacgcaaaccaagaac

SEQ ID NO: 186 - DNA, 65 bp, Meloidogyne incognita; identical to SEQ ID NO: 157

SEQ ID NO: 187 - DNA, 101 bp, Meloidogyne incognita
ttttataaacctttagatattattcaatcctcttcatcgtcaccgcctcctcctccacctcccgaagcagccttcttcaaattctttctcttgacacgtcc

SEQ ID NO: 188 - DNA, 21 bp, Meloidogyne incognita
aagctcaaactggagtaatta

SEQ ID NO: 189 - DNA, 27 bp, Meloidogyne incognita
acgccaaacttcacgtttatttcgtaa

SEQ ID NO: 190 - DNA, 23 bp, Meloidogyne incognita
agctcaaactggagtaattaaac

SEQ ID NO: 191 - DNA, 33 bp, Meloidogyne incognita
cagctttacgaatctttgccaaagttaatttga

SEQ ID NO: 192 - DNA, 61 bp, Meloidogyne incognita
cagctttacgaatctttgccaaagttaatttgacacgccaaacttcacgtttatttcgtaa

SEQ ID NO: 193 - DNA, 64 bp, Meloidogyne incognita
cagctttacgaatctttgccaaagttaatttgacacgccaaacttcacgtttatttcgtaaacc

SEQ ID NO: 194 - DNA, 57 bp, Meloidogyne incognita
ccaaactcgccaattaattttaattcctgatctaaacgctccttctcgaatggacga

SEQ ID NO: 195 - DNA, 71 bp, Meloidogyne incognita
ccaaactcgccaattaattttaattcctgatctaaacgctccttctcgaatggacgacgaggcacgtcata

SEQ ID NO: 196 - DNA, 130 bp, Meloidogyne incognita
ccaaactcgccaattaattttaattcctgatctaaacgctccttctcgaatggacgacgaggcacgtcataaactttagaaaatacatgctttatgcgaggcatcttgttttactttttaagctcaaact

SEQ ID NO: 197 - DNA, 21 bp, Meloidogyne incognita
ctcaaactggagtaattaaac

SEQ ID NO: 198 - DNA, 134 bp, Meloidogyne incognita
ctttcaacgttttataaacctttagatattattcaatcctcttcatcgtcaccgcctcctcctccacctcccgaagcagccttcttcaaattctttctcttgacacgtccaggaggtccaccagcaagataaga

SEQ ID NO: 199 - DNA, 22 bp, Meloidogyne incognita
gctcaaactggagtaattaaac

SEQ ID NO: 200 - DNA, 47 bp, Meloidogyne incognita
gtctgcaaacgacgctccaaaaagtcttcaacacgcaaaccaagaac

SEQ ID NO: 201 - DNA, 50 bp, Meloidogyne incognita; identical to SEQ ID NO: 184

SEQ ID NO: 202 - DNA, 50 bp, Meloidogyne incognita
tcgaatggacgacgaggcacgtcataaactttagaaaatacatgctttat

SEQ ID NO: 203 - DNA, 38 bp, Meloidogyne incognita
tcgccaattaattttaattcctgatctaaacgctcctt

SEQ ID NO: 204 - DNA, 65 bp, Meloidogyne incognita
tgcttttgacgaataagaacacgagcatggtgaattgatttagccaatcccaatttgaaaacttg

SEQ ID NO: 205 - DNA, 65 bp, Meloidogyne incognita; identical to SEQ ID NO: 157

SEQ ID NO: 206 - DNA, 21 bp, Meloidogyne incognita
ttttactttttaagctcaaac

SEQ ID NO: 207 - DNA, 28 bp, Meloidogyne incognita
tctcgaatggacgacgaggcacgtcata

SEQ ID NO: 208 - DNA, 29 bp, Meloidogyne incognita
ttctcgaatggacgacgaggcacgtcata

SEQ ID NO: 209 - DNA, 21 bp, Meloidogyne incognita
aagaagagcatttccttcaaa

SEQ ID NO: 210 - DNA, 21 bp, Meloidogyne incognita
aagttcacgagcagctttacg

SEQ ID NO: 211 - DNA, 23 bp, Meloidogyne incognita
aataaacgtgaagtttggcgtgt

SEQ ID NO: 212 - DNA, 23 bp, Meloidogyne incognita
ttcgtaaagctgctcgtgaactt

SEQ ID NO: 213 - DNA, 29 bp, Meloidogyne incognita
gaaggaaatgctcttcttcgtcgtttggt

SEQ ID NO: 214 - DNA, 23 bp, Meloidogyne incognita
ccttgatcattggcaatgatctc

SEQ ID NO: 215 - DNA, 26 bp, Meloidogyne incognita
gttggctcgttgatgatacgcaaaac

SEQ ID NO: 216 - DNA, 22 bp, Meloidogyne incognita
agctcaaacttgggtaattaaa

SEQ ID NO: 217 - DNA, 23 bp, Meloidogyne incognita
agctcaaacttgggtaattaaac

SEQ ID NO: 218 - DNA, 21 bp, Meloidogyne incognita
ctcaaacttgggtaattaaac

SEQ ID NO: 219 - DNA, 21 bp, Meloidogyne incognita
gctcaaacttgggtaattaaa

SEQ ID NO: 220 - DNA, 22 bp, Meloidogyne incognita
gctcaaacttgggtaattaaac

SEQ ID NO: 221 - DNA, 21 bp, Meloidogyne incognita
tagctcaaacttgggtaatta

SEQ ID NO: 222 - DNA, 24 bp, Meloidogyne incognita
tagctcaaacttgggtaattaaac

SEQ ID NO: 223 - DNA, 23 bp, Meloidogyne incognita
agctcaaacttgggtaattaaac

SEQ ID NO: 224 - DNA, 21 bp, Meloidogyne incognita
ctcaaacttgggtaattaaac

SEQ ID NO: 225 - DNA, 22 bp, Meloidogyne incognita
gctcaaacttgggtaattaaac

SEQ ID NO: 226 - DNA, 24 bp, Meloidogyne incognita
tagctcaaacttgggtaattaaac

SEQ ID NO: 227 - DNA, 21 bp, Meloidogyne incognita
agctcaaacttgggtaattaa

SEQ ID NO: 228 - DNA, 22 bp, Meloidogyne incognita
agctcaaacttgggtaattaaa

SEQ ID NO: 229 - DNA, 23 bp, Meloidogyne incognita
agctcaaacttgggtaattaaac

SEQ ID NO: 230 - DNA, 22 bp, Meloidogyne incognita
atagctcaaacttgggtaatta

SEQ ID NO: 231 - DNA, 23 bp, Meloidogyne incognita
atagctcaaacttgggtaattaa

SEQ ID NO: 232 - DNA, 24 bp, Meloidogyne incognita
atagctcaaacttgggtaattaaa

SEQ ID NO: 233 - DNA, 21 bp, Meloidogyne incognita
ctcaaacttgggtaattaaac

SEQ ID NO: 234 - DNA, 23 bp, Meloidogyne incognita
gatagctcaaacttgggtaatta

SEQ ID NO: 235 - DNA, 24 bp, Meloidogyne incognita
gatagctcaaacttgggtaattaa

SEQ ID NO: 236 - DNA, 22 bp, Meloidogyne incognita
gcagcagttggctcgttgatga

SEQ ID NO: 237 - DNA, 21 bp, Meloidogyne incognita
gctcaaacttgggtaattaaa

SEQ ID NO: 238 - DNA, 22 bp, Meloidogyne incognita
gctcaaacttgggtaattaaac

SEQ ID NO: 239 - DNA, 21 bp, Meloidogyne incognita
tagctcaaacttgggtaatta

SEQ ID NO: 240 - DNA, 22 bp, Meloidogyne incognita
tagctcaaacttgggtaattaa

SEQ ID NO: 241 - DNA, 23 bp, Meloidogyne incognita
tagctcaaacttgggtaattaaa

SEQ ID NO: 242 - DNA, 26 bp, Meloidogyne incognita
gcagcagttggctcgttgatgatacg

SEQ ID NO: 243 - DNA, 21 bp, Meloidogyne incognita
agctcaaacttgggtaattaa

SEQ ID NO: 244 - DNA, 22 bp, Meloidogyne incognita
agctcaaacttgggtaattaaa

SEQ ID NO: 245 - DNA, 21 bp, Meloidogyne incognita
gctcaaacttgggtaattaaa

SEQ ID NO: 246 - DNA, 21 bp, Meloidogyne incognita
tagctcaaacttgggtaatta

SEQ ID NO: 247 - DNA, 26 bp, Meloidogyne incognita
cgatttccttgatcattggcaatgat

SEQ ID NO: 248 - DNA, 50 bp, Meloidogyne incognita
aataagtcgttcccagatcaattccgacagcgttagctttagacattttt

SEQ ID NO: 249 - DNA, 55 bp, Meloidogyne incognita
aataagtcgttcccagatcaattccgacagcgttagctttagacatttttaaaag

SEQ ID NO: 250 - DNA, 56 bp, Meloidogyne incognita
acgttgcgctcgccttggcctttcttatccaaaccataagcaatagccgcagcagt

SEQ ID NO: 251 - DNA, 476 bp, Meloidogyne incognita
ggctcgttgatgatacgcaaaacattaagtccagaaatcgttccagcatcctttgtggcttggcgttgactgtcattaaagtacgcaggcacagtgatgacagcatccttaacggtttggcccaaaaatgcctcagccgtttccttcattttagtcaaaaccatggcggagacttcctcagggaagaacgccttcatttcacccttaacctccacttggatttttggacgagcaccttctccttgaataaccttgaacggccaatgcttcatgtcagattgaacagctggatcgtcaaatttgcgtccaatcaagcgctttgcgtcaaaaacagtattcgacgggttcattgccacttgatttttcgcagcatcaccaatcagacgttccgtgtctgtaaaagccacataagatggagtcgtgcgatttccttgatcattggcaatgatctcgacttttccatgctggaatacacc

SEQ ID NO: 252 - DNA, 21 bp, Meloidogyne incognita
tagctcaaacttgggtaatta

SEQ ID NO: 253 - DNA, 31 bp, Meloidogyne incognita
tcaaaagttccaccgcccaagtcaaaaatca

SEQ ID NO: 254 - DNA, 21 bp, Meloidogyne incognita
ctcaaacttgggtaattaaac

SEQ ID NO: 255 - DNA, 21 bp, Meloidogyne incognita
tcagccgtttccttcatttta

SEQ ID NO: 256 - DNA, 23 bp, Meloidogyne incognita
accttgaacggccaatgcttcat

SEQ ID NO: 257 - DNA, 23 bp, Meloidogyne incognita
ccttgatcattggcaatgatctc

SEQ ID NO: 258 - DNA, 21 bp, Meloidogyne incognita
ctcaaacttgggtaattaaac

SEQ ID NO: 259 - DNA, 23 bp, Meloidogyne incognita
gtcaaaatagacacatcaaaagt

SEQ ID NO: 260 - DNA, 23 bp, Meloidogyne incognita
tcgttgatgatacgcaaaacatt

SEQ ID NO: 261 - DNA, 23 bp, Meloidogyne incognita
tcgttgatgatacgcaaaacatt

SEQ ID NO: 262 - DNA, 26 bp, Meloidogyne incognita
accttgaacggccaatgcttcatgtc

SEQ ID NO: 263 - DNA, 23 bp, Meloidogyne incognita
accttgaacggccaatgcttcat

SEQ ID NO: 264 - DNA, 21 bp, Meloidogyne incognita
tagctcaaacttgggtaatta

SEQ ID NO: 265 - DNA, 26 bp, Meloidogyne incognita
aaagctaacgctgtcggaattgatct

SEQ ID NO: 266 - DNA, 26 bp, Meloidogyne incognita
cgtatcatcaacgagccaactgctgc

SEQ ID NO: 267 - DNA, 21 bp, Meloidogyne incognita
ggtttaattacccaagtttga

SEQ ID NO: 268 - DNA, 22 bp, Meloidogyne incognita
ggtttaattacccaagtttgag

SEQ ID NO: 269 - DNA, 22 bp, Meloidogyne incognita
ggtttaattacccaagtttgag

SEQ ID NO: 270 - DNA, 24 bp, Meloidogyne incognita
ggtttaattacccaagtttgagct

SEQ ID NO: 271 - DNA, 25 bp, Meloidogyne incognita
ggtttaattacccaagtttgagcta

SEQ ID NO: 272 - DNA, 21 bp, Meloidogyne incognita
gtttaattacccaagtttgag

SEQ ID NO: 273 - DNA, 22 bp, Meloidogyne incognita
gtttaattacccaagtttgagc

SEQ ID NO: 274 - DNA, 22 bp, Meloidogyne incognita
tcatcaacgagccaactgctgc

SEQ ID NO: 275 - DNA, 21 bp, Meloidogyne incognita
ttaattacccaagtttgagct

SEQ ID NO: 276 - DNA, 21 bp, Meloidogyne incognita
ttaattacccaagtttgagct

SEQ ID NO: 277 - DNA, 22 bp, Meloidogyne incognita
ttaattacccaagtttgagcta

SEQ ID NO: 278 - DNA, 23 bp, Meloidogyne incognita
ttaattacccaagtttgagctat

SEQ ID NO: 279 - DNA, 21 bp, Meloidogyne incognita
tttaattacccaagtttgagc

SEQ ID NO: 280 - DNA, 23 bp, Meloidogyne incognita
cgtgcaaagagaaagcgtgacga

SEQ ID NO: 281 - DNA, 22 bp, Meloidogyne incognita
ttgttcgcgaggttgttggttt

SEQ ID NO: 282 - DNA, 22 bp, Meloidogyne incognita
ttgttcgcgaggttgttggttt

SEQ ID NO: 283 - DNA, 26 bp, Meloidogyne incognita
aagaaggctttgaagtttttgaagaa

SEQ ID NO: 284 - DNA, 21 bp, Meloidogyne incognita
ggtttaattacctacgtttga

SEQ ID NO: 285 - DNA, 22 bp, Meloidogyne incognita
ggtttaattacctacgtttgag

SEQ ID NO: 286 - DNA, 23 bp, Meloidogyne incognita
ggtttaattacctacgtttgaga

SEQ ID NO: 287 - DNA, 24 bp, Meloidogyne incognita
ggtttaattacctacgtttgagaa

SEQ ID NO: 288 - DNA, 28 bp, Meloidogyne incognita
ggtttaattacctacgtttgagaaattt

SEQ ID NO: 289 - DNA, 25 bp, Meloidogyne incognita
agaaggctttgaagtttttgaagaa

SEQ ID NO: 290 - DNA, 21 bp, Meloidogyne incognita
agaatgacaaagttacaacat

SEQ ID NO: 291 - DNA, 21 bp, Meloidogyne incognita
atgagaaagcatcataagtga

SEQ ID NO: 292 - DNA, 25 bp, Meloidogyne incognita
atggaattgcttcgtatcagtaagg

SEQ ID NO: 293 - DNA, 34 bp, Meloidogyne incognita
atggaattgcttcgtatcagtaaggacaagaagg

SEQ ID NO: 294 - DNA, 53 bp, Meloidogyne incognita
atggaattgcttcgtatcagtaaggacaagaaggctttgaagtttttgaagaa

SEQ ID NO: 295 - DNA, 30 bp, Meloidogyne incognita
ggacaagaaggctttgaagtttttgaagaa

SEQ ID NO: 296 - DNA, 22 bp, Meloidogyne incognita
ggtttaattacctacgtttgag

SEQ ID NO: 297 - DNA, 23 bp, Meloidogyne incognita
ggtttaattacctacgtttgaga

SEQ ID NO: 298 - DNA, 24 bp, Meloidogyne incognita
ggtttaattacctacgtttgagaa

SEQ ID NO: 299 - DNA, 25 bp, Meloidogyne incognita
ggtttaattacctacgtttgagaaa

SEQ ID NO: 300 - DNA, 49 bp, Meloidogyne incognita
aacatccagctgttgagggtcttgctgttggtttggatcgtggccatcc

SEQ ID NO: 301 - DNA, 76 bp, Meloidogyne incognita
aacatccagctgttgagggtcttgctgttggtttggatcgtggccatcctgtgactaaaaatgttcgcaagccacg

SEQ ID NO: 302 - DNA, 37 bp, Meloidogyne incognita
aaggaaaacattcaatcacagttttaattgttaaaaa

SEQ ID NO: 303 - DNA, 22 bp, Meloidogyne incognita
acatccagctgttgagggtctt

SEQ ID NO: 304 - DNA, 25 bp, Meloidogyne incognita
actatggaattgcttcgtatcagta

SEQ ID NO: 305 - DNA, 68 bp, Meloidogyne incognita
actatggaattgcttcgtatcagtaaggacaagaaggctttgaagtttttgaagaagcgaattggaca

SEQ ID NO: 306 - DNA, 46 bp, Meloidogyne incognita
agaagcgaattggacaacatgtccgtgcaaagagaaagcgtgacga

SEQ ID NO: 307 - DNA, 70 bp, Meloidogyne incognita
cagctgttgagggtcttgctgttggtttggatcgtggccatcctgtgactaaaaatgttcgcaagccacg

SEQ ID NO: 308 - DNA, 74 bp, Meloidogyne incognita
catccagctgttgagggtcttgctgttggtttggatcgtggccatcctgtgactaaaaatgttcgcaagccacg

SEQ ID NO: 309 - DNA, 50 bp, Meloidogyne incognita
catcctgtgactaaaaatgttcgcaagccacgtcaatctcgtaaaaaagg

SEQ ID NO: 310 - DNA, 122 bp, Meloidogyne incognita
catgtccgtgcaaagagaaagcgtgacgaaatccaacgaatactcgttgagatgagaaagcatcataagtgagaaaatataagggaaggaaaacattcaatcacagttttaattgttaaaaa

SEQ ID NO: 311 - DNA, 129 bp, Meloidogyne incognita
catgtccgtgcaaagagaaagcgtgacgaaatccaacgaatactcgttgagatgagaaagcatcataagtgagaaaatataagggaaggaaaacattcaatcacagttttaattgttaaaaataatgtt

SEQ ID NO: 312 - DNA, 41 bp, Meloidogyne incognita
cgaattgttcgtgaagttgttcgcgaggttgttggtttttc

SEQ ID NO: 313 - DNA, 155 bp, Meloidogyne incognita
cgaattgttcgtgaagttgttcgcgaggttgttggtttttcaccttatgagcgtcgaactatggaattgcttcgtatcagtaaggacaagaaggctttgaagtttttgaagaagcgaattggacaacatgtccgtgcaaagagaaagcgtgacga

SEQ ID NO: 314 - DNA, 68 bp, Meloidogyne incognita
gaattgttcgtgaagttgttcgcgaggttgttggtttttcaccttatgagcgtcgaactatggaattg

SEQ ID NO: 315 - DNA, 26 bp, Meloidogyne incognita
gtgactaaaaatgttcgcaagccacg

SEQ ID NO: 316 - DNA, 25 bp, Meloidogyne incognita
gttgagatgagaaagcatcataagt

SEQ ID NO: 317 - DNA, 30 bp, Meloidogyne incognita
tatgcgattgaataaatttgttagaattca

SEQ ID NO: 318 - DNA, 23 bp, Meloidogyne incognita
tgagatgagaaagcatcataagt

SEQ ID NO: 319 - DNA, 38 bp, Meloidogyne incognita
ttcgtatcagtaaggacaagaaggctttgaagtttttg

SEQ ID NO: 320 - DNA, 23 bp, Meloidogyne incognita
cgtgcaaagagaaagcgtgacga

SEQ ID NO: 321 - DNA, 26 bp, Meloidogyne incognita
aacatggttccattcccacgtcttca

SEQ ID NO: 322 - DNA, 23 bp, Meloidogyne incognita
gttgaatggattccaaacaacgt

SEQ ID NO: 323 - DNA, 29 bp, Meloidogyne incognita
aacatggttccattcccacgtcttcactt

SEQ ID NO: 324 - DNA, 23 bp, Meloidogyne incognita
atggacgagatggaattcactga

SEQ ID NO: 325 - DNA, 29 bp, Meloidogyne incognita
aacatggttccattcccacgtcttcactt

SEQ ID NO: 326 - DNA, 29 bp, Meloidogyne incognita
aacatggttccattcccacgtcttcactt

SEQ ID NO: 327 - DNA, 26 bp, Meloidogyne incognita
aagattcgtgaggagtatccggaccg

SEQ ID NO: 328 - DNA, 21 bp, Meloidogyne incognita
aggtttaattacccaagtttg

SEQ ID NO: 329 - DNA, 22 bp, Meloidogyne incognita
gaggctttgtacgacatctgct

SEQ ID NO: 330 - DNA, 24 bp, Meloidogyne incognita
gaggctttgtacgacatctgcttc

SEQ ID NO: 331 - DNA, 21 bp, Meloidogyne incognita
gctttgtacgacatctgcttc

SEQ ID NO: 332 - DNA, 21 bp, Meloidogyne incognita
ggtttaattacccaagtttga

SEQ ID NO: 333 - DNA, 22 bp, Meloidogyne incognita
ggtttaattacccaagtttgag

SEQ ID NO: 334 - DNA, 23 bp, Meloidogyne incognita
ggtttaattacccaagtttgagt

SEQ ID NO: 335 - DNA, 24 bp, Meloidogyne incognita
ggtttaattacccaagtttgagtt

SEQ ID NO: 336 - DNA, 25 bp, Meloidogyne incognita
ggtttaattacccaagtttgagttt

SEQ ID NO: 337 - DNA, 21 bp, Meloidogyne incognita
gtttaattacccaagtttgag

SEQ ID NO: 338 - DNA, 22 bp, Meloidogyne incognita
gtttaattacccaagtttgagt

SEQ ID NO: 339 - DNA, 23 bp, Meloidogyne incognita
gtttaattacccaagtttgagtt

SEQ ID NO: 340 - DNA, 24 bp, Meloidogyne incognita
gtttaattacccaagtttgagttt

SEQ ID NO: 341 - DNA, 21 bp, Meloidogyne incognita
ttaattacccaagtttgagtt

SEQ ID NO: 342 - DNA, 26 bp, Meloidogyne incognita
aacatggttccattcccacgtcttca

SEQ ID NO: 343 - DNA, 30 bp, Meloidogyne incognita
aacatggttccattcccacgtcttcacttt

SEQ ID NO: 344 - DNA, 21 bp, Meloidogyne incognita
aattacccaagtttgagtttt

SEQ ID NO: 345 - DNA, 22 bp, Meloidogyne incognita
aattacccaagtttgagtttta

SEQ ID NO: 346 - DNA, 23 bp, Meloidogyne incognita
aattacccaagtttgagttttac

SEQ ID NO: 347 - DNA, 28 bp, Meloidogyne incognita
acatggttccattcccacgtcttcactt

SEQ ID NO: 348 - DNA, 21 bp, Meloidogyne incognita
aggtttaattacccaagtttg

SEQ ID NO: 349 - DNA, 22 bp, Meloidogyne incognita
aggtttaattacccaagtttga

SEQ ID NO: 350 - DNA, 23 bp, Meloidogyne incognita
aggtttaattacccaagtttgag

SEQ ID NO: 351 - DNA, 23 bp, Meloidogyne incognita
atggacgagatggaattcactga

SEQ ID NO: 352 - DNA, 26 bp, Meloidogyne incognita
atggacgagatggaattcactgaagc

SEQ ID NO: 353 - DNA, 30 bp, Meloidogyne incognita
caacatggttccattcccacgtcttcactt

SEQ ID NO: 354 - DNA, 29 bp, Meloidogyne incognita
cttgttgagaacaccgacgagacttactg

SEQ ID NO: 355 - DNA, 23 bp, Meloidogyne incognita
ggaaacaattgggccaaaggaca

SEQ ID NO: 356 - DNA, 21 bp, Meloidogyne incognita
ggttccattcccacgtcttca

SEQ ID NO: 357 - DNA, 24 bp, Meloidogyne incognita
gtcaacatggttccattcccacgt

SEQ ID NO: 358 - DNA, 27 bp, Meloidogyne incognita
gtcaacatggttccattcccacgtctt

SEQ ID NO: 359 - DNA, 32 bp, Meloidogyne incognita
gtcaacatggttccattcccacgtcttcactt

SEQ ID NO: 360 - DNA, 33 bp, Meloidogyne incognita
gtcaacatggttccattcccacgtcttcacttt

SEQ ID NO: 361 - DNA, 21 bp, Meloidogyne incognita
taattacccaagtttgagttt

SEQ ID NO: 362 - DNA, 22 bp, Meloidogyne incognita
taattacccaagtttgagtttt

SEQ ID NO: 363 - DNA, 23 bp, Meloidogyne incognita
taattacccaagtttgagtttta

SEQ ID NO: 364 - DNA, 32 bp, Meloidogyne incognita
tacaacgcaactctttctgttcatcaacttgt

SEQ ID NO: 365 - DNA, 35 bp, Meloidogyne incognita
tacaacgcaactctttctgttcatcaacttgttga

SEQ ID NO: 366 - DNA, 29 bp, Meloidogyne incognita
tactttgttgaatggattccaaacaacgt

SEQ ID NO: 367 - DNA, 21 bp, Meloidogyne incognita
tgaatggattccaaacaacgt

SEQ ID NO: 368 - DNA, 25 bp, Meloidogyne incognita
tggttccattcccacgtcttcactt

SEQ ID NO: 369 - DNA, 30 bp, Meloidogyne incognita
tgtcaacatggttccattcccacgtcttca

SEQ ID NO: 370 - DNA, 21 bp, Meloidogyne incognita
ttaattacccaagtttgagtt

SEQ ID NO: 371 - DNA, 22 bp, Meloidogyne incognita
ttaattacccaagtttgagttt

SEQ ID NO: 372 - DNA, 23 bp, Meloidogyne incognita
ttaattacccaagtttgagtttt

SEQ ID NO: 373 - DNA, 24 bp, Meloidogyne incognita
ttaattacccaagtttgagtttta

SEQ ID NO: 374 - DNA, 21 bp, Meloidogyne incognita
ttccgtggacgtatgtcaatg

SEQ ID NO: 375 - DNA, 26 bp, Meloidogyne incognita
ttggctgtcaacatggttccattccc

SEQ ID NO: 376 - DNA, 31 bp, Meloidogyne incognita
ttggctgtcaacatggttccattcccacgtc

SEQ ID NO: 377 - DNA, 35 bp, Meloidogyne incognita
ttggctgtcaacatggttccattcccacgtcttca

SEQ ID NO: 378 - DNA, 21 bp, Meloidogyne incognita
tttaattacccaagtttgagt

SEQ ID NO: 379 - DNA, 22 bp, Meloidogyne incognita
tttaattacccaagtttgagtt

SEQ ID NO: 380 - DNA, 23 bp, Meloidogyne incognita
tttaattacccaagtttgagttt

SEQ ID NO: 381 - DNA, 24 bp, Meloidogyne incognita
tttaattacccaagtttgagtttt

SEQ ID NO: 382 - DNA, 25 bp, Meloidogyne incognita
tttaattacccaagtttgagtttta

SEQ ID NO: 383 - DNA, 26 bp, Meloidogyne incognita
tttaattacccaagtttgagttttac

SEQ ID NO: 384 - DNA, 23 bp, Meloidogyne incognita
ggttccaagttctgggaagtcat

SEQ ID NO: 385 - DNA, 22 bp, Meloidogyne incognita
gaatcaatgtttactacaatga

SEQ ID NO: 386 - DNA, 23 bp, Meloidogyne incognita
gttgaatggattccaaacaacgt

SEQ ID NO: 387 - DNA, 29 bp, Meloidogyne incognita
aaccaaattggttccaagttctgggaagt

SEQ ID NO: 388 - DNA, 38 bp, Meloidogyne incognita
gaaggtatggacgagatggaattcactgaagccgagtc

SEQ ID NO: 389 - DNA, 26 bp, Meloidogyne incognita
gatgccaagaatatgatggctgcttg

SEQ ID NO: 390 - DNA, 26 bp, Meloidogyne incognita
ttcactgaagccgagtccaacatgaa

SEQ ID NO: 391 - DNA, 29 bp, Meloidogyne incognita
aaccaaattggttccaagttctgggaagt

SEQ ID NO: 392 - DNA, 38 bp, Meloidogyne incognita; identical to SEQ ID NO: 388

SEQ ID NO: 393 - DNA, 29 bp, Meloidogyne incognita
aaccaaattggttccaagttctgggaagt

SEQ ID NO: 394 - DNA, 38 bp, Meloidogyne incognita; identical to SEQ ID NO: 388

SEQ ID NO: 395 - DNA, 26 bp, Meloidogyne incognita
gatgccaagaatatgatggctgcttg

SEQ ID NO: 396 - DNA, 29 bp, Meloidogyne incognita
aaccaaattggttccaagttctgggaagt

SEQ ID NO: 397 - DNA, 32 bp, Meloidogyne incognita
atgtttgatgccaagaatatgatggctgcttg

SEQ ID NO: 398 - DNA, 22 bp, Meloidogyne incognita
ttggttccaagttctgggaagt

SEQ ID NO: 399 - DNA, 29 bp, Meloidogyne incognita
aaccaaattggttccaagttctgggaagt

SEQ ID NO: 400 - DNA, 26 bp, Meloidogyne incognita
ttcactgaagccgagtccaacatgaa

SEQ ID NO: 401 - DNA, 29 bp, Meloidogyne incognita
aaccaaattggttccaagttctgggaagt

SEQ ID NO: 402 - DNA, 32 bp, Meloidogyne incognita; identical to SEQ ID NO: 397

SEQ ID NO: 403 - DNA, 23 bp, Meloidogyne incognita
actgaagccgagtccaacatgaa

SEQ ID NO: 404 - DNA, 26 bp, Meloidogyne incognita
gatgccaagaatatgatggctgcttg

SEQ ID NO: 405 - DNA, 29 bp, Meloidogyne incognita
aaccaaattggttccaagttctgggaagt

SEQ ID NO: 406 - DNA, 26 bp, Meloidogyne incognita
aagaatatgatggctgcttgtgatcc

SEQ ID NO: 407 - DNA, 26 bp, Meloidogyne incognita
ttcactgaagccgagtccaacatgaa

SEQ ID NO: 408 - DNA, 21 bp, Meloidogyne incognita
aattacccaagtttgagtttt

SEQ ID NO: 409 - DNA, 21 bp, Meloidogyne incognita
aggtttaattacccaagtttg

SEQ ID NO: 410 - DNA, 30 bp, Meloidogyne incognita
gaacgaatcaatgtttactacaatgaggct

SEQ ID NO: 411 - DNA, 29 bp, Meloidogyne incognita
gaatggattccaaacaacgttaagactgc

SEQ ID NO: 412 - DNA, 21 bp, Meloidogyne incognita
taattacccaagtttgagttt

SEQ ID NO: 413 - DNA, 24 bp, Meloidogyne incognita
tgaacacggaattcaacctgatgg

SEQ ID NO: 414 - DNA, 25 bp, Meloidogyne incognita
tgaacacggaattcaacctgatgga

SEQ ID NO: 415 - DNA, 26 bp, Meloidogyne incognita
tggtacactggcgaaggtatggacga

SEQ ID NO: 416 - DNA, 21 bp, Meloidogyne incognita
ttaattacccaagtttgagtt

SEQ ID NO: 417 - DNA, 21 bp, Meloidogyne incognita
ttaattacccaagtttgagtt

SEQ ID NO: 418 - DNA, 22 bp, Meloidogyne incognita
tttaattacccaagtttgagtt

SEQ ID NO: 419 - DNA, 23 bp, Meloidogyne incognita
atgttccgtcgcaaggctttcct

SEQ ID NO: 420 - DNA, 23 bp, Meloidogyne incognita
gaagccgagtccaacatgaacga

SEQ ID NO: 421 - DNA, 22 bp, Meloidogyne incognita
taattacccaagtttgagtttt

SEQ ID NO: 422 - DNA, 30 bp, Meloidogyne incognita
ttcacggccatgttccgtcgcaaggctttc

SEQ ID NO: 423 - DNA, 21 bp, Meloidogyne incognita
aattacccaagtttgagtttt

SEQ ID NO: 424 - DNA, 21 bp, Meloidogyne incognita
caatgtggtaaccaaattggt

SEQ ID NO: 425 - DNA, 22 bp, Meloidogyne incognita
aaaagagagaagtttttgtttt

SEQ ID NO: 426 - DNA, 41 bp, Meloidogyne incognita
aaacgcatctctgagcaattcacggccatgttccgtcgcaa

SEQ ID NO: 427 - DNA, 23 bp, Meloidogyne incognita
aatatgatggctgcttgtgatcc

SEQ ID NO: 428 - DNA, 26 bp, Meloidogyne incognita
actctgaagcttcaaaatccaacata

SEQ ID NO: 429 - DNA, 23 bp, Meloidogyne incognita
atgatggctgcttgtgatccacg

SEQ ID NO: 430 - DNA, 89 bp, Meloidogyne incognita
atgcgtgaagtagacgatcaaatgatgtcggttcaaaacaagaattcgtcatactttgttgaatggattccaaacaacgttaagactgc

SEQ ID NO: 431 - DNA, 29 bp, Meloidogyne incognita
atgtctgcgaccttcattggcaactccac

SEQ ID NO: 432 - DNA, 47 bp, Meloidogyne incognita
attcaacctgatggatcatacaagggagaatctgacttgcaattgga

SEQ ID NO: 433 - DNA, 29 bp, Meloidogyne incognita
caggcccaaaatgttgctgaattgaccaa

SEQ ID NO: 434 - DNA, 39 bp, Meloidogyne incognita
cgaatcaatgtttactacaatgaggctcatggtggaaag

SEQ ID NO: 435 - DNA, 40 bp, Meloidogyne incognita
ctggtttcgctccactttcggctaagggcgcagctgccta

SEQ ID NO: 436 - DNA, 36 bp, Meloidogyne incognita
ctttgttgaatggattccaaacaacgttaagactgc

SEQ ID NO: 437 - DNA, 23 bp, Meloidogyne incognita
gctatcttccgtggacgtatgtc

SEQ ID NO: 438 - DNA, 59 bp, Meloidogyne incognita
gtatccgtaaccatgtctggagtgactacttgtcttcgcttcccaggccaattgaacgc

SEQ ID NO: 439 - DNA, 89 bp, Meloidogyne incognita
taccagcaatatcaagatgcgactgttgaagacgaaggggaatttgaaggtgaagataccaatcaagcaactgttgaacaagaataaaa

SEQ ID NO: 440 - DNA, 35 bp, Meloidogyne incognita
tccaagttctgggaagtcatttcggatgaacacgg

SEQ ID NO: 441 - DNA, 27 bp, Meloidogyne incognita
tgtcggttcaaaacaagaattcgtcat

SEQ ID NO: 442 - DNA, 60 bp, Meloidogyne incognita
ttattttgcttttaaccgcatatttccgaaataattcctcgcttaactttatttatgcca

SEQ ID NO: 443 - DNA, 47 bp, Meloidogyne incognita
ttccttcactggtacactggcgaaggtatggacgagatggaattcac

SEQ ID NO: 444 - DNA, 86 bp, Meloidogyne incognita
ttggctgtcaacatggttccattcccacgtcttcactttttcatgcctggtttcgctccactttcggctaagggcgcagctgccta

SEQ ID NO: 445 - DNA, 63 bp, Meloidogyne incognita
tttatctaacaaaagatctcaaaaaatgcgtgaaatcgtccatatccaggctggacaatgtgg

SEQ ID NO: 446 - DNA, 22 bp, Meloidogyne incognita
taattacccaagtttgagtttt

SEQ ID NO: 447 - DNA, 23 bp, Meloidogyne incognita
taattacccaagtttgagtttta

SEQ ID NO: 448 - DNA, 32 bp, Meloidogyne incognita
aacatggttccattcccacgtcttcacttttt

SEQ ID NO: 449 - DNA, 23 bp, Meloidogyne incognita
actacttgtcttcgcttcccagg

SEQ ID NO: 450 - DNA, 21 bp, Meloidogyne incognita
aattacccaagtttgagtttt

SEQ ID NO: 451 - DNA, 26 bp, Meloidogyne incognita
atggactcgattagagctgggccata

SEQ ID NO: 452 - DNA, 146 bp, Meloidogyne incognita
gacaatgttttggatgttgttcgaaaagaggctgagggttgtgattgtcttcaaggctttcaattgactcactcgcttggtggtggaactggctctggaatgggaactttgctcatttcaaagattcgtgaggagtatccggaccg

SEQ ID NO: 453 - DNA, 86 bp, Meloidogyne incognita
ggagaactcttccgtcctgacaactttgtctttggccagagcggtgcaggaaacaattgggccaaaggacattacactgagggagc

SEQ ID NO: 454 - DNA, 23 bp, Meloidogyne incognita
ggtggaaagtatgtcccaagagc

SEQ ID NO: 455 - DNA, 21 bp, Meloidogyne incognita
taattacccaagtttgagttt

SEQ ID NO: 456 - DNA, 24 bp, Meloidogyne incognita
aaaaaagagagaagtttttgtttt

SEQ ID NO: 457 - DNA, 23 bp, Meloidogyne incognita
aaaaagagagaagtttttgtttt

SEQ ID NO: 458 - DNA, 41 bp, Meloidogyne incognita; identical to SEQ ID NO: 426

SEQ ID NO: 459 - DNA, 26 bp, Meloidogyne incognita
accttcattggcaactccacggccat

SEQ ID NO: 460 - DNA, 30 bp, Meloidogyne incognita
ataattcctcgcttaactttatttatgcca

SEQ ID NO: 461 - DNA, 21 bp, Meloidogyne incognita
attacccaagtttgagtttta

SEQ ID NO: 462 - DNA, 30 bp, Meloidogyne incognita
atttgaaggtgaagataccaatcaagcaac

SEQ ID NO: 463 - DNA, 24 bp, Meloidogyne incognita
caaaggacattacactgagggagc

SEQ ID NO: 464 - DNA, 47 bp, Meloidogyne incognita
cgcatatttccgaaataattcctcgcttaactttatttatgccaaaa

SEQ ID NO: 465 - DNA, 35 bp, Meloidogyne incognita
cgtcctgacaactttgtctttggccagagcggtgc

SEQ ID NO: 466 - DNA, 53 bp, Meloidogyne incognita
gaactgttcaaacgcatctctgagcaattcacggccatgttccgtcgcaaggc

SEQ ID NO: 467 - DNA, 23 bp, Meloidogyne incognita
gaagacgaaggggaatttgaagg

SEQ ID NO: 468 - DNA, 62 bp, Meloidogyne incognita
gaagccgagtccaacatgaacgacttgatttctgaataccagcaatatcaagatgcgactgt

SEQ ID NO: 469 - DNA, 95 bp, Meloidogyne incognita
gacgatcaaatgatgtcggttcaaaacaagaattcgtcatactttgttgaatggattccaaacaacgttaagactgcggtttgtgacattcctcc

SEQ ID NO: 470 - DNA, 142 bp, Meloidogyne incognita
gagatggaattcactgaagccgagtccaacatgaacgacttgatttctgaataccagcaatatcaagatgcgactgttgaagacgaaggggaatttgaaggtgaagataccaatcaagcaactgttgaacaagaataaaaaa

SEQ ID NO: 471 - DNA, 27 bp, Meloidogyne incognita
gagttggtcgacaatgttttggatgtt

SEQ ID NO: 472 - DNA, 47 bp, Meloidogyne incognita
gccaagaatatgatggctgcttgtgatccacgccatggacgctattt

SEQ ID NO: 473 - DNA, 23 bp, Meloidogyne incognita
gcgaccttcattggcaactccac

SEQ ID NO: 474 - DNA, 26 bp, Meloidogyne incognita
gctgttcttgttgacttggagccggg

SEQ ID NO: 475 - DNA, 29 bp, Meloidogyne incognita
gttccgtcgcctaaggtctctgacacggt

SEQ ID NO: 476 - DNA, 37 bp, Meloidogyne incognita
gttgaacaagaataaaaaagagagaagtttttgtttt

SEQ ID NO: 477 - DNA, 21 bp, Meloidogyne incognita
taattacccaagtttgagttt

SEQ ID NO: 478 - DNA, 22 bp, Meloidogyne incognita
taattacccaagtttgagtttt

SEQ ID NO: 479 - DNA, 23 bp, Meloidogyne incognita
taattacccaagtttgagtttta

SEQ ID NO: 480 - DNA, 26 bp, Meloidogyne incognita
tctgttcatcaacttgttgagaacac

SEQ ID NO: 481 - DNA, 84 bp, Meloidogyne incognita
tggaactggctctggaatgggaactttgctcatttcaaagattcgtgaggagtatccggaccgcattatgtcttccttctctgt

SEQ ID NO: 482 - DNA, 29 bp, Meloidogyne incognita
ttattttgcttttaaccgcatatttccga

SEQ ID NO: 483 - DNA, 33 bp, Meloidogyne incognita
ttattttgcttttaaccgcatatttccgaaata

SEQ ID NO: 484 - DNA, 32 bp, Meloidogyne incognita
ttccttcactggtacactggcgaaggtatgga

SEQ ID NO: 485 - DNA, 26 bp, Meloidogyne incognita
gaatggattccaaacaacgttaagac

SEQ ID NO: 486 - DNA, 23 bp, Meloidogyne incognita
ttcatgcctggtttcgctccact

SEQ ID NO: 487 - DNA, 38 bp, Meloidogyne incognita
ttggctgtcaacatggttccattcccacgtcttcactt

SEQ ID NO: 488 - DNA, 23 bp, Meloidogyne incognita
gttgttcgaaaagaggctgaggg

SEQ ID NO: 489 - DNA, 23 bp, Meloidogyne incognita
aggtttaattacccaagtttgag

SEQ ID NO: 490 - DNA, 23 bp, Meloidogyne incognita
tactttgttgaatggattccaaa

SEQ ID NO: 491 - DNA, 26 bp, Meloidogyne incognita
actctttctgttcatcaacttgttga

SEQ ID NO: 492 - DNA, 28 bp, Meloidogyne incognita
catactttgttgaatggattccaaacaa

SEQ ID NO: 493 - DNA, 22 bp, Meloidogyne incognita
caaagattcgtgaggagtatcc

SEQ ID NO: 494 - DNA, 24 bp, Meloidogyne incognita
aatatgatggctgcttgtgatcca

SEQ ID NO: 495 - DNA, 25 bp, Meloidogyne incognita
catactttgttgaatggattccaaa

SEQ ID NO: 496 - DNA, 23 bp, Meloidogyne incognita
tggtacactggcgaaggtatgga

SEQ ID NO: 497 - DNA, 24 bp, Meloidogyne incognita
aatatgatggctgcttgtgatcca

SEQ ID NO: 498 - DNA, 25 bp, Meloidogyne incognita
catactttgttgaatggattccaaa

SEQ ID NO: 499 - DNA, 22 bp, Meloidogyne incognita
tactttgttgaatggattccaa

SEQ ID NO: 500 - DNA, 22 bp, Meloidogyne incognita
agcaaatgtttgatgccaagaa

SEQ ID NO: 501 - DNA, 22 bp, Meloidogyne incognita
ccaagttctgggaagtcatttc

SEQ ID NO: 502 - DNA, 30 bp, Meloidogyne incognita; identical to SEQ ID NO: 410

SEQ ID NO: 503 - DNA, 23 bp, Meloidogyne incognita
ggtatggacgagatggaattcac

SEQ ID NO: 504 - DNA, 23 bp, Meloidogyne incognita
atggacgagatggaattcactga

SEQ ID NO: 505 - DNA, 26 bp, Meloidogyne incognita
atggacgagatggaattcactgaagc

SEQ ID NO: 506 - DNA, 33 bp, Meloidogyne incognita
cttggtggtggaactggctctggaatgggaact

SEQ ID NO: 507 - DNA, 23 bp, Meloidogyne incognita
gatgccaagaatatgatggctgc

SEQ ID NO: 508 - DNA, 23 bp, Meloidogyne incognita
gcaactctttctgttcatcaact

SEQ ID NO: 509 - DNA, 21 bp, Meloidogyne incognita
ggaactggctctggaatggga

SEQ ID NO: 510 - DNA, 21 bp, Meloidogyne incognita
ggttccattcccacgtcttca

SEQ ID NO: 511 - DNA, 24 bp, Meloidogyne incognita
gtcaacatggttccattcccacgt

SEQ ID NO: 512 - DNA, 26 bp, Meloidogyne incognita
atggacgagatggaattcactgaagc

SEQ ID NO: 513 - DNA, 35 bp, Meloidogyne incognita; identical to SEQ ID NO: 365

SEQ ID NO: 514 - DNA, 25 bp, Meloidogyne incognita
ttaattacccaagtttgagttttac

SEQ ID NO: 515 - DNA, 21 bp, Meloidogyne incognita
ttattatttcaaaaaaaaaaa

SEQ ID NO: 516 - DNA, 23 bp, Meloidogyne incognita
tacggccaggacgtattgatcgt

SEQ ID NO: 517 - DNA, 32 bp, Meloidogyne incognita
gatatggatgaagatgaattggaaatgttatc

SEQ ID NO: 518 - DNA, 24 bp, Meloidogyne incognita
cagtgtttggaaagatatgaatat

SEQ ID NO: 519 - DNA, 23 bp, Meloidogyne incognita
gatatggatgaagatgaattgga

SEQ ID NO: 520 - DNA, 32 bp, Meloidogyne incognita; identical to SEQ ID NO: 517

SEQ ID NO: 521 - DNA, 21 bp, Meloidogyne incognita
acaaattcaagaaattaagga

SEQ ID NO: 522 - DNA, 22 bp, Meloidogyne incognita
acaaattcaagaaattaaggag

SEQ ID NO: 523 - DNA, 22 bp, Meloidogyne incognita
tttaaaaaaatcttttaaaaat

SEQ ID NO: 524 - DNA, 22 bp, Meloidogyne incognita
cacgcagttgttggtgttttgg

SEQ ID NO: 525 - DNA, 26 bp, Meloidogyne incognita
gtggaattgccacttacacatccaga

SEQ ID NO: 526 - DNA, 21 bp, Meloidogyne incognita
caaaagcacattgacttctcc

SEQ ID NO: 527 - DNA, 23 bp, Meloidogyne incognita
cacattgacttctccacaaaatc

SEQ ID NO: 528 - DNA, 21 bp, Meloidogyne incognita
cgtaaagctgctcgtgaactt

SEQ ID NO: 529 - DNA, 23 bp, Meloidogyne incognita
cgtcgtttggttcgtattggagt

SEQ ID NO: 530 - DNA, 29 bp, Meloidogyne incognita
gactttttggagcgtcgtttgcagacgca

SEQ ID NO: 531 - DNA, 26 bp, Meloidogyne incognita
gttttcaaattgggattggctaaatc

SEQ ID NO: 532 - DNA, 23 bp, Meloidogyne incognita
ttcaaattgggattggctaaatc

SEQ ID NO: 533 - DNA, 26 bp, Meloidogyne incognita
gactttttggagcgtcgtttgcagac

SEQ ID NO: 534 - DNA, 23 bp, Meloidogyne incognita
ttggagcgtcgtttgcagacgca

SEQ ID NO: 535 - DNA, 23 bp, Meloidogyne incognita
atcattgccaatgatcaaggaaa

SEQ ID NO: 536 - DNA, 23 bp, Meloidogyne incognita
atgaagcattggccgttcaaggt

SEQ ID NO: 537 - DNA, 25 bp, Meloidogyne incognita
attgcttatggtttggataagaaag

SEQ ID NO: 538 - DNA, 21 bp, Meloidogyne incognita
atttgacgatccagctgttca

SEQ ID NO: 539 - DNA, 23 bp, Meloidogyne incognita
cagcatggaaaagtcgagatcat

SEQ ID NO: 540 - DNA, 23 bp, Meloidogyne incognita
catggaaaagtcgagatcattgc

SEQ ID NO: 541 - DNA, 23 bp, Meloidogyne incognita
gacatgaagcattggccgttcaa

SEQ ID NO: 542 - DNA, 23 bp, Meloidogyne incognita
ggtgtattccagcatggaaaagt

SEQ ID NO: 543 - DNA, 21 bp, Meloidogyne incognita
attgcttatggtttggataag

SEQ ID NO: 544 - DNA, 26 bp, Meloidogyne incognita
gagatcattgccaatgatcaaggaaa

SEQ ID NO: 545 - DNA, 26 bp, Meloidogyne incognita
gagatcattgccaatgatcaaggaaa

SEQ ID NO: 546 - DNA, 22 bp, Meloidogyne incognita
tcggaattgatctgggaacgac

SEQ ID NO: 547 - DNA, 21 bp, Meloidogyne incognita
tttaaaaaaatcttttaaaaa

SEQ ID NO: 548 - DNA, 21 bp, Meloidogyne incognita
gaaatttaaagaatgacaaag

SEQ ID NO: 549 - DNA, 26 bp, Meloidogyne incognita
aattgggccaaaggacattacactga

SEQ ID NO: 550 - DNA, 23 bp, Meloidogyne incognita
cgcaaggctttccttcactggta

SEQ ID NO: 551 - DNA, 29 bp, Meloidogyne incognita
cgcaaggctttccttcactggtacactgg

SEQ ID NO: 552 - DNA, 22 bp, Meloidogyne incognita
gcaaggctttccttcactggta

SEQ ID NO: 553 - DNA, 26 bp, Meloidogyne incognita
gccatgttccgtcgcaaggctttcct

SEQ ID NO: 554 - DNA, 23 bp, Meloidogyne incognita
tttgttgaatggattccaaacaa

SEQ ID NO: 555 - DNA, 24 bp, Meloidogyne incognita
gtcaacatggttccattcccacgt

SEQ ID NO: 556 - DNA, 23 bp, Meloidogyne incognita
aaattggctgtcaacatggttcc

SEQ ID NO: 557 - DNA, 26 bp, Meloidogyne incognita
aacaattgggccaaaggacattacac

SEQ ID NO: 558 - DNA, 21 bp, Meloidogyne incognita
aacatggttccattcccacgt

SEQ ID NO: 559 - DNA, 23 bp, Meloidogyne incognita
aacatggttccattcccacgtct

SEQ ID NO: 560 - DNA, 26 bp, Meloidogyne incognita
aaggctttccttcactggtacactgg

SEQ ID NO: 561 - DNA, 22 bp, Meloidogyne incognita
agcaaatgtttgatgccaagaa

SEQ ID NO: 562 - DNA, 23 bp, Meloidogyne incognita
atcaatgtttactacaatgaggc

SEQ ID NO: 563 - DNA, 23 bp, Meloidogyne incognita
atgatggctgcttgtgatccacg

SEQ ID NO: 564 - DNA, 21 bp, Meloidogyne incognita
atggaattcactgaagccgag

SEQ ID NO: 565 - DNA, 26 bp, Meloidogyne incognita
atggttccattcccacgtcttcactt

SEQ ID NO: 566 - DNA, 21 bp, Meloidogyne incognita
atgttccgtcgcaaggctttc

SEQ ID NO: 567 - DNA, 21 bp, Meloidogyne incognita
atgttccgtcgcaaggctttc

SEQ ID NO: 568 - DNA, 21 bp, Meloidogyne incognita
atgttccgtcgcaaggctttc

SEQ ID NO: 569 - DNA, 29 bp, Meloidogyne incognita
caattgaacgctgatcttcgcaaattggc

SEQ ID NO: 570 - DNA, 21 bp, Meloidogyne incognita
catggttccattcccacgtct

SEQ ID NO: 571 - DNA, 22 bp, Meloidogyne incognita
cgtatttttttaattttatttt

SEQ ID NO: 572 - DNA, 24 bp, Meloidogyne incognita
ctgcatcgacaacgaggctttgta

SEQ ID NO: 573 - DNA, 22 bp, Meloidogyne incognita
cttactgcatcgacaacgaggc

SEQ ID NO: 574 - DNA, 32 bp, Meloidogyne incognita
gaaggtatggacgagatggaattcactgaagc

SEQ ID NO: 575 - DNA, 23 bp, Meloidogyne incognita
gagatggaattcactgaagccga

SEQ ID NO: 576 - DNA, 26 bp, Meloidogyne incognita
gatgccaagaatatgatggctgcttg

SEQ ID NO: 577 - DNA, 23 bp, Meloidogyne incognita
gccattcaagaactgttcaaacg

SEQ ID NO: 578 - DNA, 38 bp, Meloidogyne incognita
ggcgaaggtatggacgagatggaattcactgaagccga

SEQ ID NO: 579 - DNA, 23 bp, Meloidogyne incognita
ggtatggacgagatggaattcac

SEQ ID NO: 580 - DNA, 22 bp, Meloidogyne incognita
gttgtgattgtcttcaaggctt

SEQ ID NO: 581 - DNA, 21 bp, Meloidogyne incognita
tactgcatcgacaacgaggct

SEQ ID NO: 582 - DNA, 22 bp, Meloidogyne incognita
tactgcatcgacaacgaggctt

SEQ ID NO: 583 - DNA, 23 bp, Meloidogyne incognita
tactgcatcgacaacgaggcttt

SEQ ID NO: 584 - DNA, 23 bp, Meloidogyne incognita
tactttgttgaatggattccaaa

SEQ ID NO: 585 - DNA, 21 bp, Meloidogyne incognita
tccattcccacgtcttcactt

SEQ ID NO: 586 - DNA, 33 bp, Meloidogyne incognita
tccattcccacgtcttcactttttcatgcctgg

SEQ ID NO: 587 - DNA, 31 bp, Meloidogyne incognita
tggctgtcaacatggttccattcccacgtct

SEQ ID NO: 588 - DNA, 23 bp, Meloidogyne incognita
tggtacactggcgaaggtatgga

SEQ ID NO: 589 - DNA, 26 bp, Meloidogyne incognita
tggtacactggcgaaggtatggacga

SEQ ID NO: 590 - DNA, 22 bp, Meloidogyne incognita
tgtacgacatctgcttccgaac

SEQ ID NO: 591 - DNA, 21 bp, Meloidogyne incognita
ttccgtggacgtatgtcaatg

SEQ ID NO: 592 - DNA, 21 bp, Meloidogyne incognita
tttttaattttattttgcttt

SEQ ID NO: 593 - DNA, 29 bp, Meloidogyne incognita
aaggctttccttcactggtacactggcga

SEQ ID NO: 594 - DNA, 25 bp, Meloidogyne incognita
aaggtatggacgagatggaattcac

SEQ ID NO: 595 - DNA, 24 bp, Meloidogyne incognita
aatatgatggctgcttgtgatcca

SEQ ID NO: 596 - DNA, 22 bp, Meloidogyne incognita
aatttgaaggtgaagataccaa

SEQ ID NO: 597 - DNA, 26 bp, Meloidogyne incognita
accttcattggcaactccacggccat

SEQ ID NO: 598 - DNA, 32 bp, Meloidogyne incognita
tcatactttgttgaatggattccaaacaacgt

SEQ ID NO: 599 - DNA, 23 bp, Meloidogyne incognita
atggacgagatggaattcactga

SEQ ID NO: 600 - DNA, 38 bp, Meloidogyne incognita
atggttccattcccacgtcttcactttttcatgcctgg

SEQ ID NO: 601 - DNA, 21 bp, Meloidogyne incognita
atgttccgtcgcaaggctttc

SEQ ID NO: 602 - DNA, 24 bp, Meloidogyne incognita
caaattggttccaagttctgggaa

SEQ ID NO: 603 - DNA, 26 bp, Meloidogyne incognita
cgcaaggctttccttcactggtacac

SEQ ID NO: 604 - DNA, 22 bp, Meloidogyne incognita
cggccatgttccgtcgcaaggc

SEQ ID NO: 605 - DNA, 21 bp, Meloidogyne incognita
ctttgttgaatggattccaaa

SEQ ID NO: 606 - DNA, 21 bp, Meloidogyne incognita
gacaactttgtctttggccag

SEQ ID NO: 607 - DNA, 21 bp, Meloidogyne incognita
atgttccgtcgcaaggctttc

SEQ ID NO: 608 - DNA, 23 bp, Meloidogyne incognita
gccattcaagaactgttcaaacg

SEQ ID NO: 609 - DNA, 21 bp, Meloidogyne incognita
gccgagtccaacatgaacgac

SEQ ID NO: 610 - DNA, 23 bp, Meloidogyne incognita
ggcgaaggtatggacgagatgga

SEQ ID NO: 611 - DNA, 26 bp, Meloidogyne incognita
gtcaacatggttccattcccacgtct

SEQ ID NO: 612 - DNA, 21 bp, Meloidogyne incognita
atgttccgtcgcaaggctttc

SEQ ID NO: 613 - DNA, 21 bp, Meloidogyne incognita
tactgcatcgacaacgaggct

SEQ ID NO: 614 - DNA, 21 bp, Meloidogyne incognita
tactgcatcgacaacgaggct

SEQ ID NO: 615 - DNA, 21 bp, Meloidogyne incognita
tactgcatcgacaacgaggct

SEQ ID NO: 616 - DNA, 29 bp, Meloidogyne incognita
tcatactttgttgaatggattccaaacaa

SEQ ID NO: 617 - DNA, 22 bp, Meloidogyne incognita
tggagccgggaaccatggactc

SEQ ID NO: 618 - DNA, 22 bp, Meloidogyne incognita
tggagccgggaaccatggactc

SEQ ID NO: 619 - DNA, 25 bp, Meloidogyne incognita
tggctgtcaacatggttccattccc

SEQ ID NO: 620 - DNA, 26 bp, Meloidogyne incognita
tgggccaaaggacattacactgaggg

SEQ ID NO: 621 - DNA, 26 bp, Meloidogyne incognita
tgggccaaaggacattacactgaggg

SEQ ID NO: 622 - DNA, 21 bp, Meloidogyne incognita
ttccgtggacgtatgtcaatg

SEQ ID NO: 623 - DNA, 21 bp, Meloidogyne incognita
ttttactatttaatttatcta

SEQ ID NO: 624 - DNA, 32 bp, Meloidogyne incognita; identical to SEQ ID NO: 598

SEQ ID NO: 625 - DNA, 21 bp, Meloidogyne incognita
attgatatggatgaagatgaa

SEQ ID NO: 626 - DNA, 22 bp, Meloidogyne incognita
aacaaattcaagaaattaagga

SEQ ID NO: 627 - DNA, 22 bp, Meloidogyne incognita
ttcgtcgtttggttcgtattgg

SEQ ID NO: 628 - DNA, 27 bp, Meloidogyne incognita
attgcttatggtttggataagaaaggc

SEQ ID NO: 629 - DNA, 21 bp, Meloidogyne incognita
ggaacgtctgattggtgatgc

SEQ ID NO: 630 - DNA, 23 bp, Meloidogyne incognita
gagatcattgccaatgatcaagg

SEQ ID NO: 631 - DNA, 23 bp, Meloidogyne incognita
aactcttccgtcctgacaacttt

SEQ ID NO: 632 - DNA, 22 bp, Meloidogyne incognita
agcaaatgtttgatgccaagaa

SEQ ID NO: 633 - DNA, 26 bp, Meloidogyne incognita
ggtatggacgagatggaattcactga

SEQ ID NO: 634 - DNA, 26 bp, Meloidogyne incognita
atcgacaacgaggctttgtacgacat

SEQ ID NO: 635 - DNA, 29 bp, Meloidogyne incognita
atcgacaacgaggctttgtacgacatctg

SEQ ID NO: 636 - DNA, 32 bp, Meloidogyne incognita
atggacgagatggaattcactgaagccgagtc

SEQ ID NO: 637 - DNA, 21 bp, Meloidogyne incognita
caatgtggtaaccaaattggt

SEQ ID NO: 638 - DNA, 23 bp, Meloidogyne incognita
aactcttccgtcctgacaacttt

SEQ ID NO: 639 - DNA, 22 bp, Meloidogyne incognita
catactttgttgaatggattcc

SEQ ID NO: 640 - DNA, 24 bp, Meloidogyne incognita
cctgacaactttgtctttggccag

SEQ ID NO: 641 - DNA, 26 bp, Meloidogyne incognita
gacacggtcgttgagccctacaacgc

SEQ ID NO: 642 - DNA, 21 bp, Meloidogyne incognita
gcaaatgtttgatgccaagaa

SEQ ID NO: 643 - DNA, 29 bp, Meloidogyne incognita
gccatgttccgtcgcaaggctttccttca

SEQ ID NO: 644 - DNA, 21 bp, Meloidogyne incognita
gccgagtccaacatgaacgac

SEQ ID NO: 645 - DNA, 22 bp, Meloidogyne incognita
ggtatggacgagatggaattca

SEQ ID NO: 646 - DNA, 21 bp, Meloidogyne incognita
ggaaacaattgggccaaagga

SEQ ID NO: 647 - DNA, 24 bp, Meloidogyne incognita
aggtatggacgagatggaattcac

SEQ ID NO: 648 - DNA, 23 bp, Meloidogyne incognita
atggacgagatggaattcactga

SEQ ID NO: 649 - DNA, 21 bp, Meloidogyne incognita
atgttccgtcgcaaggctttc

SEQ ID NO: 650 - DNA, 26 bp, Meloidogyne incognita
ggtatggacgagatggaattcactga

SEQ ID NO: 651 - DNA, 23 bp, Meloidogyne incognita
taccagcaatatcaagatgcgac

SEQ ID NO: 652 - DNA, 26 bp, Meloidogyne incognita
tgcatcgacaacgaggctttgtacga

SEQ ID NO: 653 - DNA, 29 bp, Meloidogyne incognita
tgggccaaaggacattacactgagggagc

SEQ ID NO: 654 - DNA, 26 bp, Meloidogyne incognita
ttggctgtcaacatggttccattccc

SEQ ID NO: 655 - DNA, 22 bp, Meloidogyne incognita
ttggtggtggaactggctctgg

SEQ ID NO: 656 - DNA, 21 bp, Meloidogyne incognita
caatgtggtaaccaaattggt

SEQ ID NO: 657 - DNA, 21 bp, Meloidogyne incognita
gagtccaacatgaacgacttg

SEQ ID NO: 658 - DNA, 23 bp, Meloidogyne incognita
gccatgttccgtcgcaaggcttt

SEQ ID NO: 659 - DNA, 24 bp, Meloidogyne incognita
gccatgttccgtcgcaaggctttc

SEQ ID NO: 660 - DNA, 24 bp, Meloidogyne incognita
gccgagtccaacatgaacgacttg

SEQ ID NO: 661 - DNA, 23 bp, Meloidogyne incognita
ggtatggacgagatggaattcac

SEQ ID NO: 662 - DNA, 22 bp, Meloidogyne incognita
ttccgtcctgacaactttgtct

(19) United States
(12) Patent Application Publication
(10) Pub. No.: US 2010/0068172 A1
(43) Pub. Date: Mar. 18, 2010

(54) Nematode Control

(75) Inventor: Marc Van De Craen, Aalter (BE)

Correspondence Address: WOLF GREENFIELD & SACKS, P.C., 600 ATLANTIC AVENUE, BOSTON, MA, 02210-2206, US

(73) Assignee: Devgen N.V., Zwijnaarde (BE)

(21) Appl. No.: 12/225,207

(22) PCT Filed: Mar. 16, 2007
(86) PCT No.: PCT/EP2007/002327

§ 371 Date: Aug. 28, 2009

Related U.S. Application Data
(60) Provisional application No. 60/783,533, filed on Mar. 16, 2006.

(51) Int. Cl. A01N 25/00; A01H 5/00; A01P 5/00; C12N 15/82; C07H 21/00; C12N 5/04; C07K 14/00; C08B 31/00; A01N 33/00; C12N 1/00; C12N 1/21; C12N 1/19; A01N 63/00; A01N 37/18; A01P 19/00; A23L 1/36; A23L 1/0522; A23L 1/10; A23J 1/14; A23K 1/14; A23D 9/00; A23F 5/00; A23F 3/00

(52) U.S. Cl. 424/84; 800/301; 800/302; 800/279; 536/23.1; 435/419; 530/350; 536/102; 514/44 R; 435/243; 435/252.3; 435/254.2; 424/93.2; 514/12; 426/629; 426/549; 426/555; 426/656; 426/630; 426/601; 426/594; 426/597

(57) ABSTRACT

The present invention concerns methods for controlling nematode infestation via dsRNA mediated gene silencing, whereby the intact nematodes are contacted with a double-stranded RNA and whereby the double-stranded RNA is taken up by the intact nematodes. In one particular embodiment, the methods of the invention are used to alleviate plants from nematode pests. Alternatively, the methods are used for treating and/or preventing nematode infestation on a substrate or a subject in need of such treatment and/or prevention. Suitable nematode target genes and fragments thereof, dsRNA constructs, recombinant constructs and compositions are disclosed.

### FIELD OF THE INVENTION

The present invention relates to the field of double-stranded RNA (dsRNA) mediated gene silencing in nematode species. More particularly, the present invention relates to genetic constructs designed for the expression of dsRNA corresponding to novel target genes. These constructs are particularly useful in dsRNA-mediated nematode pest control. The invention further relates to methods for controlling nematodes, methods for preventing nematode infestation and methods for down-regulating gene expression in nematodes using dsRNA. The invention also relates to transgenic plants resistant to nematode infestation.

### BACKGROUND OF THE INVENTION

Nematodes comprise a large phylum of animals that include plant and animal parasites as well as many free living species (Maggenti, A. (1981). General Nematology. (New-York: Springer-Verlag)). Plant parasitic nematodes reduce annual U.S. agricultural production by more than $5 billion. Plant parasitic nematodes are obligate parasites, obtaining nutrition only from the cytoplasm of the living plant cells. Some plant parasitic nematodes are ectoparasites, living outside the host. Other species spend much of their lives inside roots as migratory or sedentary endoparasites. It is the sedentary endoparasites of the family Heteroderidae that cause the most economic damage worldwide.

The Heteroderidae can be divided into two groups: the cyst nematodes, which include the genera Heterodera and Globodera; and the root-knot nematodes—genus Meloidogyne. The life cycle of cyst and root-knot nematodes passes through a series of four juvenile stages, separated by molts, during which the cuticle is replaced. The infective stage is the motile, second-stage juvenile that penetrates the plant root to establish a permanent feeding site. After feeding is initiated, the nematode becomes sedentary and then undergoes three molts during development to the adult stage. Interactions between nematodes and their plant host are complex, generally last more than a month and result in major morphological and developmental changes in both organisms.

Root-knot nematodes (RKN: Meloidogyne spp.), so-called for the characteristic root galls or knots formed on the root, infect thousands of plant species and cause severe losses in yield of many food and fiber crops through the world (Mai, W. F. (1985). “Plant parasitic nematodes: Their threat to agriculture” In: An Advanced Treatise on Meloidogyne, Vol. 1, J. N. Sasser and C. C. Carter, eds. (Raleigh: North Carolina State University Graphics), pp. 11-17).

The root knot nematode Meloidogyne incognita (hereinafter M. incognita) is widespread in tropical and subtropical regions and can reproduce on more than 700 host plants including most cultivated crops and ornamentals (Sasser, J. N., (1980) Root-knot nematodes: A global menace to crop production, Plant Disease, 64: 36-41). M. incognita causes significant damage to cotton as a single pest problem and as part of the Fusarium wilt disease complex. M. incognita also causes varying degrees of damage to tomato, potato and tobacco.

Plants infected with root-knot nematodes are at a greater risk of contracting other diseases that are detrimental to its health. Fusarium oxysporum f. sp. Vasinfectum and the root-knot nematode, Meloidogyne incognita, have been associated with “frenching” or Fusarium wilt of cotton Gossypium hirsutum. Giant cells form in the host roots as the juveniles feed and as females mature they cause the roots to split and crack providing an entry into the host for the fungus. The fungus moves through the plant into the vascular system which becomes clogged reducing the transportation of water and nutrients through the xylem tissue. This in turn causes the host plant to loose its vigor and wilt.

Nematicides are widely used in M. incognita management but may, however, be less effective when nematodes are embedded in plant tissue. Because of the broad host range, crop rotation is frequently not feasible. Natural host-plant resistance genes to M. incognita occur in plant genera, such as clovers, cotton, peach, peanut, pineapple, corn, sweet potato, tobacco and tomatoes, and may be incorporated into another plant to protect it against M. incognita. However, there are many crops for which appropriate resistance loci are not available and it is uncertain that nematode resistance genes, even if it is possible to transfer them to a different species or line, will function effectively in the new host plant. Furthermore, acquisition of virulence resulting in a breakdown of the resistance offered by such a gene by M. incognita may shorten the effective utility of this approach.

To effectively control M. incognita infestation, novel strategies to engineer synthetic resistance in plants are becoming imperative.

An alternative biological agent is dsRNA. Over the last few years, downregulation of genes (also referred to as “gene silencing”) in multicellular organisms by means of RNA interference or “RNAi” has become a well-established technique.

RNA interference or “RNAi” is a process of sequence-specific down-regulation of gene expression (also referred to as “gene silencing” or “RNA-mediated gene silencing”) initiated by double-stranded RNA (dsRNA) that is complementary in sequence to a region of the target gene to be down-regulated (Fire, A. Trends Genet. Vol. 15, 358-363, 1999; Sharp, P. A. Genes Dev. Vol. 15, 485-490, 2001).

Over the last few years, down-regulation of target genes in multicellular organisms by means of RNA interference (RNAi) has become a well established technique. Reference may be made to International applications WO 99/32619 (Carnegie Institution) and WO 00/01846 (by applicant).

DsRNA gene silencing finds application in many different areas, such as for example dsRNA mediated gene silencing in clinical applications (WO2004/001013) and in plants. In plants, dsRNA constructs useful for gene silencing have also been designed to be cleaved and to be processed into short interfering RNAs (siRNAs).

RNAi has been proposed as a means of protecting plants against plant parasitic nematodes, i.e. by expressing in the plant (e.g. in the entire plant, or in a part, tissue or cell of a plant) one or more nucleotide sequences that form a dsRNA fragment that corresponds to a target gene in the plant parasitic nematode that is essential for its growth, reproduction and/or survival. Reference may be made to the International application WO 00/01846 (by applicant) and U.S. Pat. No. 6,506,559 (based on WO 99/32619). Experiments using particular genes have been reported to show some reduction in nematode reproduction (WO04/005485). However, complete efficacy was not observed.

The understanding of the biology of miRNAs and siRNAs has opened up new methods of gene silencing in the pharmaceutical and agricultural fields. Key to the application of this technique to the problem of nematode infection of plants is the observation that when nematodes ingest dsRNA molecules, these may be absorbed by the digestive tract. Silencing of genes with sequences homologous to the RNA molecules then occurs. This has been demonstrated in the model nematode C. elegans. Not only can RNA molecules be ingested, but feeding the nematodes E. coli cells harboring recombinant constructs producing such RNA molecules results in absorption of the E. coli-produced RNA molecules and silencing of homologous genes (WO 00/01846). This, combined with the availability of the complete genome sequence of C. elegans, has made possible high throughput functional genomics programs, testing the phenotypic effect of silencing essentially every gene from the genome one by one. This makes possible the assembly of a list of genes which, when silenced, result in death or lack of reproduction of the worm.

With plant parasitic nematodes such experiments are more difficult since these are obligate parasites, meaning that they normally will feed only in its natural state, that is, when infecting a susceptible plant root.

The present invention provides target genes and constructs useful in the dsRNA-mediated nematode pest control, especially the control of Meloidogyne pests.

### DESCRIPTION OF THE INVENTION

The present inventors have shown that using certain chemical treatments root-knot nematodes are able to ingest molecules in vitro such as FITC and dsRNA (outside of the root). Absorption of RNA molecules from the digestive tract leading to silencing of target genes opens up a possible new strategy for M. incognita control.

The present invention describes a novel non-compound, non-protein based approach for the control of nematode crop pests. The active ingredient is a nucleic acid, a double-stranded ribonucleic acid (dsRNA), which can be used as a nematicidal formulation, for example, as a foliar spray. In another embodiment, the dsRNA can be expressed constitutively in the host plant to protect the plant against infesting nematodes. The sequence of the dsRNA corresponds to part or whole of an essential nematode gene and causes downregulation of the nematode target via RNA interference (RNAi). As a result of the downregulation of mRNA, the dsRNA prevents expression of the target nematode protein and hence causes death, growth arrest or sterility of the nematode.

In a first embodiment, the present invention thus relates to a double stranded ribonucleotide sequence produced from the expression of a polynucleotide sequence comprising a nucleic acid sequence selected from the group comprising:

(i) sequences represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof,

(ii) sequences which are at least 70%, preferably at least 75%, 80%, 85%, 90%, more preferably at least 95%, 96%, 97%, 98% or 99% identical to a sequence represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof, and

(iii) sequences comprising at least 17, preferably at least 18, 19, 20 or 21, more preferably at least 22, 23 or 24 contiguous nucleotides of any of the sequences represented by SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof,

or wherein said nucleic acid sequence is an orthologue of a gene comprising at least 17, preferably at least 18, 19, 20 or 21, more preferably at least 22, 23 or 24 contiguous nucleotides of any of SEQ ID NOs 61 to 662, or a complement thereof,

wherein ingestion of said ribonucleotide sequence by a pathogenic pest inhibits the growth of said pest. In preferred aspects of the invention, this pathogenic pest is a nematode pest. The double stranded RNA(s) is (are) used in several aspects of the invention.

The methods of the invention can find practical application in any area of technology where it is desirable to inhibit viability, growth, development or reproduction of the nematode, or to decrease pathogenicity or infectivity of the nematode. The methods of the invention further find practical application where it is desirable to specifically down-regulate expression of one or more target genes in a nematode. Particularly useful practical applications include, but are not limited to, (1) protecting plants against plant pathogenic nematodes; (2) pharmaceutical or veterinary use in humans and animals (for example to control, treat or prevent nematode infections in humans); (3) protecting materials against damage caused by nematodes; (4) protecting perishable materials (such as foodstuffs, seed, etc.) against damage caused by nematodes; (5) functional genomics in nematodes to elucidate the gene function of nematode target genes and generally any application wherein nematodes need to be controlled and/or wherein damage caused by nematodes needs to be prevented.

In accordance with one embodiment the invention relates to a method for controlling nematode growth in or on a cell or an organism or for preventing nematode infestation of a cell or an organism susceptible to nematode infection, comprising contacting nematodes with a double-stranded RNA, wherein the double-stranded RNA comprises annealed complementary strands, one of which has a nucleotide sequence which is complementary to at least part of the nucleotide sequence of a nematode target gene, whereby the double-stranded RNA is taken up by the nematode and thereby controls growth or prevents infestation.

The expression “double-stranded RNA”, “double stranded RNA” and “double stranded ribonucleotide sequence” are used interchangeable herein.

“Controlling pests” as used in the present invention means killing pests, or preventing pests to develop, or to grow or preventing pests to infect or infest. Controlling pests as used herein also encompasses controlling pest progeny (development of eggs). Controlling pests as used herein also encompasses inhibiting viability, growth, development or reproduction of the pest, or to decrease pathogenicity or infectivity of the pest. The compounds and/or compositions described herein, may be used to keep an organism healthy and may be used curatively, preventively or systematically to control pests or to avoid pest growth or development or infection or infestation. Particular pests envisaged in the present invention are plant pathogenic nematode pests. “Controlling nematodes” as used herein thus also encompasses controlling nematode progeny (such as development of eggs). Controlling nematodes as used herein also encompasses inhibiting viability, growth, development or reproduction of the nematode, or decreasing pathogenicity or infectivity of the nematode. In the present invention, controlling nematodes may inhibit a biological activity in a nematode, resulting in one or more of the following attributes: reduction in feeding by the nematode, reduction in viability of the nematode, death of the nematode, inhibition of differentiation and development of the nematode, absence of or reduced capacity for sexual reproduction by the nematode, muscle formation, juvenile hormone formation, juvenile hormone regulation, ion regulation and transport, maintenance of cell membrane potential, amino acid biosynthesis, amino acid degradation, sperm formation, pheromone synthesis, pheromone sensing, antennae formation, wing formation, leg formation, development and differentiation, egg formation, larval maturation, digestive enzyme formation, haemolymph synthesis, haemolymph maintenance, neurotransmission, cell division, energy metabolism, respiration, apoptosis, and any component of a eukaryotic cells' cytoskeletal structure, such as, for example, actins and tubulins. The compounds and/or compositions described herein, may be used to keep an organism healthy and may be used curatively, preventively or systematically to control a nematode or to avoid nematode growth or development or infection or infestation. Thus, the invention may allow previously susceptible organisms to develop resistance against infestation by the nematode organism.

The expression “to at least part of” as used herein means that the nucleotide sequence is fully complementary to the nucleotide sequence of the target over more than two nucleotides, for instance over at least 15, 16, 17, 18, 19, 20, 21, 22, 23 or 24 contiguous nucleotides.

According to a further embodiment, the invention relates to a method for down-regulating expression of a target gene in a nematode, comprising contacting said nematode with a double-stranded RNA, wherein the double-stranded RNA comprises annealed complementary strands, one of which has a nucleotide sequence which is complementary to at least part of the nucleotide sequence of the nematode target gene to be down-regulated, whereby the double-stranded RNA is taken up into the nematode and thereby down-regulates expression of the nematode target gene.

Preferably, the present invention extends to methods as described herein, wherein said nematode target gene comprises a sequence which is selected from the group comprising:

(i) sequences which are at least 75%, at least 80% or 85% identical, preferably at least 90%, 95%, 96%, or more preferably at least 97%, 98% and still more preferably at least 99% identical to a sequence represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof, and

(ii) sequences comprising at least 17, preferably at least 18, 19, 20 or 21, more preferably at least 22, 23 or 24 contiguous nucleotides of any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof, or wherein said target gene is an orthologue of a gene comprising at least 17 contiguous nucleotides of any of SEQ ID NOs 61 to 662, or the complement thereof.

Whenever the term “a” is used within the context of “a target gene”, this means “at least one” target gene. The same applies for “a” target organism meaning “at least one” target organism, and “a” RNA molecule or host cell meaning “at least one” RNA molecule or host cell. This is also detailed further below.

According to one embodiment, the methods of the invention rely on uptake by the nematode of double-stranded RNA present outside of the nematode (e.g. by feeding) and does not require expression of double-stranded RNA within cells of the nematode. In addition, the present invention also encompasses methods as described above wherein the nematode is contacted with a composition comprising the double-stranded RNA.

The methods of the invention are applicable to all nematodes that are susceptible to gene silencing by RNA interference and that are capable of internalising double-stranded RNA from their immediate environment.

In terms of “susceptible organisms”, which benefit from the present invention, any organism which is susceptible to pest infestation is included.

In preferred, but non-limiting, embodiments of the invention the nematode is chosen from the group consisting of:

(1) a nematode which is a plant pathogenic nematode, such as but not limited to Root Knot Nematodes (Meloidogyne spp.) in rice (e.g. M. incognita, M. javanica or M. graminicola), in soybean (e.g. M. incognita or M. arenaria), in cotton (e.g. M. incognita), in potato (e.g. M. chitwoodi or M. hapla), in tomato (e.g. M. chitwoodi), in tobacco (e.g. M. incognita, M. javanica or M. arenaria), and in corn (e.g. M. incognita); Cyst Nematodes (Heterodera spp.) in rice (e.g. H. oryzae), in soybean (e.g. H. glycines) and in corn (e.g. H. zeae); Cyst nematodes (Globodera spp.) in potato (e.g. G. pallida or G. rostochiensis); Reniform Nematodes (Rotylenchulus spp.) in cotton (e.g. R. reniformis); Root lesion nematodes (Pratylenchus spp.) in banana (e.g. P. coffeae or P. goodeyi); Burrowing Nematodes (Radopholus spp.) in banana (e.g. R. similis); Other rice damaging nematodes such as rice root nematode (Hirschmaniella spp., e.g. H. oryzae);

(2) a nematode capable of infesting humans such as, but not limited to: Enterobius vermicularis, the pinworm that causes enterobiasis; Ascaris lumbridoides, the large intestinal roundworm that causes ascariasis; Necator and Ancylostoma, two types of hookworms that cause ancylostomiasis; Trichuris trichiura, the whipworm that causes trichuriasis; Strongyloides stercoralis that causes strongyloidiasis; and Trichonella spirae that causes trichinosis; Brugia malayi and Wuchereria bancrofti, the filarial nematodes associated with the worm infections known as lymphatic filariasis and its gross manifestation, elephantiasis, and Onchocerca volvulus that causes river blindness. Transfer of nematodes to humans may also occur through blood-feeding mosquitoes which have fed upon infected animals or humans;

(3) a nematode capable of infesting animals such as, but not limited to: dogs (Hookworms e.g. Ancylostoma caninum or Uncinaria stenocephala, Ascarids e.g. Toxocara canis or Toxascaris leonina, or Whipworms e.g. Trichuris vulpis), cats (Hookworms e.g. Ancylostoma tubaeforme, Ascarids e.g. Toxocara cati), fish (herring worms or cod worms e.g. Anisakid, or tapeworm e.g. Diphyllobothrium), sheep (Wire worms e.g. Haemonchus contortus) and cattle (Gastro-intestinal worms e.g. Ostertagia ostertagi, Cooperia oncophora);

(4) a nematode that causes unwanted damage to substrates or materials, such as nematodes that attack foodstuffs, seeds, wood, paint, plastic, clothing etc. Examples of such nematodes include but are not limited to Meloidogyne spp. (e.g. M. incognita, M. javanica, M. arenaria, M. graminicola, M. chitwoodi or M. hapla); Heterodera spp. (e.g. H. oryzae, H. glycines, H. zeae or H. schachtii); Globodera spp. (e.g. G. pallida or G. rostochiensis); Ditylenchus spp. (e.g. D. dipsaci, D. destructor or D. angustus); Belonolaimus spp.; Rotylenchulus spp. (e.g. R. reniformis); Pratylenchus spp. (e.g. P. coffeae, P. goodeyi or P. zeae); Radopholus spp. (e.g. R. Similis); Hirschmaniella spp. (e.g. H. oryzae); Aphelenchoides spp. (e.g. A. besseyi); Criconemoides spp.; Longidorus spp.; Helicotylenchus spp.; Hoplolaimus spp.; Xiphinema spp.; Paratrichodorus spp. (e.g. P. minor); Tylenchorhynchus spp;

(5) virus transmitting nematodes (e.g. Longidorus macrosoma: transmits prunus necrotic ring spot virus, Xiphinema americanum: transmits tobacco ring spot virus, Paratrichadorus teres: transmits pea early browning virus, or Trichodorus similis: transmits tobacco rattle virus).

Preferably plants may benefit from the present invention by protection from infestation by plant pathogenic organisms.

In a preferred embodiment the susceptible organism is a plant and the pest is a plant pathogenic nematode. In this embodiment the nematode is contacted with the RNA molecule by expressing the dsRNA molecule in a plant, plant part, plant cell or plant seed that is infested with or susceptible to infestation with the plant pathogenic pest.

According to one embodiment, the methods of the invention rely on a GMO approach wherein the double-stranded RNA is expressed by a cell or an organism infested with or susceptible to infestation by pathogenic pests, preferably plant pathogenic nematode pests. Preferably, said cell is a plant cell or said organism is a plant.

In this context the term “plant” encompasses any plant material that it is desired to treat to prevent or reduce nematode growth and/or nematode infestation. This includes, inter alia, whole plants, seedlings, propagation or reproductive material such as seeds, cuttings, grafts, explants, etc. and also plant cell and tissue cultures. The plant material should express, or have the capability to express, the RNA molecule comprising at least one nucleotide sequence that is the RNA complement of or that represents the RNA equivalent of at least part of the nucleotide sequence of the sense strand of at least one target gene of the pest organism, such that the RNA molecule is taken up by a pest upon plant-pest interaction, said RNA molecule being capable of inhibiting the target gene or down-regulating expression of the target gene by RNA interference.

The invention provides for use of a plant, plant part, plant cell or seed as defined herein for down regulation of expression of a nematode target gene. In more detailed terms, the invention provides for use of a host cell as defined herein and/or an RNA molecule comprising a nucleotide sequence that is the RNA complement of or that represents the RNA equivalent of at least part of the nucleotide sequence of a target gene from a nematode organism, as produced by transcription of a nucleic acid molecule in a plant, plant part, plant cell or seed, for instance in the manufacture of a commodity product, for down regulation of expression of a target gene. Suitable target genes and target organisms in respect of the invention are discussed below in further detail.

Nematodes of the invention are chosen from the group consisting of but not limited to: Meloidogyne spp. (e.g. M. incognita, M. javanica, M. graminicola, M. arenaria, M. chitwoodi, M. hapla or M. paranaensis); Heterodera spp. (e.g. H. oryzae, H. glycines, H. zeae or H. schachtii); Globodera spp. (e.g. G. pallida or G. rostochiensis); Rotylenchulus spp. (e.g. R. reniformis); Pratylenchus spp. (e.g. P. coffeae, P. Zeae or P. goodeyi); Radopholus spp. (e.g. R. similis); Hirschmaniella spp. (e.g. H. oryzae); Ancylostoma spp. (e.g. A. caninum, A. ceylanicum, A. duodenale or A. tubaeforme); Anisakid; Aphelenchoides spp. (e.g. A. Besseyi); Ascarids; Ascaris spp., (e.g. A. suum or A. lumbridoides); Belonolaimus spp.; Brugia spp. (e.g. B. malayi or B. pahangi); Bursaphelenchus spp. Caenorhabditis spp. (e.g. C. elegans, C. briggsae or C. remanei); Clostridium spp. (e.g. C. acetobutylicum); Cooperia spp. (e.g. C. oncophora); Criconemoides spp.; Cyathostomum spp. (e.g. C. catinatum, C. coronatum or C. pateratum); Cylicocyclus spp. (e.g. C. insigne, C. nassatus or C. radiatus); Cylicostephanus spp. (e.g. C. goldi or C. longibursatus); Diphyllobothrium; Dirofilaria spp. (e.g. D. immitis); Ditylenchus spp. (e.g. D. dipsaci, D. destructor or D. Angustus); Enterobius spp. (e.g. E. vermicularis); Haemonchus spp. (e.g. H. contortus); Helicotylenchus spp.; Hoplolaimus spp.; Litomosoides spp. (e.g. L. sigmodontis); Longidorus spp. (e.g. L. macrosoma); Necator spp. (e.g. N. americanus); Nippostrongylus spp. (e.g. N. brasiliensis); Onchocerca spp. (e.g. O. volvulus); Ostertagia spp. (e.g. O. ostertagi); Parastrongyloides spp. (e.g. P. trichosun); Paratrichodorus spp. (e.g. P. minor or P. teres); Parelaphostrongylus spp. (e.g. P. tenuis); Scutellonerna. spp.; Strongyloides spp. (e.g. S. Ratti or S. stercoralis); Teladorsagia spp. (e.g. T. circumcincta); Toxascaris spp. (e.g. T. leonina); Toxocara spp. (e.g. T. canis or T. cati); Trichinella spp. (e.g. T. britovi, T. spiralis or T. spirae); Trichodorus spp. (e.g. T. similis);Trichuris spp. (e.g. T. muris, T. vulpis or T. trichiura); Tylenchulus spp.; Tylenchorhynchus spp.; Uncinaria spp. (e.g. U. stenocephala); Wuchereria spp. (e.g. W. bancrofti); and Xiphinema spp. (e.g. X. Index or X. americanum).

Preferred plant pathogenic nematodes according to the invention include but are not limited to Root Knot Nematodes (Meloidogyne spp.) in rice (e.g. M. incognita, M. javanica or M. graminicola), in soybean (e.g. M. incognita and M. arenaria), in cotton (e.g. M. incognita), in potato (e.g. M. chitwoodi (columbia root-knot nematode), causing small, raised swellings on potato tuber surface, dark specks in the potato flesh and reduced potato quality, or M. hapla (northern root knot nematode)), in tomato (e.g. M. chitwoodi, M. incognita causing root galls), in corn (e.g. M. incognita, causing stunting and chlorosis, numerous root galls and proliferation of fibrous roots), in tobacco, sugarcane, banana and vegetables (e.g. M. javanica) and in peanut (e.g. M. arenaria); Cyst Nematodes (Heterodera spp.) in rice (e.g. H. oryzae), in soybean (e.g. H. glycines, causing yield loss, stunting and chlorosis), in corn (e.g. H. zeae, causing stunting, pale color and narrow leaves) and in sugarbeet (e.g. H. schachtii, causing stunting and yellowing of plants, misshapen and excess fibrous roots); Cyst nematodes (Globodera spp.) in potato, tomato and other Solanum species (e.g. G. pallida (white potato cyst nematode) or G. rostochiensis (golden nematode or yellow potato cyst nematode), causing root damage, poor growth, yellowing and wilting); Stem and bulb nematodes (Ditylenchus spp.) in potato (e.g. D. dipsaci, causing tubers rot; leaves and stems swell and become distorted, or D. destructor (potato rot nematode or potato tuber nematode), causing potato dry rot; tuber rot) and in rice (D. angustus); Sting nematodes (Belonolaimus spp.) in soybean and in corn, causing severe trim of the roots of growing plants or seedlings; Reniform Nematodes (Rotylenchulus spp.) in cotton, maize, cowpea and black gram and banana (e.g. R. reniformis); Root lesion nematodes (Pratylenchus spp.) in banana (e.g. P. coffeae or P. goodeyi), in rice (e.g. P. zeae) and in corn (causing severe pruning of the roots, resulting in stunting, as well as reduction in stalk diameter, stalk and root weights); Burrowing Nematodes (Radopholus spp.) in banana (e.g. R. similis, causing rhizome rot, pepper slow wilt); Other rice damaging nematodes such as rice root nematode (Hirschmaniella spp., e.g. H. oryzae) and rice white tip nematode (Aphelenchoides spp., e.g. A. besseyi); Other corn damaging nematodes such as ring nematodes (Criconemoides spp.), needle nematodes (Longidorus spp.), spiral nematodes (Helicotylenchus spp., causing mild stunting and reduced yields), lance nematodes (Hoplolaimus spp., causing stunting), dagger nematodes (Xiphinema spp.), stubby-root nematodes (Paratrichodorus spp., causing stunting, chlorosis, and reduced yields; e.g. P. minor feeds largely on the root tips, stopping terminal growth of the rootlets, and resulting in the stubby-root effect) and stunt nematodes (Tylenchorhynchus spp., causing stunting).

Preferred plant pathogenic nematodes according to the invention include but are not limited to Meloidogyne spp. (e.g. M. incognita, M. javanica, M. graminicola, M. arenaria, M. chitwoodi, M. hapla or M. paranaensis); Heterodera spp. (e.g. H. oryzae, H. glycines, H. zeae or H. schachtii); Globodera spp. (e.g. G. paffida or G. rostochiensis); Rotylenchulus spp. (e.g. R. reniformis); Pratylenchus spp. (e.g. P. coffeae, P. Zeae or P. goodeyi); Radopholus spp. (e.g. R. similis); Hirschmaniella spp. (e.g. H. oryzae); Aphelenchoides spp. (e.g. A. besseyi); Belonolaimus spp.; Criconemoides spp.; Ditylenchus spp. (e.g. D. dipsaci, D. destructor or D. Angustus); Helicotylenchus spp.; Hoplolaimus spp.; Longidorus spp. (e.g. L. macrosoma); Paratrichodorus spp. (e.g. P. minor or P. teres); Tylenchorhynchus spp.; Xiphinema spp. (e.g. X. Index or X. americanum).

In most preferred embodiments of the invention, the nematode may belong to the family of the Heteroderidae, encompassing the genera Heterodera and Globodera, including the plant pathogenic nematodes Meloidogyne spp. (e.g. M. incognita, M. javanica, M. graminicola, M. arenaria, M. chitwoodi, M. hapla or M. paranaensis) and Heterodera spp. (e.g. H. oryzae, H. glycines, H. zeae or H. schachtii).

The present invention also relates to a method for producing a plant resistant to a plant pathogenic nematode, comprising:

(a) transforming a plant cell with a recombinant construct comprising at least one regulatory sequence operably linked to a sequence complementary to at least part of
(1) a nucleotide sequence of a target nematode gene selected from the group consisting of: (i) sequences which are at least 75%, at least 80% or 85% identical, preferably at least 90%, 95%, 96%, or more preferably at least 97%, 98% and still more preferably at least 99% identical to a sequence represented by any of SEQ ID Nos 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof, (ii) sequences comprising at least 17, preferably at least 18, 19, 20 or 21, more preferably at least 22, 23 or 24 contiguous nucleotides of any of SEQ ID Nos 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof, and (iii) sequences comprising a sense strand comprising a nucleotide sequence of (i) and an antisense strand comprising the complement of said nucleotide sequence of (i), wherein the transcript encoded by said nucleotide sequence is capable of forming a double-stranded RNA, or
(2) a nucleotide sequence which is a nematode orthologue of a gene comprising at least 17, preferably at least 18, 19, 20 or 21, more preferably at least 22, 23 or 24 contiguous nucleotides of any of SEQ ID NOs 95 to 963, or the complement thereof;

(b) regenerating a plant from the transformed plant cell; and

(c) growing the transformed plant under conditions suitable for the expression of the recombinant construct, said grown transformed plant resistant to plant pathogenic nematodes compared to an untransformed plant. According to still other embodiments, in the methods of the invention, the double-stranded RNA is expressed from a recombinant construct, which construct comprises at least one regulatory sequence operably linked to said nucleotide sequence which is complementary to at least part of said nucleotide sequence of said nematode target gene to be down-regulated.

Preferred plants according to the invention include but are not limited to alfalfa, apple, apricot, artichoke, asparagus, avocado, banana, barley, beans, beet, blackberry, blueberry, broccoli, Brussels sprouts, cabbage, canola, carrot, cassaya, cauliflower, a cereal, celery, cherry, citrus, clementine, coffee, corn, cotton, cucumber, eggplant, endive, eucalyptus, figs, grape, grapefruit, groundnuts, ground cherry, kiwifruit, lettuce, leek, lemon, lime, pine, maize, mango, melon, millet, mushroom, nut oat, okra, onion, orange, an ornamental plant or flower or tree, papaya, parsley, pea, peach, peanut, peat, pepper, persimmon, pineapple, plantain, plum, pomegranate, potato, pumpkin, radicchio, radish, rapeseed, raspberry, rice, rye, sorghum, soy, soybean, spinach, strawberry, sugar beet, sugarcane, sunflower, sweet potato, tangerine, tea, tobacco, tomato, a vine, watermelon, wheat, yams, zucchini, Brassica and Arabidopsis; preferably rice, cotton, potato, tomato, corn, banana or soybean.

According to one preferred embodiment the plant is a rice plant. According to another embodiment the plant is a cotton plant. According to another embodiment the plant is a tomato plant. According to another embodiment the plant is a soybean plant. According to another embodiment, the plant is a potato plant. According to another embodiment, the plant is a corn plant. According to another embodiment, the plant is a banana plant.

The term “nematode” encompasses nematodes of all types and at all stages of development, including but not limited to eggs, free living pre-parasitic J2, parasitic J2, parasitic J3, young adults (male and female J4) and mature adults (male and female).

According to one embodiment of the present invention, the nematode which is contacted with the dsRNA is a plant pathogenic nematode in a life stage outside a plant cell, for example in the form of eggs or pre-parasitic J2. According to another embodiment of the present invention, the nematode which is contacted with the dsRNA is a plant pathogenic nematode in a life stage inside a plant cell, for example a pathogenic form of eggs, parasitic J2, parasitic J3, young adults (male and female J4) or mature adults (male and female).

The present invention relates to any gene of interest in the nematode (which may be referred to herein as the “target gene”) that can be down-regulated.

The terms “down-regulation of gene expression” and “inhibition of gene expression” are used interchangeably and refer to a measurable or observable reduction in gene expression or a complete abolition of detectable gene expression, at the level of protein product and/or mRNA product from the target gene. Preferably the down-regulation does not substantially directly inhibit the expression of other genes of the nematode. The down-regulation effect of the dsRNA on gene expression may be calculated as being at least 30%, 40%, 50%, 60%, preferably 70%, 80% or even more preferably 90% or 95% when compared with normal gene expression. Depending on the nature of the target gene, down-regulation or inhibition of gene expression in cells of a nematode can be confirmed by phenotypic analysis of the cell or the whole nematode or by measurement of mRNA or protein expression using molecular techniques such as RNA solution hybridization, PCR, nuclease protection, Northern hybridization, reverse transcription, gene expression monitoring with a microarray, antibody binding, enzyme linked immunosorbent assay (ELISA), Western blotting, radioimmunoassay (RIA), other immunoassays, or fluorescence activated cell analysis (FACS).

The “target gene” may be essentially any gene that is desirable to be inhibited because it interferes with growth or pathogenicity or infectivity of the nematode. For instance if the method of the invention is to be used to prevent nematode growth and/or infestation then it is preferred to select a target gene which is essential for viability, growth, development or reproduction of the nematode, or any gene that is involved with pathogenicity or infectivity of the nematode, such that specific inhibition of the target gene leads to a lethal phenotype or decreases or stops nematode infestation.

According to one non-limiting embodiment, the target gene is such that when its expression is down-regulated or inhibited using the method of the invention, the nematode is killed, or the reproduction or growth of the nematode is stopped or retarded. This type of target genes is considered to be essential for the viability of the nematode and is referred to as essential genes. Therefore, the present invention encompasses a method as described herein, wherein the target gene is an essential gene.

According to a further non-limiting embodiment, the target gene is such that when it is down-regulated using the method of the invention, the infestation or infection by the nematode, the damage caused by the nematode, and/or the ability of the nematode to infest or infect host organisms and/or cause such damage, is reduced. The terms “infest” and “infect” or “infestation” and “infection” are generally used interchangeably throughout. This type of target genes is considered to be involved in the pathogenicity or infectivity of the nematode. Therefore, the present invention extends to methods as described herein, wherein the target gene is involved in the pathogenicity or infectivity of the nematode. The advantage of choosing the latter type of target gene is that the nematode is blocked to infect further plants or plant parts and to form further generations.

According to one embodiment, target genes are conserved genes or nematode -specific genes.

In addition, any suitable double-stranded RNA fragment capable of directing RNAi or RNA-mediated gene silencing or inhibition of a nematode target gene may be used in the methods of the invention.

In the methods of the present invention, dsRNA is used to inhibit growth or to interfere with the pathogenicity or infectivity of the nematode.

The invention thus relates to isolated double-stranded RNA comprising annealed complementary strands, one of which has a nucleotide sequence which is complementary to at least part of a target nucleotide sequence of a target gene of a nematode. The target gene may be any of the target genes described herein, or a part thereof that exerts the same function.

According to one embodiment of the present invention, an isolated double-stranded RNA comprising annealed complementary strands, one of which has a nucleotide sequence which is complementary to at least part of a nucleotide sequence of a nematode target gene, wherein said target gene comprises a sequence which is selected from the group comprising: (i) sequences which are at least 75%, at least 80% or 85% identical, preferably at least 90%, 95%, 96%, or more preferably at least 97%, 98% and still more preferably at least 99% identical to a sequence represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof, and (ii) sequences comprising at least 17, preferably at least 18, 19, 20 or 21, more preferably at least 22, 23 or 24 contiguous nucleotides of any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement, or wherein said target gene is an orthologue of a gene comprising at least 17 contiguous nucleotides of any of SEQ ID NOs 61 to 662, or the complement thereof.

Depending on the assay used to measure gene silencing, the growth inhibition can be quantified as being greater than about 5%, 10%, more preferably about 20%, 25%, 33%, 50%, 60%, 75%, 80%, most preferably about 90%, 95%, or about 99% as compared to a pest organism that has been treated with control dsRNA.

According to another embodiment of the present invention, an isolated double-stranded RNA is provided, wherein at least one of said annealed complementary strands comprises the RNA equivalent of at least one of the nucleotide sequences represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the RNA equivalent of a fragment of at least 17 basepairs in length thereof, preferably at least 18, 19, 20 or 21, more preferably at least 22, 23 or 24 basepairs in length thereof. According to another embodiment of the present invention, an isolated double-stranded RNA is provided comprising annealed complementary strands, one of which has a nucleotide sequence which is complementary to at least part of a nucleotide sequence of a nematode target, for use as a medicament. According to another embodiment of the present invention the use as a medicament is provided for the isolated double-stranded RNA as described above.

If the method of the invention is used for specifically controlling growth or infestation of specific nematodes in or on a host cell or host organism, it is preferred that the double-stranded RNA does not share any significant homology with any host gene, or at least not with any essential gene of the host. In this context, it is preferred that the double-stranded RNA shows less than 30%, more preferably less that 20%, more preferably less than 10%, and even more preferably less than 5% nucleic acid sequence identity with any gene of the host cell. % sequence identity should be calculated across the full length of the double-stranded RNA region. If genomic sequence data is available for the host organism one may cross-check sequence identity with the double-stranded RNA using standard bioinformatics tools. In one embodiment, there is no sequence identity between the dsRNA and a host sequences over 21 contiguous nucleotides, meaning that in this context, it is preferred that 21 contiguous base pairs of the dsRNA do not occur in the genome of the host organism. In another embodiment, there is less than about 10% or less than about 12.5% sequence identity over 24 contiguous nucleotides of the dsRNA with any nucleotide sequence from a host species.

The double-stranded RNA comprises annealed complementary strands, one of which has a nucleotide sequence which corresponds to a target nucleotide sequence of the target gene to be down-regulated. The other strand of the double-stranded RNA is able to base-pair with the first strand.

The expression “target region” or “target nucleotide sequence” of the target nematode gene may be any suitable region or nucleotide sequence of the gene. The target region should comprise at least 17, at least 18 or at least 19 consecutive nucleotides of the target gene, more preferably at least 20 or at least 21 nucleotide and still more preferably at least 22, 23 or 24 nucleotides of the target gene.

It is preferred that (at least part of) the double-stranded RNA will share 100% sequence identity with the target region of the nematode target gene. However, it will be appreciated that 100% sequence identity over the whole length of the double-stranded region is not essential for functional RNA inhibition. RNA sequences with insertions, deletions, and single point mutations relative to the target sequence have also been found to be effective for RNA inhibition. The terms “corresponding to” or “complementary to” are used herein interchangeable, and when these terms are used to refer to sequence correspondence between the double-stranded RNA and the target region of the target gene, they are to be interpreted accordingly, i.e. as not absolutely requiring 100% sequence identity. However, the % sequence identity between the double-stranded RNA and the target region will generally be at least 80% or 85% identical, preferably at least 90%, 95%, 96%, or more preferably at least 97%, 98% and still more preferably at least 99%.

The term “complementary” as used herein relates to both DNA-DNA complementarity as to DNA-RNA complementarity. In analogy herewith, the term “RNA equivalent” substantially means that in the DNA sequence(s), the base “T” may be replaced by the corresponding base “U” normally present in ribonucleic acids.

Although the dsRNA contains a sequence which corresponds to the target region of the target gene it is not absolutely essential for the whole of the dsRNA to correspond to the sequence of the target region. For example, the dsRNA may contain short non-target regions flanking the target-specific sequence, provided that such sequences do not affect performance of the dsRNA in RNA inhibition to a material extent.

The dsRNA may contain one or more substitute bases in order to optimise performance in RNAi. It will be apparent to the skilled reader how to vary each of the bases of the dsRNA in turn and test the activity of the resulting siRNAs (e.g. in a suitable in vitro test system) in order to optimise the performance of a given dsRNA.

The dsRNA may further contain DNA bases, non-natural bases or non-natural backbone linkages or modifications of the sugar-phosphate backbone, for example to enhance stability during storage or enhance resistance to degradation by nucleases.

It has been previously reported that the formation of short interfering RNAs (siRNAs) of about 21 by is desirable for effective gene silencing. However, in applications of applicant it has been shown that the minimum length of dsRNA preferably is at least about 80-100 by in order to be efficiently taken up by certain pest organisms. There are indications that in invertebrates such as the free living nematode C. elegans or the plant parasitic nematode Meloidogyne incognita, these longer fragments are more effective in gene silencing, possibly due to a more efficient uptake of these long dsRNA by the invertebrate.

It has also recently been suggested that synthetic RNA duplexes consisting of either 27-mer blunt or short hairpin (sh) RNAs with 29 by stems and 2-nt 3′ overhangs are more potent inducers of RNA interference than conventional 21-mer siRNAs. Thus, molecules based upon the targets identified above and being either 27-mer blunt or short hairpin (sh) RNA's with 29-bp stems and 2-nt 3′ overhangs are also included within the scope of the invention.

Therefore, in one embodiment, the double-stranded RNA fragment (or region) will itself preferably be at least 17 by in length, preferably 18 or 19 bp in length, more preferably at least 20 bp, more preferably at least 21 bp, or at least 22 bp, or at least 23 bp, or at least 24 bp, 25 bp, 26 by or at least 27 by in length. The expressions “double-stranded RNA fragment” or “double-stranded RNA region” refer to a small entity of the double-stranded RNA corresponding with (part of) the target gene.

Generally, the double-stranded RNA is preferably between about 17-1500 bp, even more preferably between about 80-1000 by and most preferably between about 17-27 by or between about 80-250 bp; such as double-stranded RNA regions of about 17 bp, 18 bp, 19 bp, 20 bp, 21 bp, 22 bp, 23 bp, 24 bp, 25 bp, 27 bp, 50 bp, 80 bp, 100 bp, 150 bp, 200 bp, 250 bp, 300 bp, 350 bp, 400 bp, 450 bp, 500 bp, 550 bp, 600 bp, 650 bp, 700 bp, 900 bp, 100 bp, 1100 bp, 1200 bp, 1300 bp, 1400 by or 1500 bp.

The upper limit on the length of the double-stranded RNA may be dependent on i) the requirement for the dsRNA to be taken up by the nematode and ii) the requirement for the dsRNA to be processed within the cell into fragments that direct RNAi. The chosen length may also be influenced by the method of synthesis of the RNA and the mode of delivery of the RNA to the cell. Preferably the double-stranded RNA to be used in the methods of the invention will be less than 10,000 by in length, more preferably 1000 by or less more preferably 500 by or less, more preferably 300 by or less, more preferably 100 by or less. For any given target gene and nematode, the optimum length of the dsRNA for effective inhibition may be determined by experiment.

The double-stranded RNA may be fully or partially double-stranded. Partially double-stranded RNAs may include short single-stranded overhangs at one or both ends of the double-stranded portion, provided that the RNA is still capable of being taken up by nematodes and directing RNAi. The double-stranded RNA may also contain internal non-complementary regions.

The methods of the invention can encompass the simultaneous or sequential provision of two or more different double-stranded RNAs or RNA constructs to the same nematode, so as to achieve down-regulation or inhibition of multiple target genes or to achieve a more potent inhibition of a single target gene.

Alternatively, multiple targets are hit by the provision of one double-stranded RNA that hits multiple target sequences, and a single target is more efficiently inhibited by the presence of more than one copy of the double-stranded RNA fragment corresponding to the target gene. Thus, in one embodiment of the invention, the double-stranded RNA construct comprises multiple dsRNA regions, at least one strand of each dsRNA region comprising a nucleotide sequence that is complementary to at least part of a target nucleotide sequence of a nematode target gene. According to the invention, the dsRNA regions in the RNA construct may be complementary to the same or to different target genes and/or the dsRNA regions may be complementary to targets from the same or from different nematode species. Use of such dsRNA constructs in a plant host cell, thus establishes a more potent resistance to a single or to multiple nematode species in the plant.

The terms “hit”, “hits” and “hitting” are alternative wordings to indicate that at least one of the strands of the dsRNA is complementary to, and as such may bind to, the target gene or nucleotide sequence.

In one embodiment, the double-stranded RNA region comprises multiple copies of the nucleotide sequence that is complementary to the target gene. Alternatively, the dsRNA hits more than one target sequence of the same target gene. The invention thus encompasses isolated double-stranded RNA constructs comprising at least two copies of said nucleotide sequence complementary to at least part of a nucleotide sequence of a nematode target. In another embodiment the invention relates to an isolated double stranded RNA construct comprising at least two copies of the RNA equivalent of at least one of the nucleotide sequences represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or comprising at least two copies of the RNA equivalent of a fragment of at least 17 basepairs in length thereof, preferably at least 18, 19, 20 or 21, more preferably at least 22, 23 or 24 basepairs in length thereof. Preferably, said isolated double stranded RNA construct comprises at least two copies of the RNA equivalent of the nucleotide sequence as represented in SEQ ID NOs 1, 5, 6, 8, 12, 14, 18, 20, 24, 26, 28, 29, 31, 33, 35, 37, 39, 41, 43, 45, 46, 49, 51, 52, 54, 55, 57 or 59, or a fragment of at least 17, preferably at least 18, 19, 20 or 21, more preferably at least 22, 23 or 24 basepairs in length thereof.

The term “multiple” in the context of the present invention means at least two, at least three, at least four, at least five, at least six, etc.

The expressions “a further target gene” or “at least one other target gene” mean for instance a second, a third or a fourth, etc. target gene.

DsRNA that hits more than one of the above-mentioned targets, or a combination of different dsRNA against different of the above mentioned targets are developed and used in the methods of the present invention.

Accordingly the invention relates to an isolated double stranded RNA or RNA construct comprising the RNA equivalents of at least two nucleotide sequences independently chosen from the sequences represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or fragments thereof of at least 17 basepairs in length, preferably at least 18, 19, 20 or 21, more preferably at least 22, 23 or 24 basepairs in length thereof.

Accordingly, the present invention extends to methods as described herein, wherein the dsRNA comprises annealed complementary strands, one of which has a nucleotide sequence which is complementary to at least part of a target nucleotide sequence of an nematode target gene, and which comprises the RNA equivalents of at least two nucleotide sequences independently chosen from each other. In one embodiment, the dsRNA comprises the RNA equivalents of at least two, preferably at least three, four or five, nucleotide sequences independently chosen from the sequences represented by any of SEQ ID Nos 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or fragments thereof of at least 17 basepairs in length, preferably at least 18, 19, 20 or 21, more preferably at least 22, 23 or 24 basepairs in length thereof.

The at least two nucleotide sequences may be derived from the target genes herein described. According to one preferred embodiment the dsRNA hits at least one target gene that is essential for viability, growth, development or reproduction of the nematode and hits at least one gene involved in pathogenicity or infectivity as described hereinabove. Alternatively, the dsRNA hits multiple genes of the same category, for example, the dsRNA hits at least 2 essential genes or at least 2 genes involved in the same cellular function. According to a further embodiment, the dsRNA hits at least 2 target genes, which target genes are involved in a different cellular function. For example the dsRNA hits two or more genes involved in protein synthesis (e.g. ribosome subunits), intracellular protein transport, nuclear mRNA splicing, or involved in one of the functions described in Table 1.

The dsRNA regions (or fragments) in the double-stranded RNA may be combined as follows:
a) when multiple dsRNA regions targeting a single target gene are combined, they may be combined in the original order (ie the order in which the regions appear in the target gene) in the RNA construct,
b) alternatively, the original order of the fragments may be ignored so that they are scrambled and combined randomly or deliberately in any order into the double-stranded RNA construct,
c) alternatively, one single fragment may be repeated several times, for example from 1 to 10 times, e.g. 1, 2, 3, 4, 5, 6, 7, 8, 9 or 10 times, in the ds RNA construct, or
d) the dsRNA regions (targeting a single or different target genes) may be combined in the sense or antisense orientation.

In addition, the target gene(s) to be combined may be chosen from one or more of the following categories of genes:
e) “essential” genes or “pathogenicity genes” as described above encompass genes that are vital for one or more target nematodes and result in a lethal or severe (e.g. feeding, reproduction, growth) phenotype when silenced. The choice of a strong lethal target gene results in a potent RNAi effect. In the RNA constructs of the invention, multiple dsRNA regions targeting the same or different (very effective) lethal genes can be combined to further increase the potency, efficacy or speed of the RNAi effect in nematode control.
f) “weak” genes encompass target genes with a particularly interesting function in one of the cellular pathways described herein, but which result in a weak phenotypic effect when silenced independently. In the RNA constructs of the invention, multiple dsRNA regions targeting a single or different weak gene(s) may be combined to obtain a stronger RNAi effect.
g) “nematode specific” genes encompass genes that have no substantial homologous counterpart in non-nematode organisms as can be determined by bioinformatics homology searches, for example by BLAST searches. The choice of a nematode specific target gene results in a species specific RNAi effect, with no effect or no substantial (adverse) effect in non-target organisms.
h)“conserved genes” encompass genes that are conserved (at the amino acid level) between the target organism and non-target organism(s). To reduce possible effects on non-target species, such effective but conserved genes are analysed and target sequences from the variable regions of these conserved genes are chosen to be targeted by the dsRNA regions in the RNA construct. Here, conservation is assessed at the level of the nucleic acid sequence. Such variable regions thus encompass the least conserved sections, at the level of the nucleic acid sequence, of the conserved target gene(s).
i) “conserved pathway” genes encompass genes that are involved in the same biological pathway or cellular process, or encompass genes that have the same functionality in different nematode species resulting in a specific and potent RNAi effect and more efficient nematode control;
j) alternatively, the RNA constructs according to the present invention target multiple genes from different biological pathways, resulting in a broad cellular RNAi effect and more efficient nematode control.

According to the invention, all double-stranded RNA regions comprise at least one strand that is complementary to at least part or a portion of the nucleotide sequence of any of the target genes herein described. According to the invention, there is provided an isolated double-stranded RNA or RNA construct, which comprises at least one additional dsRNA region, at least one strand thereof comprising a nucleotide sequence which is complementary to at least part of a nucleotide sequence of at least one other nematode target gene.

However, provided that one of the double-stranded RNA regions comprises at least one strand that is complementary to a portion of the nucleotide sequence of any one of the target genes herein described, the other double-stranded RNA regions may comprise at least one strand that is complementary to a portion of any other nematode target gene (including known target genes).

The invention also relates to an isolated double-stranded RNA or RNA construct comprising at least two nucleotide sequences chosen from the group of sequences represented in any of SEQ ID NOs 29, 51 or 54. In a preferred embodiment, these double-stranded RNA or RNA construct(s) comprise a nucleotide sequence as represented in SEQ ID NO 59.

According to yet another embodiment of the present invention, there is provided an isolated double-stranded RNA or RNA construct, further comprising at least one additional sequence and optionally a linker. In one embodiment, the additional sequence is chosen from the group comprising (i) a sequence facilitating large-scale production of the dsRNA construct; (ii) a sequence effecting an increase or decrease in the stability of the dsRNA; (iii) a sequence allowing the binding of proteins or other molecules to facilitate uptake of the RNA construct by nematodes; (iv) a sequence which is an aptamer that binds to a receptor or to a molecule on the surface or in the cytoplasm of a nematode to facilitate uptake, endocytosis and/or transcytosis by the nematode; or (v) one or more additional sequences to catalyze processing of dsRNA regions. In one embodiment, the linker is a conditionally self-cleaving RNA sequence, preferably a pH sensitive linker or a hydrophobic sensitive linker. In one embodiment, the linker is an intron. In one embodiment of the present invention, there is provided an isolated double-stranded RNA or RNA construct for use as a medicament.

In one embodiment, the multiple dsRNA regions of the double-stranded RNA construct are connected by one or more linkers. In another embodiment, the linker is present at a site in the RNA construct, separating the dsRNA regions from another region of interest. Different linker types for the dsRNA constructs are provided by the present invention.

In another embodiment, the multiple dsRNA regions of the double-stranded RNA construct are connected without linkers.

In a particular embodiment of the invention, the linkers may be used to disconnect smaller dsRNA regions in the pest organism. Advantageously, in this situation the linker sequence may promote division of a long dsRNA into smaller dsRNA regions under particular circumstances, resulting in the release of separate dsRNA regions under these circumstances and leading to more efficient gene silencing by these smaller dsRNA regions. Examples of suitable conditionally self-cleaving linkers are RNA sequences that are self-cleaving at high pH conditions. Suitable examples of such RNA sequences are described by Borda et al. (Nucleic Acids Res. 2003 May 15; 31(10):2595-600), which document is incorporated herein by reference. This sequence originates from the catalytic core of the hammerhead ribozyme HH16.

In another aspect of the invention, a linker is located at a site in the RNA construct, separating the dsRNA regions from another, e.g. the additional, sequence of interest, which preferably provides some additional function to the RNA construct.

In one particular embodiment of the invention, the dsRNA constructs of the present invention are provided with an aptamer to facilitate uptake of the dsRNA by the nematode. The aptamer is designed to bind a substance which is taken up by the nematode. Such substances may be from a nematode or plant origin. One specific example of an aptamer, is an aptamer that binds to a transmembrane protein, for example a transmembrane protein of a nematode. Alternatively, the aptamer may bind a (plant) metabolite or nutrient which is taken up by the nematode.

Alternatively, the linkers are self-cleaving in the endosomes. This may be advantageous when the constructs of the present invention are taken up by the nematode via endocytosis or transcytosis, and are therefore compartmentalized in the endosomes of the nematode species. The endosomes may have a low pH environment, leading to cleavage of the linker.

The above mentioned linkers that are self-cleaving in hydrophobic conditions are particularly useful in dsRNA constructs of the present invention when used to be transferred from one cell to another via the transit in a cell wall, for example when crossing the cell wall of a nematode pest organism.

An intron may also be used as a linker. An “intron” as used herein may be any non-coding RNA sequence of a messenger RNA. Particular suitable intron sequences for the constructs of the present invention are (1) U-rich (35-45%); (2) have an average length of 100 by (varying between about 50 and about 500 bp) which base pairs may be randomly chosen or may be based on known intron sequences; (3) start at the 5′ end with -AG:GT- or -CG:GT- and/or (4) have at their 3′ end -AG:GC- or -AG:AA.

A non-complementary RNA sequence, ranging from about 1 base pair to about 10,000 base pairs, may also be used as a linker.

Without wishing to be bound by any particular theory or mechanism, it is thought that long double-stranded RNAs are taken up by the nematode from their immediate environment. Double-stranded RNAs taken up into the gut and transferred to the gut epithelial cells are then processed within the cell into short double-stranded RNAs, called small interfering RNAs (siRNAs), by the action of an endogenous endonuclease. The resulting siRNAs then mediate RNAi via formation of a multi-component RNase complex termed the RISC or RNA interfering silencing complex.

In order to achieve down-regulation of a target gene within a nematode cell the double-stranded RNA added to the exterior of the cell wall may be any dsRNA or dsRNA construct that can be taken up into the cell and then processed within the cell into siRNAs, which then mediate RNAi, or the RNA added to the exterior of the cell could itself be an siRNA that can be taken up into the cell and thereby direct RNAi.

siRNAs are generally short double-stranded RNAs having a length in the range of from 19 to 25 base pairs, or from 20 to 24 base pairs. In preferred embodiments siRNAs having 19, 20, 21, 22, 23, 24 or 25 base pairs, and in particular 21 or 22 base pairs, corresponding to the target gene to be down-regulated may be used. However, the invention is not intended to be limited to the use of such siRNAs.

siRNAs may include single-stranded overhangs at one or both ends, flanking the double-stranded portion. In a particularly preferred embodiment the siRNA may contain 3′ overhanging nucleotides, preferably two 3′ overhanging thymidines (dTdT) or uridines (UU). 3′ TT or UU overhangs may be included in the siRNA if the sequence of the target gene immediately upstream of the sequence included in double-stranded part of the dsRNA is AA. This allows the TT or UU overhang in the siRNA to hybridise to the target gene. Although a 3′ TT or UU overhang may also be included at the other end of the siRNA it is not essential for the target sequence downstream of the sequence included in double-stranded part of the siRNA to have AA. In this context, siRNAs which are RNA/DNA chimeras are also contemplated. These chimeras include, for example, the siRNAs comprising a double-stranded RNA with 3′ overhangs of DNA bases (e.g. dTdT), as discussed above, and also double-stranded RNAs which are polynucleotides in which one or more of the RNA bases or ribonucleotides, or even all of the ribonucleotides on an entire strand, are replaced with DNA bases or deoxynucleotides.

The dsRNA may be formed from two separate (sense and antisense) RNA strands that are annealed together by (non-covalent) basepairing. Alternatively, the dsRNA may have a foldback stem-loop or hairpin structure, wherein the two annealed strands of the dsRNA are covalently linked. In this embodiment the sense and antisense stands of the dsRNA are formed from different regions of single polynucleotide molecule that is at least partially self-complementary. RNAs having this structure are convenient if the dsRNA is to be synthesised by expression in vivo, for example in a host cell or organism as discussed below, or by in vitro transcription. The precise nature and sequence of the “loop” linking the two RNA strands is generally not material to the invention, except that it should not impair the ability of the double-stranded part of the molecule to mediate RNAi. The features of “hairpin” or “stem-loop” RNAs for use in RNAi are generally known in the art (see for example WO 99/53050, in the name of CSIRO, the contents of which are incorporated herein by reference). In other embodiments of the invention, the loop structure may comprise linker sequences or additional sequences as described above.

The double-stranded RNA or construct may be prepared in a manner known per se. For example, double-stranded RNAs may be synthesised in vitro using chemical or enzymatic RNA synthesis techniques well known in the art. In one approach the two separate RNA strands may be synthesised separately and then annealed to form double-strands. In a further embodiment, double-stranded RNAs or constructs may be synthesised by intracellular expression in a host cell or organism from a suitable expression vector. This approach is discussed in further detail below.

The amount of double-stranded RNA with which the nematode is contacted is such that specific down-regulation of the one or more target genes is achieved; such an amount is an effective amount for down-regulating genes. The RNA may be introduced in an amount which allows delivery of at least one copy per cell. However, in certain embodiments higher doses (e.g., at least 5, 10, 100, 500 or 1000 copies per cell) of double-stranded RNA may yield more effective inhibition. For any given nematode gene target the optimum amount of dsRNA for effective inhibition may be determined by routine experimentation.

The nematode can be contacted with the double-stranded RNA in any suitable manner, permitting direct uptake of the double-stranded RNA by the nematode. For example, the nematode can be contacted with the double-stranded RNA in pure or substantially pure form, for example an aqueous solution containing the dsRNA. In this embodiment, the nematode may be simply “soaked” with an aqueous solution comprising the double-stranded RNA. In a further embodiment the nematode can be contacted with the double-stranded RNA by spraying the nematode with a liquid composition comprising the double-stranded RNA.

Alternatively, the double-stranded RNA may be linked to a food component of the nematodes, such as a food component for a pathogenic nematode, in order to increase uptake of the dsRNA by the nematode.

In other embodiments the nematode may be contacted with a composition containing the double-stranded RNA. The composition may, in addition to the dsRNA, contain further excipients, diluents or carriers. Preferred features of such compositions are discussed in more detail below.

The double-stranded RNA may also be incorporated in the medium in which the nematode grows or in or on a material or substrate that is infested by the nematode or impregnated in a substrate or material susceptible to infestation by nematode.

In a further aspect of the invention, said double-stranded RNA may be expressed by a prokaryotic (for instance but not limited to a bacterial) or eukaryotic (for instance but not limited to a yeast or a plant) host cell or host organism and the prokaryotic or eukaryotic cell is taken up or eaten by the nematode species.

As illustrated in the examples, bacteria can be engineered to produce any of the dsRNA or dsRNA constructs of the invention. These bacteria can be eaten by the nematode species. When taken up, the dsRNA can initiate an RNAi response, leading to the degradation of the target mRNA and weakening or killing of the feeding nematode.

According to these embodiments, any bacterium or yeast cell that is capable of expressing dsRNA or dsRNA constructs can be used. The bacterium is chosen from the group comprising Gram-negative and Gram-positive bacteria, such as, but not limited to, Escherichia spp. (e.g. E. coli), Bacillus spp. (e.g. B. thuringiensis), Rhizobium spp., Lactobacilllus spp., Lactococcus spp., etc. The yeast may be chosen from the group comprising Saccharomyces spp., etc.

Some bacteria have a very close interaction with the host plant, such as, but not limited to, symbiotic Rhizobium with the Legminosea (for example Soy). Such recombinant bacteria could be mixed with the seeds (for instance as a coating) and used as soil improvers.

Accordingly, the present invention also encompasses a cell comprising any of the nucleotide sequences or recombinant DNA constructs described herein. The invention further encompasses prokaryotic cells (such as, but not limited to, gram-positive and gram-negative bacterial cells) and eukaryotic cells (such as, but not limited to, yeast cells or plant cells). Preferably said cell is a bacterial cell or a yeast cell or an algal cell.

In other embodiments the nematode may be contacted with a composition as described further herein. The composition may, in addition to the dsRNA or DNA contain further excipients, diluents or carriers. Preferred features of such compositions are discussed in more detail below.

Alternatively, dsRNA producing bacteria or yeast cells can be sprayed directly onto the crops.

Thus, as described above, the invention provides a host cell comprising an RNA construct and/or a DNA construct and/or an expression construct of the invention. Preferably, the host cell is a bacterial or yeast cell, but may be a virus for example. A virus may be utilised which specifically infects nematodes. This ensures safety for mammals, especially humans, since the virus will not infect the mammal, so no unwanted RNAi effect will occur.

The bacterial cell or yeast cell preferably should be inactivated before being utilised as a biological pesticide, for instance when the agent is to be used in an environment where contact with humans or other mammals is likely (such as a kitchen). Inactivation may be achieved by any means, such as by heat treatment, phenol or formaldehyde treatment for example, or by mechanical treatment.

Possible applications include intensive greenhouse cultures, for instance crops that are less interesting from a GMO point of view, as well as broader field crops such as soy.

This approach has several advantages, eg: since the problem of possible dicing by a plant host is not present, it allows the delivery of large dsRNA fragments into the gut lumen of the feeding pest; the use of bacteria as nematicides does not involve the generation of transgenic crops, especially for certain crops where transgenic variants are difficult to obtain; there is a broad and flexible application in that different crops can be simultaneously treated on the same field and/or different pests can be simultaneously targeted, for instance by combining different bacteria producing distinct dsRNAs.

The amount of targeted RNA which is taken up, preferably by ingestion, by the target organism is such that specific down-regulation of the one or more target genes is achieved. When the RNA is expressed by a bacterial or fungal host cell, an amount may be applied which allows delivery of at least one copy per host cell. However, in certain embodiments higher doses (e.g., at least 5, 10, 100, 500 or 1000 copies per cell of the target organism) of RNA may yield more effective inhibition. For any given target gene and target organism the optimum amount of the targeted RNA molecules for effective inhibition may be determined by routine experimentation.

The target organism can be contacted with the host cell expressing the RNA molecule in any suitable manner, to permit ingestion by the target organism. Preferably, the host cells expressing the dsRNA may be linked to a food component of the target organisms in order to increase uptake of the dsRNA by the target organism. The host cells expressing the dsRNA may also be incorporated in the medium in which the target organism grows or in or on a material or substrate that is infested by a pest organism or impregnated in a substrate or material susceptible to infestation by a pest organism.

In alternative embodiments, a suitable extract derived from the host cells expressing the RNA molecule may be utilised in order to achieve down regulation of a target gene in a target organism. Here, the extracts may be derived by any suitable means of lysis of the host cells expressing the RNA molecules. For example, techniques such as sonication, French press, freeze-thaw and lysozyme treatment (see Sambrook and Russell—Molecular Cloning: A laboratory manual—third edition and the references provided therein in table 15-4) may be utilised in order to prepare a crude host cell extract (lysate). Further purification of the extract may be carried out as appropriate provided the ability of the extract to mediate targeted down regulation of target gene expression is not adversely affected. Affinity purification may be utilised for example. It may also be appropriate to add certain components to the extract, to prevent degradation of the RNA molecules. For example, RNase inhibitors may be added to the extracts derived from the host cells expressing the RNA. In one example, the target organism can be contacted with the host cell expressing the RNA in pure or substantially pure form, for example an aqueous solution containing the cell extract. In this embodiment, the target organism, especially pest organisms such as nematodes may be simply “soaked” with an aqueous solution comprising the host cell extract. In a further embodiment the nematode can be contacted with the host cells expressing the RNA molecule by spraying the nematode, or the soil containing the nematodes with a liquid composition comprising the cell extract.

Another aspect of the present invention are target nucleotide sequences of the nematode target genes herein disclosed. Such target nucleotide sequences are particularly important to design the dsRNA constructs according to the present invention. Such target nucleotide sequences are preferably at least 17, preferably at least 18, 19, 20 or 21, more preferably at least 22, 23 or 24 nucleotides in length. Non-limiting examples of preferred target nucleotide sequences are given in the examples.

According to one embodiment, the present invention provides an isolated nucleotide sequence encoding a double-stranded RNA or double-stranded RNA construct as described herein.

According to a more specific embodiment, the present invention relates to an isolated nucleic acid sequence consisting of a sequence represented by any of SEQ ID NOs 61 to 662, or a fragment of at least 17 preferably at least 18, 19, 20 or 21, more preferably at least 22, 23 or 24 nucleotides thereof.

According to yet another embodiment, the present invention provides nematode target genes, which comprise a sequence as herein represented by SEQ ID No 1, 5, 6, 8, 12, 14, 18, 20, 24, 26, 28, 29, 31, 33, 35, 37, 39, 41, 43, 45, 46, 49, 51, 52, 54, 55, 57, 59, or a fragment thereof of at least 17, preferably at least 18, 19, 20 or 21, more preferably at least 22, 23 or 24 nucleotides thereof, and which target genes can be used in the methods of the present invention.

According to a more specific embodiment, the present invention relates to an isolated nucleic acid sequence consisting of a sequence represented by any of SEQ ID Nos 7, 13, 19, 25, 30, 34, 38, 42, 47, 48, 58 or 60, or the complement thereof, or a fragment of at least 17 preferably at least 18, 19, or 21, more preferably at least 22, 23 or 24 nucleotides thereof, or the complement thereof.

A person skilled in the art will recognize that homologues of these target genes can be found and that these homologues are also useful in the methods of the present invention.

Protein, or nucleotide sequences are likely to be homologous if they show a “significant” level of sequence similarity or more preferably sequence identity. Truly homologous sequences are related by divergence from a common ancestor gene. Sequence homologues can be of two types: (i) where homologues exist in different species they are known as orthologues. e.g. the a-globin genes in mouse and human are orthologues.(ii) paralogues are homologous genes in within a single species. e.g. the α- and β-globin genes in mouse are paralogues.

Preferred homologues are genes comprising a sequence which is at least about 85% or 87.5%, still more preferably about 90%, still more preferably at least about 95% and most preferably at least about 99% identical to a sequence selected from the group of sequences represented by SEQ ID Nos 1, 5, 6, 8, 12, 14, 18, 20, 24, 26, 28, 29, 31, 33, 35, 37, 39, 41, 43, 45, 46, 49, 51, 52, 54, 55, 57 or 59, or the complement thereof. Methods for determining sequence identity are routine in the art and include use of the Blast software and EMBOSS software (The European Molecular Biology Open. Software Suite (2000), Rice, P. Longden, I. and Bleasby, A. Trends in Genetics 16, (6) pp 276-277). The term “identity” as used herein refers to the relationship between sequences at the nucleotide level. The expression “% identical” is determined by comparing optimally aligned sequences, e.g. two or more, over a comparison window wherein the portion of the sequence in the comparison window may comprise insertions or deletions as compared to the reference sequence for optimal alignment of the sequences. The reference sequence does not comprise insertions or deletions. The reference window is chosen from between at least 10 contiguous nucleotides to about 50, about 100 or to about 150 nucleotides, preferably between about 50 and 150 nucleotides. “% identity” is then calculated by determining the number of nucleotides that are identical between the sequences in the window, dividing the number of identical nucleotides by the number of nucleotides in the window and multiplying by 100.

Other homologues are genes which are alleles of a gene comprising a sequence as represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57 and 59. Further preferred homologues are genes comprising at least one single nucleotide polymorphism (SNP) compared to a gene comprising a sequence as represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57 and 59. Further preferred homologues are genes comprising at least one single nucleotide polymorphism (SNP) compared to a gene comprising a sequence as represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57 and 59.

According to another embodiment, the invention encompasses target genes which are nematode orthologues of a gene comprising a nucleotide sequence as represented in any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57 and 59. By way of example, orthologues may comprise a nucleotide sequence as represented in any of SEQ ID NOs 61 to 614, or a fragment of at least 17, 18, 19, 20, 21, 22, 23, 24, 25, 26 or 27 nucleotides thereof. The invention thus encompasses any of the methods described herein for controlling nematode growth on a cell or an organism, or for preventing nematode infestation of a cell or an organism susceptible to nematode infection, comprising contacting nematodes with a double-stranded RNA, wherein the double-stranded RNA comprises annealed complementary strands, one of which has a nucleotide sequence which is complementary to at least part of the nucleotide sequence of a target gene comprising a fragment of at least 17, 18, 19, 20 or 21 nucleotides of any of the sequences as represented in SEQ ID NOs 61 to 514, whereby the double-stranded RNA is taken up by the nematode and thereby controls growth or prevents infestation. The invention also relates to nematode-resistant transgenic plants comprising a fragment of at least 17, 18, 19, 20 or 21 nucleotides of any of the sequences as represented in SEQ ID NOs 61 to 514. A non-limiting list of nematode orthologues genes of sequences comprising at least a fragment of 17 by of one of the sequences of the invention is given in Table 6.

According to another embodiment, the invention encompasses target genes which are insect or arachnida orthologues of a gene comprising a nucleotide sequence as represented in any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57 and 59. By way of example, orthologues may comprise a nucleotide sequence as represented in any of SEQ ID NOs 515 to 629, or a fragment of at least 17, 18, 19, 20, 21, 22, 23, 24, 25, 26 or 27 nucleotides thereof. According to another aspect, the invention thus encompasses any of the methods described herein for controlling insect or arachnida growth on a cell or an organism, or for preventing insect or arachnida infestation of a cell or an organism susceptible to insect or arachnida infection, comprising contacting insects with a double-stranded RNA, wherein the double-stranded RNA comprises annealed complementary strands, one of which has a nucleotide sequence which is complementary to at least part of the nucleotide sequence of a target gene comprising a fragment of at least 17, 18, 19, 20 or 21 nucleotides of any of the sequences as represented in SEQ ID NOs 515 to 629, whereby the double-stranded RNA is taken up by the insect or arachnida and thereby controls growth or prevents infestation. The invention also relates to insect-resistant or arachnida-resistant transgenic plants comprising a fragment of at least 17, 18, 19, 20 or 21 nucleotides of any of the sequences as represented in SEQ ID NOs 515 to 629. A non-limiting list of insect or arachnida orthologues genes of sequences comprising at least a fragment of 17 by of one of the sequences of the invention is given in Table 7.

According to another embodiment, the invention encompasses target genes which are fungal orthologues of a gene comprising a nucleotide sequence as represented in any of SEQ ID Nos 1, 5, 6, 8, 12, 14, 18, 20, 24, 26, 28, 29, 31, 33, 35, 37, 39, 41, 43, 45, 46, 49, 51, 52, 54, 55, 57 and 59. By way of example, orthologues may comprise a nucleotide sequence as represented in any of SEQ ID NOs 625 to 662, or a fragment of at least 17, 18, 19, 20, 21, 22, 23, 24, 25, 26 or 27 nucleotides thereof. According to another aspect, the invention thus encompasses any of the methods described herein for controlling fungal growth on a cell or an organism, or for preventing fungal infestation of a cell or an organism susceptible to fungal infection, comprising contacting fungal cells with a double-stranded RNA, wherein the double-stranded RNA comprises annealed complementary strands, one of which has a nucleotide sequence which is complementary to at least part of the nucleotide sequence of a target gene comprising a fragment of at least 17, 18, 19, 20 or 21 nucleotides of any of the sequences as represented in SEQ ID NOs 625 to 662, whereby the double-stranded RNA is taken up by the fungus and thereby controls growth or prevents infestation. The invention also relates to fungal-resistant transgenic plants comprising a fragment of at least 17, 18, 19, 20 or 21 nucleotides of any of the sequences as represented in SEQ ID NOs 625 to 662. A non-limiting list of fungal orthologues genes of sequences comprising at least a fragment of 17 by of one of the sequences of the invention is given in Table 8.

In one preferred embodiment of the invention the dsRNA may be expressed by (e.g. transcribed within) a host cell or host organism, the host cell or organism being an organism susceptible or vulnerable to infestation by a nematode. In this embodiment RNAi-mediated gene silencing of one or more target genes in the nematode may be used as a mechanism to control growth of the nematode in or on the host organism and/or to prevent or reduce nematode infestation of the host organism. Thus, expression of the double-stranded RNA within cells of the host organism may confer resistance to a particular nematode or to a class or family of nematodes. In case the dsRNA hits more than one nematode target gene (or hits a target gene of more than one nematode), expression of the double-stranded RNA within cells of the host organism may confer resistance to more than one nematode or more than one class of nematodes.

In a preferred embodiment the host organism is a plant and the nematode is a plant pathogenic nematode. In this embodiment the nematode is contacted with the double-stranded RNA by expressing the double-stranded RNA in a plant or plant cell that is infested with or susceptible to infestation with the plant pathogenic nematode.

In this context the term “plant” encompasses any plant material that it is desired to treat to prevent or reduce nematode growth and/or nematode infestation. This includes, inter alia, whole plants, seedlings, propagation or reproductive material such as seeds, cuttings, grafts, explants, etc. and also plant cell and tissue cultures. The plant material should express, or have the capability to express, dsRNA corresponding to one or more target genes of the nematode.

Therefore, in a further aspect the invention provides a plant, preferably atransgenic plant, or reproductive or propagation material for a transgenic plant or a cultured transgenic plant cell, which expresses or is capable of expressing at least one double-stranded RNA, wherein said double-stranded RNA comprises annealed complementary strands, one of which has a nucleotide sequence which is complementary to at least part of a nucleotide sequence of a nematode target gene, and wherein said double-stranded RNA is taken up by the nematode upon plant-nematode interaction, said double-stranded RNA being capable of inhibiting the target gene or down-regulating expression of the target gene by RNA interference. The target gene may be any of the target genes herein described, for instance a target gene that is essential for the viability, growth, development or reproduction of the nematode.

In this embodiment the nematode can be any nematode, but is preferably a plant pathogenic nematode. Preferred plant pathogenic nematodes include, but are not limited to, those listed above.

A plant to be used in the methods of the invention, or a transgenic plant according to the invention encompasses any plant, but is preferably a plant that is susceptible to infestation by a plant pathogenic nematode, including but not limited to the following plants: alfalfa, apple, apricot, artichoke, asparagus, avocado, banana, barley, beans, beet, blackberry, blueberry, broccoli, Brussels sprouts, cabbage, canola, carrot, cassaya, cauliflower, a cereal, celery, cherry, citrus, clementine, coffee, corn, cotton, cucumber, eggplant, endive, eucalyptus, figs, grape, grapefruit, groundnuts, ground cherry, kiwifruit, lettuce, leek, lemon, lime, pine, maize, mango, melon, millet, mushroom, nut oat, okra, onion, orange, an ornamental plant or flower or tree, papaya, parsley, pea, peach, peanut, peat, pepper, persimmon, pineapple, plantain, plum, pomegranate, potato, pumpkin, radicchio, radish, rapeseed, raspberry, rice, rye, sorghum, soy, soybean, spinach, strawberry, sugar beet, sugarcane, sunflower, sweet potato, tangerine, tea, tobacco, tomato, a vine, watermelon, wheat, yams, zucchini, Brassica and Arabidopsis.

Accordingly, the present invention extends to methods as described herein wherein the plant is rice, soybean, cotton, potato, tomato, corn, banana, tobacco, sugarcane, sugarbeet, maize, cowpea and black gram wheat, oats, sorghum, barley, Brassica and Arabidopsis. Most preferably the plant is rice, soybean, cotton, potato, tomato, corn or banana.

In one embodiment the present invention extends to methods as described herein, wherein the plant is rice and the target gene is a gene from a nematode selected from the group consisting of: Meloidogyne spp. (e.g. M. incognita, M. javanica or M. graminicola), Hirschmaniella spp. (e.g. H. oryzae), Aphelenchoides spp. (e.g. A. besseyi), Heterodera spp. (e.g. H. oryzae), Ditylenchus spp. (e.g. D. angustus) or Pratylenchus spp. (e.g. P. zeae).

In another embodiment the present invention extends to methods as described herein, wherein the plant is corn and the target gene is a gene from a nematode selected from the group consisting of: Meloidogyne spp. (e.g. M. incognita), Heterodera spp. (e.g. H. zeae), Criconemoides spp., Longidorus spp., Helicotylenchus spp., Hoplolaimus spp., Xiphinema spp., Paratrichodorus spp. (e.g. P. minor), Tylenchorhynchus spp., Belonolaimus spp. or Pratylenchus spp.

In another embodiment the present invention extends to methods as described herein, wherein the plant is cotton and the target gene is a gene from a nematode selected from the group consisting of: Rotylenchulus spp. (e.g. R. reniformis) or Meloidogyne spp. (e.g. M. incognita).

In another embodiment the present invention extends to methods as described herein, wherein the plant is potato and the target gene is a gene from a nematode selected from the group consisting of: Meloidogyne spp. (e.g. M. chitwoodi or M. hapla), Globodera spp. (e.g. G. pallida and G. rostochiensis) or Ditylenchus spp. (e.g. D. dipsaci or D. destructor).

In another embodiment the present invention extends to methods as described herein, wherein the plant is banana and the target gene is a gene from a nematode selected from the group consisting of: Meloidogyne spp. (e.g. M. javanica), Rotylenchulus spp. (e.g. R. reniformis); Pratylenchus spp. (e.g. P. coffeae and P. goodey) or Radopholus spp. (e.g. R. similis).

In another embodiment the present invention extends to methods as described herein, wherein the plant is tomato and the target gene is a gene from a nematode selected from the group consisting of: Meloidogyne spp. (e.g. M. chitwoodi and M. incognita) or Globodera spp. (e.g. G. pallida and G. rostochiensis).

In another embodiment the present invention extends to methods as described herein, wherein the plant is soybean and the target gene is a gene from a nematode selected from the group consisting of: Meloidogyne spp. (e.g. M. incognita and M. arenaria), Heterodera spp. (e.g. H. glycines) or Belonolaimus spp.

In a specific embodiment the plant is rice and the nematode is Meloidogyne spp. (e.g. M. incognita, M. javanica or M. graminicola). In another embodiment the plant is soybean and the nematode is Meloidogyne spp. (e.g. M. incognita and M. arenaria). In another embodiment the plant is cotton and the nematode is Meloidogyne spp. (e.g. M. incognita) causing root knots. In another embodiment the plant is potato and the nematode is Meloidogyne spp. (e.g. M. chitwoodi) causing e.g. small, raised swellings on potato tuber surface, dark specks in the potato flesh and reduced potato quality. In another embodiment the plant is potato and the nematode is Meloidogyne spp. (e.g. M. hapla). In another embodiment the plant is tomato and the nematode is Meloidogyne spp. (e.g. M. chitwoodi) causing e.g. root galls. In another embodiment the plant is corn and the nematode is Meloidogyne spp. (e.g. M. incognita) causing e.g. stunting and chlorosis, numerous root galls and proliferation of fibrous roots. In another embodiment the plant is tobacco, sugarcane or banana and the nematode is Meloidogyne spp. (e.g. M. javanica). In another embodiment the plant is peanut and the nematode is Meloidogyne spp. (e.g. M. arenaria). In another specific embodiment the plant is rice and the nematode is Heterodera spp. (e.g. H. oryzae). In another embodiment the plant is soybean and the nematode is Heterodera spp. (e.g. H. glycines) causing e.g. yield loss, stunting and chlorosis. In another embodiment the plant is corn and the nematode is Heterodera spp. (e.g. H. zeae) causing e.g. stunting, pale color and narrow leaves. In another embodiment the plant is sugarbeet and the nematode is Heterodera spp. (e.g. H. schachtii) causing e.g. stunting and yellowing of plants, misshapen and excess fibrous roots. In another specific embodiment the plant is potato, tomato or another Solanum species and the nematode is Globodera spp. (e.g. G. pallida or G. rostochiensis) causing e.g. root damage, poor growth, yellowing and wilting. In another embodiment the plant is potato and the nematode is Ditylenchus spp. (e.g. D. dipsaci, causing e.g. tubers rot, whereby leaves and stems swell and become distorted, or D. destructor, causing e.g. potato dry rot). In another embodiment the plant is rice and the nematode is Ditylenchus spp. (e.g. D. angustus). In another embodiment the plant is soybean and the nematode is Belonolaimus spp., causing e.g. severe trim of the roots of growing plants or seedlings. In another embodiment the plant is corn and the nematode is Belonolaimus spp., causing e.g. severe trim of the roots of growing plants or seedlings. In another embodiment the plant is cotton, maize, cowpea, black gram or banana and the nematode is Rotylenchulus spp. (e.g. R. reniformis). In another embodiment the plant is banana and the nematode is Pratylenchus spp. (e.g. P. coffeae or P. goodeyi). In another embodiment the plant is rice and the nematode is Pratylenchus spp. (e.g. P. zeae). In another embodiment the plant is corn and the nematode is Pratylenchus spp., causing e.g. severe pruning of the roots, resulting in stunting, as well as reduction in stalk diameter, stalk and root weights. In another embodiment the plant is banana and the nematode is Radopholus spp. (e.g. R. similis) causing e.g. rhizome rot, pepper slow wilt. In another embodiment the plant is rice and the nematode is Hirschmaniella spp. (e.g. H. oryzae). In another embodiment the plant is rice and the nematode is Aphelenchoides spp. (e.g. A. besseyi). In another embodiment the plant is corn and the nematode is Criconemoides spp. In another embodiment the plant is corn and the nematode is Longidorus spp. In another embodiment the plant is corn and the nematode is Helicotylenchus spp. causing e.g. mild stunting and reduced yields. In another embodiment the plant is corn and the nematode is Hoplolaimus spp. causing e.g. stunting. In another embodiment the plant is corn and the nematode is Xiphinema spp. In another embodiment the plant is corn and the nematode is Paratrichodorus spp. causing e.g. stunting, chlorosis, and reduced yields; e.g. P. minor feeds largely on the root tips, stopping terminal growth of the rootlets, and resulting in the stubby-root effect. In another embodiment the plant is corn and the nematode is Tylenchorhynchus spp. causing e.g. stunting.

In another embodiment the present invention extends to methods as described herein, wherein the plant is rice, cotton, potato, tomato, corn, tobacco or soybean and wherein said target gene is a gene coding for a nematode ortholgue, preferably a Meloidogyne incognita orthologue, of a protein selected from the group of proteins whose function is given in Table 1.

Preferred transgenic plants (or reproductive or propagation material for a transgenic plant, or a cultured transgenic plant cell) are plants (or reproductive or propagation material for a transgenic plant, or a cultured transgenic plant cell) wherein said nematode target gene comprises a sequence which is selected from the group comprising: (i) sequences which are at least 75%, at least 80% or 85% identical, preferably at least 90%, 95%, 96%, or more preferably at least 97%, 98% and still more preferably at least 99% identical to a sequence represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof, and (ii) sequences comprising at least 17, preferably at least 18, 19, 20 or 21, more preferably at least 22, 23 or 24 contiguous nucleotides of any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof, or wherein said nematode target gene is a nematode orthologue of a gene comprising at least 17 contiguous nucleotides of any of SEQ ID NOs 61 to 662, or the complement thereof.

Transgenic plants according to the invention extend to all plant species specifically described above being resistant to the respective nematode species as specifically described above.

In one embodiment the transgenic plant (or reproductive or propagation material for a transgenic plant or a cultured transgenic plant cell) is a rice plant or reproductive or propagation material for a rice plant or a cultured rice plant cell, wherein the target gene is a gene from a nematode selected from the group consisting of Meloidogyne spp., Hirschmaniella spp., Aphelenchoides spp., Heterodera spp., Ditylenchus spp. and Pratylenchus spp.

In another embodiment the transgenic plant (or reproductive or propagation material for a transgenic plant or a cultured transgenic plant cell) is a corn plant or reproductive or propagation material for a corn plant or a cultured corn plant cell, wherein the target gene is a gene from a nematode selected from the group consisting of Meloidogyne spp., Heterodera spp., Criconemoides spp., Longidorus spp., Helicotylenchus spp., Hoplolaimus spp., Xiphinema spp., Paratrichodorus spp., Tylenchorhynchus spp., Belonolaimus spp. and Pratylenchus spp.

In another embodiment the transgenic plant (or reproductive or propagation material for a transgenic plant or a cultured transgenic plant cell) is a cotton plant or reproductive or propagation material for a cotton plant or a cultured cotton plant cell, wherein the target gene is a gene from a nematode selected from the group consisting of Meloidogyne spp., and Rotylenchulus spp.

In another embodiment the transgenic plant (or reproductive or propagation material for a transgenic plant or a cultured transgenic plant cell) is a potato plant or reproductive or propagation material for a potato plant or a cultured potato plant cell, wherein the target gene is a gene from a nematode selected from the group consisting of Meloidogyne spp., Globodera spp. and Ditylenchus spp.

In another embodiment the transgenic plant (or reproductive or propagation material for a transgenic plant or a cultured transgenic plant cell) is a banana plant or reproductive or propagation material for a banana plant or a cultured banana plant cell, wherein the target gene is a gene from a nematode selected from the group consisting of Meloidogyne spp., Rotylenchulus spp., Pratylenchus spp. and Radopholus spp.

In another embodiment the transgenic plant (or reproductive or propagation material for a transgenic plant or a cultured transgenic plant cell) is a tomato plant or reproductive or propagation material for a tomato plant or a cultured tomato plant cell, wherein the target gene is a gene from a nematode selected from the group consisting of Meloidogyne spp. and Globodera spp.

In another embodiment the transgenic plant (or reproductive or propagation material for a transgenic plant or a cultured transgenic plant cell) is a soybean plant or reproductive or propagation material for a soybean plant or a cultured soybean plant cell, wherein the target gene is a gene from a nematode selected from the group consisting of Meloidogyne spp., Belonolaimus spp. and Heterodera spp.

In yet another embodiment the transgenic plant (or reproductive or propagation material for a transgenic plant or a cultured transgenic plant cell) is a rice, cotton, potato, tomato, corn, tobacco or soybean plant or reproductive or propagation material coding for a Meloidogyne incognita orthologue of a protein selected from the group of proteins whose function is given in Table 1.

The present invention also encompasses transgenic plants (or reproductive or propagation material for a transgenic plant, or a cultured transgenic plant cell) which express or are capable of expressing at least one of the nucleotides of the invention, for instance at least one of the nucleotide sequences represented in any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57 or 59, or the complement thereof, or comprising a fragment thereof comprising at least 17, preferably at least 18, 19, 20 or 21, more preferably at least 22, 23 or 24 nucleotides.

The plant may be provided in a form wherein it is actively expressing (transcribing) the double-stranded RNA in one or more cells, cell types or tissues. Alternatively, the plant may be “capable of expressing”, meaning that it is transformed with a transgene which encodes the desired dsRNA but that the transgene is not active in the plant when (and in the form in which) the plant is supplied.

Therefore, according to another embodiment, a recombinant DNA construct is provided comprising the nucleotide sequence encoding the dsRNA or dsRNA construct according to the present invention operably linked to at least one regulatory sequence. Preferably, the regulatory sequence is selected from the group comprising constitutive promoters or tissue specific promoters as described in the invention.

The target gene may be any target gene herein described. Preferably the regulatory element is a regulatory element that is active in a plant cell. More preferably, the regulatory element is originating from a plant.

The term “regulatory sequence” is to be taken in a broad context and refers to a regulatory nucleic acid capable of effecting expression of the sequences to which it is operably linked.

Encompassed by the aforementioned term are promoters and nucleic acids or synthetic fusion molecules or derivatives thereof which activate or enhance expression of a nucleic acid, so called activators or enhancers. The term “operably linked” as used herein refers to a functional linkage between the promoter sequence and the gene of interest, such that the promoter sequence is able to initiate transcription of the gene of interest.

A promoter according to the invention may be a constitutive or an inducible promoter. Preferred promoters are inducible promoters to allow tight control of expression of the RNA molecules. Promoters inducible through use of an appropriate chemical, such as IPTG are preferred. Alternatively, the transgene encoding the RNA molecule is placed under the control of a strong constitutive promoter. Preferably, any promoter which is used will direct strong expression of the RNA. The nature of the promoter utilised may, in part, be determined by the specific host cell utilised to produce the RNA. In one embodiment, the regulatory sequence comprises a bacteriophage promoter, such as a T7, T3, SV40 or SP6 promoter, most preferably a T7 promoter. In yet other embodiments of the present invention, other promoters useful for the expression of RNA are used and include, but are not limited to, promoters from an RNA Pol I, an RNA Pol II or an RNA Pol III polymerase. These promoters are typically used for in vitro-production of dsRNA, which dsRNA is then included in an anti-nematicidal agent, for example in an anti-nematicidal liquid, spray or powder.

Other promoters derived from yeast or viral genes may also be utilised as appropriate.

In an alternative embodiment, the regulatory sequence comprises a promoter selected from the well known tac, trc and lac promoters. Inducible promoters suitable for use with bacterial hosts include β-lactamase promoter, E. coli λ phage PL and PR promoters, and E. coli galactose promoter, arabinose promoter and alkaline phosphatase promoter. Therefore, the present invention also encompasses a method for generating any of the RNA molecules or RNA constructs of the invention. This method comprises the steps of introducing (e.g. by transformation, transfection or injection) an isolated nucleic acid or a recombinant (DNA) construct of the invention in a host cell of the invention under conditions that allow transcription of said nucleic acid or recombinant (DNA) construct to produce the RNA which acts to down regulate a target gene of interest (when the host cell is ingested by the target organism or when a host cell or extract derived therefrom is taken up by the target organism).

By way of example, the transgene nucleotide sequence encoding the double-stranded RNA could be placed under the control of an inducible or growth or developmental stage-specific promoter which permits transcription of the dsRNA to be turned on, by the addition of the inducer for an inducible promoter or when the particular stage of growth or development is reached.

Alternatively, the transgene encoding the double-stranded RNA is placed under the control of a strong constitutive promoter such as any selected from the group comprising the CaMV35S promoter, doubled CaMV35S promoter, ubiquitin promoter, actin promoter, rubisco promoter, GOS2 promoter, Figwort mosaic viruse (FMV) 34S promoter, cassaya vein mosaic virus (CvMv) promoter, Laccase promoter, Stawberry virus 2 (SBV2) promoter.

Alternatively, the transgene encoding the double-stranded RNA is placed under the control of a tissue specific promoter. Tissue specific promoters are advantageous in that they limit the expression of the foreign gene to the area where its activity is required, reducing the risk of obtaining gene products which are undesired or lethal to other tissues. As used herein “tissue specific” includes root, tuber, vascular tissue, mesophyl tissue, stem, stamen, fruit, seed or leaf specific promoters. Examples of tissue specific promoters include any selected from the group comprising root specific promoters of genes encoding PsMTA Class III Chitinase, photosynthetic tissue-specific promoters such as promoters of cab1 and cab2, rbcS, gapA, gapB and ST-LS1 proteins, JAS promoters, chalcone synthase promoter, pyk10 promoter (Nitz, I., et al. 2001 Plant Science, 161:337-346), TUB-1 promoter (Lilley, C. J., et al., 2004 Plant Biotechnology Journal 2:3-12), ARSK1 promoter (Lilley, C. J., et al., 2004 Plant Biotechnology Journal 2:3-12), RPL16A promoter (Lilley, C. J., et al., 2004 Plant Biotechnology Journal 2:3-12).

Furthermore, when using the methods of the present invention for developing transgenic plants resistant against nematodes, it might be beneficial to place the nucleic acid encoding the double-stranded RNA according to the present invention under the control of a nematode inducible promoter such as TobRB7 (Opperman, C. H., et al., 1994 Science 263:221-223), phosphoglycerate mutase (PGM) of Arabidopsis (Mazarei, M., et al., 2003 Plant Molecular Biology 53:513-530), auxin promoter (Mazarei, M., et al., 2003 Plant Molecular Biology 53:513-530), ABI3 promoter (De Meutter, J., et al., 2005 Molecular Plant Pathology 6:321-326), endo-1,4-β glucanase (Cel1) promoter (Mitchum, M. G., et al., 2004 Molecular Plant Pathology 5:175-181), copper amine oxidase (atao1) promoter (Møller, S. G., et al., 1998 Physiological and Molecular Plant Pathology 53: 73-79), Late embryogenesis abundant (LEA) promoter (De Meutter, J., et al., 2005 Molecular Plant Pathology 6:321-326), pyk20 promoter (Puzio, P. S., et al., 2000 Plant Science 157: 245-255).

Therefore, the present invention also encompasses a method for generating any of the double-stranded RNA or RNA constructs of the invention. This method comprises the steps of (a) contacting an isolated nucleic acid or a recombinant DNA construct of the invention with cell-free components; or (b) introducing (e.g. by transformation, transfection or injection) an isolated nucleic acid or a recombinant DNA construct of the invention in a cell, under conditions that allow transcription of said nucleic acid or recombinant DNA construct to produce the dsRNA or RNA construct.

In one embodiment of the present invention, there is provided a recombinant DNA construct as described herein for use as a medicament.

Accordingly, the present invention also encompasses a cell comprising any of the nucleotide sequences or recombinant DNA constructs described herein. The invention further encompasses prokaryotic cells (such as, but not limited to, gram-positive and gram-negative bacterial cells) or eukaryotic cells (such as, but not limited to, yeast cells or plant cells). Preferably said cell is a bacterial cell or a plant cell.

Optionally, one or more transcription termination sequences may also be incorporated in the recombinant construct of the invention. The term “transcription termination sequence” encompasses a control sequence at the end of a transcriptional unit, which signals 3′ processing and poly-adenylation of a primary transcript and termination of transcription. The transcription termination sequence is useful to prevent read through transcription such that the RNA molecule is accurately produced in or by the host cell. In one embodiment, the terminator comprises a T7, T3, SV40 or SP6 terminator, preferably a T7 terminator. Other terminators derived from yeast or viral genes may also be utilised as appropriate.

Additional regulatory elements, such as transcriptional or translational enhancers, may be incorporated in the expression construct.

The recombinant constructs of the invention may further include an origin of replication which is required for maintenance and/or replication in a specific cell type. One example is when an expression construct is required to be maintained in a bacterial cell as an episomal genetic element (e.g. plasmid or cosmid molecule) in a cell. Preferred origins of replication include, but are not limited to, f1-ori and colE1 ori.

The recombinant construct may optionally comprise a selectable marker gene. As used herein, the term “selectable marker gene” includes any gene, which confers a phenotype on a cell in which it is expressed to facilitate the identification and/or selection of cells, which are transfected or transformed, with an expression construct of the invention. Examples of suitable selectable markers include resistance genes against ampicillin (Ampr), tetracycline (Tcr), kanamycin (Kanr), phosphinothricin, and chloramphenicol (CAT) gene. Other suitable marker genes provide a metabolic trait, for example manA. Visual marker genes may also be used and include for example beta-glucuronidase (GUS), luciferase and Green Fluorescent Protein (GFP).

Plants that have been stably transformed with a transgene encoding the dsRNA may be supplied as seed, reproductive material, propagation material or cell culture material which does not actively express the dsRNA but has the capability to do so.

Accordingly, the present invention encompasses a plant (e.g. a rice plant), or a seed (e.g. a rice seed), or a cell (e.g. a bacterial or plant cell), comprising any of the nucleotide sequences encoding the dsRNA or dsRNA construct as described herein. Preferably the plant (e.g. a rice plant), or seed (e.g. a rice seed), or cell (e.g. a bacterial or plant cell) comprises at least one double-stranded RNA, at least one double-stranded RNA construct, at least one nucleotide sequence, at least one recombinant DNA construct or at least one plant cell as described herein. The present invention also encompasses a plant (e.g. a alfalfa, apple, apricot, artichoke, asparagus, avocado, banana, barley, beans, beet, blackberry, blueberry, broccoli, Brussels sprouts, cabbage, canola, carrot, cassaya, cauliflower, a cereal, celery, cherry, citrus, clementine, coffee, corn, cotton, cucumber, eggplant, endive, eucalyptus, figs, grape, grapefruit, groundnuts, ground cherry, kiwifruit, lettuce, leek, lemon, lime, pine, maize, mango, melon, millet, mushroom, nut oat, okra, onion, orange, an ornamental plant or flower or tree, papaya, parsley, pea, peach, peanut, peat, pepper, persimmon, pineapple, plantain, plum, pomegranate, potato, pumpkin, radicchio, radish, rapeseed, raspberry, rice, rye, sorghum, soy, soybean, spinach, strawberry, sugar beet, sugarcane, sunflower, sweet potato, tangerine, tea, tobacco, tomato, a vine, watermelon, wheat, yams, zucchini, Brassica or Arabidopsis plant; preferably a potato, rice, corn, cotton, potato, banana, tomato or soybean plant), or a seed or tuber (e.g. a a alfalfa, apple, apricot, artichoke, asparagus, avocado, banana, barley, beans, beet, blackberry, blueberry, broccoli, Brussels sprouts, cabbage, canola, carrot, cassaya, cauliflower, a cereal, celery, cherry, citrus, clementine, coffee, corn, cotton, cucumber, eggplant, endive, eucalyptus, figs, grape, grapefruit, groundnuts, ground cherry, kiwifruit, lettuce, leek, lemon, lime, pine, maize, mango, melon, millet, mushroom, nut oat, okra, onion, orange, an ornamental plant or flower or tree, papaya, parsley, pea, peach, peanut, peat, pepper, persimmon, pineapple, plantain, plum, pomegranate, potato, pumpkin, radicchio, radish, rapeseed, raspberry, rice, rye, sorghum, soy, soybean, spinach, strawberry, sugar beet, sugarcane, sunflower, sweet potato, tangerine, tea, tobacco, tomato, a vine, watermelon, wheat, yams, zucchini, Brassica or Arabidopsis plant; preferably a potato, rice corn, cotton or tomato seed or tuber), or a cell (e.g. a bacterial or plant cell), comprising any of the dsRNA or dsRNA constructs described herein. Preferably, these plants or seeds or cells comprise a recombinant construct wherein the nucleotide sequence encoding the dsRNA or dsRNA construct according to the present invention is operably linked to at least one regulatory element as described above. Preferably, the seed is from a plant as described herein. Preferably the plant, seed or cell is rice, cotton, potato, tomato, corn, banana, tobacco or soybean.

General techniques for expression of exogenous double-stranded RNA in plants for the purposes of RNAi are known in the art (see Baulcombe D, 2004, Nature. 431(7006):356-63. RNA silencing in plants, the contents of which are incorporated herein by reference). More particularly, methods for expression of double-stranded RNA in plants for the purposes of down-regulating gene expression in plant pests such as nematodes are also known in the art. Similar methods can be applied in an analogous manner in order to express double-stranded RNA in plants for the purposes of down-regulating expression of a target gene in a plant pathogenic nematode. In order to achieve this effect it is necessary only for the plant to express (transcribe) the double-stranded RNA in a part of the plant which will come into direct contact with the nematode, such that the double-stranded RNA can be taken up by the nematode. Depending on the nature of the nematode and its relationship with the host plant, expression of the dsRNA could occur within a cell or tissue of a plant within which the nematode is also present during its life cycle, or the RNA may be secreted into a space between cells, such as the apoplast, that is occupied by the nematode during its life cycle. Furthermore, the dsRNA may be located in the plant cell, for example in the cytosol, or in the plant cell organelles such as a chloroplast, mitochondrion, vacuole or endoplastic reticulum.

Alternatively, the dsRNA may be secreted by the plant cell and by the plant to the exterior of the plant. As such, the dsRNA may form a protective layer on the surface of the plant.

In a further aspect, the invention also provides combinations of methods and compositions for preventing or protecting plants from pest infestation. For instance, one means provides using the plant transgenic approach combining methods using expression of dsRNA molecules and methods using expression of such Bt nematicidal or insecticidal proteins.

Therefore the invention also relates to a method or a plant cell or plant described herein, wherein said plant cell or plant expressing said RNA molecule comprises or expresses a pesticidal agent selected from the group consisting of a patatin, a Bacillus thuringiensis insecticidal protein, a Bacillus thuringiensis nematicidal protein a Xenorhabdus insecticidal protein, a Photorhabdus insecticidal protein, a Bacillus laterosporous insecticidal protein, and a Bacillus sphearicus insecticidal protein. Preferably said Bacillus thuringiensis insecticidal protein is selected from the group consisting of a Cry1, a Cry3, a TIC851, a CryET170, a Cry22, a binary insecticidal protein CryET33 and CryET34, a binary insecticidal protein CryET80 and CryET76, a binary insecticidal protein TIC100 and TIC101, and a binary insecticidal protein PS149B1.

In a further embodiment, the invention relates to a composition for controlling nematode growth and/or preventing or reducing nematode infestation, comprising at least one double-stranded RNA, wherein the double-stranded RNA comprises annealed complementary strands, one of which has a nucleotide sequence which is complementary to at least part of a nucleotide sequence of a nematode target gene and optionally further comprising at least one suitable carrier, excipient or diluent. The target gene may be any target gene described herein. Preferably the nematode target gene is essential for the viability, growth, development or reproduction of the nematode.

In another aspect the invention relates to a composition as described above, wherein the nematode target gene comprises a sequence which is at least 75%, preferably at least 80%, 85%, 90%, more preferably at least 95%, 98% or 99% identical to a sequence selected from the group of sequences represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57 and 59, and sequences comprising at least 17, preferably at least 18, 19, 20 or 21, more preferably at least 22, 23 or 24 contiguous nucleotides of any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57 or 59, or the complement thereof, or wherein said nematode target gene is a nematode orthologue of a gene comprising any of SEQ ID Nos 61 to 662.

The present invention further relates to a composition comprising at least one double-stranded RNA, at least one double-stranded RNA construct, at least one nucleotide sequence and/or at least one recombinant DNA construct as described herein, optionally further comprising at least one suitable carrier, excipient or diluent. In one embodiment, the composition comprises at least one double-stranded RNA as described herein or at least one double-stranded RNA construct as described herein and further at least one suitable carrier, excipient or diluent. In another embodiment, the composition comprises at least one nucleotide sequence as described herein and/or at least one recombinant DNA construct as described herein and further at least one suitable carrier, excipient or diluent.

The composition may contain further components which serve to stabilise the dsRNA and/or prevent degradation of the dsRNA during prolonged storage of the composition.

The composition may still further contain components which enhance or promote uptake of the dsRNA by the nematode. These may include, for example, chemical agents which generally promote the uptake of RNA into cells e.g. lipofectamine etc.

The composition may be in any suitable physical form for application to nematodes, to substrates, to cells (e.g. plant cells), or to organisms infected by or susceptible to infection by nematodes. In terms of “susceptible organisms” which benefit from the present invention, any organism which is susceptible to pest infestation is included. Further to plants as described in more detail before, pests of many different organisms, for example animals such as humans, domestic animals (such as pets like cats, dogs etc) and livestock (including sheep, cows, pigs, chickens etc.) are envisaged.

The invention also relates to a spray comprising at least one composition or comprising at least one host cell as described herein, and further at least one adjuvant and optionally at least one surfactant

The effectiveness of a pesticide may depend on the effectiveness of the spray application. Adjuvants can minimize or eliminate many spray application problems associated with pesticide stability, solubility, incompatibility, suspension, foaming, drift, evaporation, volatilization, degradation, adherence, penetration, surface tension, and coverage. Adjuvants are designed to perform specific functions, including wetting, spreading, sticking, reducing evaporation, reducing volatilization, buffering, emulsifying, dispersing, reducing spray drift, and reducing foaming. No single adjuvant can perform all these functions, but different compatible adjuvants often can be combined to perform multiple functions simultaneously. These chemicals, also called wetting agents and spreaders, physically alter the surface tension of a spray droplet. For a pesticide to perform its function properly, a spray droplet must be able to wet the foliage and spread out evenly over a leaf. Surfactants enlarge the area of pesticide coverage, thereby increasing the pest's exposure to the chemical. Surfactants are particularly important when applying a pesticide to waxy or hairy leaves. Without proper wetting and spreading, spray droplets often run off or fail to adequately cover these surfaces. Too much surfactant, however, can cause excessive runoff or deposit loss, thus reducing pesticide efficacy. Pesticide formulations often contain surfactants to improve the suspension of the pesticide's active ingredient. This is especially true for emulsifiable concentrate (EC) formulations.

As used herein the term “adjuvant” means any nonpesticide material added to a pesticide product or pesticide spray mixture to improve the mixing and stability of the products in the spray tank and the application. As further used herein the term “surfactant” means a chemical that modifies surface tension. Surfactants can influence the wetting and spreading of liquids, and can modify the dispersion, suspension, or precipitation of a pesticide in water. There are nonionic surfactants (no electrical charge), anionic surfactants (negative charge), and cationic surfactants (positive charge)

In particular embodiments the host cells comprised in the spray are inactivated, for instance by heat inactivation or mechanical disruption (as discussed in greater detail herein).

The nature of the excipients and the physical form of the composition may vary depending upon the nature of the substrate that it is desired to treat. For example, the composition may be a liquid that is brushed or sprayed onto or imprinted into the material or substrate to be treated, or a coating or powder that is applied to the material or substrate to be treated. Thus, in one embodiment, the composition is in the form of a coating on a suitable surface which adheres to, and is eventually ingested by an nematode which comes into contact with the coating.

According to a preferred embodiment, the substrate is a plant or crop to be treated against nematode pest infestation. The composition is then internalized or eaten by the nematode, from where it can mediate RNA interference, thus controlling the nematode. The spray is preferably a pressurized/aerosolized spray or a pump spray. The particles may be of suitable size such that they adhere to the substrate to be treated or to the nematode, for example to the outer bodywall, of the nematode and may be absorbed therefrom.

In one embodiment, the composition is in the form of a bait. The bait is designed to lure the nematode to come into contact with the composition. Upon coming into contact therewith, the composition is then internalised by the nematode, by ingestion for example and mediates RNAi to thus kill the nematode. Said bait may comprise a food substance, such as a protein based food, for example fish meal. Boric acid may also be used as a bait. The bait may depend on the species being targeted. An attractant may also be used. The attractant may be a pheromone, such as a male or female pheromone for example. The attractant acts to lure the nematode to the bait, and may be targeted for a particular nematode or may attract a whole range of nematodes. The bait may be in any suitable form, such as a solid, paste, pellet or powdered form.

Additionally, compositions which come into contact with the nematodes may remain on the cuticle of the nematode.

The baits may be provided in a suitable “housing” or “trap”. Such housings and traps are commercially available and existing traps may be adapted to include the compositions of the invention. Any housing or trap which may attract a nematode to enter it is included within the scope of the invention. A trap is distinguished from a housing because the nematode can not readily leave a trap following entry, whereas a housing acts as a “feeding station” which provides the nematode with a preferred environment in which they can feed and feel safe from predators.

Accordingly, in a further aspect the invention provides a housing or trap for nematodes which contains a composition of the invention, which may incorporate any of the features of the composition described herein.

It is contemplated that the “composition” of the invention may be supplied as a “kit-of-parts” comprising the double-stranded RNA in one container and a suitable diluent or carrier for the RNA in a separate container. The invention also relates to supply of the double-stranded RNA alone without any further components. In these embodiments the dsRNA may be supplied in a concentrated form, such as a concentrated aqueous solution. It may even be supplied in frozen form or in freeze-dried or lyophilised form. The latter may be more stable for long term storage and may be de-frosted and/or reconstituted with a suitable diluent immediately prior to use.

The present invention further relates to the medical use or the use as a medicament of any of the double-stranded RNAs, double-stranded RNA constructs, nucleotide sequences, recombinant DNA constructs, hairpin sequences or compositions described herein. In one specific embodiment, the composition is a pharmaceutical or veterinary composition for treating or preventing nematode disease or infections of humans or animals, respectively. Such compositions will comprise at least one double-stranded RNA or RNA construct, or nucleotide sequence or recombinant DNA construct encoding the double-stranded RNA or RNA construct, wherein the double-stranded RNA comprises annealed complementary strands, one of which has a nucleotide sequence which corresponds to a target nucleotide sequence of a nematode target gene that causes the disease or infection, and at least one carrier, excipient or diluent suitable for pharmaceutical use.

In another embodiment the compositions described herein are used as a nematicide for a plant or for propagation or reproductive material of a plant. In yet another embodiment, the compositions described herein are used for controlling nematode growth. In yet another embodiment, the compositions described herein are used for preventing nematode infestation of plants susceptible of nematode infection.

The composition may be a composition suitable for topical use, such as application on the skin of an animal or human, for example as liquid composition to be applied to the skin as drops, gel, aerosol, or by brushing, or a spray, cream, ointment, etc. for topical application or as transdermal patches.

Alternatively, the nematode dsRNA is produced by bacteria (e.g. lactobacillus) which can be included in food and which functions as an oral vaccine against the nematode infection.

Other conventional pharmaceutical dosage forms may also be produced, including tablets, capsules, pessaries, transdermal patches, suppositories, etc. The chosen form will depend upon the nature of the target nematode and hence the nature of the disease it is desired to treat.

Preferred target human pathogenic and animal pathogenic nematodes include, but are not limited to the following:

In humans: Enterobius verraicularis, Ascaris lumbridoides, Necator, Ancylostoma, Trichuris trichiura, Strongyloides stercoralis, Trichonella spirae, Brugia malayi, Wuchereria bancrofti or Onchocerca volvulus.

In animals: Hookworms e.g. Ancylostoma caninum, Ancylostoma tubaeforme or Uncinaria stenocephala; Ascarids e.g. Toxocara canis, Toxocara cati or Toxascaris leonine; Whipworms e.g. Trichuris vulpis; herring worms or cod worms e.g. Anisakid; or tapeworm e.g. Diphyllobothrium.

In one specific embodiment, the composition may be a coating that can be applied to a substrate in order to protect the substrate from infestation by a nematode and/or to prevent arrest or reduce nematode growth on the substrate and thereby prevent damage caused by the nematode. In this embodiment, the composition can be used to protect any substrate or material that is susceptible to infestation by or damage caused by a nematode, for example foodstuffs and other perishable materials, and substrates such as wood. Preferred target nematode species for this embodiment include, but are not limited to, the following: Meloidogyne spp. e.g. M. incognita, M. javanica, M. Parenaria, M. graminicola, M. chitwoodi or M. hapla; Heterodera spp. e.g. H. oryzae, H. glycines, H. zeae or H. schachtii; Globodera spp. e.g. G. pallida or G. rostochiensis; Ditylenchus spp. e.g. D. dipsaci, D. destructor or D. angustus; Belonolaimus spp.; Rotylenchulus spp. e.g. R. reniformis; Pratylenchus spp. e.g. P. coffeae, P. goodeyi or P. zeae; Radopholus spp. e.g. R. Similis; Hirschmaniella spp. e.g. H. oryzae; Aphelenchoides spp. e.g. A. besseyi; Criconemoides spp.; Longidorus spp.; Helicotylenchus spp.; Hoplolaimus spp.; Xiphinema spp.; Paratrichodorus spp. e.g. P. minor; Tylenchorhynchus spp.

The nature of the excipients and the physical form of the composition may vary depending upon the nature of the substrate that is desired to treat. For example, the composition may be a liquid that is brushed or sprayed onto or imprinted into the material or substrate to be treated, or a coating that is applied to the material or substrate to be treated.

The present invention further encompasses a method for treating and/or preventing nematode infestation on a substrate comprising applying an effective amount of any of the compositions described herein to said substrate.

The invention further encompasses a method for treating and/or preventing a nematode disease or condition, comprising administering to a subject in need of such treatment and/or prevention, any of the compositions as herein described, said composition comprising at least one double-stranded RNA or double-stranded RNA construct comprising annealed complementary strands, one of which has a nucleotide sequence which is complementary to at least part of a nucleotide sequence of a nematode target gene that causes the nematode disease or condition. Therapies are being developed based on RNAi in human and in animals; dsRNA of the target genes can be used as vaccines if the targets work against a range of nematode species.

In another embodiment of the invention the compositions are used as a nematicide for a plant or for propagation or reproductive material of a plant, such as on seeds. As an example, the composition can be used as a nematicide by spraying or applying it on plant tissue or spraying or mixing it on the soil before or after emergence of the plantlets.

In a more specific embodiment, the invention relates to the use of a spray comprising at least one host cell or at least one host cell (e.g. a bacterial or a yeast) expressing a dsRNA of the invention, or a virus encoding a dsRNA described herein, or to any of the compositions comprising the same, for controlling nematode growth; for preventing nematode infestation of plants susceptible to nematode infection; or for treating nematode infection of plants. Preferably said host cell comprises at least one of the sequences represented by any of SEQ ID NOs SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662or a fragment thereof of at least 17 contiguous nucleotides.

In a further aspect, the invention also provides combinations of methods and compositions for preventing or protecting plants from pest infestation. For instance, one means provides using a combination of the transgenic approach with methods using double stranded RNA molecules and compositions with one or more Bt insecticidal proteins or chemical (organic) compounds that are toxic to the target pest. Another means provides using the transgenic approach combining methods using expression of double stranded RNA molecules in bacteria or yeast and expression of such Bt insecticidal proteins in the same or in distinct bacteria or yeast. According to these approaches, for instance, a nematode can be targeted or killed using the RNAi-based method or technology, while an insect can be targeted or killed using the Bt insecticide or the chemical (organic) insecticide.

Therefore the invention also relates to any of the compositions, sprays or methods for treating plants described herein, wherein said composition comprises a bacterial cell or yeast expressing said RNA molecule and further comprises a pesticidal agent or comprises a bacterial cell or yeast cell comprising or expressing a pesticidal agent (the bacterial or yeast cell can be the same or different from the first ones mentioned), said pesticidal agent selected from the group consisting of a chemical (organic) insecticide, a patatin, a Bacillus thuringiensis insecticidal protein, a Bacillus thuringiensis nematicidal protein a Xenorhabdus insecticidal protein, a Photorhabdus insecticidal protein, a Bacillus laterosporous insecticidal protein, and a Bacillus sphearicus insecticidal protein. Preferably said Bacillus thuringiensis insecticidal protein is selected from the group consisting of a Cry1, a Cry3, a TIC851, a CryET170, a Cry22, a binary insecticidal protein CryET33 and CryET34, a binary insecticidal protein CryET80 and CryET76, a binary insecticidal protein TIC100 and TIC101, and a binary insecticidal protein PS149B1.

The spray can be used in a greenhouse or on the field. Typical application rates for bacteria-containing biopestides (e.g. as an emulsifiable suspension) amount to 25-100 liters/ha (10-40 liters/acre) for water based sprays: comprising about 2.5-5 liter of formulated product (emulsifiable suspension) per hectare with the formulated product including about 25% (v/v) of ‘bacterial cells’ plus 75% (v/v) ‘other ingredients’. The amount of bacterial cells are measured in units, e.g. one unit is defined as 10⁹ bacterial cells in 1 ml. Depending on the crop density per hectare and the leaf surface per plant, one liter of formulated product comprises between 0.001 and 10000 units of bacteria, preferably at least 0.001, 0.003, 0.005, 0.007, 0.01, 0.03, 0.05, 0.07, 0.1, 0.3, 0.5, 0.7, more preferably at least 1, 3, 5, 7, 10, 30, 50, 70, 100, 300, 500, 700, or more preferably at least 1000, 3000, 5000, 7000 or 10000 units of bacteria.

For instance, typical plant density for potato crop plants is approximately 4.5 plants per square meter or 45,000 plants per hectare (planting in rows with spacing between rows at 75 cm and spacing between plants within rows at 30 cm). The present invention thus relates to a spray comprising at least 0.001, 0.003, 0.005, 0.007, 0.01, 0.03, 0.05, 0.07, 0.1, 0.3, 0.5, 0.7, more preferably at least 1, 3, 5, 7, 10, 30, 50, 70, 100, 300, 500, 700, or more preferably at least 1000, 3000, 5000, 7000 or 10000 units of bacteria expressing at least one of the dsRNA molecules or dsRNA constructs described herein.

In yet another embodiment, the present invention provides a method for treating and/or preventing nematode growth and/or nematode infestation of a plant or propagation or reproductive material of a plant, comprising applying an effective amount of any of the compositions herein described to a plant or to propagation or reproductive material of a plant.

In another embodiment the invention relates to the use of any double-stranded RNA or RNA construct, or nucleotide sequence or recombinant DNA construct encoding the double-stranded RNA or RNA construct described herein, or cell or to any of the compositions comprising the same, used for controlling nematode growth; for preventing nematode infestation of plants susceptible to nematode infection; or for treating nematode infection of plants. Specific plants to be treated for nematode infections caused by specific nematode species are as described earlier and are encompassed by the said use.

The invention further relates to a kit comprising at least one double-stranded RNA, or double-stranded RNA construct, or nucleotide sequence, or recombinant DNA construct, or cell, or composition as described earlier for treating nematode infection in plants. The kit may be supplied with suitable instructions for use. The instructions may be printed on suitable packaging in which the other components are supplied or may be provided as a separate entity, which may be in the form of a sheet or leaflet for example. The instructions may be rolled or folded for example when in a stored state and may then be unrolled and unfolded to direct use of the remaining components of the kit.

According to a still further embodiment, the present invention extends to a method for increasing plant yield comprising introducing in a plant any of the nucleotide sequences or recombinant DNA constructs as herein described in an expressible format. Plants encompassed by this method are as described earlier (e.g. alfalfa, apple, apricot, artichoke, asparagus, avocado, banana, barley, beans, beet, blackberry, blueberry, broccoli, Brussels sprouts, cabbage, canola, carrot, cassaya, cauliflower, a cereal, celery, cherry, citrus, clementine, coffee, corn, cotton, cucumber, eggplant, endive, eucalyptus, figs, grape, grapefruit, groundnuts, ground cherry, kiwifruit, lettuce, leek, lemon, lime, pine, maize, mango, melon, millet, mushroom, nut oat, okra, onion, orange, an ornamental plant or flower or tree, papaya, parsley, pea, peach, peanut, peat, pepper, persimmon, pineapple, plantain, plum, pomegranate, potato, pumpkin, radicchio, radish, rapeseed, raspberry, rice, rye, sorghum, soy, soybean, spinach, strawberry, sugar beet, sugarcane, sunflower, sweet potato, tangerine, tea, tobacco, tomato, a vine, watermelon, wheat, yams, zucchini, Brassica and Arabidopsis; preferably rice, cotton, potato, tomato, corn, banana or soybean). Preferably, said plant is rice, cotton, potato, tomato, corn, banana, tobacco or soybean.

In one specific embodiment, the method of the invention may also be used as a tool for experimental research, particularly in the field of functional genomics. Targeted down-regulation of nematode genes by RNAi can be used in in vitro or in vivo assays in order to study gene function, in an analogous approach to that which has been described in the art for the nematode C. elegans and also Drosophila melanogaster. Assays based on targeted down-regulation of specific nematode genes, leading to a measurable phenotype may also form the basis of compound screens for novel anti-nematode agents.

### BRIEF DESCRIPTION OF FIGURES AND TABLES

FIG. 1: RNAi effect on Meloidogyne incognita in ex vitro tomato composite plants. Egg counts (average number of eggs per μl from three samplings) from tomato composite roots expressing hairpins of different targets. The control lines were transformed with a binary vector without hairpin (empty vector control). Error bars are 95% confidence intervals.

FIG. 2: RNAi effect on Meloidogyne incognita in transgenic tomato hairy roots. Egg counts per gram of root mass (average number of eggs from three samplings) as % to egg counts per gram of control roots (100%) from tomato hairy roots expressing hairpins of different targets. The control lines were transformed with a binary vector containing a gus hairpin. Error bars are standard error bars.

FIG. 3: RNAi effect on Meloidogyne incognita in transgenic Arabidopsis thaliana plants expressing a MiCC2 hairpin. Number of galls (determined 7 days post inoculation) (average of 10 replicates) and number of egg laying females ELF (determined 8 weeks post inoculation)(average of 10 replicates) of three independent transgenic lines (MiCC2 HP expressing Ara) are presented. The control plants are wild type Arabidopsis thaliana. Error bars are standard deviations.

FIG. 4: RNAi effect on Meloidogyne incognita in transgenic Arabidopsis thaliana plants expressing a Mi40 hairpin. Number of galls (determined 7 days post inoculation) (average of 10 replicates) and number of egg laying females (ELF) (determined 8 weeks post inoculation) (average of 10 replicates) of two independent transgenic lines (Mi40 HP expressing Ara plants) are presented. The control plants are wild type Arabidopsis thaliana. Error bars are standard deviations.

FIG. 5: RNAi effect on Meloidogyne incognita in transgenic Arabidopsis thaliana plants expressing a Mi116 hairpin. Number of galls (determined 7 days post inoculation) (average of 10 replicates) and number of egg laying females (ELF) (determined 8 weeks post inoculation) (average of 10 replicates) of two independent transgenic lines (Mi116 HP expressing Ara plants) are presented. The control plants are wild type Arabidopsis thaliana. Error bars are standard deviations.

Table 1: Examples of novel identified nematode target genes. Gene function assigned is based on the Wormbase orthologue.

Table 2: Overview of cloning details of cDNA's of Meloidogyne incognita target genes including primer sequences and cDNA sequences obtained.

Table 3: Amino acid sequence of Meloidogyne incognita cDNA clones.

Table 4: Nucleotide sequences of fragments of the Meloidogyne incognita cDNA clones.

Table 5: Hairpin sequences.

Table 6: Selected sequences* of target genes. Fragments of at least 17 by of the sequences* are present in any of the orthologues sequences in nematode species (represented by GI number in the right column; several database entry numbers were found for each species but only one is given by way of example).

Table 7: Selected sequences* of target genes. Fragments of at least 17 by of the sequences* are present in any of the orthologues sequences in insect species (represented by GI number in the right column; several database entry numbers were found for each species but only one is given by way of example).

Table 8: Selected sequences* of target genes. Fragments of at least 17 by of the sequences* are present in any of the orthologues sequences in fungi species (represented by GI number in the right column; several database entry numbers were found for each species but only one is given by way of example).

The invention will be further understood with reference to the following non-limiting examples.

### EXAMPLES

### Example 1

### The Effect of Silencing the C. elegans Target Genes Mi05, Mi11, Mi38, Mi40, Mi101, Mi109, Mi111, Mi116, Mi125, Mi127, Mi128 and Mi129 in C. elegans

The C. elegans orthologs of the target genes Mi05, Mi11, Mi38, Mi40, Mi101, Mi109, Mi111, Mi116, Mi125, Mi127, Mi128 and Mi129 were isolated via PCR from genomic DNA of N2-staged wild-type worms. A fragment of these orthologs was chosen to maximize the number of exons in overlap, and this fragment was used in further dsRNA mediated silencing experiments.

Each fragment was cloned in the pGN49A vector (WO01/88121) between two identical T7-promoters and terminators, driving its expression in the sense and antisense direction upon expression of the T7 polymerase, which was induced by IPTG.

This vector was transformed into the bacterial strain AB301-105 (DE3). Subsequently these bacterial cells were fed to a nuclease deficient C. elegans strain.

Feeding the dsRNA produced in bacterial strain AB301-105 (DE3), to C. elegans nuc-1 (e1392) worms, was performed in a 96 well plate as follows nuc-1 eggs were transferred to a separate plate and allowed to hatch simultaneously at 20° C. for synchronization of the L1 generation. A 96 well plate was filled with 100 μL liquid growth medium comprising IPTG and with 10 μL bacterial cell culture of OD₆₀₀1 AB301-105 (DE3) carrying the vector with the fragment for expression of the dsRNA. To each well, 4 of the synchronized L1 worms were added and were incubated at 25° C. for at least 4 to 5 days. These experiments were performed in quadruplicate. As a negative control, C. elegans was fed with bacteria carrying a vector without the 1216 by fragment.

After 5 days the phenotype of the C. elegans nuc-1 (e1392) worms fed with the bacteria producing dsRNA was compared to the phenotype of worms fed with the empty vector.

The worms that were fed with dsRNA targeting Mi05, Mi11, Mi38, Mi40, Mi101, Mi109, Mi111, Mi116, Mi125, Mi127, Mi128 and Mi129 showed acute lethality when L1 were put in the experiment or lethal when later larval stages were tested.

The worms that were fed with dsRNA targeting Mi11 showed embryonic lethality or were sterile (based on public RNAi data (www.wormbase.org)).

Based on these experiments, it was concluded that silencing the above mentioned C. elegans target genes had a fatal effect on the growth and viability of the worm and that the target gene is essential to the viability of nematodes. Therefore these genes are good target genes to control (kill or prevent from growing) nematodes via dsRNA mediated gene silencing. Accordingly, the present invention encompasses the use of a nematode ortholog of the above C. elegans target genes, to control nematode infestation, such as nematode infestation of plants.

### Example 2

### Cloning, Identification or Assembly of M. incognita Target Genes Mi05, Mi11, Mi38, Mi40, Mi101, Mi109, Mi111, Mi116, Mi125, Mi127, Mi128 and Mi129

### Example 2.1

### Cloning of a Partial Sequence of the M. incognita Mi05 Gene Via Family PCR

To isolate cDNA sequences from M. incognita comprising a portion of the Mi05 gene, a series of PCR reactions were performed on cDNA (prepared from M. incognita total RNA), using Amplitaq Gold (Cat. NO. N8080240; Applied Biosystems) as prescribed by the manufacturer.

First, the degenerate primers oGAUH009 and oGAUH018 (represented herein as SEQ ID NO 3 and SEQ ID NO 4 respectively) were used in a PCR reaction with the following conditions: 10 minutes at 95° C., followed by 40 cycles of 30 seconds at 95° C., 40 seconds at 45° C. and 1 minute 10 seconds at 72° C., followed by 5 minutes at 72° C. The resulting PCR product was analyzed on agarose gel, isolated, cloned into the pCR4-TOPO vector (Cat. NO. K4575-40; Invitrogen) and sequenced.

Subsequently, specific primers were designed in order to perform 3′ RACE PCR in combination with SMART primers (Clontech). This resulted in the identification of more 3′ coding sequence and the 3′ UTR of Mi05. The assembled cDNA contig is herein represented by SEQ ID NO 1 and is referred to as the partial sequence of the M. incognita Mi05 gene. The corresponding partial amino acid sequence is herein represented as SEQ ID NO 2.

Accordingly, a further embodiment of the present invention includes an isolated polynucleotide comprising a sequence substantially identical to SEQ ID NO 1 or comprising any fragment thereof.

Based on the partial sequence of the Mi05 target gene, represented herein as SEQ ID NO 1, the full length sequence of Mi05 target gene is isolated from cDNA prepared from M. incognita total RNA. The “full length gene” is meant to encompass the coding region with or without the 5′ UTR and/or 3′ UTR. This is achieved via a 5′ RACE PCR where necessary and the specific primers used herein are designed based on the sequence of SEQ ID NO 1.

### Example 2.2

### Identification of a Sequence of the M. incognita Mi11 Gene

To identify a cDNA sequence from M. incognita Mi11 gene, one EST was found in the public database Genbank under accession number BM882119. Based on this EST sequence, specific primers oGAUL001 and oGAUL008 (represented herein as SEQ ID NO 10 and SEQ ID NO 11 respectively) were designed and used in a PCR reaction on cDNA (prepared from M. incognita total RNA), using Amplitaq Gold (Cat. NO. 8080240; Applied Biosystems) as prescribed by the manufacturer. Two independent PCR reactions were set up with the following conditions: 10 minutes at 95° C., followed by 35 cycles of 30 seconds at 95° C., 30 seconds at 53° C. and 30 seconds at 72° C., followed by 7 minutes at 72° C. The resulting PCR products were analyzed on agarose gel, isolated, cloned into the pCR4-TOPO vector (Cat. NO. K4575-40; Invitrogen) and sequenced. The sequences of 3 clones from each independent PCR reactions were used to assemble the contig of Mi11. The assembled cDNA contig is herein represented by SEQ ID NO 8 and is referred to as the partial sequence of the M. incognita Mi11 gene. The corresponding partial amino acid sequence is herein represented as SEQ ID NO 9.

Accordingly, a further embodiment of the present invention includes an isolated polynucleotide comprising a sequence substantially identical to SEQ ID NO 8 or comprising any fragment thereof.

Based on the partial sequence of the Mi11 target gene, represented herein as SEQ ID NO 8, the full length sequence of Mi11 target gene is isolated from cDNA prepared from M. incognita total RNA. The “full length gene” is meant to encompass the coding region with or without the 5′ UTR and/or 3′ UTR. This is achieved via a 5′ RACE PCR where necessary, and/or a 3′ RACE PCR where necessary and the specific primers used herein are designed based on the sequence of SEQ ID NO 8.

### Example 2.3

### Identification of a Sequence of the M. incognita Mi38 Gene

To identify a cDNA sequence from M. incognita Mi38 gene, one EST was found in the public database Genbank under accession number AW829338. Based on this EST sequence, specific primers oGAU072 and oGAU073 (represented herein as SEQ ID NO 16 and SEQ ID NO 17 respectively) were designed and used in a PCR reaction on cDNA (prepared from M. incognita total RNA), using PerfectShot Ex Taq (Cat. NO. RR005A , Takara) as prescribed by the manufacturer. Two independent PCR reactions were set up with the following conditions: 5 minutes at 94° C., followed by 30 cycles of 30 seconds at 94° C., 30 seconds at 45° C. and 2 minutes at 72° C., followed by 7 minutes at 72° C. The resulting PCR products were analyzed on agarose gel, isolated, cloned into the pCR4-TOPO vector (Cat. NO. K4575-40; Invitrogen) and sequenced. The sequences of 3 clones from each independent PCR reactions were used to assemble the contig of Mi38.

The assembled cDNA contig is herein represented by SEQ ID NO 14 and is referred to as the partial sequence of the M. incognita Mi38 gene. The corresponding partial amino acid sequence is herein represented as SEQ ID NO 15.

Based on the partial sequence of the Mi38 target gene, represented herein as SEQ ID NO 14, the full length sequence of Mi38 target gene is isolated from cDNA prepared from M. incognita total RNA. The “full length gene” is meant to encompass the coding region with or without the 5′ UTR and/or 3′ UTR. This is achieved via a 5′ RACE PCR where necessary, and/or a 3′ RACE PCR where necessary and the specific primers used herein are designed based on the sequence of SEQ ID NO 14.

### Example 2.4

### Identification of a Sequence of the M. incognita Mi40 Gene

To identify a cDNA sequence from M. incognita Mi40 gene, one EST was found in the public database Genbank under accession number BQ519760. Based on this EST sequence, specific primers oGAU048 and oGAU049 (represented herein as SEQ ID NO 22 and SEQ ID NO 23 respectively) were designed and used in a PCR reaction on cDNA (prepared from M. incognita total RNA), using PerfectShot Ex Taq (Cat. NO. RR005A , Takara) as prescribed by the manufacturer. Two independent PCR reactions were set up with the following conditions: 5 minutes at 94° C., followed by 30 cycles of 30 seconds at 94° C., 30 seconds at 45° C. and 2 minutes at 72° C., followed by 7 minutes at 72° C. The resulting PCR products were analyzed on agarose gel, isolated, cloned into the pCR4-TOPO vector (Cat. NO. K4575-40; Invitrogen) and sequenced. The sequences of 3 clones from each independent PCR reactions were used to assemble the contig of Mi40.

The assembled cDNA contig is herein represented by SEQ ID NO 20 and is referred to as the partial sequence of the M. incognita Mi40 gene. The corresponding partial amino acid sequence is herein represented as SEQ ID NO 21.

Accordingly, a further embodiment of the present invention includes an isolated polynucleotide comprising a sequence substantially identical to SEQ ID NO 20 or comprising any fragment thereof.

Based on the partial sequence of the Mi40 target gene, represented herein as SEQ ID NO 20, the full length sequence of Mi40 target gene is isolated from cDNA prepared from M. incognita total RNA. The “full length gene” is meant to encompass the coding region with or without the 5′ UTR and/or 3′ UTR. This is achieved via a 5′ RACE PCR where necessary, and/or a 3′ RACE PCR where necessary and the specific primers used herein are designed based on the sequence of SEQ ID NO 20.

### Example 2.5

### Identification of a Sequence of the M. incognita Mi101 Gene

To identify a cDNA sequence from M. incognita Mi101 gene, one EST was found in the public database Genbank.

The EST CF803049 is herein represented by SEQ ID NO 26 and is referred to as the partial sequence of the M. incognita Mi101 gene. The corresponding partial amino acid sequence is herein represented as SEQ ID NO 27.

Accordingly, a further embodiment of the present invention includes an isolated polynucleotide comprising a sequence substantially identical to SEQ ID NO 26 or comprising any fragment thereof.

Based on the partial sequence of the Mi101 target gene, represented herein as SEQ ID NO 26, the full length sequence of Mi101 target gene is isolated from cDNA prepared from M. incognita total RNA. The “full length gene” is meant to encompass the coding region with or without the 5′ UTR and/or 3′ UTR. This is achieved via a 5′ RACE PCR where necessary, and/or a 3′ RACE PCR where necessary and the specific primers used herein are designed based on the sequence of SEQ ID NO 26.

### Example 2.6

### Identification of a Sequence of the M. incognita Mi109 Gene

To identify a cDNA sequence from M. incognita Mi109 gene, one EST was found in the public database Genbank.

The EST CK233410 is herein represented by SEQ ID NO 31 and is referred to as the partial sequence of the M. incognita Mi109 gene. The corresponding partial amino acid sequence is herein represented as SEQ ID NO 32.

Accordingly, a further embodiment of the present invention includes an isolated polynucleotide comprising a sequence substantially identical to SEQ ID NO 31 or comprising any fragment thereof.

Based on the partial sequence of the Mi109 target gene, represented herein as SEQ ID NO 31, the full length sequence of Mi109 target gene is isolated from cDNA prepared from M. incognita total RNA. The “full length gene” is meant to encompass the coding region with or without the 5′ UTR and/or 3′ UTR. This is achieved via a 5′ RACE PCR where necessary, and/or a 3′ RACE PCR where necessary and the specific primers used herein are designed based on the sequence of SEQ ID NO 31.

### Example 2.7

### Assembly of a Sequence of the M. incognita Mi111 Gene Via Contig Building

To assemble a cDNA sequence from M. incognita Mi111 gene, several ESTs were used in the contig builder program TGICL (TIGR Gene Indices clustering tools). The ESTs used were CD749362 and CK233691, originating from the public database Genbank.

The assembled cDNA contig is herein represented by SEQ ID NO 35 and is referred to as the partial sequence of the M. incognita Mi111 gene. The corresponding partial amino acid sequence is herein represented as SEQ ID NO 36.

Accordingly, a further embodiment of the present invention includes an isolated polynucleotide comprising a sequence substantially identical to SEQ ID NO 35 or comprising any fragment thereof.

Based on the partial sequence of the Mi111 target gene, represented herein as SEQ ID NO 35, the full length sequence of Mi111 target gene is isolated from cDNA prepared from M. incognita total RNA. The “full length gene” is meant to encompass the coding region with or without the 5′ UTR and/or 3′ UTR. This is achieved via a 5′ RACE PCR where necessary, and/or a 3′ RACE PCR where necessary and the specific primers used herein are designed based on the sequence of SEQ ID NO 35.

### Example 2.8

### Assembly of a Sequence of the M. incognita Mi116 Gene Via Contig Building

To assemble a cDNA sequence from M. incognita Mi116 gene, several ESTs were used in the contig builder program TGICL (TIGR Gene Indices clustering tools). The ESTs used were BE239183, CF099482 and BM880799, originating from the public database Genbank.

The assembled cDNA contig is herein represented by SEQ ID NO 39 and is referred to as the partial sequence of the M. incognita Mi116 gene. The corresponding partial amino acid sequence is herein represented as SEQ ID NO 40.

Accordingly, a further embodiment of the present invention includes an isolated polynucleotide comprising a sequence substantially identical to SEQ ID NO 39 or comprising any fragment thereof. Based on the partial sequence of the Mi116 target gene, represented herein as SEQ ID NO 39, the full length sequence of Mi116 target gene is isolated from cDNA prepared from M. incognita total RNA. The “full length gene” is meant to encompass the coding region with or without the 5′ UTR and/or 3′ UTR. This is achieved via a 5′ RACE PCR where necessary, and/or a 3′ RACE PCR where necessary and the specific primers used herein are designed based on the sequence of SEQ ID NO 39.

### Example 2.9

### Assembly of a Sequence of the M. incognita Mi125 Gene Via Contig Building

To assemble a cDNA sequence from M. incognita Mi125 gene, several ESTs were used in the contig builder program TGICL (TIGR Gene Indices clustering tools). The ESTs used were BQ548495, CD749619, BQ625388, CK983617, CF802860, BQ519621, CN578439, CK984202, CK985275, CK985196, CN578210, CF980909, CN443-475, CK984712, CK984808, CN443437, CN443424, CN443495, CN443459 and CN578277, originating from the public database Genbank.

The assembled cDNA contig is herein represented by SEQ ID NO 43 and is referred to as the partial sequence of the M. incognita Mi125 gene. The corresponding partial amino acid sequence is herein represented as SEQ ID NO 44.

Accordingly, a further embodiment of the present invention includes an isolated polynucleotide comprising a sequence substantially identical to SEQ ID NO 43 or comprising any fragment thereof. Based on the partial sequence of the Mi125 target gene, represented herein as SEQ ID NO 43, the full length sequence of Mi125 target gene is isolated from cDNA prepared from M. incognita total RNA. The “full length gene” is meant to encompass the coding region with or without the 5′ UTR and/or 3′ UTR. This is achieved via a 5′ RACE PCR where necessary, and/or a 3′ RACE PCR where necessary and the specific primers used herein are designed based on the sequence of SEQ ID NO 43.

### Example 2.10

### Assembly of a Sequence of the M. incognita Mi127 Gene Via Contig Building

To assemble a cDNA sequence from M. incognita Mi127 gene, several ESTs were used in the contig builder program TGICL (TIGR Gene Indices clustering tools). The ESTs used were CD749453, CF980531, CF803087, CF980430, CF803126, CN578375, CN443332 and CK984842, originating from the public database Genbank.

The assembled cDNA contig is herein represented by SEQ ID NO 49 and is referred to as the partial sequence of the M. incognita Mi127 gene. The corresponding partial amino acid sequence is herein represented as SEQ ID NO 50.

Accordingly, a further embodiment of the present invention includes an isolated polynucleotide comprising a sequence substantially identical to SEQ ID NO 49 or comprising any fragment thereof. Based on the partial sequence of the Mi127 target gene, represented herein as SEQ ID NO 49, the full length sequence of Mi127 target gene is isolated from cDNA prepared from M. incognita total RNA. The “full length gene” is meant to encompass the coding region with or without the 5′ UTR and/or 3′ UTR. This is achieved via a 5′ RACE PCR where necessary, and/or a 3′ RACE PCR where necessary and the specific primers used herein are designed based on the sequence of SEQ ID NO 49.

### Example 2.11

### Assembly of a Sequence of the M. incognita Mi128 Gene Via Contig Building

To assemble a cDNA sequence from M. incognita Mi128 gene, several ESTs were used in the contig builder program TGICL (TIGR Gene Indices clustering tools). The ESTs used were CK233383, BQ519732, CN578199, CN578113, CF803167, CK233325 and CK233386, originating from the public database Genbank.

The assembled cDNA contig is herein represented by SEQ ID NO 52 and is referred to as the partial sequence of the M. incognita Mi128 gene. The corresponding partial amino acid sequence is herein represented as SEQ ID NO 53.

Accordingly, a further embodiment of the present invention includes an isolated polynucleotide comprising a sequence substantially identical to SEQ ID NO 52 or comprising any fragment thereof. Based on the partial sequence of the Mi128 target gene, represented herein as SEQ ID NO 52, the full length sequence of Mi128 target gene is isolated from cDNA prepared from M. incognita total RNA. The “full length gene” is meant to encompass the coding region with or without the 5′ UTR and/or 3′ UTR. This is achieved via a 5′ RACE PCR where necessary, and/or a 3′ RACE PCR where necessary and the specific primers used herein are designed based on the sequence of SEQ ID NO 52.

### Example 2.12

### Assembly of a Sequence of the M. incognita Mi129 Gene Via Contig Building

To assemble a cDNA sequence from M. incognita Mi129 gene, several ESTs were used in the contig builder program TGICL (TIGR Gene Indices clustering tools). The ESTs used were BM774428, CK233774, CK983824, CK984252, CK985154, CK983765, CK984305, CN443466, CK984908, CN443767 and CN443135 originating from the public database Genbank. Before running the contig builder program, the full insert of the ESTs CK984305 and CN443135 were sequenced. These full insert sequences together with BM774428, CK233774, CK983824, CK984252, CK985154, CK983765, CN443466, CK984908 and CN443767were used to assemble the contig of Mi129.

The assembled cDNA contig is herein represented by SEQ ID NO 55 and is referred to as the partial sequence of the M. incognita Mi129 gene. The corresponding partial amino acid sequence is herein represented as SEQ ID NO 56.

Accordingly, a further embodiment of the present invention includes an isolated polynucleotide comprising a sequence substantially identical to SEQ ID NO 55 or comprising any fragment thereof.

### Example 3

### Identification of a Fragment of the M. incognita Target Genes Mi05, Mi11, Mi38, Mi40, Mi101, Mi109, Mi111, Mi125, Mi116, Mi127, Mi128 and Mi129 with No Substantial Homology to Non-Target Organisms

The sequences of the Mi05, Mi11, Mi38, Mi40, Mi101, Mi109, Mi111, Mi125, Mi116, Mi127, Mi128 and Mi129 genes were analyzed for regions that show no substantial homology to non-target organisms at the siRNA level. Such regions free of non-target organism sequences were named “freefrags” herein.

For this analysis, non-target organisms used were tomato (Lycopersicum esculentum), cotton (Gossypium hirsutum), potato (Solanum tuberosum), Arabidopsis thaliana, tobacco (Nicotiana tabacum) and human (Homo sapiens). Within the freefrags not any fragment of 21 contiguous nucleotides occur in the non-target organisms.

Other examples may be fragments of 22 contiguous nucleotides allowing one mismatch, or fragments of 24 contiguous nucleotides allowing three mismatches.

A sequence of Mi05 free of non-target organism sequences was selected and was used partially in further RNA interference experiments. Similarly, a sequence of Mi11, Mi38, Mi40, Mi101, Mi109, Mi111, Mi125, Mi116, Mi127, Mi128 and Mi129 free of non-target organism sequences was selected and was each used partially in further RNA interference experiments.

The Mi05 (SEQ ID NOs 5 and 6), Mi11 (SEQ ID NO 12), Mi38 (SEQ ID NO 18), Mi40 (SEQ ID NO 24), Mi101 (SEQ ID NO 28), Mi101 used for MiCC2 (SEQ ID NO 29), Mi109 (SEQ ID NO 33), Mi111 (SEQ ID NO 37), Mi116 (SEQ ID NO 41), Mi125a (SEQ ID NO 45), Mi125b (SEQ ID NO 46), Mi127 (SEQ ID NO 51), Mi128 (SEQ ID NO 54), Mi129 (SEQ ID NO 57), freefrags represented by their respective SEQ ID NO (in brackets) and their nucleotide sequence is given in Table 4. A person skilled in the art will recognize that more freefrags, of various lengths, may be identified in the Mi11, Mi38, Mi40, Mi101, Mi109, Mi111, Mi125, Mi116, Mi127, Mi128 and Mi129 cDNA sequences, and that the present invention also extends to these freefrags. The sequences of Mi125a (SEQ ID NO 45) and Mi125b (SEQ ID NO 46) are examples of freefrags of different length in the Mi125 target gene. These sequences are suitable for nematode control by RNA interference when expressed in a plant and taken up by a nematode feeding from the plant.

### Example 4

### Assembly of the M. incognita MiCC2 Sequence Via Concatenating

MiCC2 is a concatemer that comprises sequences of targets Mi127-Mi128-Mi101. To assemble a sequence for the M. incognita MiCC2, the following freefrag sequences as described in Example 3 were concatenated: Mi127freefrag (SEQ ID NO 51), Mi128freefrag (SEQ ID NO 54) and Mi101 freefrag for MiCC2 (SEQ ID NO 29).

The assembled MiCC2 sequence is herein represented by SEQ ID NO 59 and is referred to as the sense MiCC2freefrag. The sense MiCC2freefrag sequence was generated by chemical synthesis of DNA.

Accordingly, a further embodiment of the present invention includes an isolated polynucleotide comprising a sequence substantially identical to SEQ ID NO 59 or comprising any fragment thereof.

### Example 5

### Recombination of the M. incognita Target Genes Mi05, Mi11, Mi38, Mi40, Mi101, Mi109, Mi111, Mi116, Mi125a, Mi125b, Mi129 and MiCC2 into a Destination Vector

Since the mechanism of RNA interference operates through dsRNA fragments, the Mi05 freefrag polynucleotide (see Example 3) was cloned in sense and antisense orientation, separated by an Arabidopsis-intron sequence, to form a dsRNA hairpin construct. Similarly the Mi11, Mi38, Mi40, Mi101, Mi109, Mi111, Mi116, Mi125a, Mi125b, Mi129 and MiCC2 freefrag polynucleotides (see Example 3) were each cloned in sense and antisense orientation, separated by the Arabidopsis-intron to form a dsRNA hairpin construct.

The sense sequences were isolated as a PCR product (with flanking AttB recombination sites) and ligated via BP clonase into a Gateway™ Entry clone (pDONR221) or were cloned according to Collier et al., (2005) The Plant Journal 43: 449-457. This Entry clone provides attL recombination sites flanking the cloned sense sequences.

A destination vector was made based on the binary pBIN19 plasmid, which can replicate in A. tumefaciens as well as in A. rhizogenes (RK2 broad host range origin of replication) and which comprises the RB and LB borders sequences of a Ti plasmid.

The nucleotide sequences from the Entry clone were recombined into this destination vector in sense and antisense orientation in an LR recombination reaction. The reaction products were analyzed and the correct hairpin cassettes of the format “sense-Arabidopsis:intron-antisense” were selected.

The hairpin cassette comprising the Mi05 freefrag is herein represented by SEQ ID NO 7. The hairpin cassette comprising the Mill freefrag (i.e. SEQ ID NO 12) is represented by SEQ ID NO 13. The hairpin cassette comprising the Mi38 freefrag (i.e. SEQ ID NO 18) is represented by SEQ ID NO 19. The hairpin cassette comprising the Mi40 freefrag (i.e. SEQ ID NO 24) is represented by SEQ ID NO 25. The hairpin cassette comprising the Mi101 freefrag (i.e. SEQ ID NO 28) is represented by SEQ ID NO 30. The hairpin cassette comprising the Mi109 freefrag (i.e. SEQ ID NO 33) is represented by SEQ ID NO 34. The hairpin cassette comprising the Mi111 freefrag (i.e. SEQ ID NO 37) is represented by SEQ ID NO 38. The hairpin cassette comprising the Mi116 freefrag (i.e. SEQ ID NO 41) is represented by SEQ ID NO 42. The hairpin cassette comprising the Mi125a freefrag (i.e. SEQ ID NO 45) is represented by SEQ ID NO 47; the Mi125b freefrag (i.e. SEQ ID NO 46) is represented by SEQ ID NO 48. The hairpin cassette comprising the Mi129 freefrag (i.e. SEQ ID NO 57) is represented by SEQ ID NO 58. The hairpin cassette comprising the MiCC2 freefrag is (i.e. SEQ ID NO 59) represented by SEQ ID NO 60. The sequences of the hairpin cassettes are given in Table 5.

### Example 6

### Recombinant Constructs to Produce Dsrna Derived from the M. incognita Ortholog Mi05, Mi11, Mi38, Mi40, Mi101, Mi109, Mi111, Mi116, Mi125, Mi129 and MiCC2 in Transformed Tomato, Cotton, Tobacco, Potato, Rice and Arabidopsis Plants

In the example below the following abbreviations for nucleic acid fragments comprising the different components are used: “RB” and “LB” correspond to right and left borders of the T-DNA, P35S, pCvMv and pSBV2 are constitutive promoters originating from viruses for use in plants, pUbi U4 is a constitutive promoter originating from tobacco, pTobRB7, pPGM is a nematode feeding site specific promoter originating from tobacco and Arabidopsis, respectively, pLaccase and pAct7 are constitutive promoters originating from Arabidopsis, pSU is a constitutive promoter for use in plants and is described in U.S. Publication 2003/0101478, pNOS corresponds to the nopaline synthase promoter, tNOS corresponds to the nopaline synthase terminator, t35S corresponds to the 35S terminator.

The hairpin cassettes as described in Example 5 were embedded in a binary vector, suitable for transformation into A. tumefaciens as well as in A. rhizogenes which can be used for transformation of the hairpin into any plant species such as cotton, potato, tobacco, Arabidopsis, tomato or rice. For driving the expression of the hairpin in cotton, potato, tobacco, Arabidopsis and tomato plants, different promoters were cloned preceding the hairpin cassette. Such promoters include p35S, pCvMv, pTobRB7, pLaccase, pUbi U4, pAct7, pPGM and pSBV2. Within the RB and LB borders also a selectable marker cassette of the format pNOS-selectable marker (e.g. nptII)-tNOS and a scorable marker cassette of the format promoter pSU—scorable marker (e.g. gfp)—tNOS, were cloned.

The plant expression vectors comprising the hairpin of Mi05, Mi11, Mi38, Mi40, Mi101, Mi125, Mi129, Mi109, Mi111, Mi116 and MiCC2 fragments were transformed either into Agrobacterium rhizogenes for generation of ex vitro composite plants (see example 7) or hairy roots (example 8) or into Agrobacterium tumefaciens for the regeneration of whole transgenic plants (see example 9).

### Example 7

### Bioassay of M. incognita Infection on Ex Vitro Composite Tomato Plants

Generation of Ex Vitro Composite Plants

The resulting binary constructs as described in example 6 were introduced into A. rhizogenes strain NCPPB 2659 (Combard et al., (1987) Plasmid 18: 70-75) via electroporation. A. rhizogenes strain NCPPB 2659 harboring the described binary vector was grown in Luria Bertani broth in a flask at 28° C. on a rotary shaker at 200 rpm overnight. Cells were collected by centrifugation at 3000 g for 10 min, and resuspended in ¼×Murashige and Skoog basal medium (pH 5.8) (Sigma-Aldrich, St Louis, Mo., USA) to an OD₆₀₀=0.3. Sterilized Fibrgro® cubes (Hummed International, Earth City, Mo., USA) were saturated with resuspended cells via pipetting, typically between 4 and 7 ml. Apical stem sections, excised from greenhouse-grown Lycopersicon esculentum cv. Marmande plants, were inserted into inoculated Fibrgro® cubes, placed in open Petri dishes within plant growth trays and covered with clear plastic domes and incubated overnight at room temperature. The trays were subsequently opened and the cubes were allowed to dry until the plant material was fully wilted. Cubes were saturated with deionized water and domes were returned to closed position. Cubes were checked periodically and watered when necessary for the remainder of the induction period. Nascent composite plants were incubated at room temperature in normal light conditions until roots emerged from the teratoma. Roots were analyzed for the presence of GFP using a microscope outfitted with a FITC filter (Collier et al., (2005) The Plant Journal 43: 449-457).

M. incognita Bioassay

Composite tomato plants were transferred to sand 36 days after induction; subsequently, 5 days after planting, each composite plant was infested with 1,500 M. incognita eggs (Collier et al., (2005) The Plant Journal 43: 449-457). Between 5 and 16 plant root systems, for both empty vector control as well as the RNAi Mi constructs, were assayed for M. incognita infection level with a modified egg collection protocol (Hussey and Barker (1973) Plant Disease Reports 57: 1025-1028). Eight weeks upon inoculation sand was removed from the entire root ball by a 1-min wash in Milli-Q water, followed by a 5-min wash in 5% bleach. Three samples of 100 μl were delivered to a Petri dish and counted under a dissecting microscope and number of nematode eggs (FIG. 1) was reported as an average.

### Example 8

### Bioassay of M. incognita Infection on Transformed Hairy Roots of Cotton and Tomato

Generation of Transformed Hairy Roots

Cotton (Gossypium hirsutum) or tomato (e.g. Lycopersicum esculentum cv. Marmande) cotyledons were transformed with the Agrobacterium rhizogenes strain comprising the Mi05, Mi11, Mi38, Mi40, Mi101, Mi109, Mi111, Mi116, Mi125, Mi129 and MiCC2 hairpin (see Example 6), on an agar plate according to the protocols described in Plovie et al. (Nematology 2003, vol. 5(6): 831-841). The transformed hairy roots were subsequently tested for nematode resistance.

M. incognita Bioassay

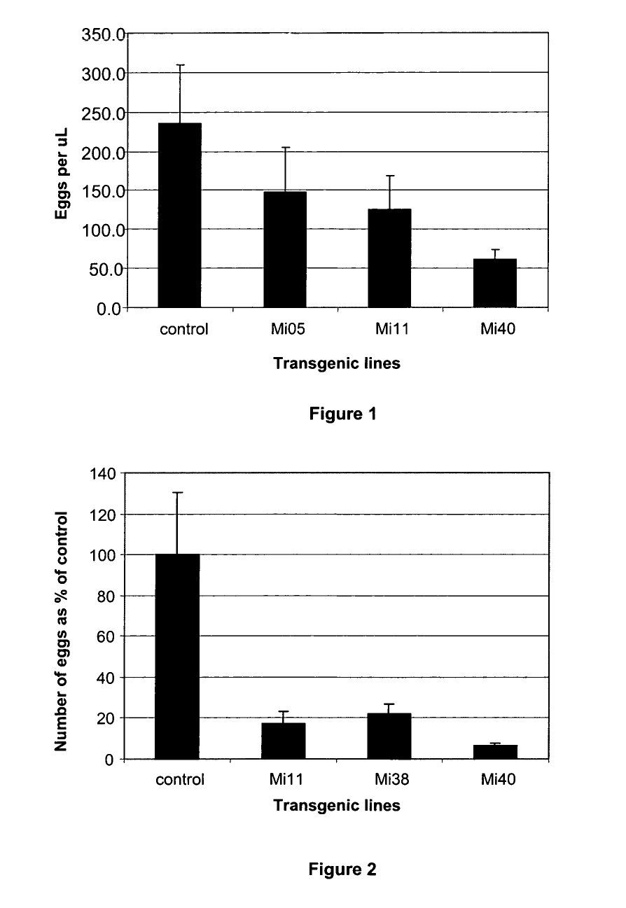

The necessary number of independent transformed lines (e.g. 15) and replicates per line (e.g. 5) were inoculated with M. incognita J2 larvae. Approximately 1,000 eggs of axenically grown root-knot nematode M. incognita species were added to the plate. The plates were incubated in the dark at a constant temperature of 20° C. Tomato roots were harvested and weighed at eight weeks post infection. Eggs were harvested from the roots using a 5% bleach solution. Eggs were separated from root material using a 50% sucrose gradient and centrifugation (130g; 750 rpm). Eggs were removed from the gradient interface and counted. All nematode egg counts were normalized to appropriate control plants being lines transformed with a binary vector containing a gus hairpin (FIG. 2).

### Example 9

### Bioassay of M. incognita Infection on Transgenic Cotton, Potato, Tobacco, Rice, Arabidopsis and Tomato Plants Expressing a Fragment of the Mi05, Mi11, Mi38, Mi40, Mi101, Mi109, Mi111, Mi116, Mi125, Mi129 and MiCC2 Genes

Generation of Transgenic Plants

Cotton, potato, tobacco, rice or tomato plant tissues are transformed with the Agrobacterium tumefaciens strain (e.g. C58) comprising the Mi05, Mi11, Mi38, Mi40, Mi101, Mi109, Mi111, Mi116, Mi125, Mi129 and MiCC2 hairpin (see Example 5), and regenerated into whole plants via protocols described for example in “Transgenic plants, Methods and Protocols. (2005) Methods in Molecular Biology, Volume 286, edited by Leandro Peña, Humana Press, Totowa N.J.”

Arabidopsis thaliana plants were transformed using the floral dip method (Clough and Bent (1998) Plant Journal 16:735-743). Aerial parts of the plants were incubated for a few seconds in a solution containing 5% sucrose, resuspended A. tumefaciens cells from an overnight culture and 0.03% of the surfactant Silwet L-77. After inoculation plants were covered for 16 hours with a transparent plastic to maintain humidity. To increase the transformation efficiency, the procedure was repeated after one week. Watering was stopped as seeds become mature and dry seeds are harvested and cold treated for two days. After sterilization seeds were plated on an antibiotic containing growth medium for selection of transformed plants. The selected plants are transferred to soil for optimal seed production.

M. incognita Bioassay

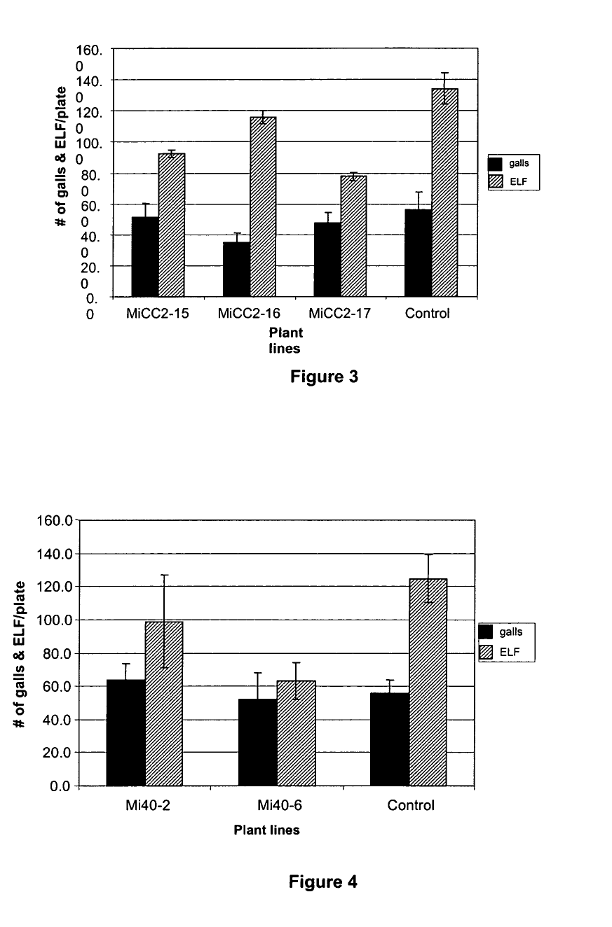

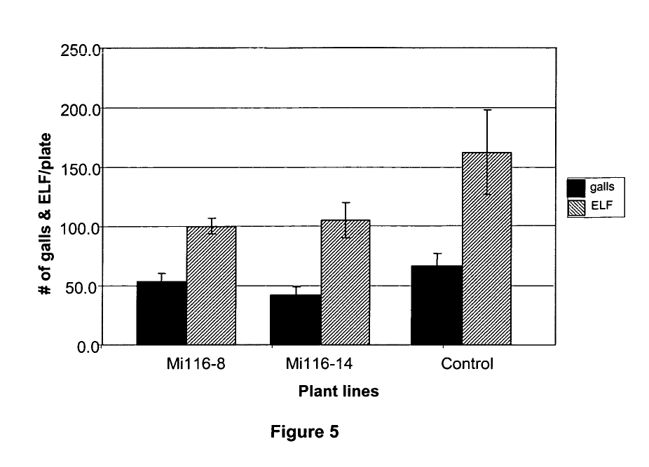

Transgenic plants were germinated on growth medium supplemented with the appropriate selectable agent and one week old plants are transferred to 10×10 cm² Petri dishes, containing Gamborg's B5 medium. For each transgenic line 10 replicate plates were made and on each plate ten plants are lined up. The Petri dishes were placed slightly tilted to promote unidirectional root growth. One week later, the roots of each plant were inoculated with 100 freshly hatched M. incognita second stage juveniles. The whole procedure was done in sterile conditions under a binocular microscope. Seven days after inoculation the number of root galls was determined and six weeks after inoculation adult females were counted and compared with the appropriate control lines (Examples are given in FIGS. 3, 4 & 5). Mature females were collected and put to hatch to check the progeny.

### Example 10

### Control of Nematodes in Transgenic Cotton, Potato, Tobacco, Arabidopsis, Rice or Tomato, Based on the Silencing of Mi05, Mi11, Mi38, Mi40, Mi101, Mi109, Mi111, Mi116, Mi125, Mi129 and MiCC2

Small plastic pots (e.g. 3 inch) or plastic seedling trays with wells of 60 mL capacity are filled with about 40 ml of soil per well, the soil originating from a plot heavily infested with M. incognita. Part of this soil is sterilized by autoclaving and is used as negative control. 5 ml of water per well is used for irrigation.

Tomato, potato, tobacco, Arabidopsis, rice or cotton seeds of the transgenic plants generated as described in Example 9 are sown in the wells, at about 2 cm deep. A light cover of vermiculite on the surface is used to avoid dehydration. Plants are irrigated by micro-nebulization 3-4 times per week at a rate of 3 L/m², weeded by hand and treated with fertilizers, fungicide and insecticides when needed.

The phenotype of the plants is monitored and the efficacy of the dsRNA complementary to Mi05, Mi11, Mi38, Mi40, Mi101, Mi109, Mi111, Mi116, Mi125, Mi129 and MiCC2 gene or gene-fragment is measured as follows. Plants are grown to reach good development of the roots, which corresponds to plant grown to about 10 cm high at about one month after emergence. After removal of the soil from the roots by rinsing with water, the roots are visually inspected and root galls are counted. The number of galls and the corresponding degree of damage is scored on the 0 to 5 infestation scale described by Lambertini (1971), Tobacco, 738: 5-10). The infestation scale is as follows:

0 is “NO ATTACK: No galls on root system (healthy plant), free from galls”;
1 is “LOW ATTACK: 1-5 small galls located in a region of root system”;
2 is “MODERATE ATTACK: 6-20 small galls, located on entire root system”;
3 is “HIGH ATTACK: More than 20 galls, located on entire root system;”
4 is “VERY HIGH ATTACK: Root system reduced and severely deformed by few and big galls, with some galls grown together”; and
5 is “EXTREMELY HEAVY ATTACK: Root system completely reduced and deformed by big gall (plant and roots dead, rotten root).”

TABLE 1

| | | | Function |
|---|---|---|---|
| | | | (identified by |
| Target | | | applicant) |
| ID | Identifier | Function (based on Wormbase) | (NA = not available) |
| Mi05 | LiN-26 | encodes a putative C2H2 zinc finger-containing protein required | Cell fate |
| | | for proper differentiation of ectodermal and mesodermal | |
| | | epithelial cells | |
| Mi11 | cdl-1 | histone stem-loop binding protein | Apoptosis |
| Mi38 | rpt-1 | 26S proteasome regulatory complex, ATPase RPT1 | NA |
| Mi40 | phi-7 | mRNA splicing protein CDC5 (Myb superfamily) | NA |
| Mi101 | SKP-1 | mRNA splicing factor/probable chromatin binding snw family | NA |
| | | nuclear protein | |
| Mi109 | rpl-19 | 60s ribosomal protein L19 | NA |
| Mi111 | RPT-2 | 26S proteasome regulatory complex, ATPase RPT2 | NA |
| Mi116 | PAS-4 | 20S proteasome, regulatory subunit alpha type PSMA7/PRE6 | NA |
| Mi125 | rps-9 | Ribosomal protein S4 | NA |
| Mi127 | hsp-1 | Molecular chaperones HSP70/HSC70, HSP70 superfamily | NA |
| Mi128 | rpl-36 | 60S ribosomal protein L36 | NA |
| Mi129 | tbb-2 | Beta tubulin | NA |

TABLE 2

| Target | Primer | Primer | cDNA Sequence (Contig) |
|---|---|---|---|
| ID | Forward | Reverse | sense strand 5′ → 3′ |
| Mi05 | SEQ ID NO: 3 | SEQ ID NO: 4 | SEQ ID NO: 1 |
| | CCAACAACAC | TCGCAGATTC | CTCCGTCACGTTATTATGATCCATTCGGACAGCAAGCCCTACCAGTGCTTCGACTGTGAT |
| | TGAATTCTAC | CATTCAGCTC | TTTATCGGGATCAAGAGCAATGTGGTCAGTCATGCGAGACAGAGCGGACACCGGAATGAC |
| | AGGAWSNMGN | KCATYTCRTC | GATGCCCTGGACATTACCACTGATGACATGAGGGCCGAATGGAATACCATGCTCCATGCT |
| | TGGAA | | TGTTTCCCTGACTATGTTAAGGCAAAGAGCCGTGGATGGCAGCCCGAAACCGAGGTCAAA |
| | | | GACGAGCCTCTGGAATCCAACAACGAACAGGACACTACCGTCAAAATTGAAGTCTAATCA |
| | | | TCTTACTCAAAAGAACATATCTCAAGAGTTGCTTTCCTTTAGTAGATTTTCTATATAACT |
| | | | GGGTCTCTGTCTCTCTCCCGCCTTGCCCCAACTGTGATCCCCCTACCGTCTTGCCTTTTT |
| | | | TCTTCCAACGAGCCCCGGGTGTATCTTCTCAACCAGCCACAAAAGTATTTCCACTTTGAT |
| | | | ATCGCATATTAGTGCTATACACCGAGCATACAAACAACCTATACATAATAAAAGTTATTA |
| | | | TTTCAAAAAAAAAAAAAAAAAAAAAAAA |
| Mi11 | SEQ ID NO: 10 | SEQ ID NO: 11 | SEQ ID NO:8 |
| | CCATTGCTCG | GAACGAGTAA | CCATTGCTCGTCGTAAAAAGGAAATCAATAAAATGAAGGATCGTCCAACTTATGAGCGTT |
| | TCGTAAAAAG | CAGGGCCATT | ACATAGAACAAGTTCCAAAGGATGACAGGAAGAAAGGTGTTCATCCTAAAACACCTAACA |
| | G | AGG | AATTTCTCAATTGGAGTAGACGATCATGGGACAAACAGATCAAATTATGGAAACTTGCAA |
| | | | TATATGAATGGGCAGGGGAATCGCCATCTTCAAGTGTTVACGGCTCTTATTGTTCATCGG |
| | | | AATGTAGTGACGAGGATCGAGCGCAGTCTTTGGAACGGCCGCATTCAGCGGAGCCTTCAA |
| | | | CATCAAAAAAGATTAAAATTGAGAGGGACGTTGAACCGAAGAATAGGATTGTTAATATTC |
| | | | CACCATCAGCCGACACTATGGCTTCATTGTTAGGTCACTTTGATTTGGACACGCTTCGTG |
| | | | AAGCACCAGCACCTATTATGAACACAACTGGAGATGAATCAACATTGAAAGATATTCCTA |
| | | | AACCTAATGGCCCTGTTACTCGTTC |
| Mi38 | SEQ ID NO: 16 | SEQ ID NO: 17 | SEQ ID NO: 14 |
| | GTGTCATATA | CGAGATGGTT | CGAGATGGTTTCGTAAATTATTCAGATGGCAAAAACAAAGAAGGCTTGTATTATTTTCTT |
| | ACTTGGAGTT | TCGTAAATTA | TGATGAAGTTGATGCTATTGGCGGAGCTCGTTTTGATGATGGAATGGGGGGAGATAATGA |
| | GAACTAAACT | TTCAGATGG | AGTTCAACGGACAATGCTCGAATTGATCAATCAGCTGGATGGATTTGACTCGCGTGGTGC |
| | TTGC | | AATCAAAGTTTTAATGGCCACAAATCGTCCTGATACTTTGGATCCTGCACTTGTACGGCC |
| | | | AGGACGTATTGATCGTCGTATCGAATTCGCCGTGCCTGATTTGGAAGCTCGTGCAAACAT |
| | | | TCTAAAGATTCATACAAAGCGGATGAGCGTCGATCGGAATATVCGTTATGAATTGATATC |
| | | | GCGTCTTTGCCCTAATACAACAGGAGCTGATATTCGCTCTGTCTGTACTGAAGCCGGAAT |
| | | | GTTTGCATTACGTGCTCGTCGTAAAGCAATAACCGAAAAGGATTTTATTGACGCTGTTCA |
| | | | GAAAGTGGTGAAAGGATATGCAAAGTTTAGTTCAACTCCAAGTTATATGACAC |
| Mi40 | SEQ ID NO: 22 | SEQ ID NO: 23 | SEQ ID NO: 20 |
| | CTGGAAGAAT | GCTGGAACTT | CTGGAAGAATACAGATTGCGAAATTCTTAAAGCTGCTATTATGAAATATGGAAAAAATCA |
| | ACAGATTGCG | GCTTTTCAAA | ATGGAGCCGAATTGCCTCATTACTTCATCGAAAATCTGCGAAACAATGTAAAGCCCGTTG |
| | AAATTCTTAA | AGGTATTC | GTATGAATGGCTTGATCCTCGAATTAAAAAGACGGAATGGTCCAGAGAAGAGGACGAAAA |
| | AG | | ACTTCTCCATTTGGCCAAAATTATGCCTACTCAATGGCGTACAATTGCTCCTATTGTTGG |
| | | | AAGAACTGCTGCTCAGTGTTTGGAAAGATATGAATATCTTCTTGACGAAGCTCAACGTCG |
| | | | TGCTGAAGGTATTGAAGAGGATGATTCATTAAAAGAGGCTAAGAAGTTAAAGCCAGGCGA |
| | | | TGTTGATCCAACGCCAGAAACTAAACCGGCTAGGCCTGATCCTATTGATATGGATGAAGA |
| | | | TGAATTGGAAATGTTATCAGAAGCCCGAGCTCGTTTGGCCAATACACAAGGAAAGAAAGC |
| | | | AAAACGCAAAGCTCGTGAAAAGCAATTAGGCGAGGCTCGAAGACTTGCTTCTCTACAAAA |
| | | | ACGAAGAGAACTTCGGGCTGCTGGAATAATTATTGGATCTGGTTGGGCTTATAAAATGAA |
| | | | GAAGCATCGTCTAATGGATTATAATAATGGAATACCTTTTGAAAAGCAAGTTCCAGC |
| Mi101 | | | SEQ ID NO: 26 |
| | | | GGTTATTACCCATGCTTGAGACAAAAGCACAATATTCTGAGAAATTTTAAACAACAACAA |
| | | | TGCATTTGTCTTTAGCTAAAGTTGTTGCTATTAAAGAGCCGCCTCTATATGATCGTCGAC |
| | | | AAGGATTTGTGCCCCGAACGCAAGATGATTTTGGTGATGGTGGGGCTTTTCCAGAAATAC |
| | | | ATATTGCTCAGTTTCCAATCGGTATGGGCGCCGATAAGCCCGGCACTGGGGCAAAGAACA |
| | | | CAGTTGCGCTTCAGTTTGACTCTGAGGGCAAACTTCGATTTGATGAGCTTACACGAATTG |
| | | | GGCATGGAAAAGACAAGATTGTTCATTCCCGCCTTTCTGATATGAAATCCAACCATATTG |
| | | | ATGATGAAGACGAAAGTTTCAAAAAACCAACAGATGAAGAAATACATGAAACAGCCGAAG |
| | | | CAACTCGTGTTTCCTTAGAGAAAATTACAGCTGTTAAGATCGCCGCATCATTACCAGTCC |
| | | | AGCATGCAAAGAAAACAGCGCCTGCACAATATATTCGTTACACTCCTAGCCAGCAGGCTG |
| | | | GTGGTTTTCATACAAGTGGAGCACAACAAAGAATTATTCGTTTGGTTGAAGAGCAGAAGG |
| | | | ATCCAATGGAGCCGCCACGTTTTCAAATAAACCAGAAA |
| Mi109 | | | SEQ ID NO: 31 |
| | | | GCGTTTGGCGGCCTCGCTTCAAAAGTGCGGCAAAGGCAAAATTTGGTTGGATCCTAATGA |
| | | | GGTCAACGTGATTTCCAACGCAAATTCTCGTCAGAACGTTCGTCGTTTGGTCAAAGATGG |
| | | | TCTTATAATTCGTAAGCCTGGCAAAGTTCATTCGCGTTTCCGAGCTCGTGAACGTTTGGA |
| | | | AGCACGCAGAAAGGGTCGTCATATGGGTTTTGGAAAACGACGCGGAACTCGTAACGCTCG |
| | | | AATGCCGGAGAAGGTCTTATGGATGCGTTTTTTTGTTGTACTCCGTCATCTTTTAAAGCG |
| | | | CTATCGAGCTGCGAAGAAGATTGACAAGCATCTTTACCACGAGCTTTACTTGAAGGCTAA |
| | | | AGGAAACGCATTTAAGAATAAGAGAAATTTGATGGAATATATCTTCAAAAAGAAGACTGA |
| | | | GAACCAGCGTTCTAAACAACTTGCTGAACAGGCTGAGGCTCGCCGAAACAAGAACAAGGA |
| | | | AACGAGAAAGAGGCGTGAGGAGCGTTTGATTTTGAAACGTAAGGAATTGCTCCACAAACT |
| | | | TTCTGAGAGCGAAAAGCAGAAGACTCCACAGCTTCAAGAAGAGGAAGCGAAGGAAGCTTC |
| | | | GCCTTCTCCACCACC |
| Mi111 | | | SEQ ID NO: 35 |
| | | | GGGAAATCAGCAATTCCAACAATTTACCCCAGGTTCAGGAGATAAGCAAGGAGAAGGAGA |
| | | | CAAGAAGCGTCGTTTTGGGCCTCCAATCCCTACACGCTTTGGGAAGCGAAAGAAGGGAAG |
| | | | TAAAGGACCTGAAGCTAGCAATAAAATGCCTAATGTGACACCAGTAACTCGTTGCAAATT |
| | | | GCGTCTTCTTAAATATGAACGGATTAAAGATTATTTGTTAATGGAAGAAGAATTTATTCG |
| | | | AAATCAGGAAAGACATAAACCTCAAGAGGAAAGGCAGGAAGAAGAACGGACAAAAGTAGA |
| | | | TGAAATGAGAGGGTCACCAATGGCTGTGGGTACATTGGAGGAGATTATTGACGATCAACA |
| | | | CGCAGTCGTTTCTACGAATGTTGGCAGCGAACATTATGTTAACATTCTTTCATTTGTGGA |
| | | | CAAGGAACAATTGGAGCCAGGCTGTGCAGTTCTTCTAAATCACAAAACACACGCAGTTGT |
| | | | TGGTGTTTTGGCTGATGACACGGATCCAATGGTTTCTGTAATGAAATTGGAAAAGGCTCC |
| | | | AACTGAAACGTACGCTGATGTTGGCGGTTTGGAACAACAAATTCAAGAAATTAAGGAGGC |
| | | | AGTGGAATTGCCACTTACACATCCAGAGTACTACGAAGAAATTGGAATCAAGCCTC |
| M1116 | | | SEQ ID NO: 39 |
| | | | CAGCCAGTGGTCTTTCCCCTTCAATCACACCAAATTCCTTGATATTTTCTTTAACATTTT |
| | | | TTTCAAACTGTGGGTCTCCAAGCAATAAGTTTTCAGGGTATAGGTCAGGTGGTGACTCAT |
| | | | TTTTCAATATTTGATAGTGGTTTGTATTTTTGACTTTACTTAACTTGTTTACTGAAATCT |
| | | | GTTCCTGTTCTTCAAGATGACCGCACAATTCGAAAGATACACAAATTGGATGATCACGTA |
| | | | ATGGCTGCTTTTGCTGGTTTGAGTGCAGATGCTCGTGTTTTGATTGACCGTGCCAGAGTT |
| | | | GCTTGTGAAAATTACAAATTGACTTTGGAGGATCCCGTCACACTTGAGCACATCTCCCGC |
| | | | ACTATTGCGGACTTGAAACATGAATTTACGCAAACTACAGGTCGTCGTCCTTTTGGTGTC |
| | | | TCTTTACTTGTTGGCGGTTTTGATCCCGATGGAACTCCTCATCTTTTTATTACTGAACCA |
| | | | TCTGGTGTGTATTACGAATTACTTGCTGGCTCAATCGGCCGGAATGAGAAGGTTGTCAAA |
| | | | GAATATTTGGAGGAGAATTATAATGATGGCAATGTTTCAAACGAAAACTTGGTCTTGAAG |
| | | | TTGGTGGTCAAAGCTCTTGTCCCAGTTGTGCAAACTGGAGCGCGTAATATGGAGATATCG |
| | | | GTGTTGAAGCGTGATAATGAGGGGAATATTGTTCAACGTGTTCTACTCACTGAGGAGTTG |
| | | | GAGGAAATTTTACGTCAAATTGAAGCTGAAACTATGGAGACTGTTTAGACTTTCTTTGAT |
| | | | TTTTTTAAAAATTCAGTCGAACCTCATTTTTAGATACTCTTGGGGCTGAAAAATCGTATC |
| | | | TTATTTGAGGTCTTATAATGAGGTCCAACTATACATACATTTGCTATTTTTGTTAATAAA |
| | | | TGTTTCGCTATTCTG |
| M1125 | | | SEQ ID NO: 43 |
| | | | GGTTTAATTACTCCAGTTTGAGCTTAAAAAGTAAAACAAGATGCCTCGCATAAAGCATGT |
| | | | ATTTTCTAAAGTTTATGACGTGCCTCGTCGTCCATTCGAGAAGGAGCGTTTAGATCAGGA |
| | | | ATTAAAATTAATTGGCGAGTTTGGTTTACGAAATAAACGTGAAGTTTGGCGTGTCAAATT |
| | | | AACTTTGGCAAAGATTCGTAAAGCTGCTCGTGAACTTTTAACATTGGAAGACAAGGATCC |
| | | | TCGACGCTTATTTGAAGGAAATGCTCTTCTTCGTCGTTTGGTTCGTATTGGAGTTCTTTT |
| | | | GGAGGATAAGATGAAATTGGACTATGTTCTTGGTTTGCGTGTTGAAGACTTTTTGGAGCG |
| | | | TCGTTTGCAGACGCAAGTTTTCAAATTGGGATTGGCTAAATCAATTCACCATGCTCGTGT |
| | | | TCTTATTCGTCAAAAGCATATTCGAATTCGTCGACAAGTAGTCAATATTCCATCATTCAT |
| | | | TGTTCGTCTCGACTCTCAAAAGCACATTGACTTCTCCACAAAATCTTATCTTGCTGGTGG |
| | | | ACCTCCTGGACGTGTCAAGAGAAAGAATTTGAAGAAGGCTGCTTCGGGAGGTGGAGGAGG |
| | | | AGGCGGTGACGATGAAGAGGATTGAATAATATCTAAAGGTTTATAAAACGTTGAAAGTTA |
| | | | ATTCTGTTAAGTTGTTAAAAGATTTTGGATTTGAAAAAATGTTTTTGTTGA |
| Mi127 | | | SEQ ID NO: 49 |
| | | | GGTTTAATTACCCAAGTTTGAGCTATCATTTTAAAAAAATCTTTTAAAAATGTCTAAAGC |
| | | | TAACGCTGTCGGAATTGATCTGGGAACGACTTATTTATGTGTCGGTGTATTCCAGCATGG |
| | | | AAAAGTCGAGATCATTGCCAATGATCAAGGAAATCGCACGACTCCATCTTATGTGGCTTT |
| | | | TACAGACACGGAACGTCTGATTGGTGATGCTGCGAAAAATCAAGTGGCAATGAACCCGTC |
| | | | GAATACTGTTTTTGACGCAAAGCGCTTGATTGGACGCAAATTTGACGATCCAGCTGTTCA |
| | | | ATCTGACATGAAGCATTGGCCGTTCAAGGTTATTCAAGGAGAAGGTGCTCGTCCAAAAAT |
| | | | CCAAGTGGAGGTTAAGGGTGAAATGAAGGCGTTCTTCCCTGAGGAAGTCTCCGCCATGGT |
| | | | TTTGACTAAAATGAAGGAAACGGCTGAGGCATTTTTGGGCCAAACCGTTAAGGATGCTGT |
| | | | CATCACTGTGCCTGCGTACTTTAATGACAGTCAACGCCAAGCCACAAAGGATGCTGGAAC |
| | | | GATTTCTGGACTTAATGTTTTGCGTATCATCAACGAGCCAACTGCTGCGGCTATTGCTTA |
| | | | TGGTTTGGATAAGAAAGGCCAAGGCGAGCGCAACGTCTTGATTTTTGACTTGGGCGGTGG |
| | | | AACTTTTGATGTGTCTATTTTGACTATTGAAGATGGTATTTTTGAGGT |
| Mi128 | | | SEQ ID NO: 52 |
| | | | GGTTTAATTACCTACGTTTGAGAAATTTAAAGAATGACAAAGTTACAACATCCAGCTGTT |
| | | | GAGGGTCTTGCTGTTGGTTTGGATCGTGGCCATCCTGTGACTAAAAATGTTCGCAAGCCA |
| | | | CGTCAATCTCGTAAAAAAGGCCGCATTACGAAGAAGACTCGAATTGTTCGTGAAGTTGTT |
| | | | CGCGAGGTTGTTGGTTTTTCACCTTATGAGCGTCGAACTATGGAATTGCTTCGTATCAGT |
| | | | AAGGACAAGAAGGCTTTGAAGTTTTTGAAGAAGCGAATTGGACAACATGTCCGTGCAAAG |
| | | | AGAAAGCGTGACGAAATCCAACGAATACTCGTTGAGATGAGAAAGCATCATAAGTGAGAA |
| | | | AATATAAGGGAAGGAAAACATTCAATCACAGTTTTAATTGTTAAAAATAATGTTTTATGC |
| | | | GATTGAATAAATTTGTTAGAATTCAAACAAAA |
| Mi129 | | | SEQ ID NO: 55 |
| | | | AGGTTTAATTACCCAAGTTTGAGTTTTACTATTVAATTTATCTAACAAAAGATCTCAAAA |
| | | | AATGCGTGAAATCGTCCATATCCAGGCTGGACAATGTGGTAACCAAATTGGTTCCAAGTT |
| | | | CTGGGAAGTCATTTCGGATGAACACGGAATTCAACCTGATGGATCATACAAGGGAGAATC |
| | | | TGACTTGCAATTGGAACGAATCAATGTTTACTACAATGAGGCTCATGGTGGAAAGTATGT |
| | | | CCCAAGAGCTGTTCTTGTTGACTTGGAGCCGGGAACCATGGACTCGATTAGAGCTGGGCC |
| | | | ATACGGAGAACTCTTCCGTCCTGACAACTTTGTCTTTGGCCAGAGCGGTGCAGGAAACAA |
| | | | TTGGGCCAAAGGACATTACACTGAGGGAGCGGAGTTGGTCGACAATGTTTTGGATGTTGT |
| | | | TCGAAAAGAGGCTGAGGGTTGTGATTGTCTTCAAGGCTTTCAATTGACTCACTCGCTTGG |
| | | | TGGTGGAACTGGCTCTGGAATGGGAACTTTGCTCATTTCAAAGATTCGTGAGGAGTATCC |
| | | | GGACCGCATTATGTCTTCCTTCTCTGTTGTTCCGTCGCCTAAGGTCTCTGACACGGTCGT |
| | | | TGAGCCCTACAACGCAACTCTTTCTGTTCATCAACTTGTTGAGAACACCGACGAGACTTA |
| | | | CTGCATCGACAACGAGGCTTTGTACGACATCTGCTTCCGAACTCTGAAGCTTCAAAATCC |
| | | | AACATATGGAGATTTGAATCATCTCGTATCCGTAACCATGTCTGGAGTGACTACTTGTCT |
| | | | TCGCTTCCCAGGCCAATTGAACGCTGATCTTCGCAAATTGGCTGTCAACATGGTTCCATT |
| | | | CCCACGTCTTCACTTTTTCATGCCTGGTTTCGCTCCACTTTCGGCTAAGGGCGCAGCTGC |
| | | | CTACCAGGCCCAAAATGTTGCTGAATTGACCAAGCAAATGTTTGATGCCAAGAATATGAT |
| | | | GGCTGCTTGTGATCCACGCCATGGACGCTATTTAACTGTTGCCGCTATCTTCCGTGGACG |
| | | | TATGTCAATGCGTGAAGTAGACGATCAAATGATGTCGGTTCAAAACAAGAATTCGTCATA |
| | | | CTTTGTTGAATGGATTCCAAACAACGTTAAGACTGCGGTTTGTGACATTCCTCCGCGTGG |
| | | | TCTCAAGATGTCTGCGACCTTCATTGGCAACTCCACGGCCATTCAAGAACTGTTCAAACG |
| | | | CATCTCTGAGCAATTCACGGCCATGTTCCGTCGCAAGGCTTTCCTTCACTGGTACACTGG |
| | | | CGAAGGTATGGACGAGATGGAATTCACTGAAGCCGAGTCCAACATGAACGACTTGATTTC |
| | | | TACCAATCAAGCAACTGTTGAACAAGAATAAAAAAGAGAGAAGTTTTTGTTGAATACCAG |
| | | | CAATATCAAGATGCGACTGTTGAAGACGAAGGGGAATTTGAAGGTGAAGATTTGAATTAC |
| | | | CACGTATTTTTTTAATTTTATTTTGCTTTTAACCGCATATTTCCGAAATAATTCCTCGCTT |
| | | | AACTTTATTTATGCCAAAAAA |

TABLE 3

| Target | Amino acid sequence of cDNA sequence |
|---|---|
| ID | (Translation of Contig) sense strand 5′ → 3′ |
| Mi05 | SEQ ID NO: 2 |
| | LRHVIMIHSDSKPYQCFDCDFIGIKSNWSHARQSGHRNDDALDI |
| | TTDDMRAEWNTMLHACFPDYVKAKSRGWQPETEVKDEPLESNNE |
| | QDTTVKIEV |
| Mi11 | SEQ ID NO: 9 |
| | IARRKKEINKMKDRPTYERYIEQVPKDDRKKGVHPKTPNKFLNW |
| | SRRSWDKQIKLWKLAIYEWAGESPSSSVYGSYCSSECSDEDRAQ |
| | SLERPHSAEPSTSKKIKIERDVEPKNRIVNIPPSADTMASLLGH |
| | FDLDTLREAPAPIMNTTGDESTLKDIPKPNGPVTR |
| Mi38 | SEQ ID NO: 15 (partial translation) |
| | IIQMAKTKKACIIFFDEVDAIGGARFDDGMGGDNEVQRTMLELI |
| | NQLDGFDSRGAIKVLMATNRPDTLDPALVRPGRIDRRIEFAVPD |
| | LEARANILKIHTKRMSVDRNIRYELISRLCPNTTGADIRSVCTE |
| | AGMFALRARRKAITEKDFIDAVQKVVKGYAKFSSTPSYMT |
| Mi40 | SEQ ID NO: 21 |
| | WKNTDCEILKAAIMKYGKNQWSRIASLLHRKSAKQCKARWYEWL |
| | DPRIKKTEWSREEDEKLLHLAKIMPTQWRTIAPIVGRTAAQCLE |
| | RYEYLLDEAQRRAEGIEEDDSLKEAKKLKPGDVDPTPETKPARP |
| | DPIDMDEDELEMLSEARARLANTQGKKAKRKAREKQLGEARRLA |
| | SLQKRRELRAAGIIIGSGWAYKMKKHRLMDYNNGIPFEKQVP |
| Mi101 | SEQ ID NO: 27 (partial translation) |
| | TTTMHLSLAKVVAIKEPPLYDRRQGFVPRTQDDFGDGGAFPEIH |
| | IAQFPIGMGADKPGTGAKNTVALQFDSEGKLRFDELTRIGHGKD |
| | KIVHSRLSDMKSNHIDDEDESFKKPTDEEIHETAEATRVSLEKI |
| | TAVKIAASLPVQHAKKTAPAQYIRYTPSQQAGGFHTSGAQQRII |
| | RLVEEQKDPMEPPRFQINQK |
| Mi109 | SEQ ID NO: 32 |
| | RLAASLQKCGKGKIWLDPNEVNVISNANSRQNVRRLVKDGLIIR |
| | KPGKVHSRFRARERLEARRKGRHMGFGKRRGTRNARMPEKVLWM |
| | RFFVVLRHLLKRYRAAKKIDKHLYHELYLKAKGNAFKNKRNLME |
| | YIFKKKTENQRSKQLAEQAEARRNKNKETRKRREERLILKRKEL |
| | LHKLSESEKQKTPQLQEEEAKEASPSPP |
| Mi111 | SEQ ID NO: 36 |
| | GNQQFQQFTPGSGDKQGEGDKKRRFGPPIPTRFGKRKKGSKGPE |
| | ASNKMPNVTPVTRCKLRLLKYERIKDYLLMEEEFIRNQERHKPQ |
| | EERQEEERTKVDEMRGSPMAVGTLEEIIDDQHAWSTNVGSEHYV |
| | NILSFVDKEQLEPGCAVLLNHKTHAVVGVLADDTDPMVSVMKLE |
| | KAPTETYADVGGLEQQIQEIKEAVELPLTHPEYYEEIGIKP |
| Mi116 | SEQ ID NO: 40 (partial translation) |
| | KSVPVLQDDRTIRKIHKLDDHVMAAFAGLSADARVLIDRARVAC |
| | ENYKLTLEDPVTLEHISRTIADLKHEFTQTTGRRPFGVSLLVGG |
| | FDPDGTPHLFITEPSGVYYELLAGSIGRNEKWKEYLEENYNDGN |
| | VSNENLVLKLVVKALVPVVQTGARNMEISVLKRDNEGNIVQRVL |
| | LTEELEEILRQIEAE |
| Mi125 | SEQ ID NO: 44 |
| | MPRIKHVFSKVYDVPRRPFEKERLDQELKLIGEFGLRNKREVWR |
| | VKLTLAKIRKAARELLTLEDKDPRRLFEGNALLRRLVRIGVLLE |
| | DKMKLDYVLGLRVEDFLERRLQTQVFKLGLAKSIHHARVLIRQK |
| | HIRIRRQVVNIPSFIVRLDSQKHIDFSTKSYLAGGPPGRVKRKN |
| | LKKAASGGGGGGGDDEED |
| Mi127 | SEQ ID NO: 50 |
| | MSKANAVGIDLGTTYLCVGVFQHGKVEIIANDQGNRTTPSYVAF |
| | TDTERLIGDAAKNQVAMNPSNTVFDAKRLIGRKFDDPAVQSDMK |
| | HSPFKVIQGEGARPKIQVEVKGEMKAFFPEEVSAMVLTKMKETA |
| | EAFLGQTVKDAVITVPAYFNDSQRQATKDAGTISGLNVLRIINE |
| | PTAAAIAYGLDKKGQGERNVLIFDLGGGTFDVSILTIEDGIFE |
| Mi128 | SEQ ID NO: 53 |
| | MTKLQHPAVEGLAVGLDRGHPVTKNVRKPRQSRKKGRITKKTRI |
| | VREVVREWGFSPYERRTMELLRISKDKKALKFLKKRIGQHVRAK |
| | RKRDEIQRILVEMRKHHK |
| Mi129 | SEQ ID NO: 56 |
| | MREIVHIQAGQCGNQIGSKFWEVISDEHGIQPDGSYKGESDLQL |
| | ERINVYYNEAHGGKYVPRAVLVDLEPGTMDSIRAGPYGELFRPD |
| | NFVFGQSGAGNNWAKGHYTEGAELVDNVLDWRKEAEGCDCLQGF |
| | QLTHSLGGGTGSGMGTLLISKIREEYPDRIMSSFSWSPKVSDTV |
| | VEPYNATLSVHQLVENTDETYCIDNEALYDICFRTLKLQNPTYG |
| | DLNHLVSVTMSGVTTCLRFPGQLNADLRKLAVNMVPFPRLHFFM |
| | PGFAPLSAKGAAAYQAQNVAELTKQMFDAKNMMAACDPRHGRYL |
| | TVAAIFRGRMSMREVDDQMMSVQNKNSSYFVEWIPNNVKTAVCD |
| | IPPRGLKMSATFIGNSTAIQELFKRISEQFTAMFRRKAFLHWYT |
| | GEGMDEMEFTEAESNMNDLISEYQQYQDATVEDEGEFEGEDTNQ |
| | ATVEQE |

TABLE 4

| Target | DNA Sequence of fragments (sense |
|---|---|
| ID | freefrag) and concatemer constructs |
| Mi05 | SEQ ID NO: 5 |
| | AGGCAAAGAGCCGTGGATGGCAGCCCGAAACCGAGGTCAAAGAC |
| | GAGCCTCTGGAATCCAACAACGAACAGGACACTACCGTCAAAAT |
| | TGAAGTCTAATCATCTTACTCAAAAGAACATATCTCAAGAGTTG |
| | CTTTCCTTTAGTAGATTTTCTATATAACTGGGTCTCTGTCTCTC |
| | TCCCGCCTTGCCCCAACTGTGATCCCCCTACCGTCTTGCCTTTT |
| | TTCTTCCAACGAGCCCCGGGTGTATCTTCTCAACCAGCCACAAA |
| | AGTATTTCCACTTTGATATCGCATATTAGTGCTATACACCGAGC |
| | ATACAAACAACCTATACATAATAAAA |
| Mi05 | SEQ ID NO: 6 |
| | TACCGGCTCAAGGCAAAGAGCCGTGGATGGCAGCCCGAAACCGA |
| | GGTCAAAGACGAGCCTCTGGAATCCAACAACGAACAGGACACTA |
| | CCGTCAAAATTA |
| Mi11 | SEQ ID NO: 12 |
| | TTTGATCTGTTTGTCCCATGATCGTCTACTCCAATTGAGAAATT |
| | TGTTAGGTGTTTTAGGATGAACACCTTTCTTCCTGTCATCCTTT |
| | GGAACTTGTTCTATGTAACGCTCATAAGTTGGACGATCCTTCAT |
| | TTTATTGATTTCCTTTTTACGACGAGCAATGG |
| Mi38 | SEQ ID NO: 18 |
| | CGATCGACGCTCATCCGCTTTGTATGAATCTTTAGAATGTTTGC |
| | ACGAGCTTCCAAATCAGGCACGGCGAATTCGATACGACGATCAA |
| | TACGTCCTGGCCGTACAAGTGCAGGATCCAAAGTATCAGGACGA |
| | TTTGTGGCCAT |
| Mi40 | SEQ ID NO: 24 |
| | AAGAAGATATTCATATCTTTCCAAACACTGAGCAGCAGTTCTTC |
| | CAACAATAGGAGCAATTGTACGCCATTGAGTAGGCATAATTTTG |
| | GCCAAATGGAGAAGTTTTTCGTCCTCTTCTCTGGACCATTCCGT |
| | CTTTTTAATTCGAGGATCAAGCCATTCATACCAACGGGCTTTAC |
| | A |
| Mi101 | SEQ ID NO: 28 |
| | GAGCCGCCTCTATATGATCGTCGACAAGGATTTGTGCCCCGAAC |
| | GCAAGATGATTTTGGTGATGGTGGGGCTTTTCCAGAAATACATA |
| | TTGCTCAGTTTCCAATCGGTATGGGCGCCGATAAGCCCGGCACT |
| | GGGGCAAAGAACACAGTTGCGCTTCAGTTTGACTCTGAGGGCAA |
| | ACTTCGATTTGATGAGCTTACACGAATTGGGCATGGAAAAGACA |
| | AGATTGTTCATTCCCGCCTTTCTGATATGAAATCCAACCATATT |
| | GATGATGAAGACGAAAGTTTCAAAAAACCAACAGATGAAGAAAT |
| | ACATGAAACAGCCGAAGCAACTCGTGTTTCCTTAGAGAAAATTA |
| | CAGCTGTTAAGATCGCCGCATCATTACCAGTCCAGCATGCAAAG |
| | AAAACAGCGCCTGCACAATATATTCG |
| Mi101 | SEQ ID NO: 29 |
| for | ACTCGTGTTTCCTTAGAGAAAATTACAGCTGTTAAGATCGCCGC |
| MiCC2 | ATCATTACCAGTCCAGCATGCAAAGAAAACAGCGCCTGCACAAT |
| | ATATTCGTTACACTCCTAGCCAGCAGGCTGGTGGTTTTCATACA |
| | AGTGGAGCACAACAAAGA |
| Mi109 | SEQ ID NO: 33 |
| | GTTGGATCCTAATGAGGTCAACGTGATTTCCAACGCAAATTCTC |
| | GTCAGAACGTTCGTCGTTTGGTCAAAGATGGTCTTATAATTCGT |
| | AAGCCTGGCAAAGTTCATTCGCGTTTCCGAGCTCGTGAACGTTT |
| | GGAAGCACGCAGAAAGGGTCGTCATATGGGTTTTGGAAAACGAC |
| | GCGGAACTCGTAACGCTCGAATGCCGGAGAAGGTCTTATGGATG |
| | CGTTTTTTTGTTGTACTCCGTCATCTTTTAAAGCGCTATCGAGC |
| | TGCGAAGAAGATTGACAAGCATCTTTACCACGAGCTTTACTTGA |
| | AGGCTAAAGGAAACGCATTTAAGAATAAGAGAAATTTGATGGAA |
| | TATATCTTCAAAAAGAAGACTGAGAACCAGCGTTCTAAACAACT |
| | TGCTGAACAGGCTGAGGCTCG |
| Mi111 | SEQ ID NO: 37 |
| | GGCCTCCAATCCCTACACGCTTTGGGAAGCGAAAGAAGGGAAGT |
| | AAAGGACCTGAAGCTAGCAATAAAATGCCTAATGTGACACCAGT |
| | AACTCGTTGCAAATTGCGTCTTCTTAAATATGAACGGATTAAAG |
| | ATTATTTGTTAATGGAAGAAGAATTTATTCGAAATCAGGAAAGA |
| | CATAAACCTCAAGAGGAAAGGCAGGAAGAAGAACGGACAAAAGT |
| | AGATGAAATGAGAGGGTCACCAATGGCTGTGGGTACATTGGAGG |
| | AGATTATTGACGATCAACACGCAGTCGTTTCTACGAATGTTGGC |
| | AGCGAACATTATGTTAACATTCTTTCATTTGTGGACAAGGAACA |
| | ATTGGAGCCAGGCTGTGCAGTTCTTCTAAATCACAAAACACACG |
| | CAGTTGTTGG |
| Mi116 | SEQ ID NO: 41 |
| | TCACACCAAATTCCTTGATATTTTCTTTTAACATTTTTTTCAAA |
| | CTGTGGGTCTCCAAGCAATAAGTTTTCAGGGTATAGGTCAGGTG |
| | GTGACTCATTTTTCAATATTTGATAGTGGTTTGTATTTTTGACT |
| | TTACTTAACTTGTTTACTGAAATCTGTTCCTGTTCTTCAAGATG |
| | ACCGCACAATTCGAAAGATACACAAATTGGATGATCACGTAATG |
| | GCTGCTTTTGCTGGTTTGAGTGCAGATGCTCGTGTTTTGATTGA |
| | CCGTGCCAGAGTTGCTTGTGAAAATTACAAATTGACTTTGGAGG |
| | ATCCCGTCACACTTGAGCACATCTCCCGCACTATTGCGGACTTG |
| | AAACATGAATTTACGCAAACTACAGGTCGTCGTCCCTTTGGTGT |
| | CTCTTTACTTGTTGGCGGTTTTGATCCCGATGGAACTCCTCATC |
| | TTTTTATTAC |
| Mi125a | SEQ ID NO: 45 |
| | ATCCTCTTCATCGTCACCGCCTCCTCCTCCACCTCCCGAAGCAG |
| | CCTTCTTCAAATTCTTTCTCTTGACACGTCCAGGAGGTCCACCA |
| | GCAAGATAAGATTTTGTGGAGAAGTCAATGTGCTTTTGAGAGTC |
| | GAGACGAACAATGAATGATGGAATATTGACTACTTGTCGACGAA |
| | TTCGAATATGCTTTTGACGAATAAGAACACGAGCATGGTGAATT |
| | GATTTAGCCAATCCCAATTTGAAAACTTGCGTCTGCAAACGACG |
| | CTCCAAAAAGTCTTCAACACGCAAACCAAGAACATAGTCCAATT |
| | TCATCTTATCCTCCAAAAGAACTCCAATACGAACCAAACGACGA |
| | AGAAGAGCATTTCCTTCAAATAAGCGTCGAGGATCCTTGTCTTC |
| | CAATGTTAAAAGTTCACGAGCAGCTTTACGAATCTTTGCCAAAG |
| | TTAATTTGACACGCCAAACTTCACGTTTATTTCGTAAACCAAAC |
| | TCGCCAATTAATTTTAATTCCTGATCTAAACGCTCCTTCTCGAA |
| | TGGACGACGAGGCAC |
| Mi125b | SEQ ID NO: 46 |
| | TTCATCTTATCCTCCAAAAGAACTCCAATACGAACCAAACGACG |
| | AAGAAGAGCATTTCCTTCAAATAAGCGTCGAGGATCCTTGTCTT |
| | CCAATGTTAAAAGTTCACGAGCAGCTTTACGAATCTTTGCCAAA |
| | GTTAATTTGACACGCCAAACTTCACGTTTATTTCGTAAACCAAA |
| | CTCGCCAATTAATTTTAATTCCTG |
| Mi127 | SEQ ID NO: 51 |
| | ATTTTTCGCAGCATCACCAATCAGACGTTCCGTGTCTGTAAAAG |
| | CCACATAAGATGGAGTCGTGCGATTTCCTTGATCATTGGCAATG |
| | ATCTCGACTTTTCCATGCTGGAATACACCGACACATAAATAAGT |
| | CGTTCCCAGATCAATTCC |
| Mi128 | SEQ ID NO: 54 |
| | TGAGAAATTTAAAGAATGACAAAGTTACAACATCCAGCTGTTGA |
| | GGGTCTTGCTGTTGGTTTGGATCGTGGCCATCCTGTGACTAAAA |
| | ATGTTCGCAAGCCACGTCAATCTCGTAAAAAAGGCCGCATTACG |
| | AAGAAGACTCGAATTGTT |
| Mi129 | SEQ ID NO: 57 |
| | CCGACGAGACTTACTGCATCGACAACGAGGCTTTGTACGACATC |
| | TGCTTCCGAACTCTGAAGCTTCAAAATCCAACATATGGAGATTT |
| | GAATCATCTCGTATCCGTAACCATGTCTGGAGTGACTACTTGTC |
| | TTCGCTTCCCAGGCCAATTGAACGCTGATCTTCGCAAATTGGCT |
| | GTCAACATGGTTCCATTCCCACGTCTTCACTTTTTCATGCCTGG |
| | TTTCGCTCCACTTTCGGCTAAGGGCGCAGCTGCCTACCAGGCCC |
| | AAAATGTTGCTGAATTGACCAAGCAAATGTTTGATGCCAAGAAT |
| | ATGATGGCTGCTTGTGATCCACGCCATGGACGCTATTTAACTGT |
| | TGCCGCTATCTTCCGTGGACGTATGTCAATGCGTGAAGTAGAC |
| MiCC2 | SEQ ID NO: 59 |
| | ATTTTTCGCAGCATCACCAATCAGACGTTCCGTGTCTGTAAAAG |
| | CCACATAAGATGGAGTCGTGCGATTTCCTTGATCATTGGCAATG |
| | ATCTCGACTTTTCCATGCTGGAATACACCGACACATAAATAAGT |
| | CGTTCCCAGATCAATTCCTGAGAAATTTAAAGAATGACAAAGTT |
| | ACAACATCCAGCTGTTGAGGGTCTTGCTGTTGGTTTGGATCGTG |
| | GCCATCCTGTGACTAAAAATGTTCGCAAGCCACGTCAATCTCGT |
| | AAAAAAGGCCGCATTACGAAGAAGACTCGAATTGTTACTCGTGT |
| | TTCCTTAGAGAAAATTACAGCTGTTAAGATCGCCGCATCATTAC |
| | CAGTCCAGCATGCAAAGAAAACAGCGCCTGCACAATATATTCGT |
| | TACACTCCTAGCCAGCAGGCTGGTGGTTTTCATACAAGTGGAGC |
| | ACAACAAAGA |

TABLE 5

| | |
|---|---|
| Target | |
| ID | Hairpin Sequence |

| | |
|---|---|
| M105 | SEQ ID NO: 7 |
| | AGGCAAAGAGCCGTGGATGGCAGCCCGAAACCGAGGTCAAAGAC |
| | GAGCCTCTGGAATCCAACAACGAACAGGACACTACCGTCAAAAT |
| | TGAAGTCTAATCATCTTACTCAAAAGAACATATCTCAAGAGTTG |
| | CTTTCCTTTAGTAGATTTTCTATATAACTGGGTCTCTGTCTCTC |
| | TCCCGCCTTGCCCCAACTGTGATCCCCCTACCGTCTTGCCTTTT |
| | TTCTTCCAACGAGCCCCGGGTGTATCTTCTCAACCAGCCACAAA |
| | AGTATTTCCACTTTGATATCGCATATTAGTGCTATACACCGAGC |
| | ATACAAACAACCTATACATAATAAAAATCGTCTCTAGACCCAGC |
| | TTTCTTGTACAAAGTGGTTGATCCTGCAGGGTCCGTCGCTTCTC |
| | TTCCATTTCTTCTCATTTTCGATTTTGATTCTTATTTCTTTCCA |
| | GTAGCTCCTGCTCTGTGAATTTCTCCGCTCACGATAGATCTGCT |
| | TATACTCCTTACATTCAACCTTAGATCTGGTCTCGATTCTCTGT |
| | TTCTCTGTTTTTTTCTTTTGGTCGAGAATCTGATGTTTGTTTAT |
| | GTTCTGTCACCATTAATAATAATGAACTCTCTCATTCATACAAT |
| | GATTAGTTTCTCTCGTCTACAAAACGATATGTTGCATTTTCACT |
| | TTTCTTCTTTTTTTCTAAGATGATTTGCTTTGACCAATTTGTTT |
| | AGATCTTTATTCTATTTTATTTTCTGGTGGGTTGGTGGAAATTG |
| | AAAAAAAAAAAACAGCATAAATTGTTATTTGTTAATGTATTCAT |
| | TTTTTGGCTATTTGTTCTGGGTAAAAATCTGCTTCTACTATTGA |
| | ATCTTTCCTGGATTTTTTACTCCTATTGGGTTTTTATAGTAAAA |
| | ATACATAATAAAAGGAAAACAAAAGTTTTATAGATTCTCTTAAA |
| | CCCCTTACGATAAAAGTTGGAATCAAAATAATTCAGGATCAGAT |
| | GCTCTTTGATTGATTCAGATGCGATTACAGTTGCATGGCAAATT |
| | TTCTAGATCCGTCGTCACATTTTATTTTCTGTTTAAATATCTAA |
| | ATCTGATATATGATGTCGACAAATTCTGGTGGCTTATACATCAC |
| | TTCAACTGTTTTCTTTTGGCTTTGTTTGTCAACTTGGTTTTCAA |
| | TACGATTTGTGATTTCGATCGCTGAATTTTTAATACAAGCAAAC |
| | TGATGTTAACCACAAGCAAGAGATGTGACCTGCCTTATTAACAT |
| | CGTATTACTTACTACTAGTCGTATTCTCAACGCAATCGTTTTTG |
| | TATTTCTCACATTATGCCGCTTCTCTACTCTTTATTCCTTTTGG |
| | TCCACGCATTTTCTATTTGTGGCAATCCCTTTCACAACCTGATT |
| | TCCCACTTTGGATCATTTGTCTGAAGACTCTCTTGAATCGTTAC |
| | CACTTGTTTCTTGTGCATGCTCTGTTTTTTAGAATTAATGATAA |
| | AACTATTCCATAGTCTTGAGTTTTCAGCTTGTTGATTCTTTTGC |
| | TTTTGGTTTTCTGCAGGTTTAAACATCAACCACTTTGTACAAGA |
| | AAGCTGGGTCTAGAGACGATTTTTATTATGTATAGGTTGTTTGT |
| | ATGCTCGGTGTATAGCACTAATATGCGATATCAAAGTGGAAATA |
| | CTTTTGTGGCTGGTTGAGAAGATACACCCGGGGCTCGTTGGAAG |
| | AAAAAAGGCAAGACGGTAGGGGGATCACAGTTGGGGCAAGGCGG |
| | GAGAGAGACAGAGACCCAGTTATATAGAAAATCTACTAAAGGAA |
| | AGCAACTCTTGAGATATGTTCTTTTGAGTAAGATGATTAGACTT |
| | CAATTTTGACGGTAGTGTCCTGTTCGTTGTTGGATTCCAGAGGC |
| | TCGTCAAGACCTCGGTTTCGGGCTGCCATCCACGGCTCTTTGCC |
| | T |
| Mi11 | SEQ ID NO: 13 |
| | TTTGATCTGTTTGTCCCATGATCGTCTACTCCAATTGAGAAATT |
| | TGTTAGGTGTTTTAGGATGAACACCTTTCTTCCTGTCATCCTTT |
| | GGAACTTGTTCTATGTAACGCTCATAAGTTGGACGATCCTTCAT |
| | TTTATTGATTTCCTTTTTACGACGAGCAATGGATCGTCTCTAGA |
| | CCCAGCTTTCTTGTACAAAGTGGTTGATCCTGCAGGGTCCGTCG |
| | CTTCTCTTCCATTTCTTCTCATTTTCGATTTTGATTCTTATTTC |
| | TTTCCAGTAGCTCCTGCTCTGTGAATTTCTCCGCTCACGATAGA |
| | TCTGCTTATACTCCTTACATTCAACCTTAGATCTGGTCTCGATT |
| | CTCTGTTTCTCTGTTTTTTTCTTTTGGTCGAGAATCTGATGTTT |
| | GTTTATGTTCTGTCACCATTAATAATAATGAACTCTCTCATTCA |
| | TACAATGATTAGTTTCTCTCGTCTACAAAACGATATGTTGCATT |
| | TTCACTTTTCTTCTTTTTTTCTAAGATGATTTGCTTTGACCAAT |
| | TTGTTTAGATCTTTATTCTATTTTATTTTCTGGTGGGTTGGTGG |
| | AAATTGAAAAAAAAAAAACAGCATAAATTGTTATTTGTTAATGT |
| | ATTCATTTTTTGGCTATTTGTTCTGGGTAAAAATCTGCTTCTAC |
| | TATTGAATCTTTCCTGGATTTTTTACTCCTATTGGGTTTTTATA |
| | GTAAAAATACATAATAAAAGGAAAACAAAAGTTTTATAGATTCT |
| | CTTAAACCCCTTACGATAAAAGTTGGAATCAAAATAATTCAGGA |
| | TCAGATGCTCTTTGATTGATTCAGATGCGATTACAGTTGCATGG |
| | CAAATTTTCTAGATCCGTCGTCACATTTTATTTTCTGTTTAAAT |
| | ATCTAAATCTGATATATGATGTCGACAAATTCTGGTGGCTTATA |
| | CATCACTTCAACTGTTTTCTTTTGGCTTTGTTTGTCAACTTGGT |
| | TTTCAATACGATTTGTGATTTCGATCGCTGAATTTTTAATACAA |
| | GCAAACTGATGTTAACCACAAGCAAGAGATGTGACCTGCCTTAT |
| | TAACATCGTATTACTTACTACTAGTCGTATTCTCAACGCAATCG |
| | TTTTTGTATTTCTCACATTATGCCGCTTCTCTACTCTTTATTCC |
| | TTTTGGTCCACGCATTTTCTATTTGTGGCAATCCCTTTCACAAC |
| | CTGATTTCCCACTTTGGATCATTTGTCTGAAGACTCTCTTGAAT |
| | CGTTACCACTTGTTTCTTGTGCATGCTCTGTTTTTTAGAATTAA |
| | TGATAAAACTATTCCATAGTCTTGAGTTTTCAGCTTGTTGATTC |
| | TTTTGCTTTTGGTTTTCTGCAGGTTTAAACATCAACCACTTTGT |
| | ACAAGAAAGCTGGGTCTAGAGACGATCCATTGCTCGTCGTAAAA |
| | AGGAAATCAATAAAATGAAGGATCGTCCAACTTATGAGCGTTAC |
| | ATAGAACAAGTTCCAAAGGATGACAGGAAGAAAGGTGTTCATCC |
| | TAAAACACCTAACAAATTTCTCAATTGGAGTAGACGATCATGGG |
| | ACAAACAGATCAAA |
| Mi38 | SEQ ID NO: 19 |
| | CGATCGACGCTCATCCGCTTTGTATGAATCTTTAGAATGTTTGC |
| | ACGAGCTTCCAAATCAGGCACGGCGAATTCGATACGACGATCAA |
| | TACGTCCTGGCCGTACAAGTGCAGGATCCAAAGTATCAGGACGA |
| | TTTGTGGCCATATCGTCTCTAGACCCAGCTTTCTTGTACAAAGT |
| | GGTTGATCCTGCAGGGTCCGTCGCTTCTCTTCCATTTCTTCTCA |
| | TTTTCGATTTTGATTCTTATTTCTTTCCAGTAGCTCCTGCTCTG |
| | TGAATTTCTCCGCTCACGATAGATCTGCTTATACTCCTTACATT |
| | CAACCTTAGATCTGGTCTCGATTCTCTGTTTCTCTGTTTTTTTC |
| | TTTTGGTCGAGAATCTGATGTTTGTTTATGTTCTGTCACCATTA |
| | ATAATAATGAACTCTCTCATTCATACAATGATTAGTTTCTCTCG |
| | TCTACAAAACGATATGTTGCATTTTCACTTTTCTTCTTTTTTTC |
| | TAAGATGATTTGCTTTGACCAATTTGTTTAGATCTTTATTCTAT |
| | TTTATTTTCTGGTGGGTTGGTGGAAATTGAAAAAAAAAAAACAG |
| | CATAAATTGTTATTTGTTAATGTATTCATTTTTTGGCTATTTGT |
| | TCTGGGTAAAAATCTGCTTCTACTATTGAATCTTTCCTGGATTT |
| | TTTACTCCTATTGGGTTTTTATAGTAAAAATACATAATAAAAGG |
| | AAAACAAAAGTTTTATAGATTCTCTTAAACCCCTTACGATAAAA |
| | GTTGGAATCAAAATAATTCAGGATCAGATGCTCTTTGATTGATT |
| | CAGATGCGATTACAGTTGCATGGCAAATTTTCTAGATCCGTCGT |
| | CACATTTTATTTTCTGTTTAAATATCTAAATCTGATATATGATG |
| | TCGACAAATTCTGGTGGCTTATACATCACTTCAACTGTTTTCTT |
| | TTGGCTTTGTTTGTCAACTTGGTTTTCAATACGATTTGTGATTT |
| | CGATCGCTGAATTTTTAATACAAGCAAACTGATGTTAACCACAA |
| | GCAAGAGATGTGACCTGCCTTATTAACATCGTATTACTTACTAC |
| | TAGTCGTATTCTCAACGCAATCGTTTTTGTATTTCTCACATTAT |
| | GCCGCTTCTCTACTCTTTATTCCTTTTGGTCCACGCATTTTCTA |
| | TTTGTGGCAATCCCTTTCACAACCTGATTVCCCACTTTGGATCA |
| | TTTGTCTGAAGACTCTCTTGAATCGTTACCACTTGTTTCTTGTG |
| | CATGCTCTGTTTTTTAGAATTAATGATAAAACTATTCCATAGTC |
| | TTGAGTTTTCAGCTTGTTGATTCTTTTGCTTTTGGTTTTCTGCA |
| | GGTTTAAACATCAACCACTTTGTACAAGAAAGCTGGGTCTAGAG |
| | ACGATATGGCCACAAATCGTCCTGATACTTTGGATCCTGCACTT |
| | GTACGGCCAGGACGTATTGATCGTCGTATCGAATTCGCCGTGCC |
| | TGATTTGGAAGCTCGTGCAAACATTCTAAAGATTCATACAAAGC |
| | GGATGAGCGTCGATCG |
| Mi40 | SEQ ID NO: 25 |
| | AAGAAGATATTCATATCTTTCCAAACACTGAGCAGCAGTTCTTC |
| | CAACAATAGGAGCAATTGTACGCCATTGAGTAGGCATAATTTTG |
| | GCCAAATGGAGAAGTTTTTCGTCCTCTTCTCTGGACCATTCCGT |
| | CTTTTTAATTCGAGGATCAAGCCATTCATACCAACGGGCTTTAC |
| | AATCGTCTCTAGACCCAGCTTTCTTGTACAAAGTGGTTGATCCT |
| | GCAGGGTCCGTCGCTTCTCTTCCATTTCTTCTCATTTTCGATTT |
| | TGATTCTTATTTCTTTCCAGTAGCTCCTGCTCTGTGAATTTCTC |
| | CGCTCACGATAGATCTGCTTATACTCCTTACATTCAACCTTAGA |
| | TCTGGTCTCGATTCTCTGTTTCTCTGTTTTTTTCTTTTGGTCGA |
| | GAATCTGATGTTTGTTTATGTTCTGTCACCATTAATAATAATGA |
| | ACTCTCTCATTCATACAATGATTAGTTTCTCTCGTCTACAAAAC |
| | GATATGTTGCATTTTCACTTTTCTTCTTTTTTTCTAAGATGATT |
| | TGCTTTGACCAATTTGTTTAGATCTTTATTCTATTTTATTTTCT |
| | GGTGGGTTGGTGGAAATTGAAAAAAAAAAAACAGCATAAATTGT |
| | TATTTGTTAATGTATTCATTTTTTGGCTATTTGTTCTGGGTAAA |
| | AATCTGCTTCTACTATTGAATCTTTCCTGGATTTTTTACTCCTA |
| | TTGGGTTTTTATAGTAAAAATACATAATAAAAGGAAAACAAAAG |
| | TTTTATAGATTCTCTTAAACCCCTTACGATAAAAGTTGGAATCA |
| | AAATAATTCAGGATCAGATGCTCTTTGATTGATTCAGATGCGAT |
| | TACAGTTGCATGGCAAATTTTCTAGATCCGTCGTCACATTTTAT |
| | TTTCTGTTTAAATATCTAAATCTGATATATGATGTCGACAAATT |
| | CTGGTGGCTTATACATCACTTCAACTGTTTTCTTTTGGCTTTGT |
| | TTGTCAACTTGGTTTTCAATACGATTTGTGATTTCGATCGCTGA |
| | ATTTTTAATACAAGCAAACTGATGTTAACCACAAGCAAGAGATG |
| | TGACCTGCCTTATTAACATCGTATTACTTACTACTAGTCGTATT |
| | CTCAACGCAATCGTTTTTGTATTTCTCACATTATGCCGCTTCTC |
| | TACTCTTTATTCCTTTTGGTCCACGCATTTTCTATTTGTGGCAA |
| | TCCCTTTCACAACCTGATTTCCCACTTTGGATCATTTGTCTGAA |
| | GACTCTCTTGAATCGTTACCACTTGTTTCTTGTGCATGCTCTGT |
| | TTTTTAGAATTAATGATAAAACTATTCCATAGTCTTGAGTTTTC |
| | AGCTTGTTGATTCTTTTGCTTTTGGTTTTCTGCAGGTTTAAACA |
| | TCAACCACTTTGTACAAGAAAGCTGGGTCTAGAGACGATTGTAA |
| | AGCCCGTTGGTATGAATGGCTTGATCCTCGAATTAAAAAGACGG |
| | AATGGTCCAGAGAAGAGGACGAAAAACTTCTCCATTTGGCCAAA |
| | ATTATGCCTACTCAATGGCGTACAATTGCTCCTATTGTTGGAAG |
| | AACTGCTGCTCAGTGTTTGGAAAGATATGAATATCTTCTT |
| Mi101 | SEQ ID NO: 30 |
| | GAGCCGCCTCTATATGATCGTCGACAAGGATTTGTGCCCCGAAC |
| | GCAAGATGATTTTGGTGATGGTGGGGCTTTTCCAGAAATACATA |
| | TTGCTCAGTTTCCAATCGGTATGGGCGCCGATAAGCCCGGCACT |
| | GGGGCAAAGAACACAGTTGCGCTTCAGTTTGACTCTGAGGGCAA |
| | ACTTCGATTTGATGAGCTTACACGAATTGGGCATGGAAAAGACA |
| | AGATTGTTCATTCCCGCCTTTCTGATATGAAATCCAACCATATT |
| | GATGATGAAGACGAAAGTTTCAAAAAACCAACAGATGAAGAAAT |
| | ACATGAAACAGCCGAAGCAACTCGTGTTTCCTTAGAGAAAATTA |
| | CAGCTGTTAAGATCGCCGCATCATTACCAGTCCAGCATGCAAAG |
| | AAAACAGCGCCTGCACAATATATTCGATCGTCTCTAGACCCAGC |
| | TTTCTTGTACAAAGTGGTTGATCCTGCAGGGTCCGTCGCTTCTC |
| | TTCCATTTCTTCTCATTTTCGATTTTGATTCTTATTTCTTTCCA |
| | GTAGCTCCTGCTCTGTGAATTTCTCCGCTCACGATAGATCTGCT |
| | TATACTCCTTACATTCAACCTTAGATCTGGTCTCGATTCTCTGT |
| | TTCTCTGTTTITTTCTTTTGGTCGAGAATCTGATGTTTGTTTAT |
| | GTTCTGTCACCATTAATAATAATGAACTCTCTCATTCATACAAT |
| | GATTAGTTTCTCTCGTCTACAAAACGATATGTTGCATTTTCACT |
| | TTTCTTCTTTTTTTCTAAGATGATTTGCTTTGACCAATTTGTTT |
| | AGATCTTTATTCTATTTTATTTTCTGGTGGGTTGGTGGAAATTG |
| | AAAAAAAAAAAACAGCATAAATTGTTATTTGTTAATGTATTCAT |
| | TTTTTGGCTATTTGTTCTGGGTAAAAATCTGCTTCTACTATTGA |
| | ATCTTTCCTGGATTTTTTACTCCTATTGGGTTTTTATAGTAAAA |
| | ATACATAATAAAAGGAAAACAAAAGTTTTATAGATTCTCTTAAA |
| | CCCCTTACGATAAAAGTTGGAATCAAAATAATTCAGGATCAGAT |
| | GCTCTTTGATTGATTCAGATGCGATTACAGTTGCATGGCAAATT |
| | TTCTAGATCCGTCGTCACATTTTATTTTCTGTTTAAATATCTAA |
| | ATCTGATATATGATGTCGACAAATTCTGGTGGCTTATACATCAC |
| | TTCAACTGTTTTCTTTTGGCTTTGTTTGTCAACTTGGTTTTCAA |
| | TACGATTTGTGATTTCGATCGCTGAATTTTTAATACAAGCAAAC |
| | TGATGTTAACCACAAGCAAGAGATGTGACCTGCCTTATTAACAT |
| | CGTATTACTTACTACTAGTCGTATTCTCAACGCAATCGTTTTTG |
| | TATTTCTCACATTATGCCGCTTCTCTACTCTTTATTCCTTTTGG |
| | TCCACGCATTTTCTATTTGTGGCAATCCCTTTCACAACCTGATT |
| | TCCCACTTTGGATCATTTGTCTGAAGACTCTCTTGAATCGTTAC |
| | CACTTGTTTCTTGTGCATGCTCTGTTTTTTAGAATTAATGATAA |
| | AACTATTCCATAGTCTTGAGTTTTCAGCTTGTTGATTCTTTTGC |
| | TTTTGGTTTTCTGCAGGTTTAAACATCAACCACTTTGTACAAGA |
| | AAGCTGGGTCTAGAGACGATCGAATATATTGTGCAGGCGCTGTT |
| | TTCTTTGCATGCTGGACTGGTAATGATGCGGCGATCTTAACAGC |
| | TGTAATTTTCTCTAAGGAAACACGAGTTGCTTCGGCTGTTTCAT |
| | GTATTTCTTCATCTGTTGGTTTTTTGAAACTTTCGTCTTCATCA |
| | TCAATATGGTTGGATTTCATATCAGAAAGGCGGGAATGAACAAT |
| | CTTGTCTTTTCCATGCCCAATTCGTTGTAAGCTCATCAAATCGA |
| | AGTTTGCCCTCAGAGTCAAACTGAAGCGCAACTGTGTTCTTTGC |
| | CCCAGTGCCGGGCTTATCGGCGCCCATACCGATTGGAAACTGAG |
| | CAATATGTATTTCTGGAAAAGCCCCACCATCACCAAAATCATCT |
| | TGCGTTCGGGGCACAAATCCTTGTCGACGATCATATAGAGGCGG |
| | CTC |
| Mi109 | SEQ ID NO: 34 |
| | GTTGGATCCTAATGAGGTCAACGTGATTTCCAACGCAAATTCTC |
| | GTCAGAACGTTCGTCGTTTGGTCAAAGATGGTCTTATAATTCGT |
| | AAGCCTGGCAAAGTTCATTCGCGTTTCCGAGCTCGTGAACGTTT |
| | GGAAGCACGCAGAAAGGGTCGTCATATGGGTTTTGGAAAACGAC |
| | GCGGAACTCGTAACGCTCGAATGCCGGAGAAGGTCTTATGGATG |
| | CGTTTTTTTGTTGTACTCCGTcATCTTTTAAAGCGCTATCGAGC |
| | TGCGAAGAAGATTGACAAGCATCTTTACCACGAGCTTTACTTGA |
| | AGGCTAAAGGAAACGCATTTAAGAATAAGAGAAATTTGATGGAA |
| | TATATCTTCAAAAAGAAGACTGAGAACCAGCGTTCTAAACAACT |
| | TGCTGAACAGGCTGAGGCTCGATCGTCTCTAGACCCAGCTTTCT |
| | TGTACAAAGTGGTTGATCCTGCAGGGTCCGTCGCTTCTCTTCCA |
| | TTTCTTCTCATTTTCGATTTTGATTCTTATTTCTTTCGAGTAGC |
| | TCCTGCTCTGTGAATTTCTCCGCTCACGATAGATCTGCTTATAC |
| | TCCTTACATTCAACCTTAGATCTGGTCTCGATTCTCTGTTTCTC |
| | TGTTTTTTTCTTTTGGTCGAGAATCTGATGTTTGTTTATGTTCT |
| | GTCACCATTAATAATAATGAACTCTCTCATTCATACAATGATTA |
| | GTTTCTCTCGTCTACAAAACGATATGTTGCATTTTCACTTTTCT |
| | TCTTTTTTTCTAAGATGATTTGCTTTGACCAATTTGTTTAGATC |
| | TTTATTCTATTTTATTTTCTGGTGGGTTGGTGGAAATTGAAAAA |
| | AAAAAAACAGCATAAATTGTTATTTGTTAATGTATTCATTTTTT |
| | GGCTATTTGTTCTGGGTAAAAATCTGCTTCTACTATTGAATCTT |
| | TCCTGGATTTTTTACTCCTATTGGGTTTTTATAGTAAAAATACA |
| | TAATAAAAGGAAAACAAAAGTTTTATAGATTCTCTTAAACCCCT |
| | TACGATAAAAGTTGGAATCAAAATAATTCAGGATCAGATGCTCT |
| | TTGATTGATTCAGATGCGATTACAGTTGCATGGCAAATTTTCTA |
| | GATCCGTCGTCACATTTTATTTTCTGTTTAAATATCTAAATCTG |
| | ATATATGATGTCGACAAATTCTGGTGGCTTATACATCACTTCAA |
| | CTGTTTTCTTTTGGCTTTGTTTGTCAACTTGGTTTTCAATACGA |
| | TTTGTGATTTCGATCGCTGAATTTTTAATACAAGCAAACTGATG |
| | TTAACCACAAGCAAGAGATGTGACCTGCCTTATTAACATCGTAT |
| | TACTTACTACTAGTCGTATTCTCAACGCAATCGTTTTTGTATTT |
| | CTCACATTATGCCGCTTCTCTACTCTTTATTCCTTTTGGTCCAC |
| | GCATTTTCTATTTGTGGCAATCCCTTTCACAACCTGATTTCCCA |
| | CTTTGGATCATTTGTCTGAAGACTCTCTTGAATCGTTACCACTT |
| | GTTTCTTGTGCATGCTCTGTTTTTTAGAATTAATGATAAAACTA |
| | TTCCATAGTCTTGAGTTTTCAGCTTGTTGATTCTTTTGCTTTTG |
| | GTTTTCTGCAGGTTTAAACATCAACCACTTTGTACAAGAAAGCT |
| | GGGTCTAGAGACGATCGAGCCTCAGCCTGTTCAGCAAGTTGTTT |
| | AGAACGCTGGTTCTCAGTCTTCTTTTTGAAGATATATTCCATCA |
| | AATTTCTCTTATTCTTAAATGCGTTTCCTTTAGCCTTCAAGTAA |
| | AGCTCGTGGTAAAGATGCTTGTCAATCTTCTTCGCAGCTCGATA |
| | GCGCTTTAAAAGATGACGGAGTACAACAAAAAAACGCATCCATA |
| | AGACCTTCTCCGGCATTCGAGCGTTACGAGTTCCGCGTCGTTTT |
| | CCAAAACCCATATGACGACCCTTTCTGCGTGCTTCCAAACGTTC |
| | ACGAGCTCGGAAACGCGAATGAACTTTGCCAGGCTTACGAATTA |
| | TAAGACCATCTTTGACCAAACGACGAACGTTCTGACGAGAATTT |
| | GCGTTGGAAATCACGTTGACCTCATTAGGATCCAAC |
| Mi111 | SEQ ID NO: 38 |
| | GGCCTCCAATCCCTACACGCTTTGGGAAGCGAAAGAAGGGAAGT |
| | AAAGGACCTGAAGCTAGCAATAAAATGCCTAATGTGACACCAGT |
| | AACTCGTTGCAAATTGCGTCTTCTTAAATATGAACGGATTAAAG |
| | ATTATTTGTTAATGGAAGAAGAATTTATTCGAAATCAGGAAAGA |
| | CATAAACCTCAAGAGGAAAGGCAGGAAGAAGAACGGACAAAAGT |
| | AGATGAAATGAGAGGGTCACCAATGGCTGTGGGTACATTGGAGG |
| | AGATTATTGACGATCAACACGCAGTCGTTTCTACGAATGTTGGC |
| | AGCGAACATTATGTTAACATTCTTTCATTTGTGGACAAGGAACA |
| | ATTGGAGCCAGGCTGTGCAGTTCTTCTAAATCACAAAACACACG |
| | CAGTTGTTGGATCGTCTCTAGACCCAGCTTTCTTGTACAAAGTG |
| | GTTGATCCTGCAGGGTCCGTCGCTTCTCTTCCATTTCTTCTCAT |
| | TTTCGATTTTGATTCTTATTTCTTTCCAGTAGCTCCTGCTCTGT |
| | GAATTTCTCCGCTCACGATAGATCTGCTTATACTCCTTACATTC |
| | AACCTTAGATCTGGTCTCGATTCTCTGTTTCTCTGTTTTTTTCT |
| | TTTGGTCGAGAATCTGATGTTTGTTTATGTTCTGTCACCATTAA |
| | TAATAATGAACTCTCTCATTCATACAATGATTAGTTTCTCTCGT |
| | CTACAAAACGATATGTTGCATTTTCACTTTTCTTCTTTTTTTCT |
| | AAGATGATTTGCTTTGACCAATTTGTTTAGATCTTTATTCTATT |
| | TTATTTTCTGGTGGGTTGGTGGAAATTGAAAAAAAAAAAACAGC |
| | ATAAATTGTTATTTGTTAATGTATTCATTTTTTGGCTATTTGTT |
| | CTGGGTAAAAATCTGCTTCTACTATTGAATCTTTCCTGGATTTT |
| | TTACTCCTATTGGGTTTTTATAGTAAAAATACATAATAAAAGGA |
| | AAACAAAAGTTTTATAGATTCTCTTAAACCCCTTACGATAAAAG |
| | TTGGAATCAAAATAATTCAGGATCAGATGCTCTTTGATTGATTC |
| | AGATGCGATTACAGTTGCATGGCAAATTTTCTAGATCCGTCGTC |
| | ACATTTTATTTTCTGTTTAAATATCTAAATCTGATATATGATGT |
| | CGACAAATTCTGGTGGCTTATACATCACTTCAACTGTTTTCTTT |
| | TGGCTTTGTTTGTCAACTTGGTTTTCAATACGATTTGTGATTTC |
| | GATCGCTGAATTTTTAATACAAGCAAACTGATGTTAACCACAAG |
| | CAAGAGATGTGACCTGCCTTATTAACATCGTATTACTTACTACT |
| | AGTCGTATTCTCAACGCAATCGTTTTTGTATTTCTCACATTATG |
| | CCGCTTCTCTACTCTTTATTCCTTTTGGTCCACGCATTTTCTAT |
| | TTGTGGCAATCCCTTTCACAACCTGATTTCCCACTTTGGATCAT |
| | TTGTCTGAAGACTCTCTTGAATCGTTACCACTTGTTTCTTGTGC |
| | ATGCTCTGTTTTTTAGAATTAATGATAAAACTATTCCATAGTCT |
| | TGAGTTTTCAGCTTGTTGATTCTTTTGCTTTTGGTTTTCTGCAG |
| | GTTTAAACATCAACCACTTTGTACAAGAAAGCTGGGTCTAGAGA |
| | CGATCCAACAACTGCGTGTGTTTTGTGATTTAGAAGAACTGCAC |
| | AGCCTGGCTCCAATTGTTCCTTGTCCACAAATGAAAGAATGTTA |
| | ACATAATGTTCGCTGCCAACATTCGTAGAAACGACTGCGTGTTG |
| | ATCGTCAATAATCTCCTCCAATGTACCCACAGCCATTGGTGACC |
| | CTCTCATTTCATCTACTTTTGTCCGTTCTTCTTCCTGCCTTTCC |
| | TCTTGAGGTTTATGTCTTTCCTGATTTCGAATAAATTCTTCTTC |
| | CATTAACAAATAATCTTTAATCCGTTCATATTTAAGAAGACGCA |
| | ATTTGCAACGAGTTACTGGTGTCACATTAGGCATTTTATTGCTA |
| | GCTTCAGGTCCTTTACTTCCCTTCTTTCGCTTCCCAAAGCGTGT |
| | AGGGATTGGAGGCC |
| Mi116 | SEQ ID NO: 42 |
| | TCACACCAAATTCCTTGATATTTTCTTTTAACATTTTTTTCAAA |
| | CTGTGGGTCTCCAAGCAATAAGTTTTCAGGGTATAGGTCAGGTG |
| | GTGACTCATTTTTCAATATTTGATAGTGGTTTGTATTTTTGACT |
| | TTACTTAACTTGTTTACTGAAATCTGTTCCTGTTCTTCAAGATG |
| | ACCGCACAATTCGAAAGATACACAAATTGGATGATCACGTAATG |
| | GCTGCTTTTGCTGGTTTGAGTGCAGATGCTCGTGTTTTGATTGA |
| | CCGTGCCAGAGTTGCTTGTGAAAATTACAAATTGACTTTGGAGG |
| | ATCCCGTCACACTTGAGCACATCTCCCGCACTATTGCGGACTTG |
| | AAACATGAATTTACGCAAACTACAGGTCGTCGTCCCTTTGGTGT |
| | CTCTTTACTTGTTGGCGGTTTTGATCCCGATGGAACTCCTCATC |
| | TTTTTATTACACCCAGCTTTCTTGTACAAAGTGGTGATATCCCG |
| | CGGGATCAGAAGCAACCTCATGGAAATGATGAGGTAAGGTTTCA |
| | TACTCTTGCCTCTTCTTACGGCTTTCTGTGTCTTCACTGTAAGT |
| | TTCTATGATTTGAGCCACCAATATATATGCTCTGGTGTGCTGAG |
| | TTATGTTTATCTGGTCACGCTTAGTGGGTAAAATTATGCTTATT |
| | TTAGCATAAACTTTAATGAGATTAGGTTTTGTATCACACCGATC |
| | TTTAGTTGTTTAGTAAGATGACAGAAATTCTTGGTAAAACACTC |
| | TAAATCGTCTTCTTTAGTGAAGTTTTCCTTAGAGTAGCATAAAT |
| | TTTGGCTTTTTTCTTGATGGTTGAATAAGGTGGCACTTGTTGGT |
| | ATGAGACTTTATTGAGAGTCATATTAAGCTGATCCACGCGTTTA |
| | CGCCCCGCCCTGCCACTCATCGCAGTACTGTTGTAATTCATTAA |
| | GCATTCTGCCGACATGGAAGCCATCACAGACGGCATGATGAACC |
| | TGAATCGCCAGCGGCATCAGCACCTTGTCGCCTTGCGTATAATA |
| | TTTGCCCATGGTGAAAACGGGGGCGAAGAAGTTGTCCATATTGG |
| | CCACGTTTAAATCAAAACTGGTGAAACTCACCCAGGGATTGGCT |
| | GAGACGAAAAACATATTCTCAATAAACCCTTTAGGGAAATAGGC |
| | CAGGTTTTCACCGTAACACGCCACATCTTGCGAATATATGTGTA |
| | GAAACTGCCGGAAATCGTCGTGGTATTCACTCCAGAGCGATGAA |
| | AACGTTTCAGTTTGCTCATGGAAAACGGTGTAACAAGGGTGAAC |
| | ACTATCCCATATCACCAGCTCACCGTCTTTCATTGCCATACGGA |
| | ATTCCGGATGAGCATTCATCAGGCGGGCAAGAATGTGAATAAAG |
| | GCCGGATAAAACTTGTGCTTATTTTTCTTTACGGTCTTTAAAAA |
| | GGCCGTAATATCCAGCTGAACGGTCTGGTTATAGGTACATTGAG |
| | CAACTGACTGAAATGCCTCAAAATGTTCTTTACGATGCCATTGG |
| | GATATATCAACGGTGGTATATCCAGTGATTTTTTTCTCCATTTT |
| | AGCTTCCTTAGCTCCTGAAAATCTCGCCGGATCAGCTTAGCGTT |
| | CATTGAATTTGATGGCCATAGGGGTTTAGATGCAACTGTTTCTT |
| | TGAACATTGTAGAAATATATAAAGATTTTACATTAGCCTACTCT |
| | TGAAAGTCAAATTGTCGAATTTGATTATATTATACTCTAGAGGT |
| | GATATTAGTTAATGAGTTTATACTCGGTTATTTACAGCTTATTC |
| | ATATACCAGTTAACGTGTCTCATATATTCTAACTTCTTAGCATT |
| | TAACGTGTTTGCAGGTCAGCTTGACACTGAACATAACAGCATCA |
| | CTAGTGCGGCCGCCTGCAGGTCGACCATATGGTCGACCTGCAGG |
| | CGGCCGCACTAGTGATATCACCACTTTGTACAAGAAAGCTGGGT |
| | GTAATAAAAAGATGAGGAGTTCCATCGGGATCAAAACCGCCAAC |
| | AAGTAAAGAGACACCAAAGGGACGACGACCTGTAGTTTGCGTAA |
| | ATTCATGTTTCAAGTCCGCAATAGTGCGGGAGATGTGCTCAAGT |
| | GTGACGGGATCCTCCAAGTCAATTTGTAATTTTCACAAGCAACT |
| | CTGGCACGGTCAATCAAAACACGAGCATCTGCACTCAAACCAGC |
| | AAAAGCAGCCATTACGTGATCATCCAATTTGTGTATCTTTCGAA |
| | TTGTGCGGTCATCTTGAAGAACAGGAACAGATTTCAGTAAACAA |
| | GTTAAGTAAAGTCAAAAATACAAACCACTATCAAATATTGAAAA |
| | ATGAGTCACCACCTGACCTATACCCTGAAAACTTATTGCTTGGA |
| | GACCCACAGTTTGAAAAAAATGTTAAAAGAAAATATCAAGGAAT |
| | TTGGTGTGA |
| Mi125a | SEQ ID NO: 47 |
| | ATCCTCTTCATCGTCACCGCCTCCTCCTCCACCTCCCGAAGCAG |
| | CCTTCTTCAAATTCTTTCTCTTGACACGTCCAGGAGGTCCACCA |
| | GCAAGATAAGATTTTGTGGAGAAGTCAATGTGCTTTTGAGAGTC |
| | GAGACGAACAATGAATGATGGAATATTGACTACTTGTCGACGAA |
| | TTCGAATATGCTTTTGACGAATAAGAACACGAGCATGGTGAATT |
| | GATTTAGCCAATCCCAATTTGAAAACTTGCGTCTGCAAACGACG |
| | CTCCAAAAAGTCTTCAACACGCAAACCAAGAACATAGTCCAATT |
| | TCATCTTATCCTCCAAAAGAACTCCAATACGAACCAAACGACGA |
| | AGAAGAGCATTTCCTTCAAATAAGCGTCGAGGATCCTTGTCTTC |
| | CAATGTTAAAAGTTCACGAGCAGCTTTACGAATCTTTGCCAAAG |
| | TTAATTTGACACGCCAAACTTCACGTTTATTTCGTAAACCAAAC |
| | TCGCCAATTAATTTTAATTCCTGATCTAAACGCTCCTTCTCGAA |
| | TGGACGACGAGGCACATCGTCTCTAGACCCAGCTTTCTTGTACA |
| | AAGTGGTTGATCCTGCAGGGTCCGTCGCTTCTCTTCCATTTCTT |
| | CTCATTTTCGATTTTGATTCTTATTTCTTTCCAGTAGCTCCTGC |
| | TCTGTGAATTTCTCCGCTCACGATAGATCTGCTTATACTCCTTA |
| | CATTCAACCTTAGATCTGGTCTCGATTCTCTGTTTCTCTGTTTT |
| | TTTCTTTTGGTCGAGAATCTGATGTTTGTTTATGTTCTGTCACC |
| | ATTAATAATAATGAACTCTCTCATTCATACAATGATTAGTTTCT |
| | CTCGTCTACAAAACGATATGTTGCATTTTCACTTTTCTTCTTTT |
| | TTTCTAAGATGATTTGCTTTGACCAATTTGTTTAGATCTTTATT |
| | CTATTTTATTTTCTGGTGGGTTGGTGGAAATTGAAAAAAAAAAA |
| | ACAGCATAAATTGTTATTTGTTAATGTATTCATTTTTTGGCTAT |
| | TTGTTCTGGGTAAAAATCTGCTTCTACTATTGAATCTTTCCTGG |
| | ATTTTTTACTCCTATTGGGTTTTTATAGTAAAAATACATAATAA |
| | AAGGAAAACAAAAGTTTTATAGATTCTCTTAAACCCCTTACGAT |
| | AAAAGTTGGAATCAAAATAATTCAGGATCAGATGCTCTTTGATT |
| | GATTCAGATGCGATTACAGTTGCATGGCAAATTTTCTAGATCCG |
| | TCGTCACATTTTATTTTCTGTTTAAATATCTAAATCTGATATAT |
| | GATGTCGACAAATTCTGGTGGCTTATACATCACTTCAACTGTTT |
| | TCTTTTGGCTTTGTTTGTCAACTTGGTTTTCAATACGATTTGTG |
| | ATTTCGATCGCTGAATTTTTAATACAAGCAAACTGATGTTAACC |
| | ACAAGCAAGAGATGTGACCTGCCTTATTAACATCGTATTACTTA |
| | CTACTAGTCGTATTCTCAACGCAATCGTTTTTGTATTTCTCACA |
| | TTATGCCGCTTCTCTACTCTTTATTCCTTTTGGTCCACGCATTT |
| | TCTATTTGTGGCAATCCCTTTCACAACCTGATTTCCCACTTTGG |
| | ATCATTTGTCTGAAGACTCTCTTGAATCGTTACCACTTGTTTCT |
| | TGTGCATGCTCTGTTTTTTAGAATTAATGATAAAACTATTCCAT |
| | AGTCTTGAGTTTTCAGCTTGTTGATTCTTTTGCTTTTGGTTTTC |
| | TGCAGGTTTAAACATCAACCACTTTGTACAAGAAAGCTGGGTCT |
| | AGAGACGATGTGCCTCGTCGTCCATTCGAGAAGGAGCGTTTAGA |
| | TCAGGAATTAAAATTAATTGGCGAGTTTGGTTTACGAAATAAAC |
| | GTGAAGTTTGGCGTGTCAAATTAATTTTGGCAAAGATTCGTAAA |
| | GCTGCTCGTGAACTTTTAACATTGGAAGACAAGGATCCTCGACG |
| | CTTATTTGAAGGAAATGCTCTTCTTCGTCGTTTGGTTCGTATTG |
| | GAGTTCTTTTGGAGGATAAGATGAAATTGGACTATGTTCTTGGT |
| | TTGCGTGTTGAAGACTTTTTGGAGCGTCGTTTGCAGACGCAAGT |
| | TTTCAAATTGGGATTGGCTAAATCAATTCACCATGCTCGTGTTC |
| | TTATTCGTCAAAAGCATATTCGAATTCGTCGACAAGTAGTCAAT |
| | ATTCCATCATTCATTGTTCGTCTCGACTCTCAAAAGCACATTGA |
| | CTTCTCCACAAAATCTTATCTTGCTGGTGGACCTCCTGGACGTG |
| | TCAAGAGAAAGAATTTGAAGAAGGCTGCTTCGGGAGGTGGAGGA |
| | GGAGGCGGTGACGATGAAGAGGAT |
| Mi125b | SEQ ID NO: 48 |
| | TTCATCTTATCCTCCAAAAGAACTCCAATACGAACCAAACGACG |
| | AAGAAGAGCATTTCCTTCAAATAAGCGTCGAGGATCCTTGTCTT |
| | CCAATGTTAAAAGTTCACGAGCAGCTTTACGAATCTTTGCCAAA |
| | GTTAATTTGACACGCCAAACTTCACGTTTATTTCGTAAACCAAA |
| | CTCGCCAATTAATTTTAATTCCTGATCGTCTCTAGACCCAGCTT |
| | TCTTGTACAAAGTGGTTGATCCTGCAGGGTCCGTCGCTTCTCTT |
| | CCATTTCTTCTCATTTTCGATTTTGATTCTTATTTCTTTCCAGT |
| | AGCTCCTGCTCTGTGAATTTCTCCGCTCACGATAGATCTGCTTA |
| | TACTCCTTACATTCAACCTTAGATCTGGTCTCGATTCTCTGTTT |
| | CTCTGTTTTTTTCTTTTGGTCGAGAATCTGATGTTTGTTTATGT |
| | TCTGTCACCATTAATAATAATGAACTCTCTCATTCATACAATGA |
| | TTAGTTTCTCTCGTCTACAAAACGATATGTTGCATTTTCACTTT |
| | TCTTCTTTTTTTCTAAGATGATTTGCTTTGACCAATTTGTTTAG |
| | ATCTTTATTCTATTTTATTTTCTGGTGGGTTGGTGGAAATTGAA |
| | AAAAAAAAAACAGCATAAATTGTTATTTGTTAATGTATTCATTT |
| | TTTGGCTATTTGTTCTGGGTAAAAATCTGCTTCTACTATTGAAT |
| | CTTTCCTGGATTTTTTACTCCTATTGGGTTTTTATAGTAAAAAT |
| | ACATAATAAAAGGAAAACAAAAGTTTTATAGATTCTCTTAAACC |
| | CCTTACGATAAAAGTTGGAATCAAAATAATTCAGGATCAGATGC |
| | TCTTTGATTGATTCAGATGCGATTACAGTTGCATGGCAAATTTT |
| | CTAGATCCGTCGTCACATTTTATTTTCTGTTTAAATATCTAAAT |
| | CTGATATATGATGTCGACAAATTCTGGTGGCTTATACATCACTT |
| | CAACTGTTTTCTTTTGGCTTTGTTTGTCAACTTGGTTTTCAATA |
| | CGATTTGTGATTTCGATCGCTGAATTTTTAATACAAGCAAACTG |
| | ATGTTAACCACAAGCAAGAGATGTGACCTGCCTTATTAACATCG |
| | TATTACTTACTACTAGTCGTATTCTCAACGCAATCGTTTTTGTA |
| | TTTCTCACATTATGCCGCTTCTCTACTCTTTATTCCTTTTGGTC |
| | CACGCATTTTCTATTTGTGGCAATCCCTTTCACAACCTGATTTC |
| | CCACTTTGGATCATTTGTCTGAAGACTCTCTTGAATCGTTACCA |
| | CTTGTTTCTTGTGCATGCTCTGTTTTTTAGAATTAATGATAAAA |
| | CTATTCCATAGTCTTGAGTTTTCAGCTTGTTGATTCT1TTGCTT |
| | TTGGTTTTCTGCAGGTTTAAACATCAACCACTTTGTACAAGAAA |
| | GCTGGGTCTAGAGACGATCAGGAATTAAAATTAATTGGCGAGTT |
| | TGGTTTACGAAATAAACGTGAAGTTTGGCGTGTCAAATTAACTT |
| | TGGCAAAGATTCGTAAAGCTGCTCGTGAACTTTTAACATTGGAA |
| | GACAAGGATCCTCGACGCTTATTTGAAGGAAATGCTCTTCTTCG |
| | TCGTTTGGTTCGTATTGGAGTTCTTTTGGAGGATAAGATGAA |
| Mi129 | SEQ ID NO: 58 |
| | CCGACGAGACTTACTGCATCGACAACGAGGCTTTGTACGACATC |
| | TGCTTCCGAACTCTGAAGCTTCAAAATCCAACATATGGAGATTT |
| | GAATCATCTCGTATCCGTAACCATGTCTGGAGTGACTACTTGTC |
| | TTCGCTTCCCAGGCCAATTGAACGCTGATCTTCGCAAATTGGCT |
| | GTCAACATGGTTCCATTCCCACGTCTTCACTTTTTCATGCCTGG |
| | TTTCGCTCCACTTTCGGCTAAGGGCGCAGCTGCCTACCAGGCCC |
| | AAAATGTTGCTGAATTGACCAAGCAAATGTTTGATGCCAAGAAT |
| | ATGATGGCTGCTTGTGATCCACGCCATGGACGCTATTTAACTGT |
| | TGCCGCTATCTTCCGTGGACGTATGTCAATGCGTGAAGTAGACA |
| | TCGTCTCTAGACCCAGCTTTCTTGTACAAAGTGGTTGATCCTGC |
| | AGGGTCCGTCGCTTCTCTTCCATTTCTTCTCATTTTCGATTTTG |
| | ATTCTTATTTCTTTCCAGTAGCTCCTGCTCTGTGAATTTCTCCG |
| | CTCACGATAGATCTGCTTATACTCCTTACATTCAACCTTAGATC |
| | TGGTCTCGATTCTCTGTTTCTCTGTTTTTTTCTTTTGGTCGAGA |
| | ATCTGATGTTTGTTTATGTTCTGTCACCATTAATAATAATGAAC |
| | TCTCTCATTCATACAATGATTAGTTTCTCTCGTCTACAAAACGA |
| | TATGTTGCATTTTCACTTTTCTTCTTTTTTTCTAAGATGATTTG |
| | CTTTGACCAATTTGTTTAGATCTTTATTCTATTTTATTTTCTGG |
| | TGGGTTGGTGGAAATTGAAAAAAAAAAAACAGCATAAATTGTTA |
| | TTTGTTAATGTATTCATTTTTTGGCTATTTGTTCTGGGTAAAAA |
| | TCTGCTTCTACTATTGAATCTTTCCTGGATTTTTTACTCCTATT |
| | GGGTTTTTATAGTAAAAATACATAATAAAAGGAAAACAAAAGTT |
| | TTATAGATTCTCTTAAACCCCTTACGATAAAAGTTGGAATCAAA |
| | ATAATTCAGGATCAGATGCTCTTTGATTGATTCAGATGCGATTA |
| | CAGTTGCATGGCAAATTTTCTAGATCCGTCGTCACATTTTATTT |
| | TCTGTTTAAATATCTAAATCTGATATATGATGTCGACAAATTCT |
| | GGTGGCTTATACATCACTTCAACTG1TTTCTTTTGGCTTTGTTT |
| | GTCAACTTGGTTTTCAATACGATTTGTGATTTCGATCGCTGAAT |
| | TTTTAATACAAGCAAACTGATGTTAACCACAAGCAAGAGATGTG |
| | ACCTGCCTTATTAACATCGTATTACTTACTACTAGTCGTATTCT |
| | CAACGCAATCGTTTTTGTATTTCTCACATTATGCCGCTTCTCTA |
| | CTCTTTATTCCTTTTGGTCCACGCATTTTCTATTTGTGGCAATC |
| | CCTTTCACAACCTGATTTCCCACTTTGGATCATTTGTCTGAAGA |
| | CTCTCTTGAATCGTTACCACTTGTTTCTTGTGCATGCTCTGTTT |
| | TTTAGAATTAATGATAAAACTATTCCATAGTCTTGAGTTTTCAG |
| | CTTGTTGATTCTTTTGCTTTTGGTTTTCTGCAGGTTTAAACATC |
| | AACCACTTTGTACAAGAAAGCTGGGTCTAGAGACGATGTCTACT |
| | TCACGCATTGACATACGTCCACGGAAGATAGCGGCAACAGTTAA |
| | ATAGCGTCCATGGCGTGGATCACAAGCAGCCATCATTTTCTTGG |
| | CATCAAACATTTGCTTGGTCAATTCAGCAACATTTTGGGCCTGG |
| | TAGGCAGCTGCGCCCTTAGCCGAAAGTGGAGCGAAACCAGGCAT |
| | GAAAAAGTGAAGACGTGGGAATGGAACCATGTTGACAGCCAATT |
| | TGCGAAGATCAGCGTTCAATTGGCCTGGGAAGCGAAGACAAGTA |
| | GTCACTCCAGACATGGTTACGGATACGAGATGATTCAAATCTCC |
| | ATATGTTGGATTTTGAAGCTTCAGAGTTCGGAAGCAGATGTCGT |
| | ACAAAGCCTCGTTGTCGATGCAGTAAGTCTCGTCGG |
| MiCC2 | SEQ ID NO: 60 |
| | ATTTTTCGCAGCATCACCAATCAGACGTTCCGTGTCTGTAAAAG |
| | CCACATAAGATGGAGTCGTGCGATTTCCTTGATCATTGGCAATG |
| | ATCTCGACTTTTCCATGCTGGAATACACCGACACATAAATAAGT |
| | CGTTCCCAGATCAATTCCTGAGAAATTTAAAGAATGACAAAGTT |
| | ACAACATCCAGCTGTTGAGGGTCTTGCTGTTGGTTTGGATCGTG |
| | GCCATCCTGTGACTAAAATGTTCGCAAGCCACGTCAATCTCGTA |
| | AAAAAGGCCGCATTACGAAGAAGACTCGAATTGTTACTCGTGTT |
| | TCCTTAGAGAAAATTACAGCTGTTAAGATCGCCGCATCATTACC |
| | AGTCCAGCATGCAAAGAAAACAGCGCCTGCACAATATATTCGTT |
| | ACACTCCTAGCCAGCAGGCTGGTGGTTTTCATACAAGTGGAGCA |
| | CAACAAAGAACCCAGCTTTCTTGTACAAAGTGGTGATATCCCGC |
| | GGGATCAGAAGCAACCTCATGGAAATGATGAGGTAAGGTTTCAT |
| | ACTCTTGCCTCTTCTTACGGCTTTCTGTGTCTTCACTGTAAGTT |
| | TCTATGATTTGAGCCACCAATATATATGCTCTGGTGTGCTGAGT |
| | TATGTTTATCTGGTCACGCTTAGTGGGTAAAATTATGCTTATTT |
| | TAGCATAACTTTAATGAGATTAGGTTTTGTATCACACCGATCTT |
| | TAGTTGTTTAGTAAGATGACAGAAATTCTTGGTAAAACACTCTA |
| | AATCGTCTTCTTTAGTGAAGTTTTCCTTAGAGTAGCATAAATTT |
| | TGGCTTTTTTCTTGATGGTTGAATAAGGTGGCACTTGTTGGTAT |
| | GAGACTTTATTGAGAGTCATATTAAGCTGATCCACGCGTTTACG |
| | CCCCGCCCTGCCACTCATCGCAGTACTGTTGTAATTCATTAAGC |
| | ATTCTGCCGACATGGAAGCCATCACAGACGGCATGATGAACCTG |
| | AATCGCCAGCGGCATCAGCACCTTGTCGCCTTGCGTATAATATT |
| | TGCCCATGGTGAAAACGGGGGCGAAGAAGTTGTCCATATTGGCC |
| | ACGTTTAAATCAAAACTGGTGAAACTCACCCAGGGATTGGCTGA |
| | GACGAAAAACATATTCTCAATAAACCCTTTAGGGAAATAGGCCA |
| | GGTTTTCACCGTAACACGCCACATCTTGCGAATATATGTGTAGA |
| | AACTGCCGGAAATCGTCGTGGTATTCACTCCAGAGCGATGAAAA |
| | CGTTTCAGTTTGCTCATGGAAAACGGTGTAACAAGGGTGAACAC |
| | TATCCCATATCACCAGCTCACCGTCTTTCATTGCCATACGGAAT |
| | TCCGGATGAGCATTCATCAGGCGGGCAAGAATGTGAATAAAGGC |
| | CGGATAAAACTTGTGCTTATTTTTCTTTACGGTCTTTAAAAAGG |
| | CCGTAATATCCAGCTGAACGGTCTGGTTATAGGTACATTGAGCA |
| | ACTGACTGAAATGCCTCAAAATGTTCTTTACGATGCCATTGGGA |
| | TATATCAACGGTGGTATATCCAGTGATTTTTTTCTCCATTTTAG |
| | CTTCCTTAGCTCCTGAAAATCTCGCCGGATCAGCTTAGCGTTCA |
| | TTGAATTTGATGGCCATAGGGGTTTAGATGCAACTGTTTCTTTG |
| | AACATTGTAGAAATATATAAAGATTTTACATTAGCCTACTCTTG |
| | AAAGTCAAATTGTCGAATTTGATTATATTATACTCTAGAGGTGA |
| | TATTAGTTAATGAGTTTATACTCGGTTATTTACAGCTTATTCAT |
| | ATACCAGTTAACGTGTCTCATATATTCTAACTTCTTAGCATTTA |
| | ACGTGTTTGCAGGTCAGCTTGACACTGAACATAACAGCATCACT |
| | AGTGCGGCCGCCTGCAGGTCGACCATATGGTCGACCTGCAGGCG |
| | GCCGCACTAGTGATATCACCACTTTGTACAAGAAAGCTGGGTTC |
| | TTTGTTGTGCTCCACTTGTATGAAAACCACCAGCCTGCTGGCTA |
| | GGAGTGTAACGAATATATTGTGCAGGCGCTGTTTTCTTTGCATG |
| | CTGGACTGGTAATGATGCGGCGATCTTAACAGCTGTAATTTTCT |
| | CTAAGGAAACACGAGTAACAATTCGAGTCTTCTTCGTAATGCGG |
| | CCTTTTTTACGAGATTGACGTGGCTTGCGAACATTTTTAGTCAC |
| | AGGATGGCCACGATCCAAACCAACAGCAAGACCCTCAACAGCTG |
| | GATGTTGTAACTTTGTCATTCTTTAAATTTCTCAGGAATTGATC |
| | TGGGAACGACTTATTTATGTGTCGGTGTATTCCAGCATGGAAAA |
| | GTCGAGATCATTGCCAATGATCAAGGAAATCGCACGACTCCATC |
| | TTATGTGGCTTTTACAGACACGGAACGTCTGATTGGTGATGCTG |
| | CGAAAAAT |

TABLE 6

| | | | |
|---|---|---|---|
| Target | SEQ | | |
| ID | ID NO | Sequence* | Example Gi-number and species |

| | | | |
|---|---|---|---|
| Mi11 | 61 | AAAAGGAAATCAATAAAATGAA | 15767577 (Meloidogyne arenaria) |
| Mi11 | 62 | ATGAACACAACTGGAGATGAATCAACATTGAAAGATATTCC | 15767577 (Meloidogyne arenaria) |
| Mi11 | 63 | GCGCAGTCTTTGGAACGGCCGCATTC | 15767577 (Meloidogyne arenaria) |
| Mi11 | 64 | GGGGAATCGCCATCTTCAAGTGTTTACGGCTCTTATTGTTCATC | 15767577 (Meloidogyne arenaria) |
| Mi11 | 65 | TAGAACAAGTTCCAAAGGATGACAGGAAGAAAGG | 15767577 (Meloidogyne arenaria) |
| Mi11 | 66 | TGGGACAAACAGATCAAAUATGGAAACTTGCAATATATGAATGGGC | 15767577 (Meloidogyne arenaria) |
| Mi38 | 67 | AACTTGGAGTTGAACTAAACTTTGCATATCCTTTCACCACTTTCTGAAC | 46983636 (Meloidogyne hapla) |
| | | AGCGTCAATAAAATC | |
| Mi38 | 68 | ACTTCATCAAAGAAAATAATACAAGCCTTCTT | 19268096 (Meloidogyne hapla) |
| Mi38 | 69 | ATACGACGATCAATACGTCCTGGCCGTACAAGTGC | 46987842 (Meloidogyne hapla) |
| Mi38 | 70 | CCCATTCCATCATCAAAACGAGCTCC | 46987842 (Meloidogyne hapla) |
| Mi38 | 71 | GCACGTAATGCAAACATTCCGGCTTC | 46987842 (Meloidogyne hapla) |
| Mi38 | 72 | GGACGATTTGTGGCCATTAAAACTTT | 19268096 (Meloidogyne hapla) |
| Mi38 | 73 | GGGCAAAGACGCGATATCAATTC | 46987842 (Meloidogyne hapla) |
| Mi38 | 74 | GTACAGACAGAGCGAATATCAGC | 46987842 (Meloidogyne hapla) |
| Mi38 | 75 | GTGTCATATAACTTGGAGTTGAACTAAACTTTGCATATCCTTTCACC | 46219310 (Meloidogyne hapla) |
| Mi38 | 76 | GTGTCATATAACTTGGAGTTGAACTAAACTTTGCATATCCTTTCACCAC | 46987842 (Meioidogyne hapla) |
| | | TTTCTGAACAGCGTCAATAAAATC | |
| Mi38 | 77 | ATACAAGCCTTCTTTGTTTTTGCCATCT | 15766735 (Meloidogyne javanica) |
| Mi38 | 78 | CCGTACAAGTGCAGGATCCAAAGTATCAGGACGATTTGTGGCCATTAAA | 15766735 (Meloidogyne javanica) |
| | | ACTTTGATTGCACCACGCGAGTCAAATCCATCCAGCTGATTGATCAATT | |
| | | CGAGCATTGTCCGTTGAACTTCATTATCTCCCCCCATTCCATCATCAAA | |
| | | ACGAGCTCCGCCAATAGCATC | |
| Mi40 | 79 | GATCCTATTGATATGGATGAAGATGAATT | 31546893 (Strongyloides ratti) |
| Mi101 | 80 | CAGTTTGACTCTGAGGGCAAACTTCGATTTGATGA | 19383725 (Meloidogyne hapla) |
| Mi101 | 81 | CATTCCCGCCTTTCTGATATGAAATC | 19383725 (Meloidogyne hapla) |
| Mi101 | 82 | GAACGCAAGATGATTTTGGTGATGGTGGGGCTTT | 19383725 (Meloidogyne hapla) |
| Mi109 | 83 | CGCAGAAAGGGTCGTCATATGGGT | 944801 (Brugia malayi) |
| Mi109 | 84 | GACAAGCATCTTTACCACGAGCTTTAC | 752661 (Caenorhabditis briggsae) |
| Mi109 | 85 | GACAAGCATCTTTACCACGAGCT | 68275989 (Caenorhabditis remanei) |
| Mi109 | 86 | AAGATTGACAAGCATCTTTACC | 18382417 (Globodera pallida) |
| | | | 21819186 (Trichinella spiralis) |
| Mi109 | 87 | ACAAGCATCTTTACCACGAGCT | 18382417 (Globodera pallida) |
| Mi109 | 88 | AAGATTGACAAGCATCTTTACCACGAGCT | 111166140 (Globodera rostochiensis) |
| Mi109 | 89 | AAGATTGACAAGCATCTTTACCACGAGCTTTAC | 111165991 (Globodera rostochiensis) |
| Mi109 | 90 | AGATTGACAAGCATCTTTACCA | 18382417 (Globodera pallida) |
| Mi109 | 91 | ATTGACAAGCATCTTTACCACG | 18382417 (Globodera pallida) |
| Mi109 | 92 | CAAGCATCTTTACCACGAGCT | 18382417 (Globodera pallida) |
| Mi109 | 93 | GACAAGCATCTTTACCACGAGC | 18382417 (Globodera pallida) |
| Mi109 | 94 | GATTGACAAGCATCTTTACCAC | 18382417 (Globodera pallida) |
| Mi109 | 95 | TGACAAGCATCTTTACCACGAG | 18382417 (Globodera pallida) |
| Mi109 | 96 | AAGATTGACAAGCATCTTTACCACGAGCT | 7143692 (Globodera rostochiensis) |
| Mi109 | 97 | AGATTGACAAGCATCTTTACCACGAGCT | 7143685 (Globodera rostochiensis) |
| Mi109 | 98 | GCTCGTGAACGTTTGGAAGCA | 28567874 (Heterodera glycines) |
| Mi109 | 99 | GCTCGTGAACGTTTGGAAGCACGCAGAAAGGG | 28566936 (Heterodera glycines) |
| | | | 33139882 (Heterodera schachtii) |
| Mi109 | 100 | GCTCGTGAACGTTTGGAAGCACGCAGA | 33139171 (Heterodera schachtii) |
| Mi109 | 101 | TGAACAGGCTGAGGCTCGCCGAAACAAGAACAAGGAAACGAGAAAGAGG | 15769093 (Meloidogyne arenaria) |
| | | CGTGAGGAGCGTTTGATTTTGAAACGTAAGGAATTGCTCCACAAACTTT | |
| | | CTGAGAGCGAAAAGCAGAAGACTCCACAGCTTCAAGAAGAGGAAGCGAA | |
| | | GGAAGCTTCGCCTTCTCCACCAC | |
| Mi109 | 102 | AGAAAGGGTCGTCATATGGGTTTTGG | 30029158 (Meloidogyne chitwoodi) |
| Mi109 | 103 | AAAGTTCATTCGCGTTTCCGAGCTCGTGAACGTTTGGAAGCACGCAGAA | 39746570 (Meloidogyne paranaensis) |
| | | AGGGTCGTCATATGGGTTTTGGAAAACG | |
| Mi109 | 104 | AAGAGAAATTTGATGGAATATATCTTCAAAAAGAAGACTGAGAACCAGC | 39746570 (Meloidogyne paranaensis) |
| | | GTTCTAAACAACTTGCTGAACA | |
| Mi109 | 105 | AAGCAGAAGACTCCACAGCTTCAAGAAGAGGAAGCGAAGGAAGCTTCGC | 39746570 (Meloidogyne paranaensis) |
| | | CTTCTCCACC | |
| Mi109 | 106 | AAGGAATTGCTCCACAAACTTTCTGAGAGCGA | 39746570 (Meloidogyne paranaensis) |
| Mi109 | 107 | ATGCCGGAGAAGGTCTTATGGATGCGT | 39746570 (Meloidogyne paranaensis) |
| Mi109 | 108 | ATTCGTAAGCCTGGCAAAGTTCATTCGCGTTTCCGAGCTCGTGAACGTT | 39746726 (Meloidogyne paranaensis) |
| | | TGGAAGCACGCAGAAAGGGTCGTCATATGGGTTTTGGAAAACGACGCGG | |
| | | AACTCGTAACGCTCGAATGCCGGAGAAGGTCTTATGGATGCGT | |
| Mi109 | 109 | CATCTTTTAAAGCGCTATCGAGCTGCGAAGAAGATTGACAAGCATCTTT | 39746570 (Meloidogyne paranaensis) |
| | | ACCACGAGCTTTAC | |
| Mi109 | 110 | GATTTCCAACGCAAATTCTCGTCAGAACGTTCGTCGTTT | 39746570 (Meloidogyne paranaensis) |
| Mi109 | 111 | GATTTCCAACGCAAATTCTCGTCAGAACGTTCGTCGTTTG | 39746726 (Meloidogyne paranaensis) |
| Mi109 | 112 | GATTTCCAACGCAAATTCTCGTCAGAACGTTCGTCGTTTGGTCAAAGAT | 39746574 (Meloidogyne paranaensis) |
| | | GGTCTTATAATTCGTAAGCCTGGCAAAGTTCATTCGCGTTTCCGAGCTC | |
| | | GTGAACGTTTGGAAGCACGCAGAAAGGGTCGTCATATGGGTTTTGGAAA | |
| | | ACGACGCGGAACTCGTAACGCTCGAATGCCGGAGAAGGTCTTATGGATG | |
| | | CGT | |
| Mi109 | 123 | GCTGAGGCTCGCCGAAACAAGAACAAGGA | 39746570 (Meloidogyne paranaensis) |
| Mi109 | 114 | GTCAAAGATGGTCTTATAATTCGTAAGCCTGG | 39746570 (Meloidogyne paranaensis) |
| Mi109 | 115 | TGGATCCTAATGAGGTCAACG | 39746574 (Meloidogyne paranaensis) |
| Mi109 | 116 | TGTACTCCGTCATCTTTTAAAGCGCTATCGAGCTGCGAAGAAGATTGAC | 39746726 (Meloidogyne paranaensis) |
| | | AAGCATCTTTACCACGAGCTTTACTTGAAGGCTAAAGGAAACGCATTTA | |
| | | AGAATAAGAGAAATTTGATGGAATATATCTTCAAAAAGAAGACTGAGAA | |
| | | CCAGCGTTCTAAACAACTTGCTGAA | |
| Mi109 | 117 | TGTACTCCGTCATCTTTTAAAGCGCTATCGAGCTGCGAAGAAGATTGAC | 39746574 (Meloidogyne paranaensis) |
| | | AAGCATCTTTACCACGAGCTTTACTTGAAGGCTAAAGGAAACGCATTTA | |
| | | AGAATAAGAGAAATTTGATGGAATATATCTTCAAAAAGAAGACTGAGAA | |
| | | CCAGCGTTCTAAACAACTTGCTGAACAGGCTGAGGCTCGCCGAAACAAG | |
| | | AACAAGGAAACGAGAAAGAGGCGTGAGGAGCGTTTGATTTTGAAACGTA | |
| | | AGGAATTGCTCCACAAACTTTCTGAGAGCGAAAAGCAGAAGACTCCACA | |
| | | GCTTCAAGAAGAGGAAGCGAAGGAAGCTTCGCCTTCTCCACCAC | |
| Mi109 | 118 | CGCAGAAAGGGTCGTCATATGGGT | 4136397 (Onchocerca volvulus), |
| | | | 2898199 (Toxocara canis) |
| M1109 | 119 | CGCAGAAAGGGTCGTCATATGGGT | 45214539 (Wuchereria bancrofti) |
| Mi109 | 120 | AATTCTCGTCAGAACGTTCGTCG | 51863033 (Xiphinema index) |
| Mi111 | 121 | CTTACACATCCAGAGTACTACGAAGAAATTGGAATCAAGCCT | 15767736 (Meloidogyne arenaria) |
| Mi111 | 122 | GGAAAAGGCTCCAACTGAAACGTACGCTGATGTTGGCGGTTTGGAACAA | 15767736 (Meloidogyne arenaria) |
| | | CAAATTCAAGAAATTAAGGAGGCAGTGGAATTGCC | |
| Mi111 | 123 | AAGCGAAAGAAGGGAAGTAAAGGACCTGAAGCTAGCAATAAAATGCC | 19384087 (Meloidogyne hapla) |
| Mi111 | 124 | ATGGAAGAAGAATTTATTCGAAATCAGGAAAGAC | 19384087 (Meloidogyne hapla) |
| Mi111 | 125 | GAACATTATGTTAACATTCTTTCA | 19384692 (Meloidogyne hapla) |
| Mi111 | 126 | GAACATTATGTTAACATTCTTTCATTTGT | 19384750 (Meloidogyne hapla) |
| Mi111 | 127 | GAATTGCCACTTACACATCCAGA | 19435681 (Meloidogyne hapia) |
| Mi111 | 128 | GATGAAATGAGAGGGTCACCAATGGCTGTGGGTACATT | 19384087 (Meloidogyne hapia) |
| Mi111 | 129 | GGTTTGGAACAACAAATTCAAGAAATTAA | 19435681 (Meloidogyne hapla) |
| Mi111 | 130 | TGTGCAGTTCTTCTAAATCACAA | 19435681 (Meloidogyne hapla) |
| Mi111 | 131 | AACATTCTTTCATTTGTGGACAAGGAACAATTGGAGCCAGGCTGTGCAG | 15767091 (Meloidogyne javanica) |
| | | TTCTTCTAAATCACAAAACACACGCAGTTGTTGGTGTTTTGGCTGATGA | |
| | | CACGGATCCAATGGT | |
| Mi111 | 132 | AAGAAGGGAAGTAAAGGACCTGAAGCTAGCAATAAAATGCCTAATGTGA | 40670817 (Meloidogyne javanica) |
| | | CACCAGTAACTCGTTGCAAATTGCG | |
| Mi111 | 133 | AAGGACCTGAAGCTAGCAATAAAATGCCTAATGTGACACCAGTAACTCG | 15767091 (Meloidogyne javanica) |
| | | TTGCAAATTGCG | |
| Mi111 | 134 | CAGGAAGAAGAACGGACAAAAGTAGATGAAATGAGAGGGTCACCAATGG | 15767091 (Meloidogyne javanica) |
| | | CTGTGGGTACATTGGAGGAGATTATTGACGATCAACACGC | |
| Mi111 | 135 | CAGGAAGAAGAACGGACAAAAGTAGATGAAATGAGAGGGTCACCAATGG | 40670817 (Meloidogyne javanica) |
| | | CTGTGGGTACATTGGAGGAGATTATTGACGATCAACACGCAGTCGTTTC | |
| Mi111 | 136 | CCAGGTTCAGGAGATAAGCAAGGAGAAGGAGACAAGAAGCGTCGTTTTG | 40670817 (Meloidogyne javanica) |
| | | GGCCTCCAATCCC | |
| Mi111 | 137 | CTTCTTAAATATGAACGGATTAAAGATTATTTGTTAATGGAAGAAGAAT | 15767091 (Meloidogyne javanica) |
| | | TTATTCGAAATCAGGAAAGACATAAACCTCAAGA | |
| Mi111 | 138 | GTTGGCAGCGAACATTATGTTAACATTCTTTCATTTGTGGACAAGGAAC | 40670817 (Meloidogyne javanica) |
| | | AATTGGAGCCAGGCTGTGCAGTTCTTCTAAATCACAAAACACACGCAGT | |
| | | TGTTGGTGTTTTTGGCTGATGACACGGATCCAATGGTTTCTGTAATGAA | |
| | | ATTGGAAAAGGCTCCAACTGAAACGTACGCTGATGTTGGCGGTTTGGAA | |
| | | CAACAAATTCAAGAA | |
| Mi111 | 139 | TCTGTAATGAAATTGGAAAAGGCTCCAACTGAAACGTACGCTGATGTTG | 15767091 (Meloidogyne javanica) |
| | | GCGGTTTGGAACAACAAATTCAAGAAATTAAGGAGGCAGTGGAATTGCC | |
| | | ACTTACACATCCAGAGTACTA | |
| Mi116 | 140 | ATTGTGCGGTCATCTTGAAGA | 1120680 (Caenorhabditis elegans) |
| Mi116 | 141 | ACGGGATCCTCCAAAGTCAATTTGTAATTTTCACA | 16004901 (Meloidogyne arenaria) |
| Mi16 | 142 | GCAACTCTGGCACGGTCAATCAAAACACGAGCATCTGCACTCAAACCAG | 16004901 (Meloidogyne arenaria) |
| | | CAAAAGCAGCCATTACGTGATCATCCAATTTGTGTATCTTTCGAATTGT | |
| | | GCGGTCATCTTGAAGAACAGGAACAGATTTC | |
| Mi116 | 143 | GGTTCAGTAATAAAAAGATGAGGAGTTCCATCGGGATCAAAACCGCCAA | 16004901 (Meloidogyne arenaria) |
| | | CAAGTAAAGAGACACCAAAAGGACGACGACCTGTAGTTTGCGTAAATTC | |
| | | ATGTTTCAAGTCCGCAATAG | |
| Mi116 | 144 | TTCTCATTCCGGCCGATTGAGCCAGCAAGTAATTCGTAATACACACCAG | 15767794 (Meloidogyne arenaria) |
| | | ATGGTTCAGTAATAAAAAGATGAGGAGTTCCATCGGGATCAAAACCGCC | |
| | | AACAAGTAAAGAGACACCAAAAGGACGACGACCTGTAGTTTGCGTAAAT | |
| | | TCATGTTTCAAGTCCGCAATAGTGCGGGAGATGTGCTCAAGTGTGACGG | |
| | | GATCCTCCAAAGTCAATTTGTAATTTTCACAAGCAACTCTGGCACGGTC | |
| | | AATCAAAACACGAGCATCTGCACTCAAACCAGCAAAAGCAGCCATTACG | |
| | | TGATCATCCAATTTGTGTATCTTTCGAATTGTGCGGTCATCTTGAAGAA | |
| | | CAGGAACAGATTTC | |
| Mi116 | 145 | AAAAAAATGTTAAAAGAAAATAT | 30049385 (Meloidogyne chitwoodi) |
| Mi116 | 146 | AAAAAATGTTAAAAGAAAATAT | 30050083 (Meloidogyne chitwoodi) |
| Mi116 | 147 | AAAAATGTTAAAAGAAAATAT | 30166852 (Meloidogyne chitwoodi) |
| Mi116 | 148 | GAAAAAAATGTTAAAAGAAAA | 31325468 (Meloidogyne chitwoodi) |
| Mi116 | 149 | GAAAAAAATGTTAAAAGAAAAT | 31325428 (Meloidogyne chitwoodi) |
| Mi116 | 150 | GAAAAAAATGTTAAAAGAAAATA | 31325427 (Meloidogyne chitwoodi) |
| Mi116 | 151 | GAAAAAAATGTTAAAAGAAAATAT | 30049285 (Meloidogyne chitwoodi) |
| Mi116 | 152 | GACAAGCATCTTTACCACGAGCT | 88681932 (Caenorhabditis remanei) |
| Mi125 | 153 | ATAAGAACACGAGCATGGTGAATTGATTTAGCCAATCCC | 16004848 (Meloidogyne arenaria) |
| Mi125 | 154 | GGAGGTCCACCAGCAAGATAAGATTTTGTGGAGAAGTCAAT | 16004848 (Meloidogyne arenaria) |
| Mi125 | 155 | TCCAAAAGAACTCCAATACGAACCAAACGACGAAGAAGAGCATTT | 16004848 (Meloidogyne arenaria) |
| Mi125 | 156 | TCCTCCACCTCCCGAAGCAGCCTTCTTCAAATTCTTTCTCTTGACACGT | 16004848 (Meloidogyne arenaria) |
| | | CC | |
| Mi125 | 157 | TGCTTTTGAGAGTCGAGACGAACAATGAATGATGGAATATTGACTACTT | 16004848 (Meloidogyne arenaria) |
| | | GTCGACGAATTCGAAT | |
| Mi125 | 158 | TTTTATAAACCTTTAGATATTATTCAATCCTCTTCAT | 16004848 (Meloidogyne arenaria) |
| Mi125 | 159 | AAAAGTTCACGAGCAGCTTTACG | 31325000 (Meloidogyne chitwoodi) |
| Mi125 | 160 | AAGAACTCCAATACGAACCAAACGACGAAGAAGAGCATTTCCTTC | 32185135 (Meloidogyne chitwoodi) |
| Mi125 | 161 | AATGATGGAATATTGACTACTTGTC | 32185135 (Meloidogyne chitwoodi) |
| Mi125 | 162 | AATTTTAATTCCTGATCTAAACGCTC | 32185135 (Meloidogyne chitwoodi) |
| Mi125 | 163 | ACACGTCCAGGAGGTCCACCAGCAAGATAAGATTTTGT | 32185135 (Meloidogyne chitwoodi) |
| Mi125 | 164 | CCAAGAACATAGTCCAATTTCAT | 32185135 (Meloidogyne chitwoodi) |
| Mi125 | 165 | CTCAAACTGGAGTAATTAAAG | 37819228 (Meloidogyne chitwoodi) |
| Mi125 | 166 | GCTCAAACTGGAGTAATTAAAC | 37819133 (Meloidogyne chitwoodi) |
| Mi125 | 167 | GTCTGCAAACGACGCTCCAAAAAGTCTT | 32183696 (Meloidogyne chitwoodi) |
| Mi125 | 168 | GTCTGCAAACGACGCTCCAAAAAGTCTTCAACACGCAA | 32185135 (Meloidogyne chitwoodi) |
| Mi125 | 169 | AGCTCAAACTGGAGTAATTAAAC | 38513039 (Meloidogyne hapla) |
| Mi125 | 170 | CTCAAACTGGAGTAATTAAAC | 37970857 (Meloidogyne hapla) |
| Mi125 | 171 | GCTCAAACTGGAGTAATTAAAC | 37971092 (Meloidogyne hapla) |
| Mi125 | 172 | TAAGCTCAAACTGGAGTAATTAAAC | 37970990 (Meloidogyne hapla) |
| Mi125 | 173 | AATAAGCGTCGAGGATCCTTGTCTTCCAATGTTAAAAGTTCACGAGC | 9829373 (Meloidogyne javanica) |
| Mi125 | 174 | AATTTTAATTCCTGATCTAAACGCTCCTTCTCGAATGGACG | 9829373 (Meloidogyne javanica) |
| Mi125 | 175 | ATAAGAACACGAGCATGGTGAATTGATTTAGCCAATCCC | 15766790 (Meloidogyne javanica) |
| Mi125 | 176 | ATAAGAACACGAGCATGGTGAATTGATTTAGCCAATCCCAATTTGAAAA | 9829373 (Meloidogyne javanica) |
| | | CTTG | |
| Mi125 | 177 | CGAGGCACGTCATAAACTTTAGAAAATACATGCTTTAT | 9829373 (Meloidogyne javanica) |
| Mi125 | 178 | CTCCTCCACCTCCCGAAGCAGCCTTCTTCAAATTCTTTCTCTTGACACG | 9829373 (Meloidogyne javanica) |
| | | TCC | |
| Mi125 | 179 | GAATCTTTGCCAAAGTTAATTTGAC | 9829639 (Meloidogyne javanica) |
| Mi125 | 180 | GACGCTCCAAAAAGTCTTCAACACGCAAACCAAGAAC | 9829564 (Meloidogyne javanica) |
| Mi125 | 181 | GCTTTACGAATCTTTGCCAAAGTTAATTTGACACGCCAAACTTCACG | 9829373 (Meloidogyne javanica) |
| Mi125 | 182 | GGAGGTCCACCAGCAAGATAAGATTTTGTGGAGAAGTCAAT | 15766790 (Meloidogyne javanica) |
| Mi125 | 183 | TCAACAAAAACATTTTTTCAAATCCAAAATCTTTTAACAACTTAACAG | 15766790 (Meloidogyne javan Ca) |
| Mi125 | 184 | TCCAAAAGAACTCCAATACGAACCAAACGACGAAGAAGAGCATTTCCTT | 15766790 (Meloidogyne javan ica) |
| | | C | |
| Mi125 | 185 | TGCAAACGACGCTCCAAAAAGTCTTCAACACGCAAACCAAGAAC | 9829373 (Meloidogyne javanica) |
| Mi125 | 186 | TGCTTTTGAGAGTCGAGACGAACAATGAATGATGGAATATTGACTACTT | 15766790 (Meloidogyne javanica) |
| | | GTCGACGAATTCGAAT | |
| Mi125 | 187 | TTTTATAAACCTTTAGATATTATTCAATCCTCTTCATCGTCACCGCCTC | 15766790 (Meloidogyne javanica) |
| | | CTCCTCCACCTCCCGAAGCAGCCTTCTTCAAATTCTTTCTCTTGACACG | |
| | | TCC | |
| Mi125 | 188 | AAGCTCAAACTGGAGTAATTA | 39746721 (Meloidogyne paranaensis) |
| Mi125 | 189 | ACGCCAAACTTCACGTTTATTTCGTAA | 39747139 (Meloidogyne paranaensis) |
| Mi125 | 190 | AGCTCAAACTGGAGTAATTAAAC | 39746611 (Meloidogyne paranaensis) |
| Mi125 | 191 | CAGCTTTACGAATCTTTGCCAAAGTTAATTTGA | 39747139 (Meloidogyne paranaensis) |
| Mi125 | 192 | CAGCTTTACGAATCTTTGCCAAAGTTAATTTGACACGCCAAACTTCACG | 46498505 (Meloidogyne paranaensis) |
| | | TTTATTTCGTAA | |
| Mi125 | 193 | CAGCTTTACGAATCTTTGCCAAAGTTAATTTGACACGCCAAACTTCACG | 39747346 (Meloidogyne paranaensis) |
| | | TTTATTTCGTAAACC | |
| Mi125 | 194 | CCAAACTCGCCAATTAATTTTAATTCCTGATCTAAACGCTCCTTCTCGA | 46498505 (Meloidogyne paranaensis) |
| | | ATGGACGA | |
| Mi125 | 195 | CCAAACTCGCCAATTAATTTTAATTCCTGATCTAAACGCTCCTTCTCGA | 39747139 (Meloidogyne paranaensis) |
| | | ATGGACGACGAGGCACGTCATA | |
| Mi125 | 196 | CCAAACTCGCCAATTAATTTTAATTCCTGATCTAAACGCTCCTTCTCGA | 46498634 (Meloidogyne paranaensis) |
| | | ATGGACGACGAGGCACGTCATAAACTTTAGAAAATACATGC1TTATGCG | |
| | | AGGCATCTTGTTTTACTTTTTAAGCTCAAACT | |
| Mi125 | 197 | CTCAAACTGGAGTAATTAAAC | 39746528 (Meloidogyne paranaensis) |
| Mi125 | 198 | CTTTCAACGTTTTATAAACCTTTAGATATTATTCAATCCTCTTCATCGT | 39747032 (Meloidogyne paranaensis) |
| | | CACCGCCTCCTCCTCCACCTCCCGAAGCAGCCTTCTTCAAATTCTTTCT | |
| | | CTTGACACGTCCAGGAGGTCCACCAGCAAGATAAGA | |
| Mi125 | 199 | GCTCAAACTGGAGTAATTAAAC | 39746628 (Meloidogyne paranaensis) |
| Mi125 | 200 | GTCTGCAAACGACGCTCCAAAAAGTCTTCAACACGCAAACCAAGAAC | 39747032 (Meloidogyne paranaensis) |
| Mi125 | 201 | TCCAAAAGAACTCCAATACGAACCAAACGACGAAGAAGAGCATTTCCTT | 39747032 (Meloidogyne paranaensis) |
| | | C | |
| Mi125 | 202 | TCGAATGGACGACGAGGCACGTCATAAACTTTAGAAAATACATGCTTTA | 39747346 (Meloidogyne paranaensis) |
| | | T | |
| Mi125 | 203 | TCGCCAATTAATTTTAATTCCTGATCTAAACGCTCCTT | 39747346 (Meloidogyne paranaensis) |
| Mi125 | 204 | TGCTTTTGACGAATAAGAACACGAGCATGGTGAATTGATTTAGCCAATC | 39747032 (Meloidogyne paranaensis) |
| | | CCAATTTGAAAACTTG | |
| Mi125 | 205 | TGCTTTTGAGAGTCGAGACGAACAATGAATGATGGAATATTGACTACTT | 39747032 (Meloidogyne paranaensis) |
| | | GTCGACGAATTCGAAT | |
| Mi125 | 206 | TTTTACTTTTTAAGCTCAAAC | 39747346 (Meloidogyne paranaensis) |
| Mi125 | 207 | TCTCGAATGGACGACGAGGCACGTCATA | 51237570 (Radopholus similis) |
| Mi125 | 208 | TTCTCGAATGGACGACGAGGCACGTCATA | 51237458 (Radopholus similis) |
| Mi125 | 209 | AAGAAGAGCATTTCCTTCAAA | 9830677 (Strongyloides stercoralis) |
| Mi125 | 210 | AAGTTCACGAGCAGCTTTACG | 9832721 (Strongyloides stercoralis) |
| Mi125 | 211 | AATAAACGTGAAGTTTGGCGTGT | 115342194 (Heterorhabditis bacteriophora) |
| Mi125 | 212 | TTCGTAAAGCTGCTCGTGAACTT | 115342194 (Heterorhabditis bacteriophora) |
| Mi125 | 213 | GAAGGAAATGCTCTTCTTCGTCGTTTGGT | 115342194 (Heterorhabditis bacteriophora) |
| Mi127 | 214 | CCTTGATCATTGGCAATGATCTC | 19556133 (Ascaris suum) |
| Mi127 | 215 | GTTGGCTCGTTGATGATACGCAAAAC | 14019989 (Ascaris suum) |
| Mi127 | 216 | AGCTCAAACTTGGGTAATTAAA | 1850670 (Brugia malayi) |
| Mi127 | 217 | AGCTCAAACTTGGGTAATTAAAC | 1893269 (Brugia malayi) |
| Mi127 | 218 | CTCAAACTTGGGTAATTAAAC | 1017391 (Brugia malayi) |
| M1127 | 219 | GCTCAAACTTGGGTAATTAAA | 1193277 (Brugia malayi) |
| Mi127 | 220 | GCTCAAACTTGGGTAATTAAAC | 1871110 (Brugia malayi) |
| Mi127 | 221 | TAGCTCAAACTTGGGTAATTA | 1850558 (Brugia malayi) |
| Mi127 | 222 | TAGCTCAAACTTGGGTAATTAAAC | 1917062 (Brugia malayi) |
| Mi127 | 223 | AGCTCAAACTTGGGTAATTAAAC | 6578024 (Brugia pahangi) |
| Mi127 | 224 | CTCAAACTTGGGTAATTAAAC | 6578007 (Brugia pahangi) |
| Mi127 | 225 | GCTCAAACTTGGGTAATTAAAC | 6578012 (Brugia pahangi) |
| Mi127 | 226 | TAGCTCAAACTTGGGTAATTAAAC | 6578013 (Brugia pahangi) |
| Mi127 | 227 | AGCTCAAACTTGGGTAATTAA | 14833886 (Caenorhabditis elegans) |
| Mi127 | 228 | AGCTCAAACTTGGGTAATTAAA | 10925161 (Caenorhabditis elegans) |
| Mi127 | 229 | AGCTCAAACTTGGGTAATTAAAC | 10924962 (Caenorhabditis elegans) |
| Mi127 | 230 | ATAGCTCAAACTTGGGTAATTA | 31233223 (Caenorhabditis elegans) |
| Mi127 | 231 | ATAGCTCAAACTTGGGTAATTAA | 14835549 (Caenorhabditis elegans) |
| Mi127 | 232 | ATAGCTCAAACTTGGGTAATTAAA | 10925347 (Caenorhabditis elegans) |
| Mi127 | 233 | CTCAAACTTGGGTAATTAAAC | 2423515 (Caenorhabditis elegans) |
| Mi127 | 234 | GATAGCTCAAACTTGGGTAATTA | 33943631 (Caenorhabditis elegans) |
| Mi127 | 235 | GATAGCTCAAACTTGGGTAATTAA | 18287564 (Caenorhabditis elegans) |
| Mi127 | 236 | GCAGCAGTTGGCTCGTTGATGA | 71986023 (Caenorhabditis elegans) |
| Mi127 | 237 | GCTCAAACTTGGGTAATTAAA | 10925190 (Caenorhabditis elegans) |
| Mi127 | 238 | GCTCAAACTTGGGTAATTAAAC | 10923462 (Caenorhabditis elegans) |
| Mi127 | 239 | TAGCTCAAACTTGGGTAATTA | 684987 (Caenorhabditis elegans) |
| Mi127 | 240 | TAGCTCAAACTTGGGTAATTAA | 14838520 (Caenorhabditis elegans) |
| Mi127 | 241 | TAGCTCAAACTTGGGTAATTAAA | 14825166 (Caenorhabditis elegans) |
| Mi127 | 242 | GCAGCAGTTGGCTCGTTGATGATACG | 68276618 (Caenorhabditis remanei) |
| Mi127 | 243 | AGCTCAAACTTGGGTAATTAA | 11068960 (Haemonchus contortus) |
| Mi127 | 244 | AGCTCAAACTTGGGTAATTAAA | 11068981 (Haemonchus contortus) |
| Mi127 | 245 | GCTCAAACTTGGGTAATTAAA | 11068764 (Haemonchus contortus) |
| Mi127 | 246 | TAGCTCAAACTTGGGTAATTA | 61116185 (Litomosoides sigmodontis) |
| Mi127 | 247 | CGATTTCCTTGATCATTGGCAATGAT | 32184674 (Meloidogyne chitwoodi) |
| Mi127 | 248 | AATAAGTCGTTCCCAGATCAATTCCGACAGCGTTAGCTTTAGACATTTT | 39747367 (Meloidogyne paranaensis) |
| | | T | |
| Mi127 | 249 | AATAAGTCGTTCCCAGATCAATTCCGACAGCGTTAGCTTTAGACATTTT | 39747357 (Meloidogyne paranaensis) |
| | | TAAAAG | |
| Mi127 | 250 | ACGTTGCGCTCGCCTTGGCCTTTCTTATCCAAACCATAAGCAATAGCCG | 39747367 (Meloidogyne paranaensis) |
| | | CAGCAGT | |
| Mi127 | 251 | GGCTCGTTGATGATACGCAAAACATTAAGTCCAGAAATCGTTCCAGCAT | 39747367 (Meloidogyne paranaensis) |
| | | CCTTTGTGGCTTGGCGTTGACTGTCATTAAAGTACGCAGGCACAGTGAT | |
| | | GACAGCATCCTTAACGGTTTGGCCCAAAAATGCCTCAGCCGTTTCCTTC | |
| | | ATTTTAGTCAAAACCATGGCGGAGACTTCCTCAGGGAAGAACGCCTTCA | |
| | | TTTCACCCTTAACCTCCACTTGGATTTTTGGACGAGCACCTTCTCCTTG | |
| | | AATAACCTTGAACGGCCAATGCTTCATGTCAGATTGAACAGCTGGATCG | |
| | | TCAAATTTGCGTCCAATCAAGCGCTTTGCGTCAAAAACAGTATTCGACG | |
| | | GGTTCATTGCCACTTGATTTTTCGCAGCATCACCAATCAGACGTTCCGT | |
| | | GTCTGTAAAAGCCACATAAGATGGAGTCGTGCGATTTCCTTGATCATTG | |
| | | GCAATGATCTCGACTTTTCCATGCTGGAATACACC | |
| Mi127 | 252 | TAGCTCAAACTTGGGTAATTA | 46498358 (Meloidogyne paranaensis) |
| Mi127 | 253 | TCAAAAGTTCCACCGCCCAAGTCAAAAATCA | 39747367 (Meloidogyne paranaensis) |
| Mi127 | 254 | CTCAAACTTGGGTAATTAAAC | 10840960 (Onchocerca volvulus) |
| Mi127 | 255 | TCAGCCGTTTCCTTCATTTTA | 4191634 (Onchocerca volvulus) |
| Mi127 | 256 | ACCTTGAACGGCCAATGCTTCAT | 20062709 (Toxocara canis) |
| Mi127 | 257 | CCTTGATCATTGGCAATGATCTC | 20499328 (Toxocara canis) |
| Mi127 | 258 | CTCAAACTTGGGTAATTAAAC | 1498474 (Toxocara canis) |
| Mi127 | 259 | GTCAAAATAGACACATCAAAAGT | 2104671 (Trichinella britovi) |
| Mi127 | 260 | TCGTTGATGATACGCAAAACATT | 2104671 (Trichinella britovi) |
| Mi127 | 261 | TCGTTGATGATACGCAAAACATT | 13199118 (Trichinella spiralis) |
| Mi127 | 262 | ACCTTGAACGGCCAATGCTTCATGTC | 17353707 (Trichuris muris) |
| Mi127 | 263 | ACCTTGAACGGCCAATGCTTCAT | 28561608 (Trichuris vulpis) |
| Mi127 | 264 | TAGCTCAAACTTGGGTAATTA | 2789663 (Wuchereria bancrofti) |
| Mi127 | 265 | AAAGCTAACGCTGTCGGAATTGATCT | 115342160 (Heterorhabditis bacteriophora) |
| Mi127 | 266 | CGTATCATCAACGAGCCAACTGCTGC | 88679042 (Caenorhabditis remanei) |
| Mi127 | 267 | GGTTTAATTACCCAAGTTTGA | 9112994Nippostrongylus brasiliensis) |
| Mi127 | 268 | GGTTTAATTACCCAAGTTTGAG | 83656004Heterorhabditis bacteriophora) |
| Mi127 | 269 | GGTTTAATTACCCAAGTTTGAG | 91103082 (Nippostrongylus brasiliensis) |
| Mi127 | 270 | GGTTTAATTACCCAAGTTTGAGCT | 91102738 (Nippostrongylus brasiliensis) |
| Mi127 | 271 | GGTTTAATTACCCAAGTTTGAGCTA | 91102815 (Nippostrongylus brasiliensis) |
| Mi127 | 272 | GTTTAATTACCCAAGTTTGAG | 91102668 (Nippostrongylus brasiliensis) |
| Mi127 | 273 | GTTTAATTACCCAAGTTTGAGC | 91103120 (Nippostrongylus brasiliensis) |
| Mi127 | 274 | TCATCAACGAGCCAACTGCTGC | 86563795 (Caenorhabditis elegans) |
| Mi127 | 275 | TTAATTACCCAAGTTTGAGCT | 3096950Brugia pahangi) |
| Mi127 | 276 | TTAATTACCCAAGTTTGAGCT | 14832559 (Caenorhabditis elegans) |
| Mi127 | 277 | TTAATTACCCAAGTTTGAGCTA | 14829900 (Caenorhabditis elegans) |
| Mi127 | 278 | TTAATTACCCAAGTTTGAGCTAT | 14842179 (Caenorhabditis elegans) |
| Mi127 | 279 | TTTAATTACCCAAGTTTGAGC | 31237454 (Caenorhabditis elegans) |
| Mi128 | 280 | CGTGCAAAGAGAAAGCGTGACGA | 808508 (Brugia malayi) |
| Mi128 | 281 | TTGTTCGCGAGGTTGTTGGTTT | 10713865 (Heterodera glycines) |
| Mi128 | 282 | TTGTTCGCGAGGTTGTTGGTTT | 35504991 (Heterodera schachtii) |
| Mi128 | 283 | AAGAAGGCTTTGAAGTTTTTGAAGAA | 32183499 (Meloidogyne chitwoodi) |
| Mi128 | 284 | GGTTTAATTACCTACGTTTGA | 37820376 (Meloidogyne chitwoodi) |
| Mi128 | 285 | GGTTTAATTACCTACGTTTGAG | 37819977 (Meloidogyne chitwoodi) |
| Mi128 | 286 | GGTTTAATTACCTACGTTTGAGA | 37819540 (Meloidogyne chitwoodi) |
| Mi128 | 287 | GGTTTAATTACCTACGTTTGAGAA | 37820112 (Meloidogyne chitwoodi) |
| Mi128 | 288 | GGTTTAATTACCTACGTTTGAGAAATTT | 37819939 (Meloidogyne chitwoodi) |
| Mi128 | 289 | AGAAGGCTTTGAAGTTTTTGAAGAA | 46988391 (Meloidogyne hapla) |
| Mi128 | 290 | AGAATGACAAAGTTACAACAT | 37972035 (Meloidogyne hapla) |
| Mi128 | 291 | ATGAGAAAGCATCATAAGTGA | 37972035 (Meloidogyne hapla) |
| Mi128 | 292 | ATGGAATTGCTTCGTATCAGTAAGG | 27540048 (Meloidogyne hapla) |
| Mi128 | 293 | ATGGAATTGCTTCGTATCAGTAAGGACAAGAAGG | 46987443 (Meloidogyne hapla) |
| Mi128 | 294 | ATGGAATTGCTTCGTATCAGTAAGGACAAGAAGGCTTTGAAGTTTTTGA | 37972035 (Meloidogyne hapla) |
| | | AGAA | |
| Mi128 | 295 | GGACAAGAAGGCTTTGAAGTTTTTGAAGAA | 27540048 (Meloidogyne hapla) |
| Mi128 | 296 | GGTTTAATTACCTACGTTTGAG | 37970947 (Meloidogyne hapla) |
| Mi128 | 297 | GGTTTAATTACCTACGTTTGAGA | 37970869 (Meloidogyne hapla) |
| Mi128 | 298 | GGTTTAATTACCTACGTTTGAGAA | 38513179 (Meloidogyne hapla) |
| Mi128 | 299 | GGTTTAATTACCTACGTTTGAGAAA | 38513227 (Meloidogyne hapla) |
| Mi128 | 300 | AACATCCAGCTGTTGAGGGTCTTGCTGTTGGTTTGGATCGTGGCCATCC | 9829663 (Meloidogyne javanica) |
| Mi128 | 301 | AACATCCAGCTGTTGAGGGTCTTGCTGTTGGTTTGGATCGTGGCCATCC | 9828974 (Meloidogyne javanica) |
| | | TGTGACTAAAAATGTTCGCAAGCCACG | |
| Mi128 | 302 | AAGGAAAACATTCAATCACAGTTTTAATTGTTAAAAA | 15003757 (Meloidogyne javanica) |
| Mi128 | 303 | ACATCCAGCTGTTGAGGGTCTT | 9829249 (Meloidogyne javanica) |
| Mi128 | 304 | ACTATGGAATTGCTTCGTATCAGTA | 9829867 (Meloidogyne javanica) |
| Mi128 | 305 | ACTATGGAATTGCTTCGTATCAGTAAGGACAAGAAGGCTTTGAAGTTTT | 9828974 (Meloidogyne javanica) |
| | | TGAAGAAGCGAATTGGACA | |
| Mi128 | 306 | AGAAGCGAATTGGACAACATGTCCGTGCAAAGAGAAAGCGTGACGA | 15003757 (Meloidogyne javanica) |
| Mi128 | 307 | CAGCTGTTGAGGGTCTTGCTGTTGGTTTGGATCGTGGCCATCCTGTGAC | 9829867 (Meloidogyne javanica) |
| | | TAAAAATGTTCGCAAGCCACG | |
| Mi128 | 308 | CATCCAGCTGTTGAGGGTCTTGCTGTTGGTTTGGATCGTGGCCATCCTG | 9829640 (Meloidogyne javanica) |
| | | TGACTAAAAATGTTCGCAAGCCACG | |
| Mi128 | 309 | CATCCTGTGACTAAAAATGTTCGCAAGCCACGTCAATCTCGTAAAAAAG | 9829249 (Meloidogyne javanica) |
| | | G | |
| Mi128 | 310 | CATGTCCGTGCAAAGAGAAAGCGTGACGAAATCCAACGAATACTCGTTG | 9829663 (Meloidogyne javanica) |
| | | AGATGAGAAAGCATCATAAGTGAGAAAATATAAGGGAAGGAAAACATTC | |
| | | AATCACAGTTTTAATTGTTAAAAA | |
| Mi128 | 311 | CATGTCCGTGCAAAGAGAAAGCGTGACGAAATCCAACGAATACTCGTTG | 9828974 (Meloidogyne javanica) |
| | | AGATGAGAAAGCATCATAAGTGAGAAAATATAAGGGAAGGAAAACATTC | |
| | | AATCACAGTTTTAATTGTTAAAAATAATGTT | |
| Mi128 | 312 | CGAATTGTTCGTGAAGTTGTTCGCGAGGTTGTTGGTTTTTC | 9828974 (Meloidogyne javanica) |
| Mi128 | 313 | CGAATTGTTCGTGAAGTTGTTCGCGAGGTTGTTGGTTTTTCACCTTATG | 9829249 (Meloidogyne javanica) |
| | | AGCGTCGAACTATGGAATTGCTTCGTATCAGTAAGGACAAGAAGGCTTT | |
| | | GAAGTTTTTGAAGAAGCGAATTGGACAACATGTCCGTGCAAAGAGAAAG | |
| | | CGTGACGA | |
| Mi128 | 314 | GAATTGTTCGTGAAGTTGTTCGCGAGGTTGTTGGTTTTTCACCTTATGA | 15003757 (Meloidogyne javanica) |
| | | GCGTCGAACTATGGAATTG | |
| Mi128 | 315 | GTGACTAAAAATGTTCGCAAGCCACG | 9829663 (Meloidogyne javanica) |
| Mi128 | 316 | GTTGAGATGAGAAAGCATCATAAGT | 9829249 (Meloidogyne javanica) |
| Mi128 | 317 | TATGCGATTGAATAAATTTGTTAGAATTCA | 9829640 (Meloidogyne javanica) |
| Mi128 | 318 | TGAGATGAGAAAGCATCATAAGT | 15003757 (Meloidogyne javanica) |
| Mi28 | 319 | TTCGTATCAGTAAGGACAAGAAGGCTTTGAAGTTTTTG | 15003757 (Meloidogyne javanica) |
| Mi128 | 320 | CGTGCAAAGAGAAAGCGTGACGA | 31229640 (Wuchereria bancrofti) |
| Mi128 | 321 | AACATGGTTCCATTCCCACGTCTTCA | 14851165 (Caenorhabditis elegans) |
| Mi128 | 322 | GTTGAATGGATTCCAAACAACGT | 14851165 (Caenorhabditis elegans) |
| Mi129 | 323 | AACATGGTTCCATTCCCACGTCTTCACTT | 21809191 (Ancylostoma caninum), |
| | | | 24559473 (Ancylostoma ceylanicum), |
| | | | 24559473 (Ancylostoma ceylanicum), |
| | | | 68303302 (Ancylostoma caninum), |
| | | | 103002658 (Caenorhabditis Elegans), |
| | | | 72003699 (Caenorhabditis elegans) |
| Mi129 | 324 | ATGGACGAGATGGAATTCACTGA | 103002658 (Caenorhabditis Elegans) |
| Mi129 | 325 | AACATGGTTCCATTCCCACGTCTTCACTT | 24559473 (Ancylostoma ceylanicum) |
| Mi129 | 326 | AACATGGTTCCATTCCCACGTCTTCACTT | 66734013 (Ancylostoma duodenale) |
| Mi129 | 327 | AAGATTCGTGAGGAGTATCCGGACCG | 17972056 (Ascaris suum) |
| Mi129 | 328 | AGGTTTAATTACCCAAGTTTG | 16746749 (Ascaris suum); |
| | | | 14854924Caenorhabditis elegans |
| Mi129 | 329 | GAGGCTTTGTACGACATCTGCT | 16747657 (Ascaris suum) |
| Mi129 | 330 | GAGGCTTTGTACGACATCTGCTTC | 11140987 (Ascaris suum) |
| Mi129 | 331 | GCTTTGTACGACATCTGCTTC | 15784432 (Ascaris suum) |
| Mi129 | 332 | GGTTTAATTACCCAAGTTTGA | 1871111 (Brugia malayi), |
| | | | 91102994 (Nippostrongylus brasiliensis), |
| | | | 10925140 (Caenorhabditis elegans), |
| | | | 6578011 (Brugia pahangi) |
| Mi129 | 333 | GGTTTAATTACCCAAGTTTGAG | 1917158 (Brugia malayi), |
| | | | 83655795 (Heterorhabditis bacteriophora), |
| | | | 91103082 (Nippostrongylus brasiliensis), |
| | | | 10923350 (Caenorhabditis elegans), |
| | | | 1498474 (Toxocara canis), |
| | | | 6578007 (Brugia pahangi) |
| M1129 | 334 | GGTTTAATTACCCAAGTTTGAGT | 1017391 (Brugia malayi), |
| | | | 91102995 Nippostrongylus brasiliensis, |
| | | | 10923353 (Caenorhabditis elegans), |
| | | | 6578016 (Brugia pahangi) |
| Mi129 | 335 | GGTTTAATTACCCAAGTTTGAGTT | 1345290 (Brugia malayi), |
| | | | 83656004 (Heterorhabditis bacteriophora), |
| | | | 91102904 (Nippostrongylus brasiliensis), |
| | | | 10924806 (Caenorhabditis elegans), |
| | | | 1498477 (Toxocara canis) |
| Mi129 | 336 | GGTTTAATTACCCAAGTTTGAGTTT | 1870969 (Brugia malayi), |
| | | | 10923537 (Caenorhabditis elegans) |
| Mi129 | 337 | GTTTAATTACCCAAGTTTGAG | 1017394 (Brugia malayi), |
| | | | 91102668 (Nippostrongylus brasiliensis), |
| | | | 2423515 (Caenorhabditis elegans) |
| Mi129 | 338 | GTTTAATTACCCAAGTTTGAGT | 1870808 (Brugia malayi) |
| Mi129 | 339 | GTTTAATTACCCAAGTTTGAGTT | 1871103 (Brugia malayi) |
| Mi129 | 340 | GTTTAATTACCCAAGTTTGAGTTT | 1893261 (Brugia malayi) |
| Mi129 | 341 | TTAATTACCCAAGTTTGAGTT | 1345262 (Brugia malayi) |
| Mi129 | 342 | AACATGGTTCCATTCCCACGTCTTCA | 14851165 (Caenorhabditis elegans) |
| Mi129 | 343 | AACATGGTTCCATTCCCACGTCTTCACTTT | 47671577 (Caenorhabditis elegans) |
| Mi129 | 344 | AATTACCCAAGTTTGAGTTTT | 40643118 (Caenorhabditis elegans) |
| Mi129 | 345 | AATTACCCAAGTTTGAGTTTTA | 47594382 (Caenorhabditis elegans) |
| Mi129 | 346 | AATTACCCAAGTTTGAGTTTTAC | 47628305 (Caenorhabditis elegans) |
| Mi129 | 347 | ACATGGTTCCATTCCCACGTCTTCACTT | 14854924 (Caenorhabditis elegans) |
| Mi129 | 348 | AGGTTTAATTACCCAAGTTTG | 14824308 (Caenorhabditis elegans) |
| Mi129 | 349 | AGGTTTAATTACCCAAGTTTGA | 10925161 (Caenorhabditis elegans) |
| Mi129 | 350 | AGGTTTAATTACCCAAGTTTGAG | 10924911 (Caenorhabditis elegans) |
| Mi129 | 351 | ATGGACGAGATGGAATTCACTGA | 277183 (Caenorhabditis elegans) |
| Mi129 | 352 | ATGGACGAGATGGAATTCACTGAAGC | 14641526 (Caenorhabditis elegans) |
| Mi129 | 353 | CAACATGGTTCCATTCCCACGTCTTCACTT | 1121008 (Caenorhabditis elegans) |
| Mi129 | 354 | CTTGTTGAGAACACCGACGAGACTTACTG | 2386822 (Caenorhabditis elegans) |
| Mi129 | 355 | GGAAACAATTGGGCCAAAGGACA | 18246795 (Caenorhabditis elegans) |
| Mi129 | 356 | GGTTCCATTCCCACGTCTTCA | 14855460 (Caenorhabditis elegans) |
| Mi129 | 357 | GTCAACATGGTTCCATTCCCACGT | 47651088 (Caenorhabditis elegans) |
| Mi129 | 358 | GTCAACATGGTTCCATTCCCACGTCTT | 18321206 (Caenorhabditis elegans) |
| Mi129 | 359 | GTCAACATGGTTCCATTCCCACGTCTTCACTT | 1120739 (Caenorhabditis elegans) |
| Mi129 | 360 | GTCAACATGGTTCCATTCCCACGTCTTCACTTT | 47656473 (Caenorhabditis elegans) |
| Mi129 | 361 | TAATTACCCAAGTTTGAGTTT | 18248382 (Caenorhabditis elegans) |
| Mi129 | 362 | TAATTACCCAAGTTTGAGTTTT | 18268138 (Caenorhabditis elegans) |
| Mi129 | 363 | TAATTACCCAAGTTTGAGTTTTA | 18269837 (Caenorhabditis elegans) |
| Mi129 | 364 | TACAACGCAACTCTTTCTGTTCATCAACTTGT | 47641436 (Caenorhabditis elegans) |
| Mi129 | 365 | TACAACGCAACTCTTTCTGTTCATCAACTTGTTGA | 1120154 (Caenorhabditis elegans) |
| Mi129 | 366 | TACTTTGTTGAATGGATTCCAAACAACGT | 30744925 (Caenorhabditis elegans) |
| Mi129 | 367 | TGAATGGATTCCAAACAACGT | 1121447 (Caenorhabditis elegans) |
| Mi129 | 368 | TGGTTCCATTCCCACGTCTTCACTT | 18323141 (Caenorhabditis elegans) |
| Mi129 | 369 | TGTCAACATGGTTCCATTCCCACGTCTTCA | 56145926 (Caenorhabditis elegans) |
| Mi129 | 370 | TTAATTACCCAAGTTTGAGTT | 14833204 (Caenorhabd itis elegans) |
| Mi129 | 371 | TTAATTACCCAAGTTTGAGTTT | 14831587 (Caenorhabditis elegans) |
| Mi129 | 372 | TTAATTACCCAAGTTTGAGTTT | 14833268 (Caenorhabditis elegans) |
| Mi129 | 373 | TTAATTACCCAAGTTTGAGTTTTA | 14837998 (Caenorhabditis elegans), |
| | | | 15767282 (Meloidogyne javanica) |
| Mi129 | 374 | TTCCGTGGACGTATGTCAATG | 14641526 (Caenorhabditis elegans) |
| Mi129 | 375 | TTGGCTGTCAACATGGTTCCATTCCC | 47725169 (Caenorhabditis elegans) |
| Mi129 | 376 | TTGGCTGTCAACATGGTTCCATTCCCACGTC | 18324147 (Caenorhabditis elegans) |
| Mi129 | 377 | TTGGCTGTCAACATGGTTCCATTCCCACGTCTTCA | 71987928 (Caenorhabditis elegans) |
| Mi129 | 378 | TTTAATTACCCAAGTTTGAGT | 10925180 (Caenorhabditis elegans), |
| | | | 91103983 (Nippostrongylus brasiliensis), |
| | | | 11068715 (Haemonchus contortus) |
| Mi129 | 379 | TTTAATTACCCAAGTTTGAGTT | 14824445 (Caenorhabditis elegans), |
| | | | 91103246 (Nippostrongylus brasiliensis) |
| Mi129 | 380 | TTTAATTACCCAAGTTTGAGTTT | 14832338 (Caenorhabditis elegans), |
| | | | 91104236 (Nippostrongylus brasiliensis) |
| Mi129 | 381 | TTTAATTACCCAAGTTTGAGTTTT | 14825571 (Caenorhabditis elegans) |
| Mi129 | 382 | TTTAATTACCCAAGTTTGAGTTTTA | 14824404 (Caenorhabditis elegans) |
| Mi129 | 383 | TTTAATTACCCAAGTTTGAGTTTTAC | 14829782 (Caenorhabditis elegans) |
| Mi129 | 384 | GGTTCCAAGTTCTGGGAAGTCAT | 68277783 (Caenorhabditis remanel) |
| Mi129 | 385 | GAATCAATGTTTACTACAATGA | 37594691 (Cooperia oncophora) |
| Mi129 | 386 | GTTGAATGGATTCCAAACAACGT | 37594691 (Cooperia oncophora), |
| | | | 14855460 (Caenorhabditis elegans), |
| Mi129 | 387 | AACCAAATTGGTTCCAAGTTCTGGGAAGT | 53748584 (Cyathostomum catinatum) |
| Mi129 | 388 | GAAGGTATGGACGAGATGGAATTCACTGAAGCCGAGTC | 50402529 (Cyathostomum catinatum) |
| Mi129 | 389 | GATGCCAAGAATATGATGGCTGCTTG | 50402529 (Cyathostomum catinatum) |
| Mi129 | 390 | TTCACTGAAGCCGAGTCCAACATGAA | 22297092 (Cyathostomum catinatum), |
| | | | 22297112 (Cyathostomum pateratum) |
| Mi129 | 391 | AACCAAATTGGTTCCAAGTTCTGGGAAGT | 22297084 (Cyathostomum coronatum) |
| Mi129 | 392 | GAAGGTATGGACGAGATGGAATTCACTGAAGCCGAGTC | 22297134 (Cyathostomum coronatum) |
| Mi129 | 393 | AACCAAATTGGTTCCAAGTTCTGGGAAGT | 22297080 (Cyathostomum pateratum) |
| Mi129 | 394 | GAAGGTATGGACGAGATGGAATTCACTGAAGCCGAGTC | 53748578 (Cyathostomum pateratum) |
| Mi129 | 395 | GATGCCAAGAATATGATGGCTGCTTG | 53748578 (Cyathostomum pateratum) |
| Mi129 | 396 | AACCAAATTGGTTCCAAGTTCTGGGAAGT | 22297100 (Cylicocyclus insigne) |
| Mi129 | 397 | ATGTTTGATGCCAAGAATATGATGGCTGCTTG | 22297100 (Cylicocyclus insigne) |
| Mi129 | 398 | TTGGTTCCAAGTTCTGGGAAGT | 22297102 (Cylicocyclus insigne) |
| Mi129 | 399 | AACCAAATTGGTTCCAAGTTCTGGGAAGT | 10179955 (Cylicocyclus nassatus) |
| Mi129 | 400 | TTCACTGAAGCCGAGTCCAACATGAA | 10179955 (Cylicocyclus nassatus) |
| Mi129 | 401 | AACCAAATTGGTTCCAAGTTCTGGGAAGT | 22297108 (Cylicocyclus radiatus) |
| Mi129 | 402 | ATGTTTGATGCCAAGAATATGATGGCTGCTTG | 22297108 (Cylicocyclus radiatus) |
| Mi129 | 403 | ACTGAAGCCGAGTCCAACATGAA | 53748590 (Cylicostephanus goldi) |
| Mi129 | 404 | GATGCCAAGAATATGATGGCTGCTTG | 53748590 (Cylicostephanus goldi) |
| Mi129 | 405 | AACCAAATTGGTTCCAAGTTCTGGGAAGT | 53748588 (Cylicostephanus longibursatus) |
| Mi129 | 406 | AAGAATATGATGGCTGCTTGTGATCC | 53748588 (Cylicostephanus longibursatus) |
| Mi129 | 407 | TTCACTGAAGCCGAGTCCAACATGAA | 53748588 (Cylicostephanus longibursatus) |
| Mi129 | 408 | AATTACCCAAGTTTGAGTTTT | 452448 (Dirofilaria immitis) |
| Mi129 | 409 | AGGTTTAATTACCCAAGTTTG | 15499065 (Haemonchus contortus); |
| | | | 91103285Nippostrongylus brasiliensis |
| Mi129 | 410 | GAACGAATCAATGTTTACTACAATGAGGCT | 159156 (Haemonchus contortus) |
| Mi129 | 411 | GAATGGATTCCAAACAACGTTAAGACTGC | 159160 (Haemonchus contortus) |
| Mi129 | 412 | TAATTACCCAAGTTTGAGTTT | 27539282 (Haemonchus contortus) |
| Mi129 | 413 | TGAACACGGAATTCAACCTGATGG | 10122229 (Haemonchus contortus) |
| Mi129 | 414 | TGAACACGGAATTCAACCTGATGGA | 27590544 (Haemonchus contortus) |
| Mi129 | 415 | TGGTACACTGGCGAAGGTATGGACGA | 27925410 (Haemonchus contortus) |
| Mi129 | 416 | TTAATTACCCAAGTTTGAGTT | 11068983 (Haemonchus contortus) |
| Mi129 | 417 | TTAATTACCCAAGTTTGAGTT | 11068983 (Haemonchus contortus) |
| Mi129 | 418 | TTTAATTACCCAAGTTTGAGTT | 11068785 (Haemonchus contortus) |
| Mi129 | 419 | ATGTTCCGTCGCAAGGCTTTCCT | 27428224 (Heterodera glycines) |
| Mi129 | 420 | GAAGCCGAGTCCAACATGAACGA | 27428224 (Heterodera glycines) |
| M1129 | 421 | TAATTACCCAAGTTTGAGTTTT | 40304404 (Heterodera glycines) |
| Mi129 | 422 | TTCACGGCCATGTTCCGTCGCAAGGCTTTC | 28706250 (Heterodera glycines) |
| Mi129 | 423 | AATTACCCAAGTTTGAGTTTT | 61116089 (Litomosoides sigmodontis) |
| Mi129 | 424 | CAATGTGGTAACCAAATTGGT | 61115778 (Litomosoides sigmodontis) |
| Mi129 | 425 | AAAAGAGAGAAGTTTTTGTTTT | 34024847 (Meloidogyne arenaria) |
| Mi129 | 426 | AAACGCATCTCTGAGCAATTCACGGCCATGTTCCGTCGCAA | 34024847 (Meloidogyne arenaria) |
| Mi129 | 427 | AATATGATGGCTGCTTGTGATCC | 15352144 (Meloidogyne arenaria) |
| Mi129 | 428 | ACTCTGAAGCTTCAAAATCCAACATA | 15767987 (Meloidogyne arenaria) |
| Mi129 | 429 | ATGATGGCTGCTTGTGATCCACG | 15767987 (Meloidogyne arenaria) |
| Mi129 | 430 | ATGCGTGAAGTAGACGATCAAATGATGTCGGTTCAAAACAAGAATTCGT | 15767987 (Meloidogyne arenaria) |
| | | CATACTTTGTTGAATGGATTCCAAACAACGTTAAGACTGC | |
| Mi129 | 431 | ATGTCTGCGACCTTCATTGGCAACTCCAC | 34024847 (Meloidogyne arenaria) |
| Mi129 | 432 | ATTCAACCTGATGGATCATACAAGGGAGAATCTGACTTGCAATTGGA | 15769029 (Meloidogyne arenaria) |
| Mi129 | 433 | CAGGCCCAAAATGTTGCTGAATTGACCAA | 15767987 (Meloidogyne arenaria) |
| Mi129 | 434 | CGAATCAATGTTTACTACAATGAGGCTCATGGTGGAAAG | 15769029 (Meloidogyne arenaria) |
| Mi129 | 435 | CTGGTTTCGCTCCACTTTCGGCTAAGGGCGCAGCTGCCTA | 34027531 (Meloidogyne arenaria) |
| Mi129 | 436 | CTTTGTTGAATGGATTCCAAACAACGTTAAGACTGC | 34024847 (Meloidogyne arenaria) |
| Mi129 | 437 | GCTATCTTCCGTGGACGTATGTC | 34027531 (Meloidogyne arenaria) |
| Mi129 | 438 | GTATCCGTAACCATGTCTGGAGTGACTACTTGTCTTCGCTTCCCAGGCC | 15767987 (Meloidogyne arenaria) |
| | | AATTGAACGC | |
| Mi129 | 439 | TACCAGCAATATCAAGATGCGACTGTTGAAGACGAAGGGGAATTTGAAG | 34027531 (Meloidogyne arenaria) |
| | | GTGAAGATACCAATCAAGCAACTGTTGAACAAGAATAAAA | |
| Mi129 | 440 | TCCAAGTTCTGGGAAGTCATTTCGGATGAACACGG | 15769029 (Meloidogyne arenaria) |
| Mi129 | 441 | TGTCGGTTCAAAACAAGAATTCGTCAT | 34024847 (Meloidogyne arenaria) |
| Mi129 | 442 | TTATTTTGCTTTTAACCGCATATTTCCGAAATAATTCCTCGCTTAACTT | 34024847 (Meloidogyne arenaria) |
| | | TATTTATGCCA | |
| Mi129 | 443 | TTCCTTCACTGGTACACTGGCGAAGGTATGGACGAGATGGAATTCAC | 34024847 (Meloidogyne arenaria) |
| Mi129 | 444 | TTGGCTGTCAACATGGTTCCATTCCCACGTCTTCACTTTTTCATGCCTG | 15767987 (Meloidogyne arenaria) |
| | | GTTTCGCTCCACTTTCGGCTAAGGGCGCAGCTGCCTA | |
| Mi129 | 445 | TTTATCTAACAAAAGATCTCAAAAAATGCGTGAAATCGTCCATATCCAG | 15769029 (Meloidogyne arenaria) |
| | | GCTGGACAATGTGG | |
| Mi129 | 446 | TAATTACCCAAGTTTGAGTTTT | 37820079 (Meloidogyne chitwoodi) |
| Mi129 | 447 | TAATTACCCAAGTTTGAGTTTTA | 30169860 (Meloidogyne chitwoodi) |
| Mi129 | 448 | AACATGGTTCCATTCCCACGTCTTCACTTTTT | 19267174 (Meloidogyne hapla) |
| Mi129 | 449 | ACTACTTGTCTTCGCTTCCCAGG | 19267247 (Meloidogyne hapla) |
| Mi129 | 450 | AATTACCCAAGTTTGAGTTTT | 4587962 (Meloidogyne javanica) |
| Mi129 | 451 | ATGGACTCGATTAGAGCTGGGCCATA | 14086909 (Meloidogyne javanica) |
| Mi129 | 452 | GACAATGTTTTGGATGTTGTTCGAAAAGAGGCTGAGGGTTGTGATTGTC | 14086909 (Meloidogyne javanica) |
| | | TTCAAGGCTTTCAATTGACTCACTCGCTTGGTGGTGGAACTGGCTCTGG | |
| | | AATGGGAACTTTGCTCATTTCAAAGATTCGTGAGGAGTATCCGGACCG | |
| Mi129 | 453 | GGAGAACTCTTCCGTCCTGACAACTTTGTCTTTGGCCAGAGCGGTGCAG | 14086909 (Meloidogyne javanica) |
| | | GAAACAATTGGGCCAAAGGACATTACACTGAGGGAGC | |
| Mi129 | 454 | GGTGGAAAGTATGTCCCAAGAGC | 14086909 (Meloidogyne javanica) |
| Mi129 | 455 | TAATTACCCAAGTTTGAGTTT | 40671282 (Meloidogyne javanica) |
| Mi129 | 456 | AAAAAAGAGAGAAGTTTTTGTTTT | 40670115 (Meloidogyne paranaensis) |
| Mi129 | 457 | AAAAAGAGAGAAGTTTTTGTTTT | 40669765 (Meloidogyne paranaensis) |
| Mi129 | 458 | AAACGCATCTCTGAGCAATTCACGGCCATGTTCCGTCGCAA | 40670115 (Meloidogyne paranaensis) |
| Mi129 | 459 | ACCTTCATTGGCAACTCCACGGCCAT | 40670115 (Meloidogyne paranaensis) |
| Mi129 | 460 | ATAATTCCTCGCTTAACTTTATTTATGCCA | 40670115 (Meloidogyne paranaensis) |
| Mi129 | 461 | ATTACCCAAG1TTGAGTTTTA | 46498709 (Meloidogyne paranaensis) |
| Mi129 | 462 | ATTTGAAGGTGAAGATACCAATCAAGCAAC | 46497880 (Meloidogyne paranaensis) |
| Mi129 | 463 | CAAAGGACATTACACTGAGGGAGC | 39746828 (Meloidogyne paranaensis) |
| Mi129 | 464 | CGCATATTTCCGAAATAATTCCTCGCTTAACTTTATTTATGCCAAAA | 40669765 (Meloidogyne paranaensis) |
| Mi129 | 465 | CGTCCTGACAACTTTGTCTTTGGCCAGAGCGGTGC | 39746808 (Meloidogyne paranaensis) |
| Mi129 | 466 | GAACTGTTCAAACGCATCTCTGAGCAATTCACGGCCATGTTCCGTCGCA | 40669765 (Meloidogyne paranaensis) |
| | | AGGC | |
| Mi129 | 467 | GAAGACGAAGGGGAATTTGAAGG | 40669765 (Meloidogyne paranaensis) |
| Mi129 | 468 | GAAGCCGAGTCCAACATGAACGACTTGATTTCTGAATACCAGCAATATC | 40669765 (Meloidogyne paraneensis) |
| | | AAGATGCGACTGT | |
| Mi129 | 469 | GACGATCAAATGATGTCGGTTCAAAACAAGAATTCGTCATACTTTGTTG | 40669436 (Meloidogyne paranaensis) |
| | | AATGGATTCCAAACAACGTTAAGACTGCGGTTTGTGACATTCCTCC | |
| Mi129 | 470 | GAGATGGAATTCACTGAAGCCGAGTCCAACATGAACGACTTGATTTCTG | 40670115 (Meloidogyne paranaensis) |
| | | AATACCAGCAATATCAAGATGCGACTGTTGAAGACGAAGGGGAATTTGA | |
| | | AGGTGAAGATACCAATCAAGCAACTGTTGAACAAGAATAAAAAA | |
| Mi129 | 471 | GAGTTGGTCGACAATGTTTTGGATGTT | 39746828 (Meloidogyne paranaensis) |
| Mi129 | 472 | GCCAAGAATATGATGGCTGCTTGTGATCCACGCCATGGACGCTATTT | 40669436 (Meloidogyne paranaensis) |
| Mi129 | 473 | GCGACCTTCATTGGCAACTCCAC | 40669765 (Meloidogyne paranaensis) |
| Mi129 | 474 | GCTGTTCTTGTTGACTTGGAGCCGGG | 39746779 (Meloidogyne paranaensis) |
| Mi129 | 475 | GTTCCGTCGCCTAAGGTCTCTGACACGGT | 40669436 (Meloidogyne paranaensis) |
| Mi129 | 476 | GTTGAACAAGAATAAAAAAGAGAGAAGTTTTTGTTTT | 46497880 (Meloidogyne paranaensis) |
| Mi129 | 477 | TAATTACCCAAGTTTGAGTTT | 39747478 (Meloidogyne paranaensis) |
| Mi129 | 478 | TAATTACCCAAGTTTGAGTTTT | 46498746 (Meloidogyne paranaensis) |
| Mi129 | 479 | TAATTACCCAAGTTTGAGTTTTA | 46498594 (Meloidogyne paranaensis) |
| Mi129 | 480 | TCTGTTCATCAACTTGTTGAGAACAC | 40669436 (Meloidogyne paranaensis) |
| Mi129 | 481 | TGGAACTGGCTCTGGAATGGGAACTTTGCTCATTTCAAAGATTCGTGAG | 40669436 (Meloidogyne paranaensis) |
| | | GAGTATCCGGACCGCATTATGTCTTCCTTCTCTGT | |
| Mi129 | 482 | TTATTTTGCTTTTAACCGCATATTTCCGA | 40670147 (Meloidogyne paranaensis) |
| Mi129 | 483 | TTATTTTGCTTTTAACCGCATATTTCCGAAATA | 46497880 (Meloidogyne paranaensis) |
| Mi129 | 484 | TTCCTTCACTGGTACACTGGCGAAGGTATGGA | 40669765 (Meloidogyne paranaensis) |
| Mi129 | 485 | GAATGGATTCCAAACAACGTTAAGAC | 22527837 (Necator americanus) |
| Mi129 | 486 | TTCATGCCTGGTTTCGCTCCACT | 22528338 (Necator americanus) |
| Mi129 | 487 | TTGGCTGTCAACATGGTTCCATTCCCACGTCTTCACTT | 33242934 (Necator americanus) |
| Mi129 | 488 | GTTGTTCGAAAAGAGGCTGAGGG | 21393141 (Nippostrongylus brasiliensis) |
| Mi129 | 489 | AGGTTTAATTACCCAAGTTTGAG | 10840960 (Onchocerca volvulus) |
| Mi129 | 490 | TACTTTGTTGAATGGATTCCAAA | 3046904 (Onchocerca volvulus) |
| Mi129 | 491 | ACTCTTTCTGTTCATCAACTTGTTGA | 15765704 (Parastrongyloides trichosuri) |
| Mi129 | 492 | CATACTTTGTTGAATGGATTCCAAACAA | 15340288 (Parastrongyloides trichosuri) |
| Mi129 | 493 | CAAAGATTCGTGAGGAGTATCC | 31250007 (Parelaphostrongylus tenuis) |
| Mi129 | 494 | AATATGATGGCTGCTTGTGATCCA | 14494074 (Strongyloides ratti) |
| Mi129 | 495 | CATACTTTGTTGAATGGATTCCAAA | 19252051 (Strongyloides ratti) |
| Mi129 | 496 | TGGTACACTGGCGAAGGTATGGA | 15002387 (Strongyloides ratti) |
| Mi129 | 497 | AATATGATGGCTGCTTGTGATCCA | 12713662 (Strongyloides stercoralis) |
| Mi129 | 498 | CATACTTTGTTGAATGGATTCCAAA | 12713662 (Strongyloides stercoralis) |
| Mi129 | 499 | TACTTTGTTGAATGGATTCCAA | 9830375 (Strongyloides stercoralis) |
| Mi129 | 500 | AGCAAATGTTTGATGCCAAGAA | 27758042 (Teladorsagia circumcincta) |
| Mi129 | 501 | CCAAGTTCTGGGAAGTCATTTC | 27756411 (Teladorsagia circumcincta) |
| Mi129 | 502 | GAACGAATCAATGTTTACTACAATGAGGCT | 27756411 (Teladorsagia circumoincta) |
| Mi129 | 503 | GGTATGGACGAGATGGAATTCAC | 19559988 (Toxocara canis) |
| Mi129 | 504 | ATGGACGAGATGGAATTCACTGA | 2372083 (Caenorhabditis elegans) |
| Mi129 | 505 | ATGGACGAGATGGAATTCACTGAAGC | 1116997 (Caenorhabditis elegans) |
| Mi129 | 506 | CTTGGTGGTGGAACTGGCTCTGGAATGGGAACT | 108965893 (Bursaphelenchus xylophilus) |
| Mi129 | 507 | GATGCCAAGAATATGATGGCTGC | 108983689 (Bursaphelenchus xyiophilus) |
| Mi129 | 508 | GCAACTCTTTCTGTTCATCAACT | 115342288 (Heterorhabditis bacteriophora) |
| Mi129 | 509 | GGAACTGGCTCTGGAATGGGA | 108976152 (Bursaphelenchus mucronatus) |
| Mi129 | 510 | GGTTCCATTCCCACGTCTTCA | 14855460 (Caenorhabditis elegans) |
| Mi129 | 511 | GTCAACATGGTTCCATTCCCACGT | 103038769 (Caenorhabditis elegans) |
| Mi129 | 512 | ATGGACGAGATGGAATTCACTGAAGC | 71983644 (Caenorhabditis elegans) |
| Mi129 | 513 | TACAACGCAACTCTTTCTGTTCATCAACTTGTTGA | 1120154 (Caenorhabditis elegans) |
| Mi129 | 514 | TTAATTACCCAAGTTTGAGTTTTAC | 14829782 (Caenorhabditis elegans) |

TABLE 7

| Target | SEQ | | |
|---|---|---|---|
| ID | ID NO | Sequence* | Example Gi-number and species |
| Mi05 | 515 | TTATTATTTCAAAAAAAAAAA | 50829840 (Apis mellifera) |
| M138 | 516 | TACGGCCAGGACGTATTGATCGT | 47532028 (Acyrthosiphon pisum) |
| Mi40 | 517 | GATATGGATGAAGATGAATTGGAAATGTTATC | 66530331 (Apis mellifera) |
| Mi40 | 518 | CAGTGTTTGGAAAGATATGAATAT | 84647988 (Myzus persicae) |
| Mi40 | 519 | GATATGGATGAAGATGAATTGGA | 124238642 (Solenopsis invicta) |
| Mi40 | 520 | GATATGGATGAAGATGAATTGGAAATGTTATC | 110764965 (Apis mellifera) |
| Mi111 | 521 | ACAAATTCAAGAAATTAAGGA | 66509031 (Apis mellifera) |
| Mi111 | 522 | ACAAATTCAAGAAATTAAGGAG | 77329902 (Chironomus tentans) |
| Mi111 | 523 | TTTAAAAAAATCTTTTAAAAAT | 82830959 (Boophilus microplus) |
| Mi111 | 524 | CACGCAGTTGTTGGTGTTTTGG | 94429207 (Bombyx mori) |
| Mi111 | 525 | GTGGAATTGCCACTTACACATCCAGA | 78539405 (Glossina morsitans) |
| Mi125 | 526 | CAAAAGCACATTGACTTCTCC | 37952462 (Ips pini) |
| Mi125 | 527 | CACATTGACTTCTCCACAAAATC | 56156455 (Rhynchosciara americana) |
| Mi125 | 528 | CGTAAAGCTGCTCGTGAACTT | 77332739 (Chironomus tentans) |
| M1125 | 529 | CGTCGTTTGGTTCGTATTGGAGT | 62240069 (Diabrotica virgifera), |
| | | | 94438358 (Bombyx mori) |
| Mi125 | 530 | GACTTTTTGGAGCGTCGTTTGCAGACGCA | 41405201 (Drosophila melanogaster) |
| Mi125 | 531 | GTTTTCAAATTGGGATTGGCTAAATC | 31364772 (Toxoptera citricida) |
| Mi125 | 532 | TTCAAATTGGGATTGGCTAAATC | 37622137 (Sarcophaga crassipalpis) |
| Mi125 | 533 | GACTTTTTGGAGCGTCGTTTGCAGAC | 91826622 (Bombyx mori) |
| Mi125 | 534 | TTGGAGCGTCGTTTGCAGACGCA | 114056346 (Drosophila melanogaster) |
| Mi127 | 535 | ATCATTGCCAATGATCAAGGAAA | 60315026 (Tricholepisma aurea) |
| Mi127 | 536 | ATGAAGCATTGGCCGTTCAAGGT | 61951993 (Tribolium castaneum) |
| Mi127 | 537 | ATTGCTTATGGTTTGGATAAGAAAG | 33298660 (Glossina morsitans) |
| Mi127 | 538 | ATTTGACGATCCAGCTGTTCA | 60303218 (Julodis onopordi) |
| Mi127 | 539 | CAGCATGGAAAAGTCGAGATCAT | 61955760 (Tribolium castaneum) |
| Mi127 | 540 | CATGGAAAAGTCGAGATCATTGC | 77326551 (Chironomus tentans) |
| Mi127 | 541 | GACATGAAGCATTGGCCGTTCAA | 60308798 (Silpha atrata) |
| Mi127 | 542 | GGTGTATTCCAGCATGGAAAAGT | 16285055 (Apis mellifera) |
| Mi127 | 543 | ATTGCTTATGGTTTGGATAAG | 78525195 (Glossina morsitans) |
| Mi127 | 544 | GAGATCATTGCCAATGATCAAGGAAA | 107248738 (Drosophila melanogaster) |
| Mi127 | 545 | GAGATCATTGCCAATGATCAAGGAAA | 99005779 (Leptinotarsa decemlineata) |
| Mi127 | 546 | TCGGAATTGATCTGGGAACGAC | 113208384 (Mamestra brassicae) |
| Mi127 | 547 | TTTAAAAAAATCTTTTAAAAA | 124012837 (Oncopeltus fasciatus) |
| Mi128 | 548 | GAAATTTAAAGAATGACAAAG | 77321879 (Chironomus tentans) |
| Mi128 | 549 | AATTGGGCCAAAGGACATTACACTGA | 84114197 (Blomia tropicalis) |
| Mi128 | 550 | CGCAAGGCTTTCCTTCACTGGTA | 82849727 (Boophilus microplus) |
| Mi128 | 551 | CGCAAGGCTTTCCTTCACTGGTACACTGG | 82831082 (Boophilus microplus) |
| Mi128 | 552 | GCAAGGCTTTCCTTCACTGGTA | 82835811 (Boophilus microplus) |
| Mi128 | 553 | GCCATGTTCCGTCGCAAGGCTTTCCT | 110889758 (Argas monolakensis) |
| Mi128 | 554 | TTTGTTGAATGGATTCCAAACAA | 84114847 (Blomia tropicalis) |
| Mi128 | 555 | GTCAACATGGTTCCATTCCCACGT | 84114197 (Blomia tropicalis) |
| Mi129 | 556 | AAATTGGCTGTCAACATGGTTCC | 62239796 (Diabrotica virgifera) |
| Mi129 | 557 | AACAATTGGGCCAAAGGACATTACAC | 37659666 (Bombyx mori) |
| Mi129 | 558 | AACATGGTTCCATTCCCACGT | 22038826 (Ctenocephalides felis); |
| | | | 67884361 (Drosophila pseudoobscura) |
| Mi129 | 559 | AACATGGTTCCATTCCCACGTCT | 67841791 (Drosophila pseudoobscura); |
| | | | 67895980 (Drosophila pseudoobscura) |
| Mi129 | 560 | AAGGCTTTCCTTCACTGGTACACTGG | 55894639 (Locusta migratonia) |
| Mi129 | 561 | AGCAAATGTTTGATGCCAAGAA | 40917832 (Bombyx mori); |
| | | | 158744 (Drosophila hydei) |
| Mi129 | 562 | ATCAATGTTTACTACAATGAGGC | 50560478 (Homalodisca coagulata) |
| Mi129 | 563 | ATGATGGCTGCTTGTGATCCACG | 56150501 (Rhynchosciara americana), |
| | | | 123264037 (Laupala kohalensis) |
| Mi129 | 564 | ATGGAATTCACTGAAGCCGAG | 16898208 (Ctenocephalides felis) |
| Mi129 | 565 | ATGGTTCCATTCCCACGTCTTCACTT | 77324743 (Chironomus tentans); |
| | | | 67880163 (Drosophila pseudoobscura) |
| Mi129 | 566 | ATGTTCCGTCGCAAGGCTTTC | 77642397 (Aedes aegypti), |
| | | | 92040999 (Drosophila willistoni), |
| | | | 67893984 (Drosophila pseudoobscura), |
| | | | 56151969 (Rhynchosciara americana),, |
| | | | 45244777 (Bombyx mori), |
| Mi129 | 567 | ATGTTCCGTCGCAAGGCTTTC | 67893984 (Drosophila pseudoobscura) |
| Mi129 | 568 | ATGTTCCGTCGCAAGGCTTTC | 56151969 (Rhynchosciara americana) |
| Mi129 | 569 | CAATTGAACGCTGATCTTCGCAAATTGGC | 56150501 (Rhynchosciara americana) |
| Mi129 | 570 | CATGGTTCCATTCCCACGTCT | 67894145 (Drosophila pseudoobscura) |
| Mi129 | 571 | CGTATTTTTTTAATTTTATTTT | 55798845 (Acyrthosiphon pisum) |
| Mi129 | 572 | CTGCATCGACAACGAGGCTTTGTA | 24650775 (Drosophila melanogaster) |
| Mi129 | 573 | CTTACTGCATCGACAACGAGGC | 48095546 (Apis mellifera) |
| Mi129 | 574 | GAAGGTATGGACGAGATGGAATTCACTGAAGC | 73612788 (Aphis gossypii), |
| | | | 99009385 (Leptinotarsa decemlineata) |
| Mi129 | 575 | GAGATGGAATTCACTGAAGCCGA | 77759798 (Aedes aegypti), |
| | | | 83663743 (Myzus persicae) |
| Mi129 | 576 | GATGCCAAGAATATGATGGCTGCTTG | 77753173 (Aedes aegypti) |
| Mi129 | 577 | GCCATTCAAGAACTGTTCAAACG | 33429239 (Glossina morsitans) |
| Mi129 | 578 | GGCGAAGGTATGGACGAGATGGAATTCACTGAAGCCGA | 75466135 (Tribolium castaneum) |
| Mi129 | 579 | GGTATGGACGAGATGGAATTCAC | 33427916 (Glossina morsitans), |
| | | | 83930827 (Lutzomyia longipalpis) |
| Mi129 | 580 | GTTGTGATTGTCTTCAAGGCTT | 62239916 (Diabrotica ving ifera) |
| Mi129 | 581 | TACTGCATCGACAACGAGGCT | 29535296 (Bombyx mori); |
| | | | 4463876 (Drosophila melanogaster); |
| | | | 38047746 (Drosophila yakuba) |
| Mi129 | 582 | TACTGCATCGACAACGAGGCTT | 3944276 (Drosophila melanogaster) |
| Mi129 | 583 | TACTGCATCGACAACGAGGCTTT | 3719603 (Manduca sexta) |
| Mi129 | 584 | TACTTTGTTGAATGGATTCCAAA | 55882682 (Locusta migratoria) |
| Mi129 | 585 | TCCATTCCCACGTCTTCACTT | 67840488 (Drosophila pseudoobscura) |
| Mi129 | 586 | TCCATTCCCACGTCTTCACTTTTTCATGCCTGG | 33429092 (Glossina morsitans) |
| Mi129 | 587 | TGGCTGTCAACATGGTTCCATTCCCACGTCT | 77706291 (Aedes aegypti); |
| | | | 67842756 (Drosophila pseudoobscura) |
| Mi129 | 588 | TGGTACACTGGCGAAGGTATGGA | 56151969 (Rhynchosciara americana) |
| Mi129 | 589 | TGGTACACTGGCGAAGGTATGGACGA | 47534792 (Acyrthosiphon pisum) |
| Mi129 | 590 | TGTACGACATCTGCTTCCGAAC | 40918504 (Bombyx mori) |
| Mi129 | 591 | TTCCGTGGACGTATGTCAATG | 77324743 (Chironomus tentans), |
| | | | 118158757 (Lysiphlebus testaceipes) |
| Mi129 | 592 | TTTTTAATTTTATTTTGCTTT | 24641385 (Drosophila melanogaster) |
| Mi129 | 593 | AAGGCTTTCCTTCACTGGTACACTGGCGA | 124242563 (Solenopsis invicta) |
| Mi129 | 594 | AAGGTATGGACGAGATGGAATTCAC | 113022997 (Bemisia tabaci) |
| Mi129 | 595 | AATATGATGGCTGCTTGTGATCCA | 118158757 (Lysiphlebus testaceipes) |
| Mi129 | 596 | AATTTGAAGGTGAAGATACCAA | 123262489 (Laupala kohalensis) |
| Mi129 | 597 | ACCTTCATTGGCAACTCCACGGCCAT | 92944161 (Drosophila ananassae) |
| Mi129 | 598 | TCATACTTTGTTGAATGGATTCCAAACAACGT | 118158757 (Lysiphlebus testaceipes) |
| Mi129 | 599 | ATGGACGAGATGGAATTCACTGA | 91086094 (Tribolium castaneum) |
| Mi129 | 600 | ATGGTTCCATTCCCACGTCTTCACTTTTTCATGCCTGG | 78543404 (Glossina morsitans) |
| Mi129 | 601 | ATGTTCCGTCGCAAGGCTTTC | 93002796 (Drosophila grimshawi) |
| Mi129 | 602 | CAAATTGGTTCCAAGTTCTGGGAA | 118158037 (Lysiphlebus testaceipes) |
| Mi129 | 603 | CGCAAGGCTTTCCTTCACTGGTACAC | 110557210 (Drosophila ananassae) |
| Mi129 | 604 | CGGCCATGTTCCGTCGCAAGGC | 92940755 (Drosophila virilise) |
| Mi129 | 605 | CTTTGTTGAATGGATTCCAAA | 118158492 (Lysiphlebus testaceipes) |
| Mi129 | 606 | GACAACTTTGTCTTTGGCCAG | 92479391 (Drosophila erecta) |
| Mi129 | 607 | ATGTTCCGTCGCAAGGCTTTC | 114949473 (Aedes aegypti) |
| Mi129 | 608 | GCCATTCAAGAACTGTTCAAACG | 78532419 (Glossina morsitans) |
| Mi129 | 609 | GCCGAGTCCAACATGAACGAC | 83930973 (Lutzomyia longipalpis) |
| Mi129 | 610 | GGCGAAGGTATGGACGAGATGGA | 90982659 (Aedes aegypti) |
| Mi129 | 611 | GTCAACATGGTTCCATTCCCACGTCT | 109290429 (Culex pipiens), |
| | | | 93001606 (Drosophila grimshawi) |
| Mi129 | 612 | ATGTTCCGTCGCAAGGCTTTC | 93001606 (Drosophila grimshawi |
| Mi129 | 613 | TACTGCATCGACAACGAGGCT | 92952298 (Drosophila ananassae) |
| Mi129 | 614 | TACTGCATCGACAACGAGGCT | 92473113 (Drosophila erecta) |
| Mi129 | 615 | TACTGCATCGACAACGAGGCT | 107330711 (Drosophila melanogaster) |
| Mi129 | 616 | TCATACTTTGTTGAATGGATTCCAAACAA | 119363347 (Rhodnius prolixus) |
| Mi129 | 617 | TGGAGCCGGGAACCATGGACTC | 92931833 (Drosophila virilise) |
| Mi129 | 618 | TGGAGCCGGGAACCATGGACTC | 123283502 (Laupala kohalensis) |
| Mi129 | 619 | TGGCTGTCAACATGGTTCCATTCCC | 92047961 (Drosophila willistoni) |
| Mi129 | 620 | TGGGCCAAAGGACATTACACTGAGGG | 103793359 (Heliconius erato) |
| Mi129 | 621 | TGGGCCAAAGGACATTACACTGAGGG | 103793359 (Heliconius erato) |
| Mi129 | 622 | TTCCGTGGACGTATGTCAATG | 118159629 (Lysiphlebus testaceipes) |
| Mi129 | 623 | TTTTACTATTTAATTTATCTA | 83935851 (Lutzomyia longipalpis) |
| Mi129 | 624 | TCATACTTTGTTGAATGGATTCCAAACAACGT | 118159629 (Lysiphlebus testaceipes) |

TABLE 8

| Target | SEQ | | |
|---|---|---|---|
| ID | ID NO | Sequence* | Example Gi-number and species |
| Mi40 | 625 | ATTGATATGGATGAAGATGAA | 90555342 (Lentinula edodes) |
| Mi111 | 626 | AACAAATTCAAGAAATTAAGGA | 110464534 (Rhizopus oryzae) |
| Mi125 | 627 | TTCGTCGTTTGGTTCGTATTGG | 110460324 (Rhizopus oryzae) |
| Mi127 | 628 | ATTGCTTATGGTTTGGATAAGAAAGGC | 119201164 (Saccharomyces cerevisiae) |
| Mi127 | 629 | GGAACGTCTGATTGGTGATGC | 90538259 (Gloeophyllum trabeum) |
| Mi127 | 630 | GAGATCATTGCCAATGATCAAGG | 58160145 (Phytophthora infestans) |
| Mi129 | 631 | AACTCTTCCGTCCTGACAACTTT | 110459609 (Rhizopus oryzae) |
| Mi129 | 632 | AGCAAATGTTTGATGCCAAGAA | 98997118 (Spizellomyces punctatus) |
| Mi129 | 633 | GGTATGGACGAGATGGAATTCACTGA | 98997118 (Spizellomyces punctatus) |
| Mi129 | 634 | ATCGACAACGAGGCTTTGTACGACAT | 119495801 (Neosartorya fischeri) |
| Mi129 | 635 | ATCGACAACGAGGCTTTGTACGACATCTG | 110118467 (Saitoella complicate) |
| Mi129 | 636 | ATGGACGAGATGGAATTCACTGAAGCCGAGTC | 114217954 (Ophiostoma clavigerum) |
| Mi129 | 637 | CAATGTGGTAACCAAATTGGT | 116079742 (Botryotinia fuckeliana), |
| | | | 90392956 (Cunninghamella elegans), |
| | | | 110465715 (Rhizopus oryzae), |
| | | | 117792906 (Trichophyton rubrum) |
| Mi129 | 638 | AACTCTTCCGTCCTGACAACTTT | 110465715 (Rhizopus oryzae) |
| Mi129 | 639 | CATACTTTGTTGAATGGATTCC | 119184208 (Coccidioides immitis) |
| Mi129 | 640 | CCTGACAACTTTGTCTTTGGCCAG | 116002727 (Trichoderma atroviride) |
| Mi129 | 641 | GACACGGTCGTTGAGCCCTACAACGC | 90624483 (Corynascus heterothallicus) |
| Mi129 | 642 | GCAAATGTTTGATGCCAAGAA | 116360500 (Puccinia striiformis) |
| Mi129 | 643 | GCCATGTTCCGTCGCAAGGCTTTCCTTCA | 90610729 (Ophiostoma piliferum) |
| Mi129 | 644 | GCCGAGTCCAACATGAACGAC | 90610729 (Ophiostoma piliferum), |
| | | | 117422543 (Blastocladiella emersonii) |
| Mi129 | 645 | GGAAACAATTGGGCCAAAGGA | 90644351 (Trametes versicolor) |
| Mi129 | 646 | GGTATGGACGAGATGGAATTCA | 50551818 (Yarrowia lipolytica) |
| Mi129 | 647 | AGGTATGGACGAGATGGAATTCAC | 70689303 (Gibberella monil iformis) |
| Mi129 | 648 | ATGGACGAGATGGAATTCACTGA | 46343419 (Paracoccidloides brasiliensis) |
| Mi129 | 649 | ATGTTCCGTCGCAAGGCTTTC | 70995399 (Aspergillus fumigatus); |
| | | | 49086565 (Aspergillus nidulans FGSCA4); |
| | | | 70825342 (Aspergillus niger), |
| | | | 119495801 (Neosartorya fischeri), |
| | | | 70995399 (Aspergillus fumigatus), |
| | | | 70825342 (Aspergillus niger), |
| | | | 84573702 (Aspergillus oryzae), |
| | | | 116202308 (Chaetomium globosum), |
| | | | 116002543 (Trichoderma atroviride), |
| | | | 119184208 (Coccidloides immitis) |
| Mi129 | 650 | GGTATGGACGAGATGGAATTCACTGA | 98996315 (Spizellomyces punctatus) |
| Mi129 | 651 | TACCAGCAATATCAAGATGCGAC | 45739462 (Hebeloma cylindrosporum) |
| Mi129 | 652 | TGCATCGACAACGAGGCTTTGTACGA | 50980793 (Paxillus involutus) |
| Mi129 | 653 | TGGGCCAAAGGACATTACACTGAGGGAGC | 83922458 (Phakopsora pachyrhizi) |
| Mi129 | 654 | TTGGCTGTCAACATGGTTCCATTCCC | 90385723 (Amorphotheca resinae), |
| | | | 45743270 (Hebeloma cylindrosporurn) |
| Mi129 | 655 | TTGGTGGTGGAACTGGCTCTGG | 90535726 (Gloeophyllum trabeum), |
| | | | 90546970 (Lentinula edodes) |
| Mi129 | 656 | CAATGTGGTAACCAAATTGGT | 54397728 (Trichophyton rubrum) |
| Mi129 | 657 | GAGTCCAACATGAACGACTTG | 66909865 (Phaeosphaeria nodorum), |
| | | | 90357303 (Aureobasidium pullulans) |
| Mi129 | 658 | GCCATGTTCCGTCGCAAGGCTTT | 70709731 (Gibberella moniliformis) |
| Mi129 | 659 | GCCATGTTCCGTCGCAAGGCTTTC | 70662385 (Gibberella moniliformis), |
| | | | 90624483 (Corynascus heterothallicus), |
| | | | 121701810 (Aspergillus clavatus), |
| | | | 115491490 (Aspergillus terreus), |
| | | | 90357303 (Aureobasidium pullulans), |
| | | | 90618069 (Corynascus heterothallicus), |
| | | | 89975356 (Hypocrea lixii), |
| Mi129 | 660 | GCCGAGTCCAACATGAACGACTTG | 60674404 (Alternaria brassicicola), |
| | | | 116002543 (Trichoderma atroviride), |
| | | | 89975356 (Hypocrea lixii) |
| Mi129 | 661 | GGTATGGACGAGATGGAATTCAC | 27735638 (Aspergillus flavus); |
| | | | 70662385 (Gibberella moniliformis); |
| | | | 22499956 (Gibberella zeae), |
| | | | 84573811 (Aspergillus oryzae), |
| | | | 46124466 (Gibberella zeae) |
| Mi129 | 662 | TTCCGTCCTGACAACTTTGTCT | 21649805 (Conidiobolus coronatus) |

1. A double stranded ribonucleotide sequence produced from the expression of a polynucleotide sequence comprising a nucleic acid sequence selected from the group comprising
(i) sequences represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof,
(ii)) sequences which are at least 70%, preferably at least 75%, 80%, 85%, 90%, more preferably at least 95%, 96%, 97%, 98% or 99% identical to a sequence represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof, and
(iii) sequences comprising at least 17, preferably at least 18, 19, 20 or 21, more preferably at least 22, 23 or 24 contiguous nucleotides of any of the sequences represented by SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof,
or wherein said nucleic acid sequence is an orthologue of a gene comprising at least 17, preferably at least 18, 19, 20 or 21, more preferably at least 22, 23 or 24 contiguous nucleotides of any of SEQ ID NOs 61 to 662, or a complement thereof,
wherein ingestion of said ribonucleotide sequence by a pathogenic pest inhibits the growth of said pest.

2. The ribonucleotide sequence of claim 1, wherein ingestion of said sequence inhibits expression of a nucleotide sequence substantially complementary to said sequence

3. The ribonucleotide sequence of claim 1, wherein said pathogenic pest is chosen from the group consisting of a nematode, a plant pathogenic nematode, an insect, a plant pathogenic insect, a fungus and a plant pathogenic fungus.

4. A cell transformed with a polynucleotide comprising a nucleic acid sequence as defined in claim 1, optionally operably linked to a regulatory sequence.

5. The cell of claim 4, wherein said cell is a plant cell.

6. A plant transformed with a polynucleotide having a nucleic acid sequence as defined in claim 1, said nucleic acid sequence optionally operably linked to a regulatory sequence.

7. The plant of claim 6, wherein said sequence inhibits a plant pathogenic pest biological activity.

8. The plant of claim 6, wherein said sequence inhibits expression of a target sequence.

9. The plant of claim 8 wherein said target sequence is a sequence from a plant pathogenic nematode, a plant pathogenic insect or a plant pathogenic fungus.

10. The plant of claim 6, wherein said plant is cytoplasmic male sterile.

11. The plant of claim 6, wherein said plant further comprises or expresses at least one pesticidal agent selected from the group consisting of a patatin, a Bacillus thuringiensis insecticidal protein, a Bacillus thuringiensis nematicidal protein, a Xenorhabdus insecticidal protein, a Photorhabdus insecticidal protein, a Bacillus laterosporous insecticidal protein, and a Bacillus sphearicus insecticidal protein.

12. The plant of claim 11 wherein said Bacillus thuringiensis insecticidal protein is selected from the group consisting of a Cry1, a Cry3, a TIC851, a CryET170, a Cry22, a binary insecticidal protein CryET33 and CryET34, a binary insecticidal protein CryET80 and CryET76, a binary insecticidal protein TIC100 and TIC101, and a binary insecticidal protein PS149B1.

13. The plant of claim 6, wherein said plant is chosen from the group comprising alfalfa, apple, apricot, Arabidopsis, artichoke, asparagus, avocado, banana, barley, beans, beet, blackberry, blueberry, Brassica, broccoli, Brussels sprouts, cabbage, canola, carrot, cassaya, cauliflower, a cereal, celery, cherry, citrus, Clementine, coffee, corn, cotton, cucumber, eggplant, endive, eucalyptus, figs, grape, grapefruit, groundnuts, ground cherry, kiwifruit, lettuce, leek, lemon, lime, pine, maize, mango, melon, millet, mushroom, nut oat, okra, onion, orange, an ornamental plant or flower or tree, papaya, parsley, pea, peach, peanut, peat, pepper, persimmon, pineapple, plantain, plum, pomegranate, potato, pumpkin, radicchio, radish, rapeseed, raspberry, rice, rye, sorghum, soy, soybean, spinach, strawberry, sugar beet, sugarcane, sunflower, sweet potato, tangerine, tea, tobacco, tomato, a vine, watermelon, wheat, yams and zucchini.

14. The plant of claim 6, wherein said plant is resistant against infestation by a plant pathogenic nematode chosen from the group consisting of Meloidogyne spp. (e.g. M. incognita, M. javanica, M. graminicola, M. arenaria, M. chitwoodi, M. hapla or M. paranaensis), Heterodera spp. (e.g. H. oryzae, H. glycines, H. zeae or H. schachtii), Globodera spp. (e.g. G. pallida or G. rostochiensis), Rotylenchulus spp. (e.g. R. reniformis), Pratylenchus spp. (e.g. P. coffeae, P. Zeae or P. goodeyi), Radopholus spp. (e.g. R. similis), Hirschmaniella spp. (e.g. H. oryzae), Aphelenchoides spp. (e.g. A. Besseyi),Belonolaimus spp., Criconemoides spp., Ditylenchus spp. (e.g. D. dipsaci, D. destructor or D. Angustus), Helicotylenchus spp., Hoplolaimus spp., Longidorus spp. (e.g. L. macrosoma), Paratrichodorus spp. (e.g. P. minor or P. Teres), Tylenchorhynchus spp., and Xiphinema spp. (e.g. X. Index or X. americanum).

15. A seed or reproductive or propagation material for a plant transformed with a polynucleotide having a nucleic acid sequence as defined in claim 1, said nucleic acid sequence optionally operably linked to a regulatory sequence, wherein said seed or reproductive or propagation material comprises a polynucleotide having a nucleic acid sequence as defined in claim 1 or wherein said seed comprises a double stranded ribonucleotide sequence produced from the expression of said polynucleotide sequence.

16. A product produced from the plant transformed with a polynucleotide having a nucleic acid sequence as defined in claim 1, said nucleic acid sequence optionally operably linked to a regulatory sequence, wherein said product comprises a polynucleotide having a nucleic acid sequence as defined in claim 1 or wherein said seed comprises a double stranded ribonucleotide sequence produced from the expression of said polynucleotide sequence.

17. The product of claim 16, wherein said product is selected from the group consisting of food, feed, fibre, paper, meal, protein, starch, flour, silage, coffee, tea, and oil.

18. A pesticide comprising a product expressing a nucleic acid sequence as defined in claim 1.

19. A method for controlling or preventing nematode growth comprising providing a nematode pest with plant material derived from the plant of claim 6, wherein said plant comprises a polynucleotide sequence that inhibits a nematode biological activity.

20. A method for controlling or preventing nematode growth comprising providing a nematode pest with the pesticide of claim 18.

21. A method for producing a plant resistant against a plant pathogenic organism comprising:
transforming a plant cell with a polynucleotide having a nucleic acid sequence as defined in claim 1, said nucleic acid sequence optionally operably linked to a regulatory sequence,
regenerating a plant from the transformed plant cell; and
growing the transformed plant under conditions suitable for the expression of an RNA molecule from said polynucleotide, said grown transformed plant resistant to said plant pathogenic organism compared to an untransformed plant.

22. A method for improving yield, comprising:
transforming a plant cell with a polynucleotide having a nucleic acid sequence as defined in claim 1, said nucleic acid sequence optionally operably linked to a regulatory sequence,
regenerating a plant from the transformed plant cell; and
growing the transformed plant under conditions suitable for the expression of an RNA molecule from said polynucleotide, said expression inhibiting feeding by a plant pathogenic organism and loss of yield due to pest infestation.

23. The method of claim 21, wherein said plant pathogenic organism is a plant pathogenic nematode, preferably a nematode chosen from the group of nematodes consisting of Meloidogyne spp. (e.g. M. incognita, M. javanica, M. graminicola, M. arenaria, M. chitwoodi, M. hapla or M. paranaensis), Heterodera spp. (e.g. H. oryzae, H glycines, H. zeae or H. schachtii), Globodera spp. (e.g. G. pallida or G. rostochiensis), Rotylenchulus spp. (e.g. R. reniformis), Pratylenchus spp. (e.g. P. coffeae, P. zeae or P. goodeyi), Radopholus spp. (e.g. R. similis), Hirschmaniella spp. (e.g. H. oryzae), Aphelenchoides spp. (e.g. A. Besseyi), Belonolaimus spp., Criconemoides spp., Ditylenchus spp. (e.g. D. dipsaci, D. destructor or D. Angustus), Helicotylenchus spp., Hoplolaimus spp., Longidorus spp. (e.g. L. macrosoma), Paratrichodorus spp. (e.g. P. minor or P. Teres), Tylenchorhynchus spp., and Xiphinema spp. (e.g. X. Index or X. americanum).

24. The method of claim 19, wherein polynucleotide expression produces an RNA molecule that suppresses a target gene in a plant pathogenic nematode upon ingestion of said RNA molecule, wherein said target gene performs at least one essential function selected from the group consisting of feeding by the nematode, viability of the nematode, nematode cell apoptosis, differentiation and development of the nematode or any nematode cell, sexual reproduction by the nematode, muscle formation, muscle twitching, muscle contraction, juvenile hormone formation and/or reduction, juvenile hormone regulation, ion regulation and transport, maintenance of cell membrane potential, amino acid biosynthesis, amino acid degradation, sperm formation, pheromone synthesis, pheromone sensing, antennae formation, wing formation, leg formation, egg formation, larval maturation, digestive enzyme formation, haemolymph synthesis, haemolymph maintenance, neurotransmission, larval stage transition, pupation, emergence from pupation, cell division, energy metabolism, respiration, cytoskeleton structure synthesis and maintenance, nucleotide metabolism, nitrogen metabolism, water use, water retention, and sensory perception.

25. The method of claim 19, wherein the plant is rice, cotton, potato, tomato, corn, tobacco or soybean and wherein said target gene is a gene coding for a nematode ortholog, preferably a Meloidogyne incognita orthologue, of a protein selected from the group of proteins whose function is given in Table 1.

26. The method of claim 19, wherein the plant is rice and wherein:
the nucleic acid sequence is chosen from the group comprising:
(i) sequences represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof,
(ii) sequences which are at least 70%, preferably at least 75%, 80%, 85%, 90%, more preferably at least 95%, 96%, 97%, 98% or 99% identical to a sequence represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof, and
(iii) sequences comprising at least 17 contiguous nucleotides of any of the sequences represented by SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof,
or wherein said nucleic acid sequence is an ortholog of a gene comprising at least 17 contiguous nucleotides of any of SEQ ID NOs 61 to 662, or the complement thereof, and
the nematode is chosen from the group consisting of Meloidogyne spp., Hirschmaniella spp., Aphelenchoides spp., Heterodera spp., Ditylenchus spp. and Pratylenchus spp.

27. The method of claim 19, wherein the plant is corn and wherein:
the nucleic acid sequence is chosen from the group comprising:
(i) sequences represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof,
(ii) sequences which are at least 70%, preferably at least 75%, 80%, 85%, 90%, more preferably at least 95%, 96%, 97%, 98% or 99% identical to a sequence represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof, and
(iii) sequences comprising at least 17 contiguous nucleotides of any of the sequences represented by SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662 or the complement thereof,
or wherein said nucleic acid sequence is an ortholog of a gene comprising at least 17 contiguous nucleotides of any of SEQ ID NOs 61 to 662, or the complement thereof,
and
the nematode is chosen from the group consisting of Meloidogyne spp., Heterodera spp., Criconemoides spp., Longidorus spp., Helicotylenchus spp., Hoplolaimus spp., Xiphinema spp., Paratrichodorus spp., Tylenchorhynchus spp., Belonolaimus spp. and Pratylenchus spp.

28. The method according to claim 20 wherein the plant is cotton and wherein:
the nucleic acid sequence is chosen from the group comprising:
(i) sequences represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof,
(ii) sequences which are at least 70%, preferably at least 75%, 80%, 85%, 90%, more preferably at least 95%, 96%, 97%, 98% or 99% identical to a sequence represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof, and
(iii) sequences comprising at least 17 contiguous nucleotides of any of the sequences represented by SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662 or the complement thereof,
or wherein said nucleic acid sequence is an ortholog of a gene comprising at least 17 contiguous nucleotides of any of SEQ ID NOs 61 to 662, or the complement thereof,
and
the nematode is chosen from the group consisting of Meloidogyne spp. and Rotylenchulus spp.

29. The method of claim 19, wherein the plant is potato and wherein:
the nucleic acid sequence is chosen from the group comprising:
(i) sequences represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof,
(ii) sequences which are at least 70%, preferably at least 75%, 80%, 85%, 90%, more preferably at least 95%, 96%, 97%, 98% or 99% identical to a sequence represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof, and
(iii) sequences comprising at least 17 contiguous nucleotides of any of the sequences represented by SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof,
or wherein said nucleic acid sequence is an ortholog of a gene comprising at least 17 contiguous nucleotides of any of SEQ ID NOs 61 to 662, or the complement thereof,
and
the nematode is chosen from the group consisting of Meloidogyne spp., Globodera spp. and Ditylenchus spp.

30. The method of claim 19, wherein the plant is banana and wherein:
the nucleic acid sequence is chosen from the group comprising:
(i) sequences represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof,
(ii) sequences which are at least 70%, preferably at least 75%, 80%, 85%, 90%, more preferably at least 95%, 96%, 97%, 98% or 99% identical to a sequence represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof, and
(iii) sequences comprising at least 17 contiguous nucleotides of any of the sequences represented by SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662 or the complement thereof,
or wherein said nucleic acid sequence is an ortholog of a gene comprising at least 17 contiguous nucleotides of any of SEQ ID NOs 61 to 662, or the complement thereof,
and
the nematode is chosen from the group consisting of Meloidogyne spp., Rotylenchulus spp., Pratylenchus spp. and Radopholus spp.

31. The method of claim 19, wherein the plant is tomato and wherein:
the nucleic acid sequence is chosen from the group comprising:
(i) sequences represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof,
(ii) sequences which are at least 70%, preferably at least 75%, 80%, 85%, 90%, more preferably at least 95%, 96%, 97%, 98% or 99% identical to a sequence represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof, and
(iii) sequences comprising at least 17 contiguous nucleotides of any of the sequences represented by SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662 or the complement thereof,
or wherein said nucleic acid sequence is an ortholog of a gene comprising at least 17 contiguous nucleotides of any of SEQ ID NOs 61 to 662, or the complement thereof,
and
the nematode is chosen from the group consisting of Meloidogyne spp. and Globodera spp.

32. The method of claim 19, wherein the plant is soybean and wherein:
the nucleic acid sequence is chosen from the group comprising:
(i) sequences represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof,
(ii) sequences which are at least 70%, preferably at least 75%, 80%, 85%, 90%, more preferably at least 95%, 96%, 97%, 98% or 99% identical to a sequence represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof, and
(iii) sequences comprising at least 17 contiguous nucleotides of any of the sequences represented by SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662 or the complement thereof,
or wherein said nucleic acid sequence is an ortholog of a gene comprising at least 17 contiguous nucleotides of any of SEQ ID NOs 61 to 662, or the complement thereof,
and
the nematode is chosen from the group consisting of Meloidogyne spp., Belonolaimus spp. and Heterodera spp.

33. A transgenic plant, or reproductive or propagation material for a transgenic plant or a cultured transgenic plant cell, which is a rice plant or reproductive or propagation material for a rice plant or a cultured rice plant cell, which is resistant to a nematode pest comprising a polynucleotide comprising a nucleic acid sequence selected from the group comprising:
(i) sequences represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof,
(ii) sequences which are at least 70%, preferably at least 75%, 80%, 85%, 90%, more preferably at least 95%, 96%, 97%, 98% or 99% identical to a sequence represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof, and
(iii) sequences comprising at least 17 contiguous nucleotides of any of the sequences represented by SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662 or the complement thereof,
or wherein said nucleic acid sequence is an ortholog of a gene comprising at least 17 contiguous nucleotides of any of SEQ ID NOs 61 to 662, or the complement thereof,
and wherein said nematode is chosen from the group consisting of Meloidogyne spp., Heterodera spp., Criconemoides spp., Longidorus spp., Helicotylenchus spp., Hoplolaimus spp., Xiphinema spp., Paratrichodorus spp., Tylenchorhynchus spp., Belonolaimus spp. and Pratylenchus spp.

34. A transgenic plant, or reproductive or propagation material for a transgenic plant or a cultured transgenic plant cell, which is a corn plant or reproductive or propagation material for a corn plant or a cultured corn plant cell, resistant to a nematode pest comprising a polynucleotide comprising a nucleic acid sequence selected from the group comprising:
(i) sequences represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof,
(ii) sequences which are at least 70%, preferably at least 75%, 80%, 85%, 90%, more preferably at least 95%, 96%, 97%, 98% or 99% identical to a sequence represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof, and
(iii) sequences comprising at least 17 contiguous nucleotides of any of the sequences represented by SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662 or the complement thereof,
or wherein said nucleic acid sequence is an ortholog of a gene comprising at least 17 contiguous nucleotides of any of SEQ ID NOs 61 to 662, or the complement thereof,
and wherein said nematode is chosen from the group consisting of Meloidogyne spp., Heterodera spp., Criconemoides spp., Longidorus spp., Helicotylenchus spp., Hoplolaimus spp., Xiphinema spp., Paratrichodorus spp., Tylenchorhynchus spp., Belonolaimus spp. and Pratylenchus spp.

35. A transgenic plant, or reproductive or propagation material for a transgenic plant or a cultured transgenic plant cell, which is a cotton plant or reproductive or propagation material for a cotton plant or a cultured cotton plant cell, resistant to a nematode pest comprising a polynucleotide comprising a nucleic acid sequence selected from the group comprising:
(i) sequences represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof,
(ii) sequences which are at least 70%, preferably at least 75%, 80%, 85%, 90%, more preferably at least 95%, 96%, 97%, 98% or 99% identical to a sequence represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof, and
(iii) sequences comprising at least 17 contiguous nucleotides of any of the sequences represented by SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof,
or wherein said nucleic acid sequence is an ortholog of a gene comprising at least 17 contiguous nucleotides of any of SEQ ID NOs 61 to 662, or the complement thereof,
and wherein said nematode is chosen from the group consisting of Meloidogyne spp. and Rotylenchulus spp.

36. A transgenic plant, or reproductive or propagation material for a transgenic plant or a cultured transgenic plant cell, which is a potato plant or reproductive or propagation material for a potato plant or a cultured potato plant cell, resistant to a nematode pest comprising a polynucleotide comprising a nucleic acid sequence selected from the group comprising:
(i) sequences represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof,
(ii) sequences which are at least 70%, preferably at least 75%, 80%, 85%, 90%, more preferably at least 95%, 96%, 97%, 98% or 99% identical to a sequence represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof, and
(iii) sequences comprising at least 17 contiguous nucleotides of any of the sequences represented by SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof,
or wherein said nucleic acid sequence is an ortholog of a gene comprising at least 17 contiguous nucleotides of any of SEQ ID NOs 61 to 662, or the complement thereof,
and wherein said nematode is chosen from the group consisting of Meloidogyne spp., Globodera spp. and Ditylenchus spp.

37. A transgenic plant, or reproductive or propagation material for a transgenic plant or a cultured transgenic plant cell, which is a banana plant or reproductive or propagation material for a banana plant or a cultured banana plant cell, resistant to a nematode pest comprising a polynucleotide comprising a nucleic acid sequence selected from the group comprising:
(i) sequences represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof,
(ii) sequences which are at least 70%, preferably at least 75%, 80%, 85%, 90%, more preferably at least 95%, 96%, 97%, 98% or 99% identical to a sequence represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof, and
(iii) sequences comprising at least 17 contiguous nucleotides of any of the sequences represented by SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof,
or wherein said nucleic acid sequence is an ortholog of a gene comprising at least 17 contiguous nucleotides of any of SEQ ID NOs 61 to 662, or the complement thereof,
and wherein said nematode is chosen from the group consisting of Meloidogyne spp., Rotylenchulus spp., Pratylenchus spp. and Radopholus spp.

38. A transgenic plant, or reproductive or propagation material for a transgenic plant or a cultured transgenic plant cell, which is a tomato plant or reproductive or propagation material for a tomato plant or a cultured tomato plant cell, resistant to a nematode pest comprising a polynucleotide comprising a nucleic acid sequence selected from the group comprising:
(i) sequences represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof,
(ii) sequences which are at least 70%, preferably at least 75%, 80%, 85%, 90%, more preferably at least 95%, 96%, 97%, 98% or 99% identical to a sequence represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof, and
(iii) sequences comprising at least 17 contiguous nucleotides of any of the sequences represented by SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof,
or wherein said nucleic acid sequence is an ortholog of a gene comprising at least 17 contiguous nucleotides of any of SEQ ID NOs 61 to 662, or the complement thereof,
and wherein said nematode is chosen from the group consisting of Meloidogyne spp. and Globodera spp.

39. A transgenic plant, or reproductive or propagation material for a transgenic plant or a cultured transgenic plant cell, which is a soybean plant or reproductive or propagation material for a soybean plant or a cultured soybean plant cell, resistant to a nematode pest comprising a polynucleotide comprising a nucleic acid sequence selected from the group comprising:
(i) sequences represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof,
(ii) sequences which are at least 70%, preferably at least 75%, 80%, 85%, 90%, more preferably at least 95%, 96%, 97%, 98% or 99% identical to a sequence represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof, and
(iii) sequences comprising at least 17 contiguous nucleotides of any of the sequences represented by SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof,
or wherein said nucleic acid sequence is an ortholog of a gene comprising at least 17 contiguous nucleotides of any of SEQ ID NOs 61 to 662, or the complement thereof,
and wherein said nematode is chosen from the group consisting of Meloidogyne spp., Belonolaimus spp. and Heterodera spp.

40. The transgenic plant of claim 33 further comprising or expressing a pesticidal agent selected from the group consisting of a patatin, a Bacillus thuringiensis insecticidal protein, a Bacillus thuringiensis nematicidal protein, a Xenorhabdus insecticidal protein, a Photorhabdus insecticidal protein, a Bacillus laterosporous insecticidal protein, and a Bacillus sphearicus insecticidal protein.

41. The transgenic plant of claim 40 wherein said Bacillus thuringiensis insecticidal protein is selected from the group consisting of a Cry1, a Cry3, a TIC851, a CryET170, a Cry22, a binary insecticidal protein CryET33 and CryET34, a binary insecticidal protein CryET80 and CryET76, a binary insecticidal protein TIC100 and TIC101, and a binary insecticidal protein PS149B1.

42. The isolated double-stranded RNA of claim 1, wherein at least one of the strands comprises the RNA equivalent of at least one of the nucleotide sequences represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the RNA equivalent of a fragment of at least 17 nucleotides in length thereof.

43. The isolated double-stranded RNA of claim 1 comprising at least two copies of said nucleotide sequence.

44. The isolated double-stranded RNA of claim 43 comprising at least two copies of the RNA equivalent of at least one of the nucleotide sequences represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or comprising at least two copies of the RNA equivalent of a fragment of at least 17 nucleotides in length thereof.

45. The isolated double-stranded RNA of claim 43 comprising at least two copies of the RNA equivalent of the nucleotide sequence as represented in any of SEQ ID NOs 5, 6, 12, 18, 24, 28, 29, 33, 37, 41, 45, 46, 51, 54, 57 and 59.

46. An isolated double-stranded RNA comprising the RNA equivalents of at least two nucleotide sequences independently chosen from the sequences represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or fragments thereof of at least 17 base pairs in length.

47. The isolated double-stranded RNA of claim 1, which comprises at least one additional dsRNA region, at least one strand thereof comprising a nucleotide sequence which is complementary to at least part of a nucleotide sequence of at least one other nematode target gene.

48. The isolated double-stranded RNA of claim 47 comprising at least two nucleotide sequences chosen from the group of sequences represented in any of SEQ ID NOs 29, 51 or 54.

49. The isolated double-stranded RNA of claim 48 comprising a nucleotide sequence as represented in SEQ ID NO 59.

50. The isolated double-stranded RNA of claim 1, further comprising at least one additional sequence and optionally a linker sequence.

51. The isolated double-stranded RNA of claim 50 wherein said additional sequence is chosen from the group comprising (i) a sequence facilitating large-scale production of the dsRNA construct; (ii) a sequence effecting an increase or decrease in the stability of the dsRNA; (iii) a sequence allowing the binding of proteins or other molecules to facilitate uptake of the RNA construct by a nematode; or (iv) a sequence which is an aptamer that binds to a receptor or to a molecule on the surface or in the cytoplasm of a nematode to facilitate uptake, endocytosis and/or transcytosis by the nematode or (v) one or more additional sequences to catalyze processing of dsRNA regions.

52. (canceled)

53. An isolated nucleotide sequence encoding a double-stranded RNA according to claim 1.

54. An isolated nucleotide sequence of claim 53 comprising a sequence represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57 or 59, or a fragment thereof of at least 17, preferably at least 18, 19, 20 or 21, more preferably at least 22, 23 or 24 contiguous nucleotides.

55. An isolated nucleotide sequence consisting of a sequence represented by any of SEQ ID NOs 7, 13, 19, 25, 30, 34, 38, 42, 47, 48, 58 or 60, or the complement thereof, or a fragment of at least 17 preferably at least 18, 19, 20 or 21, more preferably at least 22, 23 or 24 nucleotides thereof, or the complement thereof.

56. A recombinant DNA construct comprising the nucleotide sequence of claim 53 operably linked to at least one regulatory sequence.

57. The recombinant DNA construct of claim 56, wherein said regulatory sequence is selected from the group comprising:
(i) constitutive promoters such as any promoter selected from the group comprising the CaMV35S promoter, the doubled CaMV35S promoter, the ubiquitin promoter, the actin promoter, the GOS2 promoter, the Figwort mosaic virus (FMV) 34S promoter, the cassaya vein mosaic virus (CvMv) promoter, the Laccase promoter, and the Strawberry virus 2 (SBV2) promoter;
(ii) tissue specific promoters such as any promoter selected from the group comprising root specific promoters of the genes encoding PsMTA or Class III Chitinase, the pyk10 promoter, the promoter of the Solanum tuberosum gene encoding the leaf and stem specific (ST-LS1) protein
(iii) photosynthetic tissue-specific promoters such as the promoters of the two chlorophyll binding proteins (cab1 and cab2) from sugar beet, the ribulose-bisphosphate carboxylase (Rubisco) promoter encoded by rbcS, phospho-enol-pyruvate carboxylase (PEPC), the promoter of the A (gapA) and B (gapB) subunits of chloroplast glyceraldehyde-3-phosphate dehydrogenase,
(iv) defense-inducible genes, such as the JAS promoters,
(v) the chalcone synthase promoter, the TUB-1 promoter, the ARSK1 promoter and the RPL16A promoter;
(vi) nematode inducible promoters such as any promoter selected from the group comprising the TobRB7 promoter, the phosphoglycerate mutase (PGM) promoter of Arabidopsis, the auxin promoter, ABI3 promoter, the endo-1,4-β glucanase (Cell) promoter, the copper amine oxidase (atao1) promoter, the Late embryogenesis abundant (LEA) promoter, the pyk20 promoter; and
(vii) promoters such as any promoter selected from RNA PolI, RNA PolIII promoter, or the T7 or SP6 RNA polymerase promoter;
or most preferably a promoter chosen from the group consisting of the doubled 35S promoter, the ubiquitin promoter, the actin promoter, the CvMv promoter, the rubisco promoter, the PEPC promoter and the TobRB7 promoter.

58. (canceled)

59. A composition comprising at least one double-stranded RNA, wherein said double-stranded RNA comprises annealed complementary strands, one of which has a nucleotide sequence which is complementary to at least part of a nucleotide sequence of a nematode target gene and optionally further comprising at least one suitable carrier, excipient or diluent.

60. A composition comprising at least one double-stranded RNA of claim 1 and further comprising at least one suitable carrier, excipient or diluent.

61. A composition comprising at least one nucleotide sequence according to claim 53; and further comprising at least one suitable carrier, excipient or diluent.

62. (canceled)

63. A cell comprising the nucleotide sequence of claim 53.

64. The cell of claim 4 wherein said cell is a prokaryotic cell or an eukaryotic cell.

65. The cell of claim 4 wherein said cell is a bacterial cell.

66. The cell of claim 4 wherein said cell is a yeast cell.

67. A composition comprising at least one bacterial or yeast cell, said cell comprising at least one double stranded ribonucleotide sequence of claim 1.

68. The composition of claim 67 wherein ingestion of said ribonucleotide sequence by a pathogenic pest inhibits the growth of said pest.

69. The composition of claim 67 wherein said bacterial or yeast cell is inactivated or killed, for instance by heat treatment or mechanical treatment.

70. A composition comprising at least one bacterial or yeast cell expressing at least one double-stranded RNA according to claim 1, and optionally further comprising at least one suitable carrier, excipient or diluent.

71. The composition of claim 59, said composition further comprising at least one pesticidal agent selected from the group consisting of a chemical insecticide, a patatin, a Bacillus thuringiensis insecticidal protein, a Bacillus thuringiensis nematicidal protein, Xenorhabdus insecticidal protein, a Photorhabdus insecticidal protein, a Bacillus laterosporous insecticidal protein, and a Bacillus sphearicus insecticidal protein, wherein the composition is active against a single pathogenic pest, or wherein said pesticidal agent is active against one or more other pathogenic pests.

72. The composition of claim 67, wherein said at least one bacterial or yeast cell further comprises or further expresses at least one pesticidal agent selected from the group consisting of a chemical insecticide, a patatin, a Bacillus thuringiensis insecticidal protein, a Bacillus thuringiensis nematicidal protein, a Xenorhabdus insecticidal protein, a Photorhabdus insecticidal protein, a Bacillus laterosporous insecticidal protein, and a Bacillus sphearicus insecticidal protein, wherein the composition is active against a single pathogenic pest, or wherein said pesticidal agent is active against one or more other pathogenic pests.

73. The composition of claim 67, further comprising at least one further bacterial or yeast cell comprising or expressing at least one pesticidal agent selected from the group consisting of a chemical insecticide, a patatin, a Bacillus thuringiensis insecticidal protein, a Bacillus thuringiensis nematicidal protein, a Xenorhabdus insecticidal protein, a Photorhabdus insecticidal protein, a Bacillus laterosporous insecticidal protein, and a Bacillus sphearicus insecticidal protein, wherein the composition is active against a single pathogenic pest, or wherein said pesticidal agent is active against one or more other pathogenic pests.

74. The composition of claim 71 wherein said Bacillus thuringiensis insecticidal protein is selected from the group consisting of a Cry1, a Cry3, a TIC851, a CryET170, a Cry22, a binary insecticidal protein CryET33 and CryET34, a binary insecticidal protein CryET80 and CryET76, a binary insecticidal protein TIC100 and TIC 101, and a binary insecticidal protein PS149B1.

75-76. (canceled)

77. A spray comprising at least one composition of claim 59 and optionally further comprising at least one adjuvant and at least one surfactant.

78. A housing or trap or bait for a pest containing a composition as defined in claim 59.

79-81. (canceled)

82. A plant comprising at least one nucleotide sequence according to claim 53.

83. The plant of claim 82 wherein said plant is chosen from the group comprising alfalfa, apple, apricot, Arabidopsis, artichoke, asparagus, avocado, banana, barley, beans, beet, blackberry, blueberry, Brassica, broccoli, Brussels sprouts, cabbage, canola, carrot, cassaya, cauliflower, a cereal, celery, cherry, citrus, Clementine, coffee, corn, cotton, cucumber, eggplant, endive, eucalyptus, figs, grape, grapefruit, groundnuts, ground cherry, kiwifruit, lettuce, leek, lemon, lime, pine, maize, mango, melon, millet, mushroom, nut oat, okra, onion, orange, an ornamental plant or flower or tree, papaya, parsley, pea, peach, peanut, peat, pepper, persimmon, pineapple, plantain, plum, pomegranate, potato, pumpkin, radicchio, radish, rapeseed, raspberry, rice, rye, sorghum, soy, soybean, spinach, strawberry, sugar beet, sugarcane, sunflower, sweet potato, tangerine, tea, tobacco, tomato, a vine, watermelon, wheat, yams and zucchini.

84. The plant of claim 82 wherein said plant is cotton, rice, soybean, corn, tomato, tobacco, potato or banana.

85. A seed comprising at least one nucleotide sequence of claim 46.

86. A seed of a plant as defined in claim 83.

87. A method for preventing nematode growth on a plant or for preventing nematode infestation of a plant comprising applying a composition of claim 59.

88. A method for improving yield, comprising applying to a plant an effective amount of a composition of claim 59 to said plant.

89. The method of claim 87 wherein said plant is chosen from the group comprising alfalfa, apple, apricot, Arabidopsis, artichoke, asparagus, avocado, banana, barley, beans, beet, blackberry, blueberry, Brassica, broccoli, Brussels sprouts, cabbage, canola, carrot, cassaya, cauliflower, a cereal, celery, cherry, citrus, Clementine, coffee, corn, cotton, cucumber, eggplant, endive, eucalyptus, figs, grape, grapefruit, groundnuts, ground cherry, kiwifruit, lettuce, leek, lemon, lime, pine, maize, mango, melon, millet, mushroom, nut oat, okra, onion, orange, an ornamental plant or flower or tree, papaya, parsley, pea, peach, peanut, peat, pepper, persimmon, pineapple, plantain, plum, pomegranate, potato, pumpkin, radicchio, radish, rapeseed, raspberry, rice, rye, sorghum, soy, soybean, spinach, strawberry, sugar beet, sugarcane, sunflower, sweet potato, tangerine, tea, tobacco, tomato, a vine, watermelon, wheat, yams and zucchini.

90. A method for treating and/or preventing nematode infestation on a substrate comprising applying an effective amount of a composition according to claim 59 to said substrate.

91. A kit comprising a double-stranded RNA of claim 1 for treating nematode infection of plants.

92. An isolated nucleotide sequence comprising a nucleic acid sequence selected from the group comprising:
(i) sequences represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof,
(ii) sequences which are at least 70%, preferably at least 75%, 80%, 85%, 90%, more preferably at least 95%, 96%, 97%, 98% or 99% identical to a sequence represented by any of SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof, and
(iii) sequences comprising at least 17 contiguous nucleotides of any of the sequences represented by SEQ ID NOs 1, 5, 6, 8, 12, 14, 18, 20, 24, 26, 28, 29, 31, 33, 35, 37, 39, 41, 43, 45, 46, 49, 51, 52, 54, 55, 57, 59, or any of SEQ ID NOs 61 to 662, or the complement thereof,
or wherein said nucleic acid sequence is an orthologue of a gene comprising at least 17 contiguous nucleotides of any of SEQ ID NOs 61 to 662, or the complement thereof.
